SEQUENCE LISTING (170 sequences)

SEQ ID NO: 1 - protein, 524 aa, Homo sapiens
MHSLDEPLDLKLSITKLRAAREKRERTLGVVRPRALHRELGLVDDSPTPGSPGSPPSGFLLNSKFPEKVEGRFSAAPLVDLSLSPPSGLDSPNGSSSLSPERQGNGDLPPVPSASDFQPLRYLDGVPSSFQFFLPLGSGGALHLPASSFLTPPKDKCLSPDLPLPKQLVCRWAKCNQLFELLQDLVDHVNDYHVKPEKDAGYCCHWEGCARHGRGFNARYKMLIHIRTHTNEKPHRCPTCSKSFSRLENLKIHNRSHTGEKPYVCPYEGCNKRYSNSSDRFKHTRTHYVDKPYYCKMPGCHKRYTDPSSLRKHIKAHGHFVSHEQQELLQLRPPPKPPLPAPDGGPYVSGAQIIIPNPAALFGGPGLPGLPLPLAPGPLDLSALACGNGGGSGGGGGMGPGLPGPVLPLNLAKNPLLPSPFGAGGLGLPVVSLLAGAAGGKAEGEKGRGSVPTRALGMEGHKTPLERTESSCSRPSPDGLPLLPGTVLDLSTGVNSAASSPEALAPGWVVIPPGSVLLKPAVVN

SEQ ID NO: 2 - protein, 4303 aa, Homo sapiens
MPPAAPARLALALGLGLWLGALAGGPGRGCGPCEPPCLCGPAPGAACRVNCSGRGLRTLGPALRIPADATALDVSHNLLRALDVGLLANLSALAELDISNNKISTLEEGIFANLFNLSEINLSGNPFECDCGLAWLPRWAEEQQVRVVQPEAATCAGPGSLAGQPLLGIPLLDSGCGEEYVACLPDNSSGTVAAVSFSAAHEGLLQPEACSAFCFSTGQGLAALSEQGWCLCGAAQPSSASFACLSLCSGPPPPPAPTCRGPTLLQHVFPASPGATLVGPHGPLASGQLAAFHIAAPLPVTATRWDFGDGSAEVDAAGPAASHRYVLPGRYHVTAVLALGAGSALLGTDVQVEAAPAALELVCPSSVQSDESLDLSIQNRGGSGLEAAYSIVALGEEPARAVHPLCPSDTEIFPGNGHCYRLVVEKAAWLQAQEQCQAWAGAALAMVDSPAVQRFLVSRVTRSLDVWIGFSTVQGVEVGPAPQGEAFSLESCQNWLPGEPHPATAEHCVRLGPTGWCNTDLCSAPHSYVCELQPGGPVQDAENLLVGAPSGDLQGPLTPLAQQDGLSAPHEPVEVMVFPGLRLSREAFLTTAEFGTQELRRPAQLRLQVYRLLSTAGTPENGSEPESRSPDNRTQLAPACMPGGRWCPGANICLPLDASCHPQACANGCTSGPGLPGAPYALWREFLFSVPAGPPAQYSVTLHGQDVLMLPGDLVGLQHDAGPGALLHCSPAPGHPGPRAPYLSANASSWLPHLPAQLEGTWACPACALRLLAATEQLTVLLGLRPNPGLRLPGRYEVRAEVGNGVSRHNLSCSFDVVSPVAGLRVIYPAPRDGRLYVPTNGSALVLQVDSGANATATARWPGGSVSARFENVCPALVATFVPGCPWETNDTLFSVVALPWLSEGEHVVDVVVENSASRANLSLRVTAEEPICGLRATPSPEARVLQGVLVRYSPVVEAGSDMVFRWTINDKQSLTFQNVVFNVIYQSAAVFKLSLTASNHVSNVTVNYNVTVERMNRMQGLQVSTVPAVLSPNATLALTAGVLVDSAVEVAFLWTFGDGEQALHQFQPPYNESFPVPDPSVAQVLVEHNVMHTYAAPGEYLLTVLASNAFENLTQQVPVSVRASLPSVAVGVSDGVLVAGRPVTFYPHPLPSPGGVLYTWDFGDGSPVLTQSQPAANHTYASRGTYHVRLEVNNTVSGAAAQADVRVFEELRGLSVDMSLAVEQGAPVVVSAAVQTGDNITWTFDMGDGTVLSGPEATVEHVYLRAQNCTVTVGAASPAGHLARSLHVLVFVLEVLRVEPAACIPTQPDARLTAYVTGNPAHYLFDWTFGDGSSNTTVRGCPTVTHNFTRSGTFPLALVLSSRVNRAHYFTSICVEPEVGNVTLQPERQFVQLGDEAWLVACAWPPFPYRYTWDFGTEEAAPTRARGPEVTFIYRDPGSYLVTVTASNNISAANDSALVEVQEPVLVTSIKVNGSLGLELQQPYLFSAVGRGRPASYLWDLGDGGWLEGPEVTHAYNSTGDFTVRVAGWNEVSRSEAWLNVTVKRRVRGLVVNASRTVVPLNGSVSFSTSLEAGSDVRYSWVLCDRCTPIPGGPTISYTFRSVGTFNIIVTAENEVGSAQDSIFVYVLQLIEGLQVVGGGRYFPTNHTVQLQAVVRDGTNVSYSWTAWRDRGPALAGSGKGFSLTVLEAGTYHVQLRATNMLGSAWADCTMDFVEPVGWLMVAASPNPAAVNTSVTLSAELAGGSGVVYTWSLEEGLSWETSEPFTTHSFPTPGLHLVTMTAGNPLGSANATVEVDVQVPVSGLSIRASEPGGSFVAAGSSVPFWGQLATGTNVSWCWAVPGGSSKRGPHVTMVFPDAGTFSIRLNASNAVSWVSATYNLTAEEPIVGLVLWASSKVVAPGQLVHFQILLAAGSAVTFRLQVGGANPEVLPGPRFSHSFPRVGDHVVSVRGKNHVSWAQAQVRIVVLEAVSGLQVPNCCEPGIATGTERNFTARVQRGSRVAYAWYFSLQKVQGDSLVILSGRDVTYTPVAAGLLEIQVRAFNALGSENRTLVLEVQDAVQYVALQSGPCFTNRSAQFEAATSPSPRRVAYHWDFGDGSPGQDTDEPRAEHSYLRPGDYRVQVNASNLVSFFVAQATVTVQVLACREPEVDVVLPLQVLMRRSQRNYLEAHVDLRDCVTYQTEYRWEVYRTASCQRPGRPARVALPGVDVSRPRLVLPRLALPVGHYCFVFVVSFGDTPLTQSIQANVTVAPERLVPIIEGGSYRVWSDTRDLVLDGSESYDPNLEDGDQTPLSFHWACVASTQREAGGCALNFGPRGSSTVTIPRERLAAGVEYTFSLTVWKAGRKEEATNQTVLIRSGRVPIVSLECVSCKAQAVYEVSRSSYVYLEGRCLNCSSGSKRGRWAARTFSNKTLVLDETTTSTGSAGMRLVLRRGVLRDGEGYTFTLTVLGRSGEEEGCASIRLSPNRPPLGGSCRLFPLGAVHALTTKVHFECTGWHDAEDAGAPLVYALLLRRCRQGHCEEFCVYKGSLSSYGAVLPPGFRPHFEVGLAVVVQDQLGAAVVALNRSLAITLPEPNGSATGLTVWLHGLTASVLPGLLRQADPQHVIEYSLALVTVLNEYERALDVAAEPKHERQHRAQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVASMAFQTQAGAQIPIERLASERAITVKVPNNSDWAARGHRSSANSANSVVVQPQASVGAVVTLDSSNPAAGLHLQLNYTLLDGHYLSEEPEPYLAVYLHSEPRPNEHNCSASRRIRPESLQGADHRPYTFFISPGSRDPAGSYHLNLSSHFRWSALQVSVGLYTSLCQYFSEEDMVWRTEGLLPLEETSPRQAVCLTRHLTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 3 - protein, 968 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 4 - DNA, 19 bp, synthetic construct
  source 1..19: mol_type = other DNA
ctggttgtcattggccagg

SEQ ID NO: 5 - DNA, 21 bp, synthetic construct
  source 1..21: mol_type = other DNA
gcatcgaccggtaatgcaggc

SEQ ID NO: 6 - DNA, 21 bp, synthetic construct
  source 1..21: mol_type = other DNA
ccatgtctagactggacaaga

SEQ ID NO: 7 - DNA, 21 bp, synthetic construct
  source 1..21: mol_type = other DNA
ctccaggccacatatgattag

SEQ ID NO: 8 - DNA, 20 bp, synthetic construct
  source 1..20: mol_type = other DNA
gcagagctcgtttagtgaac

SEQ ID NO: 9 - DNA, 19 bp, synthetic construct
  source 1..19: mol_type = other DNA
tcgaccagtttagttaccc

SEQ ID NO: 10 - DNA, 22 bp, synthetic construct
  source 1..22: mol_type = other DNA
cacaaccacttcctgcttggtg

SEQ ID NO: 11 - DNA, 21 bp, synthetic construct
  source 1..21: mol_type = other DNA
ccagcattctcgacccacaag

SEQ ID NO: 12 - DNA, 20 bp, synthetic construct
  source 1..20: mol_type = other DNA
gggtgctgaagagatggttc

SEQ ID NO: 13 - DNA, 20 bp, synthetic construct
  source 1..20: mol_type = other DNA
tccacaaaagctgcaatgaa

SEQ ID NO: 14 - DNA, 20 bp, synthetic construct
  source 1..20: mol_type = other DNA
ccatttgtgagggttgcttt

SEQ ID NO: 15 - DNA, 20 bp, synthetic construct
  source 1..20: mol_type = other DNA
gttttgtgctggggatggta

SEQ ID NO: 16 - DNA, 21 bp, synthetic construct
  source 1..21: mol_type = other DNA
gaccacctttttagcctcctg

SEQ ID NO: 17 - DNA, 21 bp, synthetic construct
  source 1..21: mol_type = other DNA
agggaagggacttaggaatga

SEQ ID NO: 18 - DNA, 21 bp, synthetic construct
  source 1..21: mol_type = other DNA
tctgtgagtttgtgaccagcc

SEQ ID NO: 19 - DNA, 19 bp, synthetic construct
  source 1..19: mol_type = other DNA
ggtgggagctgcaagaggg

SEQ ID NO: 20 - DNA, 20 bp, synthetic construct
  source 1..20: mol_type = other DNA
actgggggtagggtcctaga

SEQ ID NO: 21 - DNA, 20 bp, synthetic construct
  source 1..20: mol_type = other DNA
ccctgtcaccatctccaact

SEQ ID NO: 22 - DNA, 19 bp, synthetic construct
  source 1..19: mol_type = other DNA
gacgtcacccgttctgttg

SEQ ID NO: 23 - DNA, 20 bp, synthetic construct
  source 1..20: mol_type = other DNA
aggcaaattttggtgtacgg

SEQ ID NO: 24 - DNA, 19 bp, synthetic construct
  source 1..19: mol_type = other DNA
aggttcgttcactcatgga

SEQ ID NO: 25 - DNA, 19 bp, synthetic construct
  source 1..19: mol_type = other DNA
tcgaccagtttagttaccc

SEQ ID NO: 26 - DNA, 23 bp, synthetic construct
  source 1..23: mol_type = other DNA
cactgatattgtaagtagtttgc

SEQ ID NO: 27 - DNA, 22 bp, synthetic construct
  source 1..22: mol_type = other DNA
ctagtgcgaagtagtgatcagg

SEQ ID NO: 28 - DNA, 20 bp, synthetic construct
  source 1..20: mol_type = other DNA
cgtgttcgtgcaagttgagt

SEQ ID NO: 29 - DNA, 20 bp, synthetic construct
  source 1..20: mol_type = other DNA
attgcatcgcattgtctgag

SEQ ID NO: 30 - DNA, 20 bp, synthetic construct
  source 1..20: mol_type = other DNA
cgtgttcgtgcaagttgagt

SEQ ID NO: 31 - DNA, 20 bp, synthetic construct
  source 1..20: mol_type = other DNA
ccgaaaatctgtgggaagtc

SEQ ID NO: 32 - DNA, 27 bp, synthetic construct
  source 1..27: mol_type = other DNA
gtgctacttccatttgtcacgtcctgc

SEQ ID NO: 33 - DNA, 20 bp, synthetic construct
  source 1..20: mol_type = other DNA
ctgattcacatgccccaggt

SEQ ID NO: 34 - DNA, 27 bp, synthetic construct
  source 1..27: mol_type = other DNA
gtgctacttccatttgtcacgtcctgc

SEQ ID NO: 35 - DNA, 20 bp, synthetic construct
  source 1..20: mol_type = other DNA
gggaaaaccatggaaaatcg

SEQ ID NO: 36 - DNA, 23 bp, synthetic construct
  source 1..23: mol_type = other DNA
cagtctgcctggcctgccattac

SEQ ID NO: 37 - DNA, 22 bp, synthetic construct
  source 1..22: mol_type = other DNA
cagaccctggtggaatgaccac

SEQ ID NO: 38 - DNA, 21 bp, synthetic construct
  source 1..21: mol_type = other DNA
ttctatcgccttcttgacgag

SEQ ID NO: 39 - DNA, 21 bp, synthetic construct
  source 1..21: mol_type = other DNA
gcgcctttccaatgagtcttc

SEQ ID NO: 40 - DNA, 20 bp, synthetic construct
  source 1..20: mol_type = other DNA
actgggggtagggtcctaga

SEQ ID NO: 41 - DNA, 20 bp, synthetic construct
  source 1..20: mol_type = other DNA
ccctgtcaccatctccaact

SEQ ID NO: 42 - DNA, 23 bp, synthetic construct
  source 1..23: mol_type = other DNA
tcgtatagcatacattatacgaa

SEQ ID NO: 43 - DNA, 20 bp, synthetic construct
  source 1..20: mol_type = other DNA
aatagcgaagtggctggaaa

SEQ ID NO: 44 - DNA, 20 bp, synthetic construct
  source 1..20: mol_type = other DNA
tagagcttgcggaacccttc

SEQ ID NO: 45 - DNA, 20 bp, synthetic construct
  source 1..20: mol_type = other DNA
ctttaagcctgcccagaaga

SEQ ID NO: 46 - DNA, 20 bp, synthetic construct
  source 1..20: mol_type = other DNA
agggagctgcagtggagtag

SEQ ID NO: 47 - DNA, 20 bp, synthetic construct
  source 1..20: mol_type = other DNA
ctttaagcctgcccagaaga

SEQ ID NO: 48 - DNA, 20 bp, synthetic construct
  source 1..20: mol_type = other DNA
gggaagagaagggttgaagg

SEQ ID NO: 49 - DNA, 20 bp, synthetic construct
  source 1..20: mol_type = other DNA
actcagcaggccaggaagta

SEQ ID NO: 50 - DNA, 20 bp, synthetic construct
  source 1..20: mol_type = other DNA
cacgctgttttgacctccat

SEQ ID NO: 51 - DNA, 20 bp, synthetic construct
  source 1..20: mol_type = other DNA
ctctgcccctcgactctaga

SEQ ID NO: 52 - DNA, 22 bp, synthetic construct
  source 1..22: mol_type = other DNA
tcacggaagagcagcctgcctt

SEQ ID NO: 53 - DNA, 22 bp, synthetic construct
  source 1..22: mol_type = other DNA
tctgtgtactggggcacagcct

SEQ ID NO: 54 - DNA, 22 bp, synthetic construct
  source 1..22: mol_type = other DNA
agcacctgagctgttgtcaggg

SEQ ID NO: 55 - DNA, 21 bp, synthetic construct
  source 1..21: mol_type = other DNA
ctagacctgtcccacaaccta

SEQ ID NO: 56 - DNA, 23 bp, synthetic construct
  source 1..23: mol_type = other DNA
gcaaacacgccttcttctaatgt

SEQ ID NO: 57 - DNA, 21 bp, synthetic construct
  source 1..21: mol_type = other DNA
ggggaacaagactcatggaag

SEQ ID NO: 58 - DNA, 21 bp, synthetic construct
  source 1..21: mol_type = other DNA
gccgtaggtcaagatgcacaa

SEQ ID NO: 59 - DNA, 21 bp, synthetic construct
  source 1..21: mol_type = other DNA
gtgtcgatgggccaagtgtaa

SEQ ID NO: 60 - DNA, 21 bp, synthetic construct
  source 1..21: mol_type = other DNA
tgacagcagtatcgagcatcc

SEQ ID NO: 61 - DNA, 22 bp, synthetic construct
  source 1..22: mol_type = other DNA
atgccgatcaataagtcggaga

SEQ ID NO: 62 - DNA, 21 bp, synthetic construct
  source 1..21: mol_type = other DNA
gttcccctcatttcatccacg

SEQ ID NO: 63 - DNA, 21 bp, synthetic construct
  source 1..21: mol_type = other DNA
aggagacgagtcacatctcag

SEQ ID NO: 64 - DNA, 22 bp, synthetic construct
  source 1..22: mol_type = other DNA
tgtgtcatactgcctcatgttc

SEQ ID NO: 65 - DNA, 21 bp, synthetic construct
  source 1..21: mol_type = other DNA
agtgcctgatgccacctatac

SEQ ID NO: 66 - DNA, 22 bp, synthetic construct
  source 1..22: mol_type = other DNA
cggccaagatagagatgggttg

SEQ ID NO: 67 - DNA, 20 bp, synthetic construct
  source 1..20: mol_type = other DNA
gggaagcactacaagggtcc

SEQ ID NO: 68 - DNA, 21 bp, synthetic construct
  source 1..21: mol_type = other DNA
ccacagtccgattccaagaaa

SEQ ID NO: 69 - DNA, 19 bp, synthetic construct
  source 1..19: mol_type = other DNA
agctcagcgatgaagccag

SEQ ID NO: 70 - DNA, 21 bp, synthetic construct
  source 1..21: mol_type = other DNA
ctgtgtgaagatccaagggga

SEQ ID NO: 71 - DNA, 21 bp, synthetic construct
  source 1..21: mol_type = other DNA
ggtcactgacaatatcctggc

SEQ ID NO: 72 - DNA, 23 bp, synthetic construct
  source 1..23: mol_type = other DNA
aggtaggtgaaaccactttcttg

SEQ ID NO: 73 - DNA, 21 bp, synthetic construct
  source 1..21: mol_type = other DNA
atgtcggtcagcagaaaggac

SEQ ID NO: 74 - DNA, 21 bp, synthetic construct
  source 1..21: mol_type = other DNA
ctgtggttgaactcaggttgc

SEQ ID NO: 75 - DNA, 20 bp, synthetic construct
  source 1..20: mol_type = other DNA
gcaggcttgaatggagtagc

SEQ ID NO: 76 - DNA, 21 bp, synthetic construct
  source 1..21: mol_type = other DNA
gcagcgttgcatttatcacac

SEQ ID NO: 77 - DNA, 21 bp, synthetic construct
  source 1..21: mol_type = other DNA
aggtcggtgtgaacggatttg

SEQ ID NO: 78 - DNA, 23 bp, synthetic construct
  source 1..23: mol_type = other DNA
tgtagaccatgtagttgaggtca

SEQ ID NO: 79 - DNA, 20 bp, synthetic construct
  source 1..20: mol_type = other DNA
gtaacccgttgaaccccatt

SEQ ID NO: 80 - DNA, 20 bp, synthetic construct
  source 1..20: mol_type = other DNA
ccatccaatcggtagtagcg

SEQ ID NO: 81 - DNA, 20 bp, synthetic construct
  source 1..20: mol_type = other DNA
agtacgatgtggggcttttg

SEQ ID NO: 82 - DNA, 20 bp, synthetic construct
  source 1..20: mol_type = other DNA
actcctgggccttgaaagtt

SEQ ID NO: 83 - DNA, 20 bp, synthetic construct
  source 1..20: mol_type = other DNA
ttggtgatgctcagctttag

SEQ ID NO: 84 - DNA, 20 bp, synthetic construct
  source 1..20: mol_type = other DNA
tcgatgcaaagcatgatgcc

SEQ ID NO: 85 - DNA, 20 bp, synthetic construct
  source 1..20: mol_type = other DNA
ggaaacctggtggtggagag

SEQ ID NO: 86 - DNA, 20 bp, synthetic construct
  source 1..20: mol_type = other DNA
tcagggaatttggggttcag

SEQ ID NO: 87 - DNA, 20 bp, synthetic construct
  source 1..20: mol_type = other DNA
caagggggctgcagaaaagc

SEQ ID NO: 88 - DNA, 20 bp, synthetic construct
  source 1..20: mol_type = other DNA
gagggtggtgacaagctgag

SEQ ID NO: 89 - DNA, 20 bp, synthetic construct
  source 1..20: mol_type = other DNA
cagtaggcagtggaggcaag

SEQ ID NO: 90 - DNA, 20 bp, synthetic construct
  source 1..20: mol_type = other DNA
aacccaggggcaagaagaac

SEQ ID NO: 91 - DNA, 20 bp, synthetic construct
  source 1..20: mol_type = other DNA
atcgacacaccagctgcttg

SEQ ID NO: 92 - DNA, 20 bp, synthetic construct
  source 1..20: mol_type = other DNA
gctcaaagagctggttacac

SEQ ID NO: 93 - DNA, 20 bp, synthetic construct
  source 1..20: mol_type = other DNA
tcgttgacatggtcaaccag

SEQ ID NO: 94 - DNA, 20 bp, synthetic construct
  source 1..20: mol_type = other DNA
ctgttcaggcttgacatgat

SEQ ID NO: 95 - DNA, 20 bp, synthetic construct
  source 1..20: mol_type = other DNA
ccaatgacagcagtatcgag

SEQ ID NO: 96 - DNA, 20 bp, synthetic construct
  source 1..20: mol_type = other DNA
tcttgtacctggcattgaag

SEQ ID NO: 97 - DNA, 20 bp, synthetic construct
  source 1..20: mol_type = other DNA
tgtgtccggatgtgaatgag

SEQ ID NO: 98 - DNA, 20 bp, synthetic construct
  source 1..20: mol_type = other DNA
ttgtggatcttcaggttctc

SEQ ID NO: 99 - DNA, 20 bp, synthetic construct
  source 1..20: mol_type = other DNA
tggagtaacgcttgttgcag

SEQ ID NO: 100 - DNA, 20 bp, synthetic construct
  source 1..20: mol_type = other DNA
tgtgcttgaagcggtcactg

SEQ ID NO: 101 - DNA, 20 bp, synthetic construct
  source 1..20: mol_type = other DNA
ttgtctacgtagtgggtacg

SEQ ID NO: 102 - DNA, 20 bp, synthetic construct
  source 1..20: mol_type = other DNA
cgggcatcttgcagtagtag

SEQ ID NO: 103 - DNA, 20 bp, synthetic construct
  source 1..20: mol_type = other DNA
tccgtgtaacgcttgtgaca

SEQ ID NO: 104 - DNA, 20 bp, synthetic construct
  source 1..20: mol_type = other DNA
tgatgtgtttgcgcagtgaa

SEQ ID NO: 105 - DNA, 20 bp, synthetic construct
  source 1..20: mol_type = other DNA
atgtgacacaaagtggccat

SEQ ID NO: 106 - DNA, 20 bp, synthetic construct
  source 1..20: mol_type = other DNA
gacataggagccactgtcag

SEQ ID NO: 107 - DNA, 20 bp, synthetic construct
  source 1..20: mol_type = other DNA
ttcgggatgatgatctgagc

SEQ ID NO: 108 - DNA, 20 bp, synthetic construct
  source 1..20: mol_type = other DNA
gaggtagaggtaatggcagg

SEQ ID NO: 109 - DNA, 20 bp, synthetic construct
  source 1..20: mol_type = other DNA
cagagcactgaggtcaaggg

SEQ ID NO: 110 - DNA, 20 bp, synthetic construct
  source 1..20: mol_type = other DNA
caaagggtgagggcaacagc

SEQ ID NO: 111 - DNA, 20 bp, synthetic construct
  source 1..20: mol_type = other DNA
aggagagaaaccacaggcag

SEQ ID NO: 112 - DNA, 20 bp, synthetic construct
  source 1..20: mol_type = other DNA
tgtggacaggtccagtacag

SEQ ID NO: 113 - DNA, 20 bp, synthetic construct
  source 1..20: mol_type = other DNA
gagttaacacctctggactg

SEQ ID NO: 114 - DNA, 20 bp, synthetic construct
  source 1..20: mol_type = other DNA
acagaccctggtggaatgac

SEQ ID NO: 115 - DNA, 20 bp, synthetic construct
  source 1..20: mol_type = other DNA
tttaccacagctggtttgag

SEQ ID NO: 116 - DNA, 20 bp, synthetic construct
  source 1..20: mol_type = other DNA
cacagcgaaaattcccactg

SEQ ID NO: 117 - DNA, 20 bp, synthetic construct
  source 1..20: mol_type = other DNA
agtgtcatccaaacagtctc

SEQ ID NO: 118 - DNA, 20 bp, synthetic construct
  source 1..20: mol_type = other DNA
tcggaacaatccttatcctg

SEQ ID NO: 119 - DNA, 20 bp, synthetic construct
  source 1..20: mol_type = other DNA
gtactcgatgggaacacact

SEQ ID NO: 120 - DNA, 20 bp, synthetic construct
  source 1..20: mol_type = other DNA
aatagcagtttctctcgtca

SEQ ID NO: 121 - DNA, 20 bp, synthetic construct
  source 1..20: mol_type = other DNA
gtcatggtcacagacatagg

SEQ ID NO: 122 - DNA, 20 bp, synthetic construct
  source 1..20: mol_type = other DNA
catcgctgttatcttcacag

SEQ ID NO: 123 - DNA, 20 bp, synthetic construct
  source 1..20: mol_type = other DNA
ccattctcttgggtaacatg

SEQ ID NO: 124 - DNA, 20 bp, synthetic construct
  source 1..20: mol_type = other DNA
cgggaactccatcacaaact

SEQ ID NO: 125 - DNA, 20 bp, synthetic construct
  source 1..20: mol_type = other DNA
ccaggatataaccttcttca

SEQ ID NO: 126 - DNA, 20 bp, synthetic construct
  source 1..20: mol_type = other DNA
tcgttggatttgcaatgctg

SEQ ID NO: 127 - DNA, 20 bp, synthetic construct
  source 1..20: mol_type = other DNA
caaatcccgaccattagaga

SEQ ID NO: 128 - DNA, 20 bp, synthetic construct
  source 1..20: mol_type = other DNA
tttcttccatgaagatctcc

SEQ ID NO: 129 - DNA, 20 bp, synthetic construct
  source 1..20: mol_type = other DNA
atcaagggcaattcctctag

SEQ ID NO: 130 - DNA, 20 bp, synthetic construct
  source 1..20: mol_type = other DNA
gattgctaccatccatgaag

SEQ ID NO: 131 - DNA, 20 bp, synthetic construct
  source 1..20: mol_type = other DNA
cgcttggatacaagatccag

SEQ ID NO: 132 - DNA, 20 bp, synthetic construct
  source 1..20: mol_type = other DNA
aaacggatgagggacgaggg

SEQ ID NO: 133 - DNA, 20 bp, synthetic construct
  source 1..20: mol_type = other DNA
atggtaaactgtcactccga

SEQ ID NO: 134 - DNA, 20 bp, synthetic construct
  source 1..20: mol_type = other DNA
cagccgtttgtgatgagaag

SEQ ID NO: 135 - DNA, 20 bp, synthetic construct
  source 1..20: mol_type = other DNA
agagattccttgagatccag

SEQ ID NO: 136 - DNA, 20 bp, synthetic construct
  source 1..20: mol_type = other DNA
cagcttcatgacagtgacac

SEQ ID NO: 137 - DNA, 20 bp, synthetic construct
  source 1..20: mol_type = other DNA
tcgagggttgtttaagttgc

SEQ ID NO: 138 - DNA, 20 bp, synthetic construct
  source 1..20: mol_type = other DNA
gactatgggcattaggtgag

SEQ ID NO: 139 - DNA, 20 bp, synthetic construct
  source 1..20: mol_type = other DNA
aacggtgagtccaaacggat

SEQ ID NO: 140 - DNA, 20 bp, synthetic construct
  source 1..20: mol_type = other DNA
gtttggaactgggaagcaga

SEQ ID NO: 141 - DNA, 20 bp, synthetic construct
  source 1..20: mol_type = other DNA
ctctgaagcttcattccata

SEQ ID NO: 142 - DNA, 20 bp, synthetic construct
  source 1..20: mol_type = other DNA
ctgttgtcatggcaatcatc

SEQ ID NO: 143 - DNA, 20 bp, synthetic construct
  source 1..20: mol_type = other DNA
cagtcattctgtttgtcaca

SEQ ID NO: 144 - DNA, 20 bp, synthetic construct
  source 1..20: mol_type = other DNA
cactctttggggatacacat

SEQ ID NO: 145 - DNA, 20 bp, synthetic construct
  source 1..20: mol_type = other DNA
ctgatccatccgaacaatca

SEQ ID NO: 146 - DNA, 20 bp, synthetic construct
  source 1..20: mol_type = other DNA
cttgaactgagagcttgtgc

SEQ ID NO: 147 - DNA, 20 bp, synthetic construct
  source 1..20: mol_type = other DNA
tagatgcagtttccattgtc

SEQ ID NO: 148 - DNA, 20 bp, synthetic construct
  source 1..20: mol_type = other DNA
aaaaggctgggtaggacagt

SEQ ID NO: 149 - DNA, 20 bp, synthetic construct
  source 1..20: mol_type = other DNA
tttgtcacgactggctacaa

SEQ ID NO: 150 - DNA, 20 bp, synthetic construct
  source 1..20: mol_type = other DNA
tttaccctccaataagtcac

SEQ ID NO: 151 - DNA, 20 bp, synthetic construct
  source 1..20: mol_type = other DNA
ttgtcggttccgttttgaaa

SEQ ID NO: 152 - DNA, 20 bp, synthetic construct
  source 1..20: mol_type = other DNA
tatccagtctactgcaatca

SEQ ID NO: 153 - DNA, 20 bp, synthetic construct
  source 1..20: mol_type = other DNA
tctgtccagtaaatgttgcg

SEQ ID NO: 154 - DNA, 20 bp, synthetic construct
  source 1..20: mol_type = other DNA
tacattgtcacccattctag

SEQ ID NO: 155 - DNA, 20 bp, synthetic construct
  source 1..20: mol_type = other DNA
ccatgaagtagatgaggcga

SEQ ID NO: 156 - DNA, 20 bp, synthetic construct
  source 1..20: mol_type = other DNA
tggggaacgctgtacattac

SEQ ID NO: 157 - DNA, 20 bp, synthetic construct
  source 1..20: mol_type = other DNA
tcactctgtgaatttcacct

SEQ ID NO: 158 - DNA, 20 bp, synthetic construct
  source 1..20: mol_type = other DNA
ggagtgtcaacacctcaatg

SEQ ID NO: 159 - DNA, 20 bp, synthetic construct
  source 1..20: mol_type = other DNA
taatcagggttttcccatac

SEQ ID NO: 160 - DNA, 20 bp, synthetic construct
  source 1..20: mol_type = other DNA
taaaggaaggagccgtggac

SEQ ID NO: 161 - DNA, 20 bp, synthetic construct
  source 1..20: mol_type = other DNA
attggaagagtcagcagcat

SEQ ID NO: 162 - DNA, 20 bp, synthetic construct
  source 1..20: mol_type = other DNA
aggcagcatggaaacgacta

SEQ ID NO: 163 - DNA, 20 bp, synthetic construct
  source 1..20: mol_type = other DNA
gtgatcagacaactccaagc

SEQ ID NO: 164 - DNA, 16 bp, synthetic construct
  source 1..16: mol_type = other DNA
ccttataagcttctgc

SEQ ID NO: 165 - DNA, 16 bp, synthetic construct
  source 1..16: mol_type = other DNA
acgataacggtcagta

SEQ ID NO: 166 - protein, 68 aa, Homo sapiens
SPRHSSTRSHSARSKKRALSLSPLSDGIGIDFNTIIRTSPTSLVAYINGSRASPANLSPQPEVYGHFL

SEQ ID NO: 167 - protein, 61 aa, Homo sapiens
GPLFSSPRSAVKLTKKRALSISPLSDASLDLQTVIRTSPSSLVAFINSRCTSPGGSYGHLS

SEQ ID NO: 168 - protein, 63 aa, Homo sapiens
VSRFSSPRVTPRLSRKRALSISPLSDASLDLQRMIRTSPNSLVAYINNSRSSSAASGSYGHLS

SEQ ID NO: 169 - protein, 63 aa, Homo sapiens
STRFSSPRLSARPSRKRTLSISPLSDHSFDLQTMIRTSPNSLVTILNNSRSSSSASGSYGHLS

SEQ ID NO: 170 - protein, 20 aa, synthetic construct
  VAR_SEQ 3: note = X can be any amino acid residues
  VAR_SEQ 5..10: note = Each X can be any amino acid residues
  VAR_SEQ 11: note = X= R or K
  VAR_SEQ 14..18: note = Each X can be any amino acid residues
  VAR_SEQ 20: note = X = Y or L
SSXRXXXXXXXKRXXXXXPX

(19) United States
(12) Patent Application Publication
(10) Pub. No.: US 2025/0263700 A1
(43) Pub. Date: Aug. 21, 2025

(54) METHOD OF TREATING, AMELIORATING AND/OR PREVENTING POLYCYSTIC KIDNEY DISEASE

(71) Applicant: Yale University, New Haven, CT (US)

(72) Inventor: Stefan Somlo, New Haven, CT (US)

(73) Assignee: Yale University, New Haven, CT (US)

(21) Appl. No.: 18/867,351

(22) PCT Filed: May 26, 2023
(86) PCT No.: PCT/US2023/023718

§ 371 Date: Nov. 19, 2024

Related U.S. Application Data
(60) Provisional application No. 63/365,359, filed on May 26, 2022.

(51) Int. Cl. C12N 15/113; A61K 38/17; A61K 48/00; A61P 13/12; C07K 16/28; C12N 9/22; C12N 15/11; G01N 33/50

(52) U.S. Cl. CPC C12N 15/113; A61K 38/1709; A61K 48/005; A61P 13/12; C07K 16/28; C12N 9/222; C12N 15/111; G01N 33/5023; G01N 33/5044; C12N 2310/11; C12N 2310/12; C12N 2310/14; C12N 2310/20; G01N 2333/4703; G01N 2500/10

(57) ABSTRACT

Described herein is a method for treating, ameliorating and/or preventing polycystic kidney disease in a subject in need thereof. The method including administering to the subject an effective amount of a compound that downregulates GLIS2. Also described herein are a composition and a kit for performing the method, and a method of screening suppressors of downstream effects of loss of PKD1 or PKD2.

### CROSS-REFERENCE TO RELATED APPLICATIONS

The present application claims priority under 35 U.S.C. § 119(e) to U.S. Provisional Patent Application No. 63/365,359, filed May 26, 2022, which is incorporated herein by reference in its entirety.

### STATEMENT REGARDING FEDERALLY SPONSORED RESEARCH

This invention was made with government support under DK100592 and DK120911 awarded by the National Institute of Diabetes and Digestive and Kidney Diseases under the National Institutes of Health. The government has certain rights in the invention.

### SEQUENCE LISTING

The XML named “047162-7379WO1(01984)_Seq Listing.xml” created on May 26, 2023, comprising 154.9 Kbytes, is hereby incorporated by reference in its entirety.

### BACKGROUND

Polycystic kidney disease (PKD) is a genetic disorder that causes fluid-filled cysts to grow in the kidneys. Autosomal dominant polycystic kidney disease (ADPKD) is a highly penetrant inherited PKD which causes cysts and deformation of the kidneys, typically over the span of decades, and eventually leads to kidney failure requiring dialysis or transplantation in the majority of patients after the fifth decade of life.

Although ADPKD is sometimes considered an orphan disease, the number of ADPKD patients is in fact significant. There are estimated to be over 600,000 affected individuals with ADPKD in the US alone and over 12 million worldwide. Furthermore, since ADPKD is not subject to founder mutations but rather de novo mutations that occur all the time, the population of ADPKD patients is expected to further grow as the world population expands.

Currently, there is only one approved medication for ADPKD, tolvaptan. Unfortunately, tolvaptan has significant side effects. The drug causes polyuria (6.0±1.8 L of urine per day, Kramers et al., BMC Nephrol. 2018; 19: 157), carries a black box warning (i.e., FDA's most stringent warning for drugs on the market) for hepatic injury, and is being subjected to the Risk Evaluation and Mitigation Strategy (REMS) by the FDA. Considering that many ADPKD patients require long-term treatments, the significant side effects of tolvaptan are especially undesirable.

Therefore, there is a need for novel treatments of polycystic kidney diseases, such as ADPKD. The present invention addresses this need.

### SUMMARY

In some embodiments, the instant specification is directed to a method of treating, ameliorating and/or preventing a polycystic kidney disease caused by or involving a mutant PKD1 or PKD2 in a subject in need thereof.

In some embodiments, the method includes: administering to the subject an effective amount of a compound that downregulates GLIS2.

In some embodiments, the polycystic kidney disease is an autosomal dominant polycystic kidney disease (ADPKD).

In some embodiments, the compound that downregulates GLIS2 includes: a protein inhibitor of GLIS2; a nucleic acid that down regulates GLIS2 by RNA interference, and/or an expression vector expressing the nucleic acid that down regulates GLIS2 by RNA interference; a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; a trans-dominant negative mutant protein of GLIS2, and/or an expression vector that expresses the trans-dominant negative mutant protein of GLIS2, or combinations thereof.

In some embodiments, the compound that downregulates GLIS2 comprises an antisense oligonucleotide (ASO), an siRNA, or an shRNA downregulating GLIS2 expression, and/or an expression vector expressing the ASO, siRNA or shRNA.

In some embodiments the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

In some embodiments, the instant specification is directed to a composition for treating, ameliorating and/or preventing a polycystic kidney disease caused by or involving a mutant PKD1 or PKD2 in a subject in need thereof.

In some embodiments, the composition includes a compound that downregulates GLIS2; and a pharmaceutically acceptable carrier.

In some embodiments, the polycystic kidney disease is an autosomal dominant polycystic kidney disease (ADPKD).

In some embodiments, the compound that downregulates GLIS2 includes a protein inhibitor of GLIS2; a nucleic acid that down regulates GLIS2 by RNA interference, and/or an expression vector expressing the nucleic acid that down regulates GLIS2 by RNA interference;
a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; a trans-dominant negative mutant protein of GLIS2, and/or an expression vector that expresses the trans-dominant negative mutant protein of GLIS2; or combinations thereof.

In some embodiments, the compound that downregulates GLIS2 comprises an antisense oligonucleotide (ASO), an siRNA, or an shRNA downregulating GLIS2 expression, and/or an expression vector expressing the ASO, siRNA or shRNA.

In some embodiments, the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

In some embodiments, the instant specification is directed to a kit for treating, ameliorating and/or preventing a polycystic kidney disease caused by or involves a mutant PKD I or PKD2 in a subject in need thereof.

In some embodiments, the kit includes a compound that downregulates GLIS2; and a manual instructing that the compound that downregulates GLIS2 is to be administer to the subject in an effective amount.

In some embodiments, the polycystic kidney disease is an autosomal dominant polycystic kidney disease (ADPKD).

In some embodiments, the compound that downregulates GLIS2 includes: a protein inhibitor of GLIS2; a nucleic acid that down regulates GLIS2 by RNA interference, and/or an expression vector expressing the nucleic acid that down regulates GLIS2 by RNA interference; a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; a trans-dominant negative mutant protein of GLIS2, and/or an expression vector that expresses the trans-dominant negative mutant protein of GLIS2, or combinations thereof.

In some embodiments, the compound that downregulates GLIS2 comprises an antisense oligonucleotide (ASO), an siRNA, or an shRNA downregulating GLIS2 expression, and/or an expression vector expressing the ASO, siRNA or shRNA.

In some embodiments, the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

In some embodiments, the instant specification is directed to a method of screening suppressors of downstream effects of loss of PKD1 or PKD2.

In some embodiments, the method includes: detecting a first GLIS2 expression level in the nucleus of a cell expressing PKD1 and PKD2; contacting the cell with a compound; and detecting a second GLIS2 expression level in the nucleus of the cell.

In some embodiments, a difference between the first GLIS2 expression level and the second GLIS2 expression level indicates that the compound is a suppressors of downstream effects of loss of PKD1 or PKD2.

In some embodiments, the cell is a kidney cell or a kidney-derived cell.

In some embodiments, the cell is a primary cell or a cell line.

In some embodiments, the cell is a mammalian cell or a mammalian cell-derived cell, optionally a human cell or a human cell-derived cell.

In some embodiments, that the second GLIS2 expression level is lower or higher than the first GLIS2 expression level indicates that compound is a suppressors of downstream effects of loss of PKD1 or PKD2.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1: Loss of renal cilia has been shown to suppress cyst growth in autosomal dominant polycystic kidney disease (ADPKD) (Ma et al., Nal Genel 45, 1004-1012 (2013)).

FIGS. 2A-2D illustrate certain aspects of the cilia-dependent cyst activating (CDCA) pathway in accordance with some embodiments. CDCA is repressed by polycystins and dependent on intact cilia. FIG. 2A: The PC1-PC2 complex is expressed in cilia and maintains CDCA in a physiologically regulated quiescent state with epithelial cells retaining a columnar shape and the tubule with normal lumen diameter. Normal physiological input would adjust CDCA to physiological needs of cell shape, lumen morphology, cell transport, metabolic properties, etc. WT, Wild type. FIG. 2B: Reduced PC1 dosage in heterozygous cells of autosomal-dominant polycystic kidney disease (ADPKD) patients may lead to weak constitutive activation of CDCA and a modest steady-state change in cell shape (less columnar) and lumen diameter (increased). FIG. 2C: Loss of PC1 in the presence of intact cilia, the condition for cyst initiation in ADPKD, leads to inexorable activation of CDCA, profound changes in cells to a more squamoid shape, low-level proliferation, active remodeling of surrounding kidney parenchyma, and growth of cysts. Note that the images of cells and tubules (cysts) in FIG. 2C are at a much lower illustrative “magnification” than FIGS. 2A, 2B and 2D. FIG. 2D: Loss of cilia in the absence of PC1 markedly reduces the activation of CDCA and maintains cell shape and tubule lumen diameter in a more normal range.

FIG. 3 illustrates certain aspects of a transcriptome analysis for CDCA targets, in accordance with some embodiments. The present study hypothesized that discovering and targeting transcriptional changes resulting from CDCA activation could provide novel functionally validated downstream targets for slowing polycystic disease progression.

FIG. 4 illustrates certain aspects of a cell-type specific transcriptome analysis in accordance with some embodiments. The analysis is termed “Translating Ribosome Affinity Purification (TRAP).” Referring to FIG. 4, the 60S ribosomal protein L10a (RPL10A) is fused with EGFP (EGFP-L10a) and knocked in using Rosa26. The EGFP-L10a is not expressed in Cre-negative cells due to the presence of the STOP sequence. In Cre-positive cells, the STOP sequence is removed and EGFP-L10a is expressed. Ribosomes containing EGFP-L10a were pulled-down together with the translating mRNA molecules, allowing the in vivo profiling of selective cell populations from native tissues, and the profiling of the actively translating mRNA.

FIG. 5 illustrates certain aspects of animal models for transcriptome profiling by TRAP RNA sequencing, in accordance with some embodiments. Wild-type (Pkd1^{fl/fl}; Pax8^{rtTA}) mice, Pkd1-Pax8 (Pkd1^{fl/fl}; Pax8^{rtTA}; TetO^{Cre}) mice, and Pkd1/Kif3a-Pax8 (Pkd1^{fl/fl}; Kif3a^{fl/fl}; Pax8^{rtTA}; TetO^{Cre}) mice were administered with doxycycline between postnatal days 28 and 42 to induce the expression of Cre recombinase. The actively translating mRNA were pulled down at either postnatal day 49 (7 weeks) or postnatal day 70 (10 weeks). Representative kidney tissue images of the mice are shown indicating that there is minimal or absent cyst formation at selected time points.

FIG. 6 illustrates some aspects of the information of the mRNAs identified in the TRAP analysis, in accordance with some embodiments. Overlap results of the 7-week mRNAs and the 10-week mRNAs identified in the TRAP analysis

FIG. 7 illustrates some aspects of the GLIS 2 sequence information in accordance with some embodiments. DBD: DNA binding domain. AD: Activation domain. NCR: N-terminal conserved region. CLS: Cilia localization signal. SUFU: SUFU binding domain. The sequences shown in FIG. 7: human GLIS3 amino acid residues 130-197 (SPRHSSTRSHSARSKKRALSLSPLSDGIGIDFNTIIRTSPTSLVAYINGSRASPANLSPQPEV YGHFL, SEQ ID NO:166), human GLIl amino acid residues 65-125 (GPLFSSPRSAVKLTKKRALSISPLSDASLDLQTVIRTSPSSLVAFINSRCTSPGGSYGHLS, SEQ ID NO:167), human GLI2 amino residues 215-277 (VSRFSSPRVTPRLSRKRALSISPLSDASLDLQRMIRTSPNSLVAYINNSRSSSAASGSYGH LS, SEQ ID NO:168), human GLI3 amino acid residues 276-338 (STRFSSPRLSARPSRKRTLSISPLSDHSFDLQTMIRTSPNSLVTILNNSRSSSSASGSYGHLS, SEQ ID NO:169), and Transportin 1 binding motif (SSXR-XXXXXX-R/K-KR-XXXXX-P-Y/L, SEQ ID NO:170).

FIG. 8 illustrates some aspects of the in vitro confirmation of genotype dependent GLIS2 expression changes in the Pkd1 and Pkd2 knockout IMCD3 inner medullary collecting duct (IMCD) cell line. The IMCD3 cells were cultured in medium containing 5% fetal bovine serum (FBS), and were starved for 24 hours in 0.1% FBS DMEM before harvest.

FIGS. 9A-9B illustrate some aspects of the in vitro genotype dependent GLIS2 expression changes in the primary kidney cells of Pkd1 and Pkd2 knockout animals, in accordance with some embodiments. FIG. 9A: Results from Pkd1 knockout primary kidney cells. FIG. 9B: Results from Pkd2 knockout primary kidney cells.

FIG. 10 demonstrates that the Glis2 protein is upregulated in nuclear fraction of Pkd1 knockout primary cells, in accordance with some embodiments.

FIG. 11 demonstrates that the Glis2 protein is upregulated in Pkd2 knockout primary cells, in accordance with some embodiments.

FIGS. 12A-12D demonstrate that Glis2 knockout reduces cyst formation in the kidneys of some model animals of polycystic kidney disease, in accordance with some embodiments. FIG. 12A: Images of kidneys of wild-type, Glis2^{-/-}, Pkd1^{fl/fl}; Pkhd1-Cre, and GLIS2^{-/-}; Pkd1^{fl/fl}; Pkhd1-Cre animals. FIG. 12B: Kidney to body weight ratio of the animals. FIG. 12C: Cystic index of the kidneys of the animals. FIG. 12D: Serum urea nitrogen level of the animals.

FIGS. 13A-13D demonstrate that Glis2 knockout reduces cyst formation in the kidneys of some model animals of polycystic kidney disease, in accordance with some embodiments. FIG. 13A: Images of kidneys of wild-type, GLIS2^{-/-}, Pkd1^{fl/fl}; Pax8; TetO-Cre, and GLIS2^{-/-}; Pkd1^{fl/fl}; Pax8; TetO-Cre animals. FIG. 13B: Kidney to body weight ratio of the animals. FIG. 13C: Cystic index of the kidneys of the animals. FIG. 13D: Serum urea nitrogen level of the animals.

FIGS. 14A-14E demonstrate that Glis2 knockout reduces cyst formation in the kidneys of some model animals of polycystic kidney disease, in accordance with some embodiments. FIG. 14A: Images of kidneys of wild-type, Glis2^{-/-}, Pkd2^{WS25/-}, and GLIS2^{-/-}; Pkd2^{WS2/-} animals. FIG. 14B: Kidney to body weight ratio of the animals. FIG. 14C: Cystic index of the kidneys of the animals. FIG. 14D: Serum urea nitrogen level of the animals. Open symbols, female; fill symbols, male. FIG. 14E: Additional images of kidneys of the animals.

FIGS. 15A-15D demonstrate that the Glis2 ASO (“ASO 972”) specifically knocks down Glis2, in accordance with some embodiments. In situ experiments with RNAScope (FIGS. 15A-15B) and single molecule FISH (FIGS. 15C-15D). FIG. 15A: Specificity of the GLIS2 RNAScope probe (green) shows low level signal in wild type kidneys and absent signal in GLIS2 knockout kidney. FIG. 15B: ASO 972 specifically knocked down the expression of GLIS2 in the kidney of 18 weeks old UBC-CreER™; Pkd1 fl/fl animal, while the control ASO (“Scrambled ASO”) did not. FIGS. 15C-15D: Single molecule fluorescence in situ hybridization (smFISH) assays show that the control ASO did not reduce the level Glis2 mRNA (FIG. 15C) while ASO 972 did (FIG. 15D).

FIGS. 16A-16B demonstrate that treatment with antisense oligonucleotides (ASO) targeting Glis2 was able to alleviate the abnormally high kidney to body weight ratio caused by Pkd1 knockout in the proximal tubules in male animals, in accordance with some embodiments. FIG. 16A: timelines of the knockout and treatment. FIG. 16B: Kidney to body weight ratio of the animals. M, male mice showed increased kidney to body weight ratio with control ASO and the reduction with the GLIS2 specific ASO; F, female mice did not show increased kidney to body weight ratio at baseline.

FIGS. 17A-17B demonstrate that treatment with antisense oligonucleotides (ASO) targeting GLIS2 was able to alleviate the abnormally high kidney to body weight ratio and kidney cystic index caused by Pkd1 knockout in the proximal tubules in male animals, in accordance with some embodiments. FIG. 17A: Kidney to body weight ratio and cystic index of the animals. FIG. 17B: Images of the kidneys of control animals (“Control ASO”) and animals treated with the Glis2 ASO (“Glis2 ASO”).

FIGS. 18A-18G: Translating Ribosome Affinity Purification (TRAP) RNASeq in Pkd1 mouse models, in accordance with some embodiments. FIG. 18A: Schematic outline of the TRAP RNASeq strategy. Male and female mice with the indicated genotypes were induced with doxycycline from P28-P42 to inactivate the respective conditional alleles and to activate the EGFP-L10a ribosomal fusion protein in a Cre recombinase dependent cell specific manner. Labelled ribosomes were isolated from fresh kidney tissues and processed together by RNASeq. Bioinformatic analysis of three pairwise comparisons for each sex identified differentially expressed genes (DEG). In the example shown, only Genes A and B show the same relative direction change in both the Pkd1^{KO} vs noncystic and the Pkd1^{KO} vs the Pkd1^{KO}+ cilia^{KO} comparisons. Genes A and B are said to have the ‘CDCA pattern’ of expression whereas Genes C-F do not. FIG. 18B: Table showing numbers of DEGs identified for each sex in the indicated pairwise analyses and in Pkd1^{KO} compared to both other groups. FIG. 18C: Volcano plots of DEGs in males (upper panels) and females (lower panels) for the indicated pairwise comparisons. Genes with significant differential expression (FDR≤0.05) and same change direction in Pkd1^{KO} compared to both noncystic and Pkd1^{KO}+ cilia^{KO} groups are indicated by red dots and numbers (upregulated) and blue dots and numbers (downregulated). Gray dots and NS represent genes that have no statistical significance. X-axis, fold change; y-axis, Benjamini-Hochberg adjusted P-value for the differences in gene expression. Red dashed line, adjusted P-value≤0.05 threshold; blue dashed line, 2-fold change threshold. FIG. 18D: Heat map showing hierarchical clustering of TRAP RNASeq expression data for 73 genes that showed significant same relative direction change in expression in Pkd1^{KO} compared to noncystic and Pkd1^{KO}+ cilia^{KO} in both male and female mice with expression TPM>1 reported in a majority of microdissected kidney tubule segments in normal kidney. Color scale indicates relative gene expression value. FIGS. 18E-18F: Selected genes discussed in the text and shown in red in FIG. 18D are presented in a heat map with hierarchal clustering (FIG. 18E), and labeled on volcano plots (FIG. 18F) with the indicated pairwise comparisons in male (left) and female (right) mice.

FIG. 18G: Quantitative RT-PCR (qRT-PCR) for the indicated genes from primary cells cultured from kidneys of Pkd1^{fl/fl}; Pax8^{rtTA}; TetO^{Cre} mice. Cells were either treated with doxycycline (red) to knockout Pkd1 or left untreated (blue) without Pkd1 knockout. Expression of each gene following Pkd1 knockout recapitulated the relative direction change compared to non-knockout controls as was observed by TRAP RNASeq in vivo (FIGS. 18D-18F).

FIGS. 19A-19M: Glis2 transcript and nuclear protein expression are in vitro and in vivo indicators of polycystin dependent cyst forming potential, in accordance with some embodiments. a,b, Quantitative RT-PCR (qRT-PCR) for Glis2. FIG. 19A: Kidney tissues from 13-week-old noncystic control (blue) and cystic Pkd2 knockout (red) and from kidneys at the indicated number of days after starting Pkd2 re-expression at 13 weeks. Fold-change Glis2 relative to its expression in noncystic mice which is set to 1.0. All mice except noncystic have the Pkd2^{fl/-}; Pkd2^{FSF}; Pax^{rtTA}; TetO^{Cre}; Rosa26^{FlpoER} genotype; noncystic mice lack Pax8^{rtTA}. All mice received oral doxycycline from P28-42. Mice with re-expression of Pkd2 were started on daily tamoxifen at 13 weeks and kidney tissue was obtained after 2, 4 and 6 days of tamoxifen treatment. Glis2 expression decreases with re-expression of Pkd2. FIG. 19B: Wild type kidney tissues at the indicated postnatal ages showing fold-change Glis2 relative to its expression at P40 which is set to 1.0. a,b, Multiple-group comparisons are done by one-way ANOVA followed by Tukey's multiple-comparison test and are presented as the mean±s.e.m. FIG. 19C: Glis2 immunoblot in cytosolic and nuclear fractionated kidney tissue lysates at the indicated postnatal ages. Lamin A/C, loading control and relative enrichment for nuclear fractions; Ponceau, overall loading of lysates. FIGS. 19D, 19F and 19H qRT-PCR and FIGS. 19E, 19G and 19I immunoblots for the indicated proteins from primary cell cultures of kidneys of mice with the indicated genotypes without and with doxycycline treatment in vitro to inactivate Pkd1 (FIGS. 19D-19E), Pkd2 (FIGS. 19F-19G) and Pkd1 without or with Kif3 (FIGS. 19H-19I). FIG. 19I, * is non-specific band. FIGS. 19D, 19F and 19H, Target genes for qRT-PCR are indicated on the abscissa; fold change following doxycycline (red) is presented relative to expression of the same gene without doxycycline (blue) which is set to 1.0. Statistical significance for each gene is determined by unpaired two-tailed Student's t-test and presented as mean±s.e.m. Glis2 transcript (FIGS. 19D, 19F and 19H) and nuclear protein expression (FIGS. 19E and 19G) is upregulated following inactivation of Pkd1 (FIGS. 19D, 19E and 19H) or Pkd2 (FIGS. 19F and 19G). Increases in Glis2 transcript (FIG. 19H) and protein (FIG. 19I) expression is suppressed by dual inactivation in Pkd1^{KO}+ cilia^{KO} primary cells. FIG. 19E, Lamin B and g, Lamin A/C, loading control and relative enrichment for nuclear fractions; Ponceau, overall loading of lysates. FIG. 19J, Dual inactivation of Pkd2 and Ift88 and k, dual inactivation of Pkd1 and Tulp3 in primary kidney cell cultures treated with doxycycline results in suppression of the increase in Glis2 protein expression that occurs with inactivation of Pkd2 (FIG. 19J) or Pkd1 (FIG. 19K) alone; FIG. 19I, * is non-specific band. All mice have Pax8^{rtTA}; TetO^{Cre} in addition to the indicated alleles. FIGS. 19I and 19J, Hsp90 and Ponceau, loading controls. FIG. 19L, Immunoblots for the indicated proteins in total kidney tissue lysates from mice with Pkd1^{KO} and Pkd1^{KO}+ cilia^{KO} and m, nuclear fractionated kidney tissue lysates from mice with Pkd1^{KO} and Pkd1^{KO}+ Tulp3^{K}O. All mice have Pax8^{rtTA}; TetO^{Cre} in addition to the indicated alleles, received oral doxycycline from P28-42 and kidney tissue was obtained at 10 weeks (FIG. 19L) and 12 weeks (FIG. 19M). FIG. 19L, Hsp90 and Ponceau, loading controls; FIG. 19M, Lamin A/C, loading control and relative enrichment for nuclear fractions; Ponceau, overall loading of lysates. Experiments in FIGS. 19C, 19G, 19J, and 19M were done two times with primary cell cultures from different mice; FIGS. 19I and 19K were done three times; FIGS. 19E and 19L were done more than three times. Full-length blots are provided as source data.

FIGS. 20A-20P demonstrate that Glis2 inactivation suppresses progression in mouse models of polycystic kidney disease, in accordance with some embodiments. FIGS. 20A, 20E, 20I and 20M, Representative images of kidneys with the specified genotypes and FIGS. 20B-20D, 20F-20H, 20J-20L, and 20N-20P show corresponding kidney structural (FIGS. 20B, 20F, 20J and 20N, kidney weight to body weight ratio; FIGS. 20C, 20G, 20K and 20O, cystic index) and functional (FIGS. 20D, 20H, 20L and 20P, blood urea nitrogen) measures for male (closed symbols) and female (open symbols) mice. n, refer to number of mice except in FIG. 20O, where it is the number of kidneys. Multiple-group comparisons were performed by one-way ANOVA followed by Tukey's multiple-comparison test, presented as mean±s.e.m. FIGS. 20A, 20E, 20I and 20M, Scale bars, 1 mm. FIGS. 20A-20D, Pkd1 models based on the collecting duct selective Pkhd1-Cre at P24. Genotypes in FIGS. 20B-20D correspond to symbol color and shape coding in FIG. 20A. FIGS. 20E-20H, Pkd1 models based on Pax8^{rtTA} induced with oral doxycycline from P1-14 and examined at P14. Genotypes in FIGS. 20F-20H correspond to symbol color and shape coding in FIG. 20E. FIGS. 20I-20L, Pkd1 models based Pax8^{rtTA} induced with oral doxycycline from P28-42 and examined at 18 weeks. Genotypes in FIGS. 20J-20L correspond to symbol color and shape coding in FIG. 20I. FIGS. 20M-20P, Pkd2 models based on the Pkd2^{WS25} allele examined at 14 weeks. Genotypes in FIGS. 20N-20P correspond to symbol color and shape coding in FIG. 20M.

FIGS. 21A-21N demonstrate that Glis2 antisense oligonucleotide (ASO) reduces kidney cyst growth in an adult onset ADPKD model, in accordance with some embodiments. FIG. 21A, Representative kidney images, FIG. 21B, kidney weight to body weight ratio, FIG. 21C, cyst index and FIG. 21D, blood urea nitrogen from mice with the indicated genotypes in a and matched symbol color and shape codes in FIGS. 21B-21D at 18 weeks age. Pkd1 inactivation was induced with tamoxifen administration from P28-35 and control- or Glis2-ASO was administered twice in week 5 and then weekly through 17 weeks age. All mice received tamoxifen regardless of genotype. One group of noncystic controls also received Glis2-ASO; the other did not. FIGS. 21E-21L, Representative images of two-color fluorescent in situ hybridization (FISH) for Glis2 mRNA (green) and the proximal tubule marker gene Lrp2 (red) encoding megalin. FIGS. 21B-21D, Male mice, closed symbols; female mice, open symbols. n, refer to number of mice. FIGS. 21E-21H, Single molecule FISH (smFISH); FIGS. 21I-21L, RNAScope-FISH. Glis2 expression is detected by both methods in control mouse kidneys (FIGS. 21E and 21I) and the specificity of both probes is demonstrated by the absence of signal for Glis2 in kidneys from Glis2^{-/-} mice (FIGS. 21F and 21J). Glis2 signal by both FISH methods is increased relative to control kidney in Lrp2 positive proximal tubule derived cysts in mice treated with control-ASO (FIGS. 21G and 21K). Glis2 signal by both FISH approaches is decreased relative to control-ASO in proximal tubules of mice treated with Glis2-ASO (FIGS. 21H and 21L). All mice, except FIGS. 21F and 21J, received tamoxifen IP daily from P28-35; all kidneys were examined at age 18 weeks. FIGS. 21M and 21N, Treatment with Glis2-ASO significantly decreased Glis2 mRNA levels in proximal tubules of Pkd1^{fl/fl}; UBC^{Cre-ERT2} male mice compared to control-ASO treated counterparts when measured both smFISH (FIG. 21M) and RNAScope-FISH (FIG. 21N). n=3 for all groups. Multiple-group comparisons (FIGS. 21B-21D and 21M) were performed by one-way ANOVA followed by Tukey's multiple-comparison test. Comparison between two groups (FIG. 21N) was performed by unpaired Student's t-test. All data are presented as mean±s.e.m. Scale bar, 1 mm (FIG. 21A); 50 μm (FIGS. 21E-21L).

FIGS. 22A-22N demonstrate that Glis2-ASO treatment decreases cyst cell proliferation, inflammation and fibrotic changes in an adult-onset model of ADPKD, in accordance with some embodiments. FIGS. 22A-22N, Analyses of kidney tissues from noncystic control mice (red symbols) and Pkd1 mouse models based on UBC^{Cre-ERT2}, treated with either control-ASO (blue) or Glis2-ASO (green). All mice were male and received tamoxifen from P28-35. Tamoxifen induced Pkd1 knockout in the proximal tubules in mice with UBC^{Cre-ERT2} which then received treatment with the respective ASO from weeks 5 to 17. Kidney tissue was examined at 18 weeks age. FIGS. 22A-22D, Aggregate quantitative data (FIGS. 22A and 22C) and representative images (FIGS. 22B and 22D) showing the percentage of EdU-positive nuclei (FIGS. 22A and 22B) and Ki67-positive nuclei (FIGS. 22C and 22D) in the Lotus tetragonolobus agglutinin (LTA) positive proximal tubules from kidneys of mice with the indicated genotypes and treatments. FIG. 22E, Adgre1, the gene encoding F4/80, mRNA expression in whole kidney lysates normalized to Gapdh and expressed as fold-change relative to the mean for control kidneys which is set to 1.0. FIG. 22F, Representative images of F4/80 expression in the region of proximal tubules. FIGS. 22G-22J, Changes in inflammatory responses indicated by changes in TNF-α (FIGS. 22J and 22H) and cleaved caspase-1 (FIGS. 22I and 22J) expression in kidneys from mice with the indicated genotypes and treatments. Immunoblots are shown with quantitation using densitometric ratios (FIGS. 22G and 22I) that include both bands for TNF-α (FIG. 22J) and cleaved caspase-1 (FIG. 22I) normalized to Hsp90. Fold-change is shown relative to the mean of the ratio in the non-cystic samples, which is set to 1.0. Multiple-group comparisons were performed by one-way ANOVA followed by Tukey's multiple-comparison test, presented as mean±s.e.m. h,j Representative images of TNF-α (FIG. 22H) and cleaved caspase-1 (FIG. 22J) show reduced expression in the region of proximal tubules following treatment with Glis2-ASO. FIGS. 22K-22N, Changes in fibrotic responses indicated by changes in α-SMA (FIGS. 22K and 22J) and PDGFRβ. (FIGS. 22M and 22N) expression following control- or Glis2-ASO treatment. Immunoblots are shown with quantitation using densitometric ratios (FIGS. 22K and 22M) for α-SMA (FIG. 22K) and PDGFRP (FIG. 22M) normalized to Hsp90. Fold-change is shown relative to the mean of the ratio in the non-cystic samples, which is set to 1.0. Multiple-group comparisons were performed by one-way ANOVA followed by Tukey's multiple-comparison test, presented as mean±s.e.m. l,n Representative images of α-SMA (FIG. 22L) and PDGFRβ (FIG. 22N) show reduced expression in the region of proximal tubules following treatment with Glis2-ASO. (FIGS. 22B, 22D, 22F, 22H, 22J, 22L and 22N), At least one section from 3 kidneys for each group were examined for all representative images. Scale bars: 150 μm (FIGS. 22B and 22D); 50 μm (FIGS. 22F, 22H, 22J, 22L and 22N). Full-length blots are provided as source data.

FIGS. 23A-23C: Early stage Pkd1^{KO} and Pkd1^{KO}+ cilia^{KO} show mild tubule dilation without cyst formation, in accordance with some embodiments. FIG. 23A, Quantitative data showing the kidney weight-to-body weight ratio from 7 weeks-old mice of both sexes. Noncystic (red circle), Pkd1^{KO}(blue square) and Pkd1^{KO}+ cilia^{KO} (green triangle). All mice received doxycycline from P28-42. Male mice, filled symbols; female mice, open symbols. ‘n’, number of mice in each group. Multiple-group comparisons were performed using one-way ANOVA followed by Tukey's multiple-comparison test and are presented as the mean±s.e.m. FIG. 23B, Representative images of kidneys from 7 weeks-old male mice with the indicated genotypes. Scale bar=500 μm. At least one section from 3 kidneys for each group were examined for all representative images. FIG. 23C, The aggregate semi-quantitative data showing the Pkd1^{fl} allele deletion efficiency (see Methods) in kidney from mice with the indicated genotypes and sex. These are the same mice used for the TRAP studies and colors and n, symbol shapes correspond to genotype and sex denoted in FIG. 23A.

FIGS. 24A-24B: Flow diagram for TRAP RNASeq analysis, in accordance with some embodiments. FIG. 24A, Raw sequencing reads were trimmed using Fastp tool. The trimmed sequencing reads were aligned to the mouse reference genome using STAR. Gene-level expression quantification was performed using Subread/featureCount. Non-expressed/low-expressed genes were filtered using the criteria that the raw read counts of each gene should be no less than 6 in at least 20% of samples. The filtered read counts matrix was normalized using the TPM method (Transcripts Per Million). The filtered read counts matrix was also used to identify DEGs with R package DESeq2. The TPM normalized relative expression matrix was used to perform classic Principal Component Analysis (PCA) and robust PCA analysis to identify potential problematic samples (FIGS. 25A-25B). FIG. 24B, The steps used to filter out non/low-expressed genes and non-coding genes in male and female mice are shown starting from average number of genes across samples from each sex. The numbers of genes obtained after performing each step are indicated.

FIGS. 25A-25B: PCA analysis for TRAP RNASeq, in accordance with some embodiments. The percentages on each axis represent the percentages of variants explained by the respective principal components. FIG. 25A, PCA plot of male and female samples. Data points are colored by genotype group, and almost all samples have values close to their expected genotype groups. FIG. 25B, PCA plot showing sex differences of the TRAP RNASeq translatome profiles in 7-week kidneys.

FIG. 26A, Venn diagrams showing numbers of DEGs identified for each sex in the indicated pairwise analyses and in Pkd1^{KO} compared to both other groups. The number in parentheses (FIG. 26A) indicates Pkd1^{KO} DEGs changed in the same direction relative to the other two groups, when different from the total number of DEG. FIG. 26B, The DEGs in Pkd1^{KO} compared to both other groups and shared between the sexes (n=167).

FIG. 27: Clustered heatmap of Pearson correlation coefficients, in accordance with some embodiments. Clustering of correlations coefficients based on TPM values of 799 unique DEGs in the combined male and female DEG sets DEGs comparing Pkd1^{KO} to both Pkd1^{KO}+ cilia^{KO} and noncystic kidneys, showing grouping of Pkd1^{KO} together and further sub-clustering by sex within this group.

FIGS. 28A-28C: Functional enrichment analysis of DEGs using Metascape, in accordance with some embodiments. X-axis represents enrichment magnitude. FIG. 28A, The statistically significantly enriched set of 440 genes with same direction in Pkd1^{KO} compared to both noncystic and Pkd1^{KO}+ cilia^{KO} in male mice. FIG. 28B, The statistically significantly enriched set of 526 genes with same direction in Pkd1^{KO} compared to both noncystic and Pkd1^{KO}+ cilia^{KO} in female mice. FIG. 28C, The statistically significantly enriched set of 167 genes with same direction in Pkd1^{KO} compared to both noncystic and Pkd1^{KO}+ cilia^{KO} in common between male and female mouse kidneys.

FIGS. 29A-29D: Heatmaps showing expression of ‘CDCA pattern’ DEG in male (FIGS. 29A and 29B) and female (FIGS. 29C and 29D) mice. FIGS. 29A and 29C, Heatmaps using published bulk RNASeq expression data from 14 microdissected normal nephron segments showing the subset of (FIG. 29A) 185 out of 440 and (FIG. 29C) 252 out of 526 of the CDCA pattern DEG with TPM>1.0 across most of the kidney tubule for male and female mice, respectively. The heatmaps present bulk RNASeq data also provides the key to identification the abbreviations for the 14 nephron segments. FIGS. 29B and 29D, Heatmaps showing hierarchical clustering TRAP RNASeq expression data for the FIGS. 29A and 29C, respectively. These are the 185 male and 252 female genes that showed significant same relative direction change in expression in Pkd1^{KO} compared to noncystic and Pkd1^{KO}+ cilia^{KO} in both male and female mice with expression TPM>1 reported in a majority of microdissected kidney tubule segments in normal kidney.

FIGS. 30A-30F demonstrate that Glis2 is undetectable in primary cilia, in accordance with some embodiments. FIG. 30A, mRNA expression of Glis2, Pkd1 and Pkd2 in IMCD3 cells with Pkd1 knockout (clones 3F5 and 3F6) compared to the parental control cell line (Cas9). Fold-change is shown relative to the mean of the ratio in the control cell line, which is set to 1.0. Multiple-group comparisons were performed using two-way ANOVA followed by Dunnett's multiple-comparison test, presented as the mean±s.e.m. FIG. 30B, Representative images from wildtype IMCD3 cells expressing C-terminal Glis2-EGFP (green) and the cilia marker protein Nphp3¹⁻²⁰⁰-mApple (red) under live cell imaging conditions. Left three panels are maximum intensity projections. Right three panels are a single plane from a z-stack. Glis2-EGFP epifluorescence is seen in the nucleus but is absence from cilia marked by Nphp3¹⁻²⁰⁰-mApple. FIG. 30C, Representative images from transiently transfected HEK-293T cells expressing C-terminal Glis2-FLAG (green) with immunostaining of anti-FLAG and the cilia marker anti-Arl13b (red). Left three panels are maximum intensity projections. Right three panels are a single plane from a z-stack. FIG. 30D, Representative images of IMCD3 cells expressing C-terminal Glis3-EGFP (green) epifluorescence and immunostaining with acetylated α-tubulin (red). Nuclei are stained in blue with Hoechst. Glis3-EGFP epifluorescence of readily detectable in cilia. FIG. 30E, Representative images of IMCD3-Cas9 control cells expressing Glis2-EGFP (green) and NPHP3¹⁻²⁰⁰-mApple (red) under live cell imaging conditions. Left three panels are maximum intensity projections. Right three panels are a single plane from a z-stack. FIG. 30F, Representative images of IMCD3-3F6 Pkd1^{KO} cell line expressing Glis2-EGFP (green) and NPHP3¹⁻²⁰⁰-mApple (red) under live cell imaging conditions. Left three panels are maximum intensity projections. Right three panels are a single plane from a z-stack. Glis2-EGFP epifluorescence is seen in the nucleus in both FIG. 30E and FIG. 30F, but is absence from cilia marked by NPHP3¹⁻²⁰⁰-mApple. All experiments were done in cell lines transduced with lentiviral vectors; all experiments were repeated at least 3 times and showed consistent results for the representative images shown. Scale bars, 25 μm.

FIGS. 31A-31D demonstrate that the polyclonal anti-Glis2 antibody YNG2 detects mouse and human Glis2, in accordance with some embodiments. FIG. 31A, Total cell lysates of HEK-293T cells stably expressing doxycycline inducible C-terminal mouse Glis2-FLAG (mGlis2-FLAG) were immunoblotted with anti-FLAG antibodies and anti-Glis2 (YNG2) antibodies without (−) and with (+) doxycycline treatment. Ponceau staining is shown as loading control. FIG. 31B, Nuclear lysates of HEK-293T cells transfected with C-terminal mouse Glis2-Halo (mGlis2-Halo) or N-terminal human Halo-Glis2 (Halo-hGLIS2) were immunoblotted with anti-Halo and anti-Glis2 (YNG2) antibodies. Lamin A/C is shown for nuclear loading. Ponceau staining is shown for total loading control. FIG. 31C, SV40 transformed cell lines were made from primary cell cultures of kidneys of Glis2^{-/-} null and Glis2^{+/+} wild type mice and lysates of cytosolic and nuclear fractions were obtained from each cell line. YNG2 detects a band migrating as expected for Glis2 only in the nuclear fraction of the wild type cell line. Lamin A/C is shown for nuclear enrichment and loading control. Total loading was controlled by Ponceau staining. FIG. 31D, Lysates of cytosolic and nuclear fractions from kidney tissue of Glis2^{-/-} (GIis2^{fl/fl}; Actb^{Cre}) null and Glis2^{+/+} wild type mice were immunoblotted with YNG2 antibodies. Lamin A/C is shown as nuclear enrichment and loading control. Glis2 is specifically detected only in lysates of the nuclear fraction of wild type kidney. Ponceau staining is shown for total loading. Full-length blots are provided as source data.

FIG. 32: Depletion of PCI in primary cells from kidneys of Pkd1^{fl/fl}; Pax8^{rtTA}, TetO^{Cre} mice after doxycycline treatment, in accordance with some embodiments. Immunoblot showing the Endo H resistant (PC1-NTR) and Endo H sensitive (PC1-NTS)N-terminal fragments of PC1 detected by anti-PC1 clone 7e12 antibody in primary cells with indicated genotype without or with doxycycline treatment for 3 days. Doxycycline induced knockout results in absence of PC1. Hsp90 and Ponceau staining are shown as loading controls. Full-length blots are provided as source data.

FIGS. 33A-33B demonstrate that doxycycline treatment results in absence of cilia in Pkd1^{KO}+ cilia^{KO} and Pkd2^{KO}+ cilia^{KO} primary cells, in accordance with some embodiments. FIG. 33A, Representative images of primary cells cultured from kidneys of mice with the indicated genotypes without or with doxycycline treatment for 3 days. Cells were fixed after 1 day serum starvation at the end of 20 days in culture cells were stained for ZO1 (red) to mark tight junctions and Arl13b (green) to mark cilia. Cilia are absent in double knockout cells after doxycycline treatment. FIG. 33B, Representative images of primary cells cultured from kidneys of mice with the indicated genotypes without or with doxycycline treatment for 3 days. Cells were serum starved for one day and stained for the cilia markers Ift88 (green) and acetylated α-tubulin (red) and the nuclear Hoechst stain (blue). Scale bars, 25 μm.

FIGS. 34A-34B: Images of all the kidney histological sections used in FIGS. 20A-20D. Scale bar, 1 mm.

FIGS. 35A-35D: Glis2 inactivation shows sustained suppression polycystic kidney disease in an early onset mouse model, in accordance with some embodiments. FIG. 35A, Representative images of kidneys with the indicated genotypes at P49. Scale bar, 1 mm. FIGS. 35B-35D, Aggregate quantitative data for the indicated parameters. n, number of mice in each group. Colors and symbol shapes correspond to genotypes defined in FIG. 35A. FIG. 35E, All kidney histological sections used in FIGS. 35B-35D. FIGS. 35B-35D, Male mice, closed symbols; female mice, open symbols. Multiple-group comparisons by one-way ANOVA followed by Tukey's multiple-comparison test, presented as mean±s.e.m. e, Scale bar, 1 mm.

FIGS. 36A-36B: Images of all the kidney histological sections used in FIGS. 20E-20H. Scale bar, 1 mm.

FIGS. 37A-37F demonstrate that adult inactivation of Glis2 does not result in a nephronophthisis-like phenotype, in accordance with some embodiments. FIGS. 37A-37F, Representative images of kidney cortex (FIGS. 37A, 37C and 37E) and medulla (FIGS. 37B, 37B and 37F) from mice with the indicated genotypes at 18 weeks age. All mice with the Pax8^{rtTA}; TetO^{Cre} alleles received oral doxycycline to induce gene knockouts from P28-42. FIGS. 37A and 37B, Comparison of germline null Glis2^{ΔEx3/ΔEx3} and adult inducible Glis2 inactivation. Glis2^{-/-} kidneys show inflammatory infiltrates (arrowhead, H&E) and interstitial fibrosis (arrows, blue areas in Masson's Trichrome and red areas in Picrosirius Red) in both cortex (FIG. 37A) and medulla (FIG. 37B). These changes are absent in tubule selective adult conditional inactivation of Glis2 (Glis^{fl/fl}; Pax8^{rtTA}; TetO^{Cre}). FIGS. 37C-37F, Fibrosis is present in the pericystic areas in the cortex (FIGS. 37C and 37E) and medulla (FIGS. 37D and 37F) of Pkd1^{fl/fl}; Pas8^{rtTA}; TetO^{Cre} kidneys (arrows in FIGS. 37E and 37F) but absent from the Glsi2^{fl/fl}; Pkd1^{fl/fl}; Pas8^{rtTA}; TetO^{Cre} double knockout kidneys which are protected from cyst formation. Scale bars: 250 μm (FIGS. 37A-37D); 50 μm (FIGS. 37E and 37F).

FIGS. 38A-38B: Images of all the kidney histological sections used in FIGS. 20I-20L. Genotypes and sexes for each kidney are shown at the top. Scale bar, 1 mm

FIGS. 39A-39D: Concomitant inactivation of Glis2 decreases cyst cell proliferation in adult onset ADPKD, in accordance with some embodiments. FIGS. 39A-39D, Representative images and aggregate quantitative data showing the percentage of EdU-positive nuclei (FIGS. 39A and 39B) and Ki67-positive nuclei (FIGS. 39C and 39D) in Lotus tetragonolobus agglutinin (LTA) positive proximal tubules (FIGS. 39A and 39C) and Dolichos biflorus agglutinin (DBA) positive collecting ducts (FIGS. 39B and 39D) in kidneys of mice with the indicated genotypes at 18 weeks. All mice received doxycycline from P28-42. The percentage of EdU and Ki67 positive nuclei was determined by counting at least 1,000 LTA or DBA positive nuclei marked by Hoechst 33342 (blue) in each mouse (n=3 mice). Multiple-group comparisons were performed by one-way ANOVA followed by Tukey's multiple-comparison test and data are presented as mean±s.e.m. Scale bars, 150 μm.

FIGS. 40A-40E demonstrate that inducible inactivation of Glis2 does not result in senescence in kidney epithelial cells, in accordance with some embodiments. FIGS. 40A-40E Representative bright-field microscopy images of kidney sections showing senescence-associated β-galactosidase (SA-β-Gal) activity with the specified genotypes at 18 weeks with lower (upper panel) and higher (lower panel) magnification. Blue staining indicative of cellular senescence is present in the cortical region of germline null Glis2^{ΔEx3/ΔEx3} kidneys (FIG. 40B) but is absent from kidneys of Glis2^{fl/fl}; Pkd1^{fl/fl}; Pax8^{rtTA}, TetO^{Cre} and Gli2^{fl/fl}; Pax8^{rtTA}; TetO^{Cre} kidneys (FIGS. 40B and 40E). Occasional cysts showing evidence of cellular senescence as observed in cystic Pkd1^{fl/fl}; Pax8^{rtTA}; TetO^{Cre} kidneys (FIG. 40C; boxed region is shown at higher magnification in lower panel). Histological sections from two mice for each genotype were examined to identify representative images. Scale bars: 500 μm (upper panel); 250 μm (lower panel).

FIGS. 41A-41B: Images of all the kidney histological sections used in FIGS. 20M-20P, in accordance with some embodiments. Pairs of kidneys from for each mouse are shown and each kidney is used as a separate datapoint in the cystic index data in FIG. 20M due to the variation in cyst burden between two kidneys from the same mouse in the Pkd2^{WS25/-} models. Scale bar, 1 mm

FIGS. 42A-42B demonstrate that UBC^{CreERT2} is active only in proximal tubule in mouse kidneys, in accordance with some embodiments. FIGS. 42A-42B, Colocalization of Cre activity (mEGFP) with proximal tubule marker megalin. Mice with UBC^{Cre/ERT2} and the Cre reporter ROSA^{mT/mG} allele (UBC^{Cre/ERT2}; ROSA^{mT/mG}) received tamoxifen daily from P28-35 and kidney tissue was examined at P35. Segments with Cre activity are marked by mEGFP (green); absence of Cre activity is marked by mtdTomato (red). Proximal tubules are marked by anti-megalin antibody staining (blue). There is complete overlap between EGFP and anti-megalin indicating that UBC^{Cre/ERT2} is only active in the proximal tubule in the kidney. Image of representative of sections from 3 independent mice; mice aged past P35 show the same pattern (data not shown). Scale bars: 400 μm (FIG. 42A); 25 μm (FIG. 42B).

FIGS. 43A-43B: Images of all the kidney histological sections used in FIGS. 21A-21N. Genotypes, treatments and sexes for each kidney are shown at the top. Scale bar, 1 mm.

FIGS. 44A-44F: Aggregate quantitative data in FIGS. 21B-21D separated by sex, in accordance with some embodiments. FIGS. 44A-44F, Aggregate quantitative data for the indicated parameters in male (FIGS. 44A-44C) and female (FIGS. 44D-44F) mice. n, number of mice in each group. Colors and symbol shapes correspond to indicated genotypes and treatment groups. Multiple-group comparisons are done by one-way ANOVA followed by Tukey's multiple-comparison test presented as mean±s.e.m.

FIGS. 45A-45B: Approach to quantifying Glis2 mRNA expression in proximal tubules, in accordance with some embodiments. FIGS. 45A-45B, Representative images showing the workflow and key steps in masking strategies used for quantitation in both smFISH (FIG. 45A) and RNAScope-FISH (FIG. 45B). A mask showing the regions of interest (ROI) was created solely based on the megalin (Lrp2) channel (red). The resulting mask was applied to the Glis2 channel (green). Signals in areas outside the ROI in the Glis2 channel were removed. The Glis2 signals remaining in the ROT were quantified with Cell Profiler. DAPT stained nuclei in the ROT were also counted by Cell Profiler. Scale bar, 50 μm.

FIGS. 46A-46D demonstrate that Glis2 ASO reduces cellular senescence in adult onset ADPKD, in accordance with some embodiments. FIGS. 46A-46D, Representative bright-field microscopy images of kidney sections showing senescence-associated β galactosidase (SA-β-Gal) activity with the specified genotypes and treatments at 18 weeks age with lower (upper panel) and higher (lower panel) magnification. FIGS. 46A-46B, Blue staining indicative of cellular senescence is absent from noncystic kidneys, including those treated with Glis2-ASO (FIG. 46B). Evidence of cellular senescence is present sporadically in polycystic Pkd1^{fl/fl}; UBC^{Cre-ERT2} kidneys treated with control-ASO (FIG. 46C) and is qualitatively reduced following treatment with Glis2-ASO (FIG. 46D). At least one histological section from two mice for each genotype and treatment group were examined to identify representative images. Boxed regions in upper image are shown in the respective lower panels. Scale bars: 500 μm (upper panels); 250 μm (lower panels).

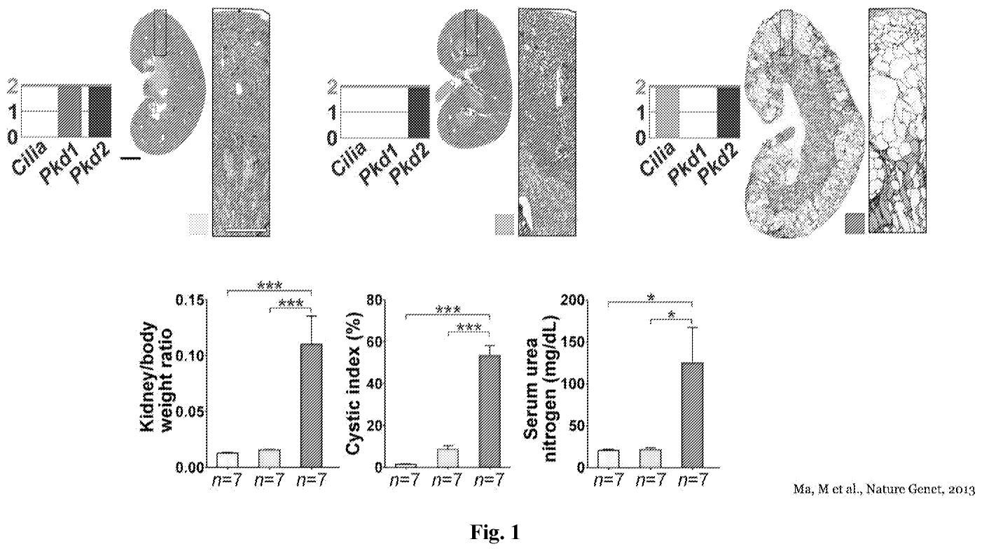

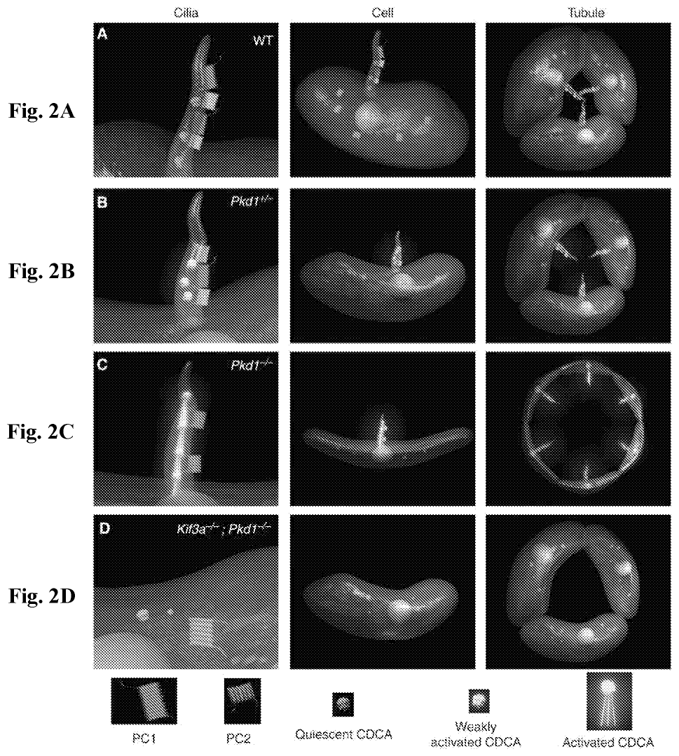

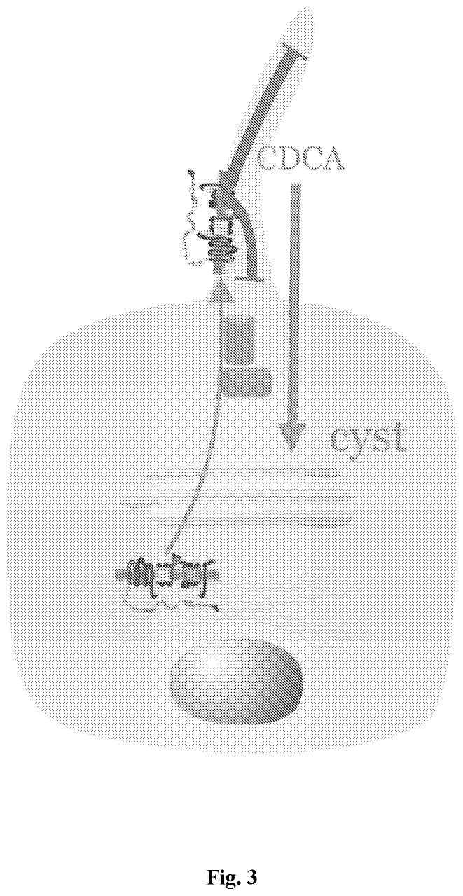

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

ADPKD is caused by mutations in either of two genes, PKD1 or PKD2, which result in similar disease features but with greater severity and earlier onset associated primarily with PKD1. These genes encode the respective proteins polycystin-1 (PC1) and polycystin-2 (PC2) whose precise biological function remains largely unknown.

A feature of these two proteins is that they are expressed on primary cilia. Primary cilia are solitary minute sensory organelles protruding from the apical surface of many cell types including kidney tubule cells from which they protrude into the lumen of the nephron. PC1 has structural features suggestive of a receptor protein and PC2 is a member of a cation channel family; together they are thought to form a receptor channel complex on the primary cilia but the precise molecular functions of this complex are unknown. From a genetic standpoint, although ADPKD is inherited as a dominant (heterozygous) trait, the initiation of cyst growth often follows somatic second hit mutations to the normal allele in individual cells.

The study described herein (“the present study”) identified GLIS2 as a potential downstream effector of PC1/PC2 in ADPKD. The present study further discovered that downregulating the expression of GLIS2, such as by genetic deletion or knockdown with antisense oligonucleotide (ASO) compounds counteracts the effects of Pkd1 and Pkd2 downregulation, and resulted in reduced progression of ADPKD.

It is worth noting that although Lu et al. (Kidney Int. 2016 June; 89(6):1307-23) describes that genetic ablation of GLIS2 is able to partially rescue the cystic phenotype in kidneys of Kif3a knockout mice, since no Kif3a mutations have been found in ADPKD patients, GLIS2 was not known to be involved in ADPKD pathogenesis.

Here, the present study discovered GLIS2 in an unbiased screen and validated in several pertinent biological models. The present study further provides validation that the increased GLIS2 protein in nucleus is a feature of PKD gene inactivation in vitro which is the first in vivo discovered molecular attribute of ADPKD that is clearly reproducible in in vitro cell-based models, and that the inactivation of GLIS2 through either genetic deletion or ASO small molecule therapy reduces cyst progression significantly in orthologous animal models.

It is envisioned that GLIS2 is a promising target. Patients homozygous for loss of function of GLIS2 are viable and have no manifestations outside of the kidney (Attanasio et al., Nat. Genet. 39 (8), 1018-1024, 2007; Halbritter et al., Hum. Genet. 132 (8), 865-884, 2013; and Al-Alawi et al., Front. Genet., 30 Nov. 2021). As such, down-regulating GLIS2 in patients is unlikely to cause side-effects outside kidney, and the delivery of agents that down-regulate GLIS2 does not have to be tissue specific.

Accordingly, in some embodiments, the present invention is directed to a method of treating, ameliorating and/or preventing a polycystic kidney disease, such as an autosomal dominant polycystic kidney disease, such as a polycystic kidney disease caused by or involves a mutation in the PKD1 gene or the PKD2 gene.

In some embodiments, the present invention is directed to a kit for treating, ameliorating and/or preventing a polycystic kidney disease, such as an autosomal dominant polycystic kidney disease, such as a polycystic kidney disease caused by or involves a mutation in the PKD1 gene or the PKD2 gene.

Furthermore, the present study discovered that the readout of increased GLIS2 expression in the nuclei or the opposite, reduction of GLIS2 expression in nuclei, could be a basis for high throughput screen for suppressors of the downstream effects of loss of PKDs. Such suppressors can then be tested as potential treatment for polycystic kidney disease.

Accordingly, in some embodiments, the present invention is directed to a method of screening suppressors of the downstream effects of loss of PKD1 or PKD2.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “GLIS2” refers to the protein having the amino acid sequence set forth in SEQ ID NO 1, the gene that encodes the protein having the amino acid sequence set forth in SEQ ID NO 1, genes that occupy the same allele as the gene encoding the protein having the amino acid sequence set forth in SEQ ID NO 1 in the human genome, all protein products thereof, as well as the orthologs of the human proteins or genes in other species.

SEQ ID NO 1: Zinc finger protein GLIS2 [Homo sapiens]
MHSLDEPLDLKLSITKLRAAREKRERTLGVVRPRALHRELGLVDDSPTPGSPGSPPSGF
LLNSKFPEKVEGRFSAAPLVDLSLSPPSGLDSPNGSSSLSPERQGNGDLPPVPSASDFQP
LRYLDGVPSSFQFFLPLGSGGALHLPASSFLTPPKDKCLSPDLPLPKQLVCRWAKCNQ
LFELLQDLVDHVNDYHVKPEKDAGYCCHWEGCARHGRGFNARYKMLIHIRTHTNEK
PHRCPTCSKSFSRLENLKIHNRSHTGEKPYVCPYEGCNKRYSNSSDRFKHTRTHYVDK
PYYCKMPGCHKRYTDPSSLRKHIKAHGHFVSHEQQELLQLRPPPKPPLPAPDGGPYVS
GAQIIIPNPAALFGGPGLPGLPLPLAPGPLDLSALACGNGGGSGGGGGMGPGLPGPVLP
LNLAKNPLLPSPFGAGGLGLPVVSLLAGAAGGKAEGEKGRGSVPTRALGMEGHKTPL
ERTESSCSRPSPDGLPLLPGTVLDLSTGVNSAASSPEALAPGWVVIPPGSVLLKPAVVN

As used herein, the term “Pkd1” refers to the protein having the amino acid sequence set forth in SEQ ID NO 2, the gene that encodes the protein having the amino acid sequence set forth in SEQ ID NO 2, genes that occupy the same allele as the gene encoding the protein having the amino acid sequence set forth in SEQ ID NO 2 in the human genome, all protein products thereof, as well as the orthologs of the human proteins or genes in other species. The specification sometimes refer to the protein products as polycystin 1 or PC1.

SEQ ID NO 2: Polycystin 1 (PC1) protein [Homo sapiens]
MPPAAPARLALALGLGLWLGALAGGPGRGCGPCEPPCLCGPAPGAACRVNCSGRGL
RTLGPALRIPADATALDVSHNLLRALDVGLLANLSALAELDISNNKISTLEEGIFANLF
NLSEINLSGNPFECDCGLAWLPRWAEEQQVRVVQPEAATCAGPGSLAGQPLLGIPLLD
SGCGEEYVACLPDNSSGTVAAVSFSAAHEGLLQPEACSAFCFSTGQGLAALSEQGWC
LCGAAQPSSASFACLSLCSGPPPPPAPTCRGPTLLQHVFPASPGATLVGPHGPLASGQL
AAFHIAAPLPVTATRWDFGDGSAEVDAAGPAASHRYVLPGRYHVTAVLALGAGSAL
LGTDVQVEAAPAALELVCPSSVQSDESLDLSIQNRGGSGLEAAYSIVALGEEPARAVH
PLCPSDTEIFPGNGHCYRLVVEKAAWLQAQEQCQAWAGAALAMVDSPAVQRFLVSR
VTRSLDVWIGFSTVQGVEVGPAPQGEAFSLESCQNWLPGEPHPATAEHCVRLGPTGW
CNTDLCSAPHSYVCELQPGGPVQDAENLLVGAPSGDLQGPLTPLAQQDGLSAPHEPV
EVMVFPGLRLSREAFLTTAEFGTQELRRPAQLRLQVYRLLSTAGTPENGSEPESRSPDN
RTQLAPACMPGGRWCPGANICLPLDASCHPQACANGCTSGPGLPGAPYALWREFLFS
VPAGPPAQYSVTLHGQDVLMLPGDLVGLQHDAGPGALLHCSPAPGHPGPRAPYLSA
NASSWLPHLPAQLEGTWACPACALRLLAATEQLTVLLGLRPNPGLRLPGRYEVRAEV
GNGVSRHNLSCSFDVVSPVAGLRVIYPAPRDGRLYVPTNGSALVLQVDSGANATATA
RWPGGSVSARFENVCPALVATFVPGCPWETNDTLFSVVALPWLSEGEHVVDVVVEN
SASRANLSLRVTAEEPICGLRATPSPEARVLQGVLVRYSPVVEAGSDMVFRWTINDKQ
SLTFQNVVFNVIYQSAAVFKLSLTASNHVSNVTVNYNVTVERMNRMQGLQVSTVPA
VLSPNATLALTAGVLVDSAVEVAFLWTFGDGEQALHQFQPPYNESFPVPDPSVAQVL
VEHNVMHTYAAPGEYLLTVLASNAFENLTQQVPVSVRASLPSVAVGVSDGVLVAGR
PVTFYPHPLPSPGGVLYTWDFGDGSPVLTQSQPAANHTYASRGTYHVRLEVNNTVSG
AAAQADVRVFEELRGLSVDMSLAVEQGAPVVVSAAVQTGDNITWTFDMGDGTVLS
GPEATVEHVYLRAQNCTVTVGAASPAGHLARSLHVLVFVLEVLRVEPAACIPTQPDA
RLTAYVTGNPAHYLFDWTFGDGSSNTTVRGCPTVTHNFTRSGTFPLALVLSSRVNRA
HYFTSICVEPEVGNVTLQPERQFVQLGDEAWLVACAWPPFPYRYTWDFGTEEAAPTR
ARGPEVTFIYRDPGSYLVTVTASNNISAANDSALVEVQEPVLVTSIKVNGSLGLELQQP
YLFSAVGRGRPASYLWDLGDGGWLEGPEVTHAYNSTGDFTVRVAGWNEVSRSEAW
LNVTVKRRVRGLVVNASRTVVPLNGSVSFSTSLEAGSDVRYSWVLCDRCTPIPGGPTI
SYTFRSVGTFNIIVTAENEVGSAQDSIFVYVLQLIEGLQVVGGGRYFPTNHTVQLQAV
VRDGTNVSYSWTAWRDRGPALAGSGKGFSLTVLEAGTYHVQLRATNMLGSAWADC
TMDFVEPVGWLMVAASPNPAAVNTSVTLSAELAGGSGVVYTWSLEEGLSWETSEPF
TTHSFPTPGLHLVTMTAGNPLGSANATVEVDVQVPVSGLSIRASEPGGSFVAAGSSVP
FWGQLATGTNVSWCWAVPGGSSKRGPHVTMVFPDAGTFSIRLNASNAVSWVSATYN
LTAEEPIVGLVLWASSKVVAPGQLVHFQILLAAGSAVTFRLQVGGANPEVLPGPRFSH
SFPRVGDHVVSVRGKNHVSWAQAQVRIVVLEAVSGLQVPNCCEPGIATGTERNFTAR
VQRGSRVAYAWYFSLQKVQGDSLVILSGRDVTYTPVAAGLLEIQVRAFNALGSENRT
LVLEVQDAVQYVALQSGPCFTNRSAQFEAATSPSPRRVAYHWDFGDGSPGQDTDEPR
AEHSYLRPGDYRVQVNASNLVSFFVAQATVTVQVLACREPEVDVVLPLQVLMRRSQ
RNYLEAHVDLRDCVTYQTEYRWEVYRTASCQRPGRPARVALPGVDVSRPRLVLPRL
ALPVGHYCFVFVVSFGDTPLTQSIQANVTVAPERLVPIIEGGSYRVWSDTRDLVLDGS
ESYDPNLEDGDQTPLSFHWACVASTQREAGGCALNFGPRGSSTVTIPRERLAAGVEYT
FSLTVWKAGRKEEATNQTVLIRSGRVPIVSLECVSCKAQAVYEVSRSSYVYLEGRCLN
CSSGSKRGRWAARTFSNKTLVLDETTTSTGSAGMRLVLRRGVLRDGEGYTFTLTVLG
RSGEEEGCASIRLSPNRPPLGGSCRLFPLGAVHALTTKVHFECTGWHDAEDAGAPLVY
ALLLRRCRQGHCEEFCVYKGSLSSYGAVLPPGFRPHFEVGLAVVVQDQLGAAVVAL
NRSLAITLPEPNGSATGLTVWLHGLTASVLPGLLRQADPQHVIEYSLALVTVLNEYER
ALDVAAEPKHERQHRAQIRKNITETLVSLRVHTVDDIQQIAAALAQCMGPSRELVCRS
CLKQTLHKLEAMMLILQAETTAGTVTPTAIGDSILNITGDLIHLASSDVRAPQPSELGA
ESPSRMVASQAYNLTSALMRILMRSRVLNEEPLTLAGEEIVAQGKRSDPRSLLCYGGA
PGPGCHFSIPEAFSGALANLSDVVQLIFLVDSNPFPFGYISNYTVSTKVASMAFQTQAG
AQIPIERLASERAITVKVPNNSDWAARGHRSSANSANSVVVQPQASVGAVVTLDSSNP
AAGLHLQLNYTLLDGHYLSEEPEPYLAVYLHSEPRPNEHNCSASRRIRPESLQGADHR
PYTFFISPGSRDPAGSYHLNLSSHFRWSALQVSVGLYTSLCQYFSEEDMVWRTEGLLP
LEETSPRQAVCLTRHLTAFGASLFVPPSHVRFVFPEPTADVNYIVMLTCAVCLVTYMV
MAAILHKLDQLDASRGRAIPFCGQRGRFKYEILVKTGWGRGSGTTAHVGIMLYGVDS
RSGHRHLDGDRAFHRNSLDIFRIATPHSLGSVWKIRVWHDNKGLSPAWFLQHVIVRD
LQTARSAFFLVNDWLSVETEANGGLVEKEVLAASDAALLRFRRLLVAELQRGFFDKH
IWLSIWDRPPRSRFTRIQRATCCVLLICLFLGANAVWYGAVGDSAYSTGHVSRLSPLS
VDTVAVGLVSSVVVYPVYLAILFLFRMSRSKVAGSPSPTPAGQQVLDIDSCLDSSVLD
SSFLTFSGLHAEQAFVGQMKSDLFLDDSKSLVCWPSGEGTLSWPDLLSDPSIVGSNLR
QLARGQAGHGLGPEEDGFSLASPYSPAKSFSASDEDLIQQVLAEGVSSPAPTQDTHME
TDLLSSLSSTPGEKTETLALQRLGELGPPSPGLNWEQPQAARLSRTGLVEGLRKRLLPA
WCASLAHGLSLLLVAVAVAVSGWVGASFPPGVSVAWLLSSSASFLASFLGWEPLKVL
LEALYFSLVAKRLHPDEDDTLVESPAVTPVSARVPRVRPPHGFALFLAKEEARKVKRL
HGMLRSLLVYMLFLLVTLLASYGDASCHGHAYRLQSAIKQELHSRAFLAITRSEELWP
WMAHVLLPYVHGNQSSPELGPPRLRQVRLQEALYPDPPGPRVHTCSAAGGFSTSDYD
VGWESPHNGSGTWAYSAPDLLGAWSWGSCAVYDSGGYVQELGLSLEESRDRLRFLQ
LHNWLDNRSRAVFLELTRYSPAVGLHAAVTLRLEFPAAGRALAALSVRPFALRRLSA
GLSLPLLTSVCLLLFAVHFAVAEARTWHREGRWRVLRLGAWARWLLVALTAATALV
RLAQLGAADRQWTRFVRGRPRRFTSFDQVAQLSSAARGLAASLLFLLLVKAAQQLRF
VRQWSVFGKTLCRALPELLGVTLGLVVLGVAYAQLAILLVSSCVDSLWSVAQALLVL
CPGTGLSTLCPAESWHLSPLLCVGLWALRLWGALRLGAVILRWRYHALRGELYRPA
WEPQDYEMVELFLRRLRLWMGLSKVKEFRHKVRFEGMEPLPSRSSRGSKVSPDVPPP
SAGSDASHPSTSSSQLDGLSVSLGRLGTRCEPEPSRLQAVFEALLTQFDRLNQATEDV
YQLEQQLHSLQGRRSSRAPAGSSRGPSPGLRPALPSRLARASRGVDLATGPSRTPLRA
KNKVHPSST

As used herein, the term “Pkd2” refers to the protein having the amino acid sequence set forth in SEQ ID NO 3, the gene that encodes the protein having the amino acid sequence set forth in SEQ ID NO 3, genes that occupy the same allele as the gene encoding the protein having the amino acid sequence set forth in SEQ TD NO 3 in the human genome, all protein products thereof, as well as the orthologs of the human proteins or genes in other species. The specification sometimes refer to the protein products as polycystin 2 or PC2.

SEQ ID NO3: Polycystin-2 (PC2) [Homo sapiens]
MVNSSRVQPQQPGDAKRPPAPRAPDPGRLMAGCAAVGASLAAPGGLCEQRGLEIEM
QRIRQAAARDPPAGAAASPSPPLSSCSRQAWSRDNPGFEAEEEEEEVEGEEGGMVVE
MDVEWRPGSRRSAASSAVSSVGARSRGLGGYHGAGHPSGRRRRREDQGPPCPSPVG
GGDPLHRHLPLEGQPPRVAWAERLVRGLRGLWGTRLMEESSTNREKYLKSVLRELVT
YLLFLIVLCILTYGMMSSNVYYYTRMMSQLFLDTPVSKTEKTNFKTLSSMEDFWKFTE
GSLLDGLYWKMQPSNQTEADNRSFIFYENLLLGVPRIRQLRVRNGSCSIPQDLRDEIKE
CYDVYSVSSEDRAPFGPRNGTAWIYTSEKDLNGSSHWGIIATYSGAGYYLDLSRTREE
TAAQVASLKKNVWLDRGTRATFIDFSVYNANINLFCVVRLLVEFPATGGVIPSWQFQP
LKLIRYVTTFDFFLAACEIIFCFFIFYYVVEEILEIRIHKLHYFRSFWNCLDVVIVVLSVV
AIGINIYRTSNVEVLLQFLEDQNTFPNFEHLAYWQIQFNNIAAVTVFFVWIKLFKFINFN
RTMSQLSTTMSRCAKDLFGFAIMFFIIFLAYAQLAYLVFGTQVDDFSTFQECIFTQFRII
LGDINFAEIEEANRVLGPIYFTTFVFFMFFILLNMFLAIINDTYSEVKSDLAQQKAEMEL
SDLIRKGYHKALVKLKLKKNTVDDISESLRQGGGKLNFDELRQDLKGKGHTDAEIEAI
FTKYDQDGDQELTEHEHQQMRDDLEKEREDLDLDHSSLPRPMSSRSFPRSLDDSEED
DDEDSGHSSRRRGSISSGVSYEEFQVLVRRVDRMEHSIGSIVSKIDAVIVKLEIMERAK
LKRREVLGRLLDGVAEDERLGRDSEIHREQMERLVREELERWESDDAASQISHGLGTP
VGLNGQPRPRSSRPSSSQSTEGMEGAGGNGSSNVHV

It should be noted that, although in the art human genes are often named with all capital letters (e.g., “GLIS2”) while mouse genes are often named with only the first letter being capital letter (e.g., “Glis2”), the instant specification does not strictly follow this rule. Gene/protein names such as “GLIS2” and “Glis2” are used interchangeably herein and do not indicate the original species.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Abbreviations: ADPKD: autosomal dominant polycystic kidney disease. PKD: polycystic kidney disease. Doxy: doxycycline. DT: diphtheria toxin.

Method of Treating/Ameliorating and/or Preventing Polycystic Kidney Disease

In some aspects, the present invention is directed to a method of treating, ameliorating, and/or preventing a polycystic kidney disease in a subject in need thereof.

In some embodiments, the polycystic kidney disease is an autosomal dominant polycystic kidney disease (ADPKD).

In some embodiments, the polycystic kidney disease is caused by or involves a mutant PKD1 gene or a mutant PKD2 gene.

In some embodiments, the subject is a mammal, such as a human.

In some embodiments, the method includes administering to the subject an effective amount of a compound that downregulates GLIS2. As used herein, the term “downregulating GLIS2” means reducing the expression level of the protein and/or the mRNA of GLIS2, or reducing the activity of the GLIS2 protein.

As detailed elsewhere herein, the expression level of GLIS2 can be downregulated at the genomic level, such as by disrupting the genomic DNA sequences that encodes GLIS2; at the transcriptional level, such as by reducing the mRNA level of GLIS2; at the translational level, such as by reducing the binding affinity between the GLIS2 mRNA and the ribosome, or promoting the detachment of ribosome from the GLIS2 mRNA; or at the post-translational level, such as promoting the degradation of Glis2 protein using a neutralizing antibody.

As detailed elsewhere herein, the activity of GLIS2 protein can be reduced by, for example, GLIS2 specific antibodies.

In some embodiments, the compound that downregulates GLIS2 includes a protein inhibitor of GLIS2; a nucleic acid that down regulates the expression level and/or the activity of GLIS2 by RNA interference, and/or an expression vector expressing the nucleic acid that down regulates the expression level and/or the activity of GLIS2 by RNA interference; a ribozyme that down regulates the expression level and/or the activity of GLIS2, and/or an expression vector expressing the ribozyme, an expression vector comprising an expression cassette, wherein the expression cassette expresses CRISPR components that down regulates the expression level and/or the activity of GLIS2 by CRISPR knockout, and a trans-dominant negative mutant protein of GLIS2, and/or an expression vector that expresses the trans-dominant negative mutant protein of GLIS2.

### Downregulating GLIS2 by Protein Inhibitors of GLIS2

In some embodiments, the compound that downregulates the expression level or the activity of GLIS2 includes a protein that downregulates GLIS2.

In some embodiments, the protein that downregulates GLIS2 includes a monoclonal antibody, a polyclonal antibody, antigen binding fragments thereof, and combinations thereof. One of ordinary skill in the art would understand that, since GLIS2 protein is intracellular in nature, modifications of the antibodies or fragments are sometimes needed to make the antibodies cell-penetrating. Such modifications are described in, for example, Fonseca et al. (Advanced Drug Delivery Reviews Volume 61, Issue 11, 30 Sep. 2009, Pages 953-964) and Muller et al. (Expert Opinion on Biological Therapy Volume 5, 2005—Issue 2).

Examples of monoclonal and/or polyclonal antibodies that target GLIS2 include 9HCLC and OTI1F9 by Abcam, PA5-72849 by Invetrogen, NBP2-41311 by Novus Biologicals, and any humanized derivatives thereof.

In some embodiments, the protein that downregulates the expression level and/or activity of GLIS2 is administered in the form of a protein. In some embodiments, the protein that downregulates the expression level and/or activity of GLIS2 is administered in form of a nucleic acid that expresses the protein, such as an expression vector. The expression vector is described in the “Vector” section elsewhere in the instant specification.

### Downregulating GLIS2 by RNA Interference

In some embodiments, the compound that downregulates the activity or expression level of GLIS2 includes a nucleic acid that downregulates GLIS2 by the means of RNA interreference.

In some embodiments, the nucleic acid that downregulates the expression level of GLIS2 by the means of RNA interreference includes an isolated nucleic acid. In other embodiments, the modulator is an RNAi molecule (such as but not limited to siRNA and/or shRNA and/or miRNAs) or antisense molecule, which inhibits GLIS2 expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of GLIS2. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of GLIS2 using RNAi technology.

In certain embodiments, the instant specification provides a vector comprising an siRNA or antisense polynucleotide (such as an antisense oligonucleotide or ASO). In other embodiments, the siRNA or antisense polynucleotide inhibits the expression of GLIS2. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In certain embodiments, an antisense nucleic acid sequence expressed by a plasmid vector is used to inhibit GLIS2 protein expression. The antisense expressing vector is used to transfect a mammalian cell or the mammal itself, thereby causing reduced endogenous expression of GLIS2.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

In various embodiments, the an antisense oligonucleotide (ASO) comprises at least a portion of the 5′-CCTTATAAGCTTCTGC-3′ (SEQ ID NO 164). In various embodiments, the ASO comprises at least 5, at least 6, at least 7, at least 8, at least 9, at least 10, at least 11, at least 12, at least 13, at least 14, at least 15 or all 16 contiguous nucleotides of SEQ ID NO 164.

### Downregulating GLIS2 by Ribozyme

In some embodiments, the compound that down regulates the activity or expression level of GLIS2 includes a ribosome that inhibits GLIS2 protein expression.

A ribozyme is used to inhibit GLIS2 protein expression. Ribozymes useful for inhibiting the expression of a target molecule may be designed by incorporating target sequences into the basic ribozyme structure which are complementary, for example, to the mRNA sequence encoding GLIS2. Ribozymes are antisense RNAs which have a catalytic site capable of specifically cleaving complementary RNAs. Therefore, ribozymes having sequence complementary to GLIS2 mRNA sequences are capable of downregulating the expression of GLIS2 by reduces the level of GLIS2 mRNA. Ribozymes targeting GLIS2, may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

### Downregulating GLIS2 by CRISPR Knockout and Other Knockouts/Knockdown Techniques

In some embodiments, the compound that down regulates the activity or expression level of GLIS2 comprises a nucleic acid that down regulates the expression level of GLIS2 by the means of CRISPR knockout.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and an HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the present invention includes any other methods for effecting gene knockdown and/editing, which allow for deletion and/or inactivation of GLIS2, such as but not limited to those described in WO 2018/236840 (which is incorporated herein in its entirety by reference).

Downregulating GLIS2 by Inactivating and/or Sequestering

In some embodiments, the compound that downregulates GLIS2 includes a protein that downregulates the activity of GLIS2 by inactivating and/or sequestering GLIS2. In some embodiment, the compound includes a nucleic acid that expresses the protein that downregulates the activity of GLIS2 by inactivating and/or sequestering GLIS2. In some embodiments, the compound includes an expression vector that expresses the protein that downregulates the activity of GLIS2 by inactivating and/or sequestering GLIS2 (see “Vector” section for descriptions on vectors).

In some embodiments, the compound that downregulates GLIS2 is a trans-dominant negative mutant of GLIS2, and/or a nucleic acid or a vector expressing the trans-dominant negative mutant of GLIS2.

Composition for Treating, Ameliorating and/or Preventing Polycystic Kidney Disease

In some aspects, the present invention is directed to a composition for treating, ameliorating, and/or preventing a polycystic kidney disease in a subject in need thereof.

In some embodiments, the polycystic kidney disease is an autosomal dominant polycystic kidney disease (ADPKD).

In some embodiments, the polycystic kidney disease is caused by or involves a mutant Pkd1 gene or a mutant Pkd2 gene.

In some embodiments, the subject is a mammal, such as a human.

In some embodiments, the composition includes a compound that downregulates GLIS2; and a pharmaceutically acceptable carrier.

In some embodiments, the compound that downregulates GLIS2 is the same as or similar to those described elsewhere herein, such as in the “Method of Treating/Ameliorating and/or Preventing Polycystic Kidney Disease” section.

Kit for Treating, Ameliorating and/or Preventing Polycystic Kidney Disease

In some aspects, the present invention is directed to a kit for treating, ameliorating, and/or preventing a polycystic kidney disease in a subject in need thereof.

In some embodiments, the polycystic kidney disease is an autosomal dominant polycystic kidney disease (ADPKD).

In some embodiments, the polycystic kidney disease is caused by or involves a mutant Pkd1 gene or a mutant Pkd2 gene.

In some embodiments, the subject is a mammal, such as a human.

In some embodiments, the kit includes a compound that downregulates GLIS2; and a manual instructing that the compound that downregulates GLIS2 is to be administered to a subject in need thereof in an effective amount.

In some embodiments, the compound that downregulates GLIS2 is the same as or similar to those described elsewhere herein, such as in the “Method of Treating/Ameliorating and/or Preventing Polycystic Kidney Disease” section.

### Method of Screening Suppressors of Downstream Effects of Loss of PKD1 or PKD2

In some aspects, the present invention is directed to a method of screening suppressors of downstream effects of loss of PKD1 or PKD2.

In some embodiments, the method includes: detecting a first GLIS2 expression level in the nucleus of a cell expressing PKD1 and PKD2; contacting the cell with a compound; and detecting a second GLIS2 expression level in the nucleus of the cell.

In some embodiments, a difference between the first GLIS2 expression level and the second GLIS2 expression level indicates that the compound is a suppressors of downstream effects of loss of PKD1 or PKD2.

In some embodiments, the cell is a kidney cell or a kidney-derived cell.

In some embodiments, the cell is a primary cell or a cell line.

In some embodiments, the cell is a mammalian cell or is derived from a mammalian cell. In some embodiments, the cell is a human cell or is derived from a human cell.

In some embodiments, that the second GLIS2 expression level is lower than the first GLIS2 expression level indicates that compound is a suppressors of downstream effects of loss of PKD1 or PKD2. In some embodiments, that the second GLIS2 expression level is higher than the first GLIS2 expression level indicates that compound is a suppressors of downstream effects of loss of PKD1 or PKD2.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that down regulates the activity or expression level of GLIS2 is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that downregulates GLIS2 described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the polycystic kidney disease includes administering to the subject the effective amount of at least one compound and/or composition contemplated within the disclosure.

In some embodiments, the composition for treating, ameliorating, and/or preventing the polycystic kidney disease includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating polycystic kidney disease.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of polycystic kidney disease in a patient.

Formulations may be employed in admixtures with conventional excipients, i.e., pharmaceutically acceptable organic or inorganic carrier substances suitable for intracranially, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the polycystic disease in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1-1: Transcriptome Analysis for Cilia-Dependent Cyst Activating (CDCA) Pathway Targets

The present study performed a targeted transcriptomic analysis using the translating ribosome affinity purification (TRAP) assay to identify mRNA molecules in cells that have lost the expressions of the polycystin proteins and are destined to become cysts. The analysis was done at an early stage following loss of polycystins so that cyst formation, and its attendant secondary changes, had not yet begun.

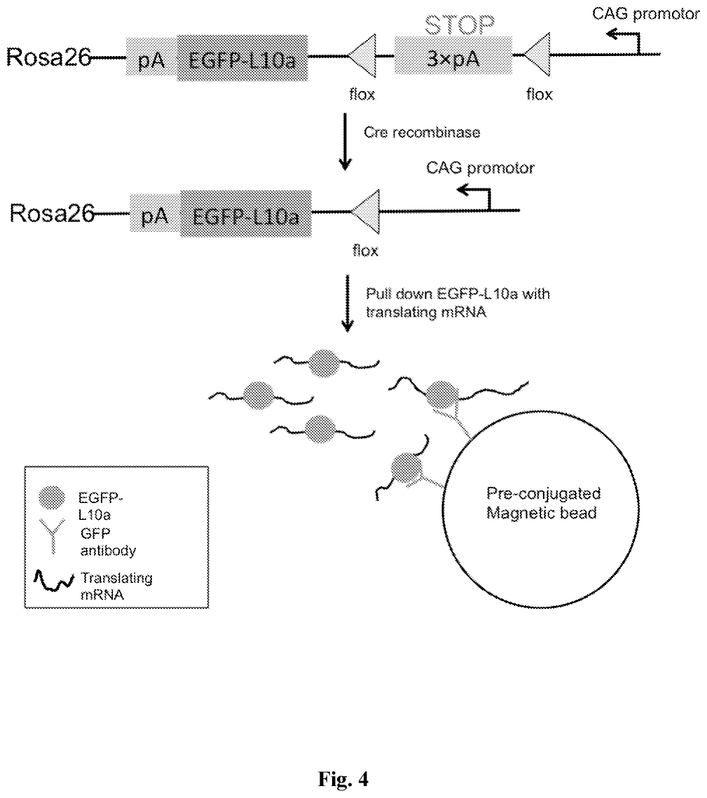

Referring to FIG. 4, to perform the TRAP assay, the 60S ribosomal protein L10a (RPL10A) was fused with EGFP (EGFP-L10a) and knocked-in into mice of different genotypes using Rosa26. The EGFP-L10a does not express in Cre-negative cells due to the presence of the STOP sequence. In Cre-positive cells, the STOP sequence is removed by recombination and EGFP-L10a is expressed. EGFP-L10a was then pulled-down together with the translating mRNA molecules in binding with L10a. This allows the mRNA molecules to be identified. This way, the in vivo profiling of selective cell populations from native tissues, and the profiling of the actively translating mRNA were achieved.

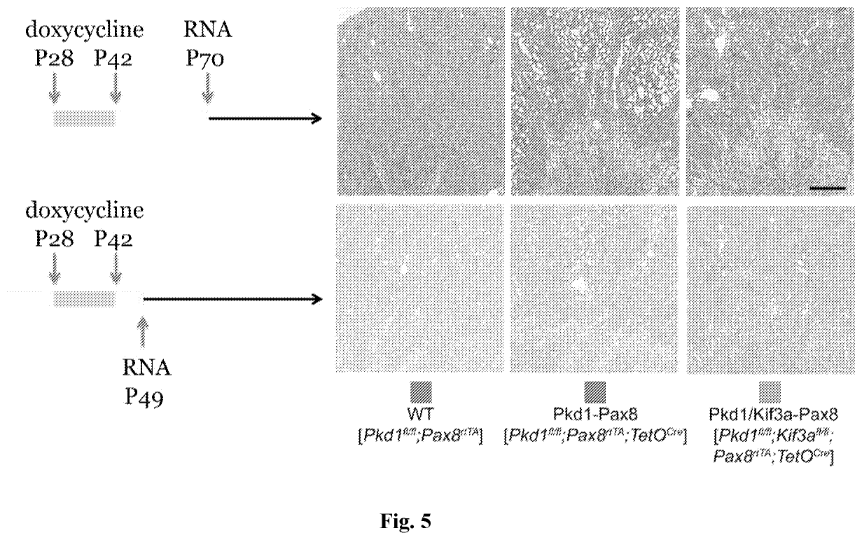

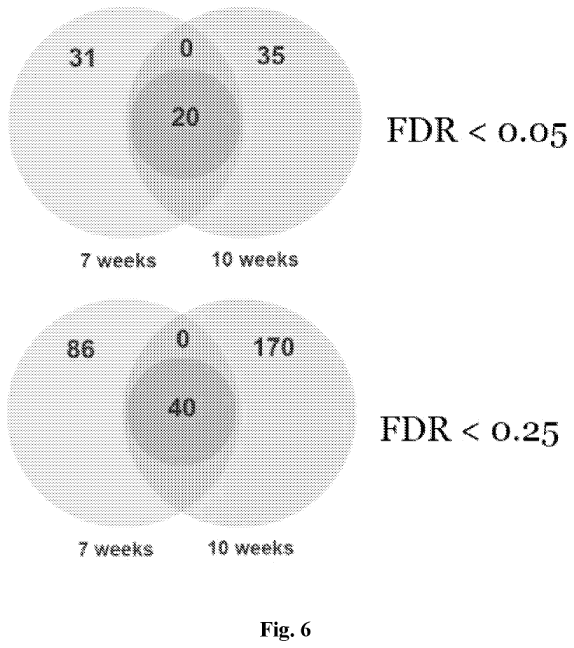

Referring to FIG. 5, the three different genotypes of mice were the noncystic controls (“WT”), Pkd1—only knockouts that are destined to form typical ADPKD (“Pkd1-Pax8”), and Pkd1-cilia double knockouts that will not form cysts despite the lack of PC l (“Pkd1/Kif3a-Pax8”). Genes that were significantly and specifically dysregulated in polycystin-only knockouts when compared to both noncystic and polycystin-cilia double knockouts were identified (FIG. 6).

In examining this unique transcriptional profile, the present study identified a transcription factor, GLIS2, that was significantly and consistently only upregulated in the Pkd1-only knockouts destined to form cysts.

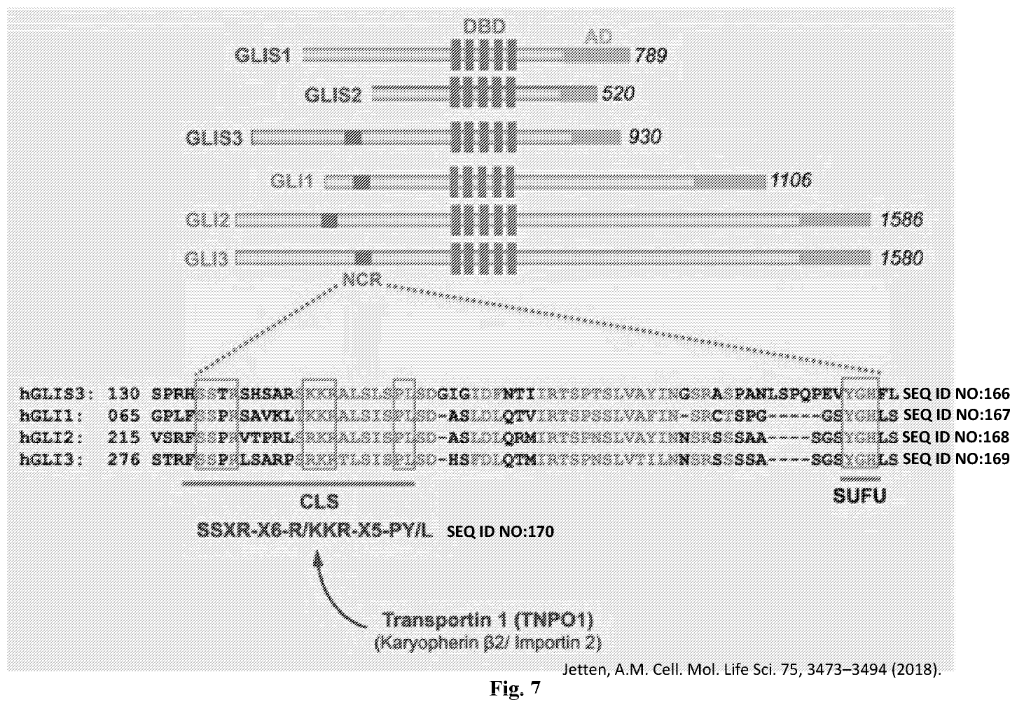

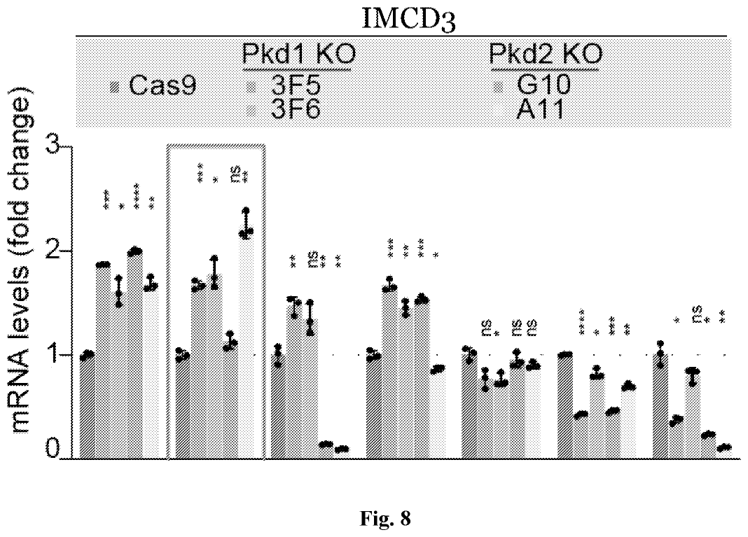

GLIS2 is a Krupple type zinc finger protein that has previously been implicated in a recessive fibrocystic kidney disease in humans called nephronophthisis and has been localized to cilia. This combination of upregulation in polycystin knockouts (but not, for example, polycystin-cilia double knockouts), localization to cilia, and implication in fibrocystic kidney diseases raised the possibility of this was a good candidate molecule for an effector acting in some portion of the CDCA pathway. Referring to FIG. 7, GLIS2 has similarity to the Gli family of transcription factors (the name GLIS is derived from ‘Gli similar’) which are the central transcription factors in the Hedgehog pathway.

Although literature claims GLIS2 is a cilia protein, the present study confirmed that GLIS2 is not expressed in cilia (data not shown). As such, the direct interaction between GLIS with the polycystins, which are located in cilia, is unlikely.

### Example 1-2: PCI (Protein Product of Pkd1) or PC2 (Protein Product of Pkd2) Inactivation Upregulates GLIS2 and Causes Nucleus Re-Localization in Epithelial Cell Culture Models

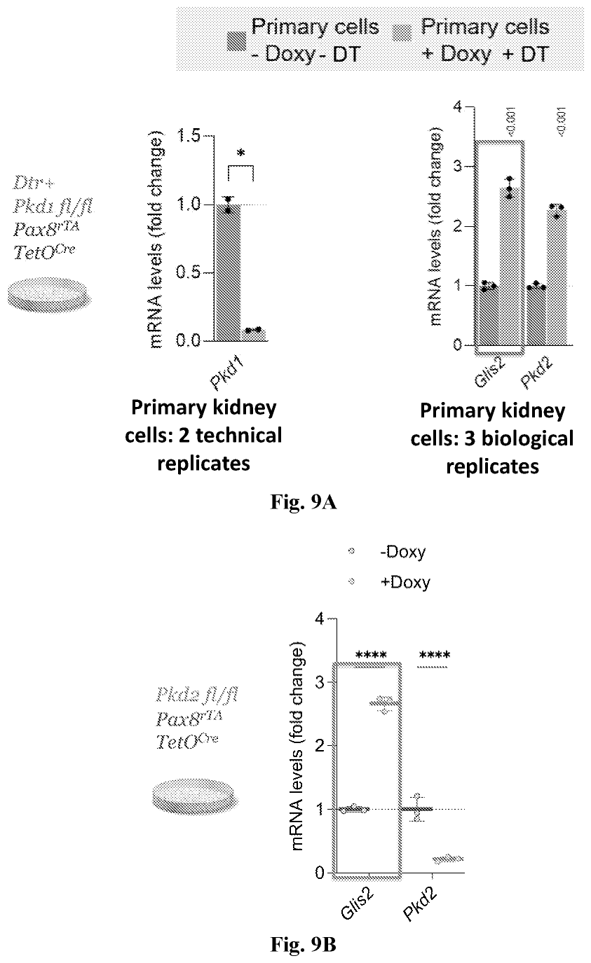

Referring to FIGS. 9A-9B, 10 and 11, the present study isolated primary kidney cells from Dtr+; Pkd1^{fl/fl}; Pax8^{rTA}, TetO^{Cre} knockout mice, which become Pkd1 knockout cells upon treatment with diphtheria toxin (DT) and doxycycline (Doxy), and primary kidney cells from Pkd2^{fl/fl}; Pax8^{rtTA}; TetO^{Cre}, which become Pkd2 knockout cells upon treatment with Doxy.

Referring to FIG. 9A, in comparison to the control cells (“−Doxy−DT”), the Pkd1 knockout (“+Doxy+DT”) primary kidney cells express significantly higher levels of GLIS2 mRNA.

Referring to FIG. 9B, in comparison to the control cells (“−Doxy”), the Pkd2 knockout (“+Doxy”) primary kidney cells express significantly higher levels of GLIS2.

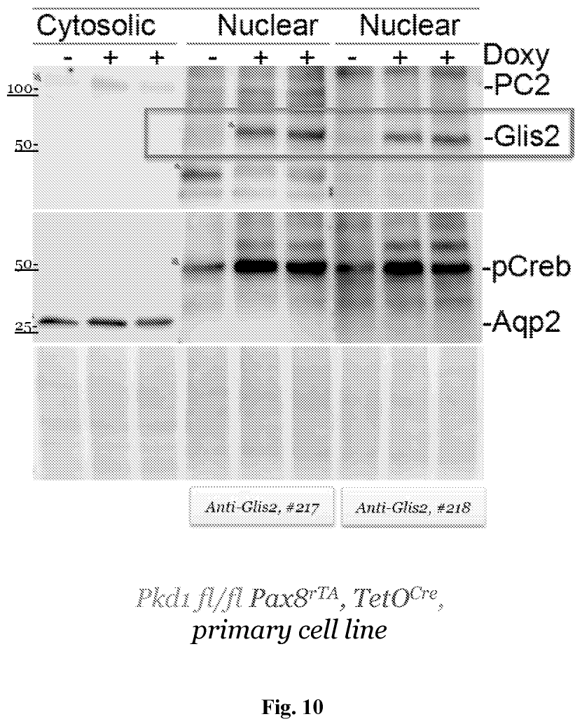

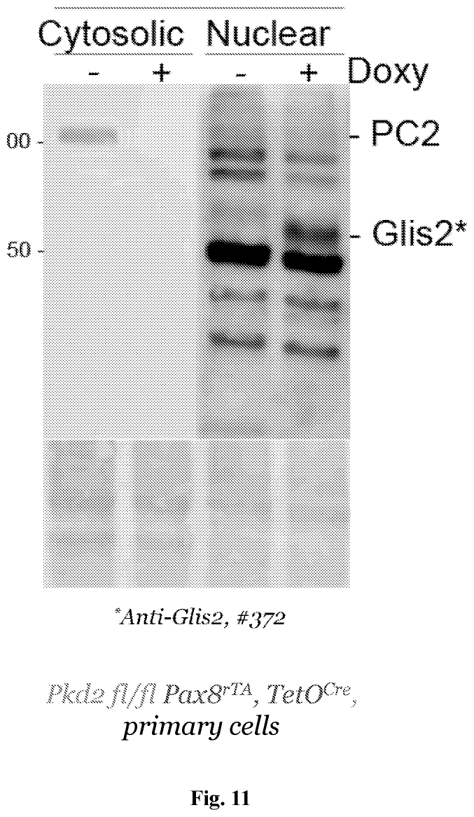

Referring to FIG. 10, knocking out Pkd1 (with doxycycline) causes the enrichment of GLIS2 protein in the nucleus of the primary cells. Referring to FIG. 11, knocking out Pkd2 (with doxycycline) causes the enrichment of GLIS2 protein in the nucleus of the primary cells, as well.

The in vitro read out for polycystin (protein products of Pkd1/2) inactivation has been a “holy grail” in the PKD field. The present study showed that cell models recapitulate the in vivo phenotype in that Pkd1 or Pkd2 knockouts have increased GLIS2 that expressed in nuclei and that Pkd1—cilia double knockouts in cell culture do not upregulated GLIS2. While the discovery of GLIS2 is based on Pkd1 mouse models, the dysregulation of GLIS2 occurs in either Pkd1 or Pkd2 knockout as would be expected in ADPKD. Furthermore, the readout of increased GLIS2 expression in the nuclei or the opposite, reduction of GLIS2 expression in nuclei, could be a basis for high throughput screen for suppressors of the downstream effects of loss of polycystins.

### Example 1-3: Pkd1 Inactivation Results in Increased GLIS2 Transcripts in Affected Nephron Segments In Vivo

The present study performed semiquantitative in situ hybridization in tissues to demonstrate that Pkd1 inactivation results in increased GLIS2 transcripts in affected nephron segments in vivo (data not shown).

### Example 1-4: Downregulation of GLIS2 in Mouse Model Kidneys Ameliorates the ADPKD Phenotype Caused by Pkd1 or Pkd2 Inactivation

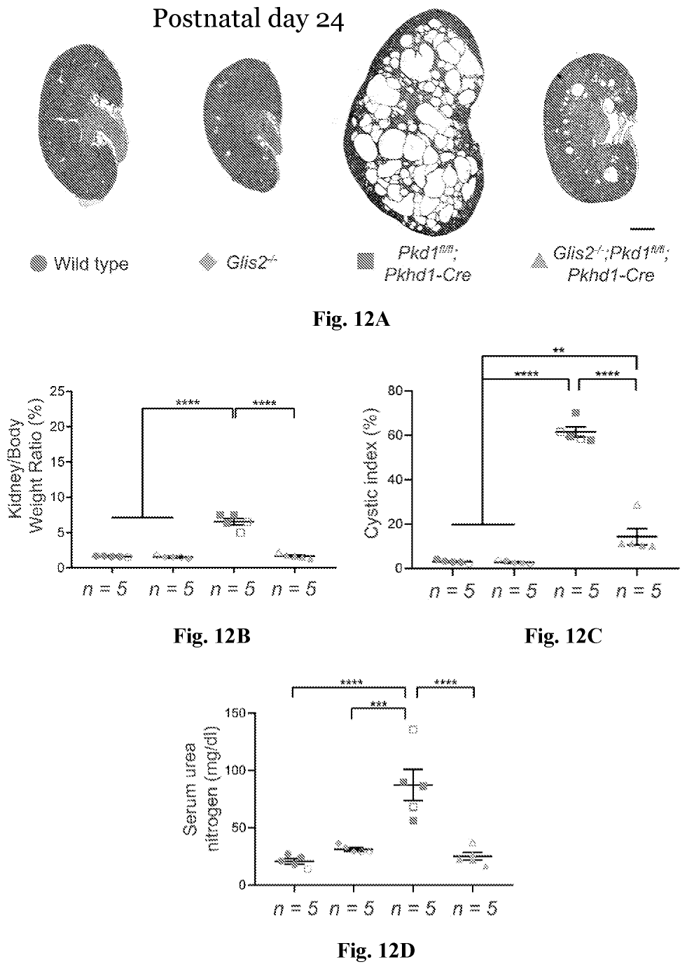

Referring to FIGS. 12A-12D, 13A-13D and 14A-14E, genetic deletion of GLIS2 ameliorates the ADPKD phenotype caused by Pkd1 or Pkd2 inactivation.

Referring to FIGS. 12A-12D, at postnatal day 24, Pkhd1 promoter driven Pkd1 knockout (Pkd1^{fl/fl}; Pkhd1-Cre) mice showed polycystic phenotypes in the kidney (FIG. 12A), as well as high kidney to body weight ratios, cystic indices and serum urea nitrogen contents (FIGS. 12B-12D). Introducing GLIS2 knockout to mice of the same background (GLIS2^{-/-}; Pkd1^{fl/fl}; Pkhd1-Cre) significantly ameliorated these symptoms (FIGS. 12A-12D).

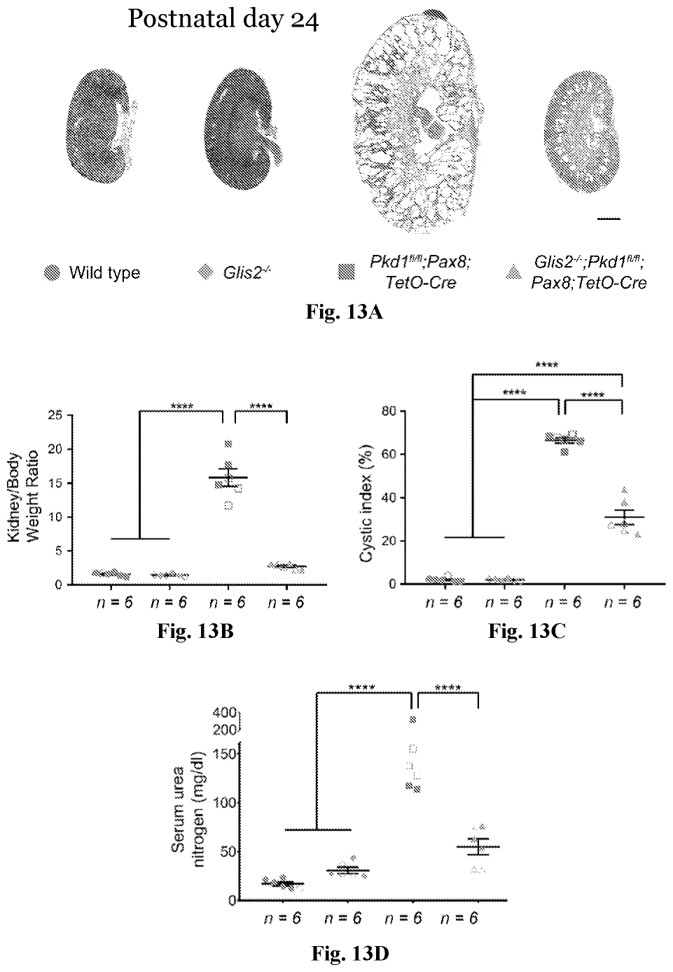

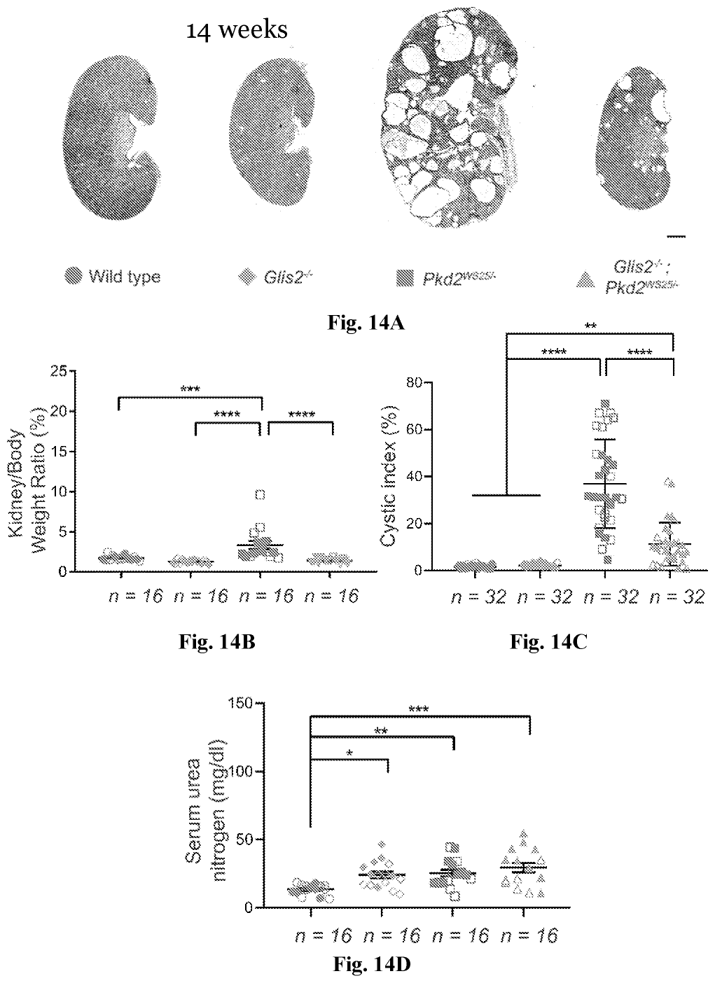

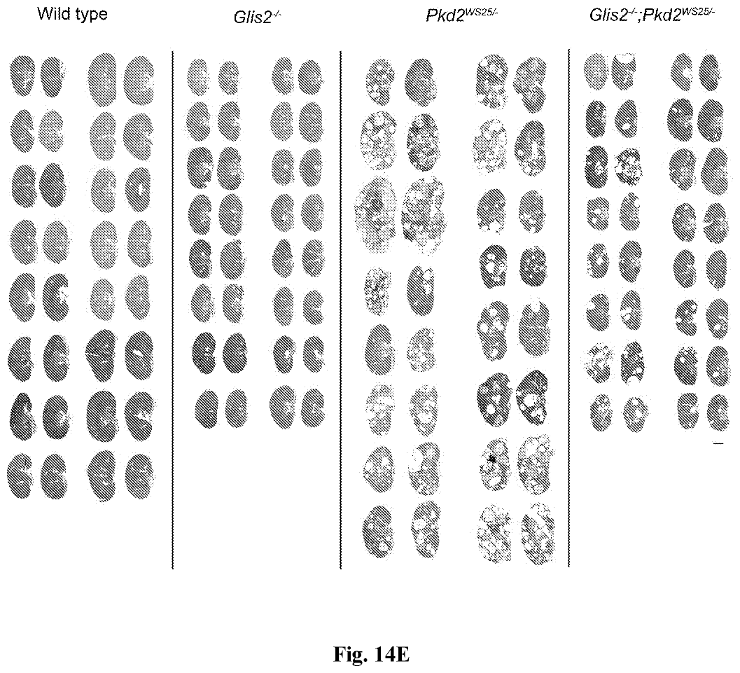

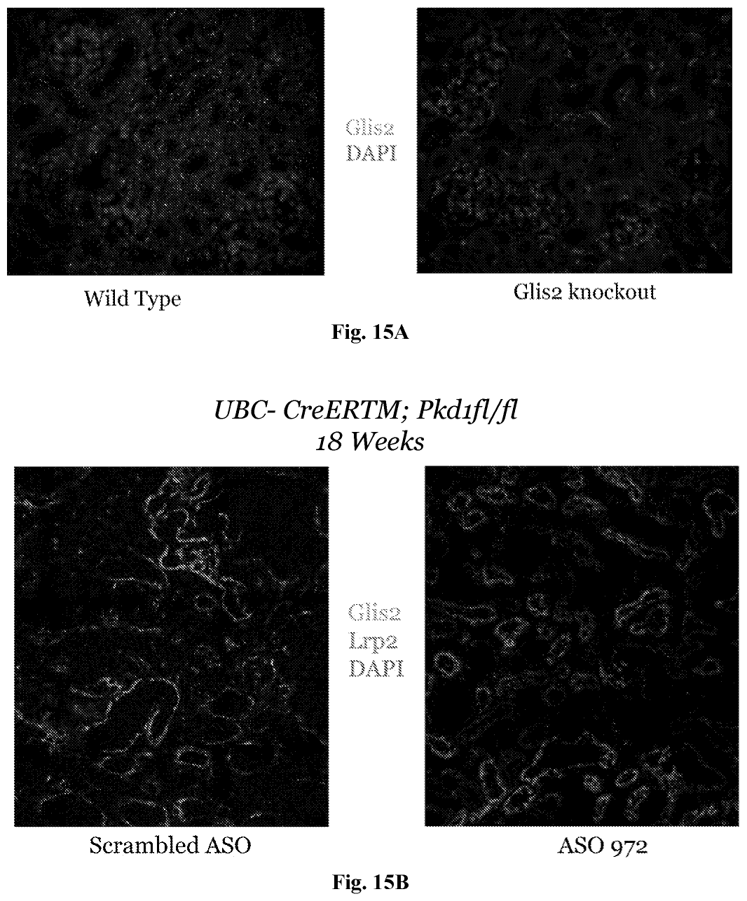

Referring to FIGS. 13A-13D, at postnatal day 24, Pax8 promoter driven Pkd1 knockout (Pkd1^{fl/fl}; Pax8; TetO-Cre) mice showed polycystic phenotypes in the kidney (FIG. 13A), as well as high kidney to body weight ratios, cystic indices and serum urea nitrogen contents (FIGS. 13B-13D). Introducing GLIS2 knockout to mice of the same background (GLIS2^{-/-}; Pkd1^{fl/fl}; Pax8; TetO-Cre) significantly ameliorated these symptoms (FIGS. 13A-13D). The Pkd2^{WS25/-} mice are an animal model of ADPKD. The mice have one Pkd2—true null mutant (Pkd2⁻) allele, and one unstable recombinant-sensitive Pkd2 allele (Pkd2^{ws25}). Pkd2^{WS25/-} mice develop liver cysts slowly and progressively due to somatic mutation of the Pkd2^{WS25} allele. Referring to FIGS. 14A-14E, at 14 weeks, Pkd2^{ws25/-} mice showed polycystic phenotypes in the kidney (FIGS. 14A and 14E), as well as high kidney to body weight ratios, cystic indices and serum urea nitrogen contents (FIGS. 14B-14D). Introducing GLIS2 knockout to mice of the same background (GLIS2^{-/-}; Pkd2^{ws25}) significantly ameliorated at least some of these symptoms (FIGS. 14A-14E).

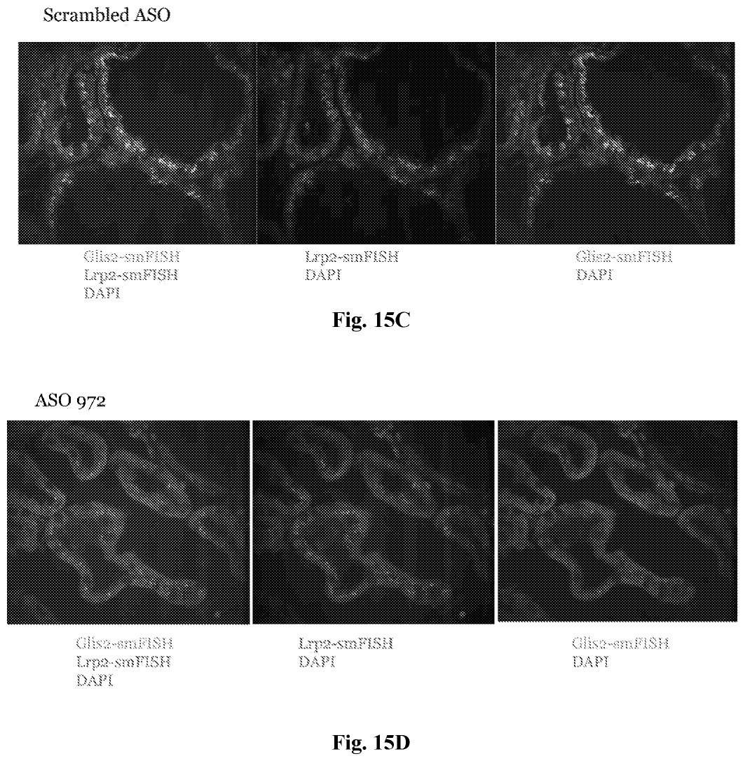

Referring to FIGS. 15A-15D, 16A-16B, and 17A-17B, downregulation of GLIS2 with an antisense oligonucleotide (ASO) targeting the GLIS2 mRNA ameliorates the ADPKD phenotype caused by Pkd1 inactivation.

Referring to FIGS. 15A-15D the present study generated ASO 972, an antisense oligonucleotide (ASO) targeting the GLIS2 mRNA. ASO 972 was able to specifically silence GLIS2 mRNA, while the control Scrambled ASO cannot.

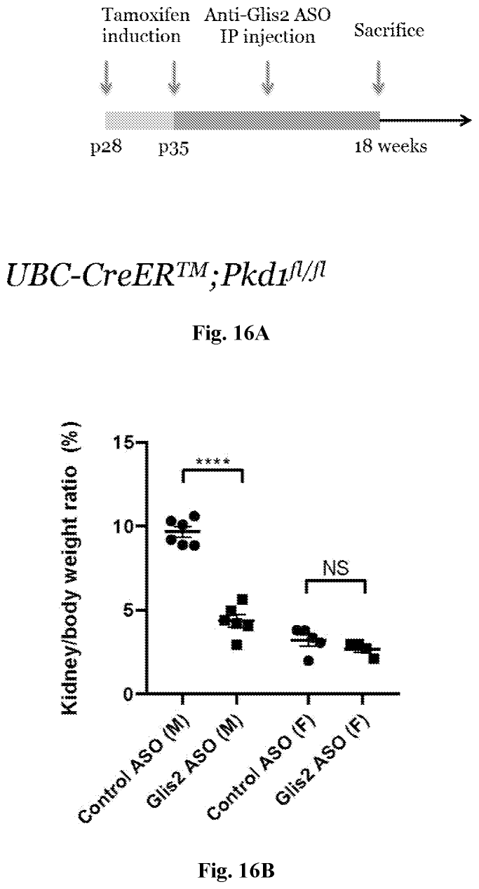

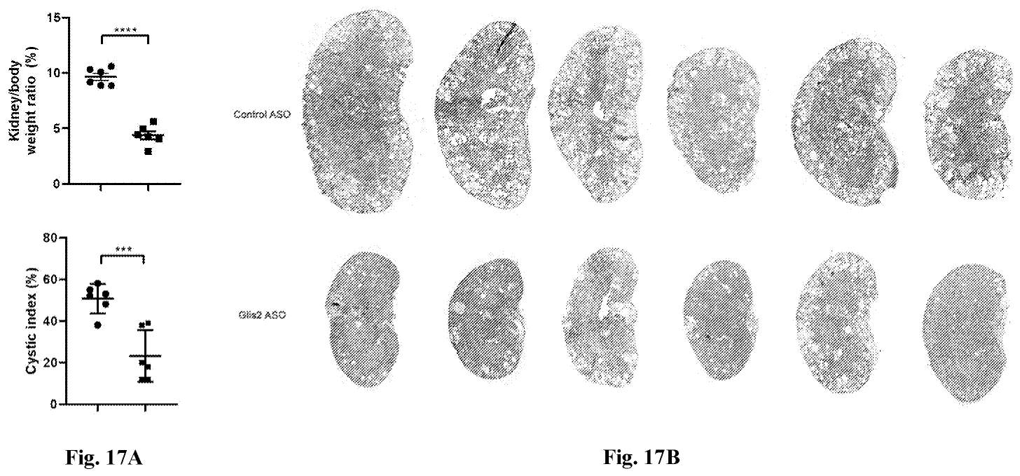

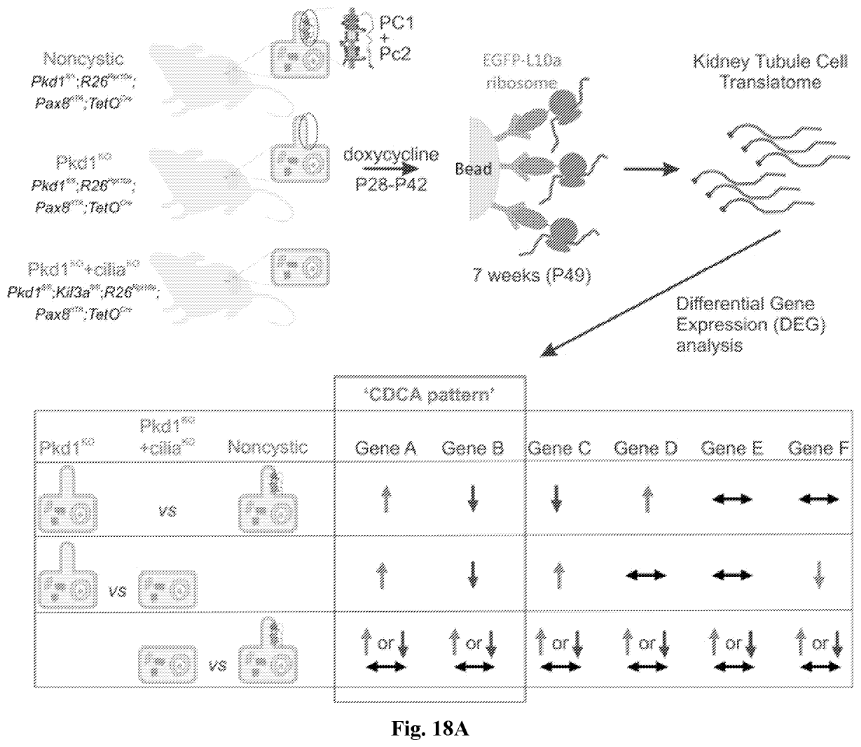

Referring to FIGS. 16A-16B, UBC-CreER™; Pkd1^{fl/fl} mice were injected with tamoxifen at postnatal days 28 and 35 to induce the deletion of Pkd1. The animals were then injected with either the control ASO or ASO 972 intraperitoneally (IP) and sacrificed at 18 weeks. Male animals showed significantly improved kidney to body weight ratios, and cystic indices (FIGS. 16B and 17A), as well as significantly lower polycystic phenotype in the kidneys.

### Example 2: Glis2 is an Early Effector of Polycystin Signaling and a Target for Therapy in Polycystic Kidney Disease

Studies in mouse models of autosomal dominant polycystic kidney disease (ADPKD) have shown that cyst formation following inactivation of polycystin-1 or polycystin-2 requires the presence of structurally and functionally intact primary cilia, a feature termed cilia dependent cyst activation (CDCA) herein. The molecular components of CDCA are not known. The present study applied unbiased cell type specific transcriptional profiling by translating ribosome affinity purification (TRAP) to mouse kidneys with polycystin-1 and cilia inactivation at a stage prior kidney tubule cyst formation. The present study identified 167 differentially expressed actively translating mRNA that correlated with the CDCA pattern common to male and female mice. This differential translatome offers opportunities for mechanistic discovery in polycystin and cilia related kidney phenotypes. From amongst this gene set, the present study investigated Glis2 as a candidate functional effector of polycystin signaling and CDCA. Glis2 transcript and protein expression in cell cultures followed the same polycystin and cilia dependent changes as occurred in kidney tissue, thereby establishing Glis2 as an in vivo-validated in vitro assay of polycystin function specifically related to cyst formation. Genetic inactivation of Glis2 in mouse kidneys suppressed polycystic kidney disease in early and adult mouse models of ADPKD based on both Pkd1 and Pkd2. Pharmacological targeting of Glis2 using antisense oligonucleotides suppressed polycystic kidney disease and associated secondary changes in a mouse model of ADPKD based on Pkd1. The present study found that Glis2 transcript and protein is an in vivo and in vitro biomarker of polycystin function, a functional target of CDCA and a potential therapeutic target for treating polycystic kidney disease.

### Example 2-1

Autosomal dominant polycystic kidney disease (ADPKD) is a common genetic disease most often caused by mutations in polcysytin-1 (PC1) or polycystin-2 (PC2). ADPKD presents with expanding fluid-filled cysts arising from kidney tubules that enlarge and deform the kidney, usually over the course of decades. Cysts grow by altering tubule cell shape and function, taking on a secretory rather than resorptive phenotype while also remodeling the surrounding interstitium and undergoing a low-level cyst cell proliferation. These changes are associated with increased inflammatory stimuli fibrotic changes in the surrounding kidney tissue. ADPKD presents with several clinical symptoms including hypertension, chronic and acute pain, urinary infections, and kidney stones. Cysts in the liver arising from the bile ducts are a common extrarenal manifestation while intracranial aneurysms are a less common but potentially catastrophic association that may have familial risk underpinnings. In the kidney, ADPKD results in impaired kidney function requiring dialysis or transplantation in the fifth decade of life for about half of affected individuals. While the disease is inherited as a dominant trait, cyst initiation requires somatic second hit mutations that further reduce or eliminate the functional dosage of the respective polycystin protein in kidney tubule or bile duct cells. ADPKD can also manifest in individuals inheriting recessive hypomorphic alleles for PKD1. The nature of the germline mutations affects the clinical severity of the disease with truncating mutations in PKD1 having the most severe course and minimally hypomorphic mutations in PKD1 and most mutations in PKD2 having a relatively more indolent course. However, the significant intrafamilial variation in severity of ADPKD indicates that additional factors beyond the inherited mutation have important roles in prognosis. Larger kidneys at an earlier age are at present the most accepted biomarker suggesting a more severe clinical course.

In more than two decades since the identification of PKD1 and PKD2, there has been significant progress in understanding the functions of the polycystin proteins. Several signaling pathways have been reported as aberrantly affected in ADPKD cystic epithelia including cyclic-AMP signaling, mammalian target of rapamycin (mTOR) signaling, G-protein coupled receptor signaling, and extracellular matrix signaling to name a few. Several cellular and organ level processes including metabolic pathways, mitochondrial function and immune modulation have been found to be affected by polycystin function and to affect the course of ADPKD. Despite, or perhaps because of, these pleiotropic findings, there is a lack of consensus on the roles of the polycystins most directly related to the ADPKD phenotype in the kidney. There are, however, also several areas of general agreement. There is consensus that in vivo mouse models based on Pkd1 and Pkd2, the orthologs of the respective human disease genes, are the best laboratory-based models for validation of functional pathways and putative therapeutic targets for ADPKD. There is also agreement that polycystins are localized to the membranes overlying the primary cilia, solitary microtubule based cellular sensory organelles protruding from the apical luminal surface of kidney tubule and cyst cells. Primary cilia play a central role in the pathogenesis of ADPKD. The genetic relationship between cilia and polycystins in ADPKD was first defined in a study showing that cyst growth following the loss of polycystins is suppressed by concomitant genetic removal of cilia. Cyst growth was also suppressed in adult models of ADPKD following inactivation of Tulp3, which affects the membrane associated protein composition of cilia without dismantling the organelle entirely. These series of findings have defined the presence of a cilia-dependent cyst activating (CDCA) pathway that is regulated by the polycystin complex. CDCA drives cyst formation after inactivation of polycystins in the presence of otherwise functionally intact cilia but is suppressed if cilia are absent. The lack of coalescence toward a unifying functional pathway in polycystin biology and the persistence of gaps in understanding of polycystin function in vivo suggest that critical components of the most proximal components of the polycystin-CDCA signaling cascades have yet to be identified. The molecular events most closely related to the primary function of polycystins in cilia are potentially the most effective and most specific targets for therapy in ADPKD.

The current study began with the premise that these gaps in understanding can be approached through unbiased discovery strategies followed by validation steps in mouse models based on orthologous genes. The present study based the discovery step on kidney tubule selective Translating Ribosome Affinity Purification (TRAP) transcriptional profiling. The present study applied TRAP to models of polycystin and cilia inactivation in mouse kidneys and made use of the unique feature of mouse models that allows interrogation of genetically defined kidney tubule cell populations before the onset of cyst formation and its attendant confounding secondary non-cell autonomous biological effects. The present study identified novel group of 167 transcripts whose expression in vivo is specifically altered in the same direction of change in Pkd1 knockout cells destined form cysts when compared to both wild type cells and cells with dual Pkd1 and cilia inactivation which are protected from cyst formation. The present study applied biologically informed selection criteria to further refine this list to 71 genes from which 11 genes were identified with the highest statistical significance and biological plausibility. The present study selected one candidate gene, Glis2, for biological validation. Glis2 encodes one of three Gli-similar (Glis1-3) Kruppel-like zinc finger transcription factor proteins. Glis2 is most abundantly expressed in the kidney along the entire nephron and is the causative gene for nephronophthisis type 7 (NPHP7). The present study found that Glis2 transcript and protein expression changes in kidney cell cultures in vitro are a robust surrogate for in vivo polycystin-dependent cyst formation. Inactivation of Glis2 in early onset and adult mouse models of Pkd1 and Pkd2 resulted in suppression of cyst formation. Furthermore, pharmacological targeting of Glis2 using antisense oligonucleotides suppressed cyst formation and associated secondary changes in the mouse kidney. In aggregate, the present study developed a discovery platform for transcriptional targets of polycystin function and discovered Glis2 as a novel and potentially tractable therapeutic target for treatment of ADPKD.

### Example 2-2: Translating Ribosome Affinity Purification Transcriptional Profiling in Early-Stage Cyst Cells In Vivo

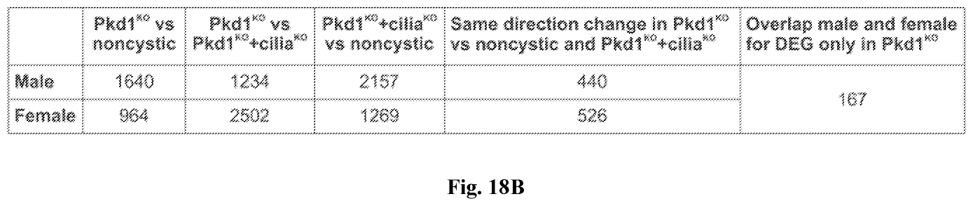

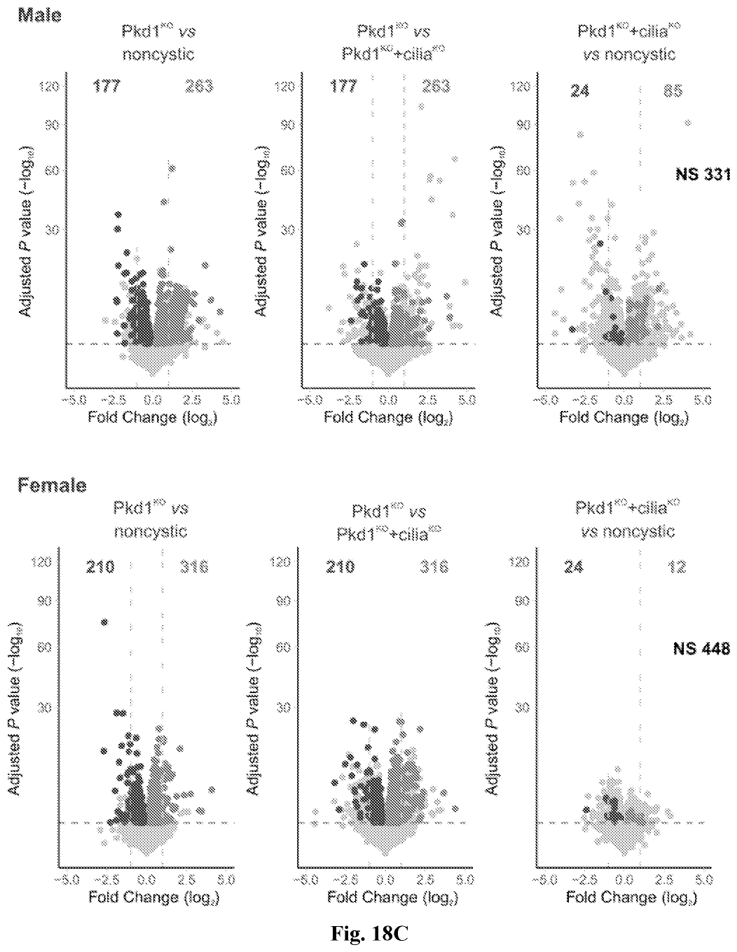

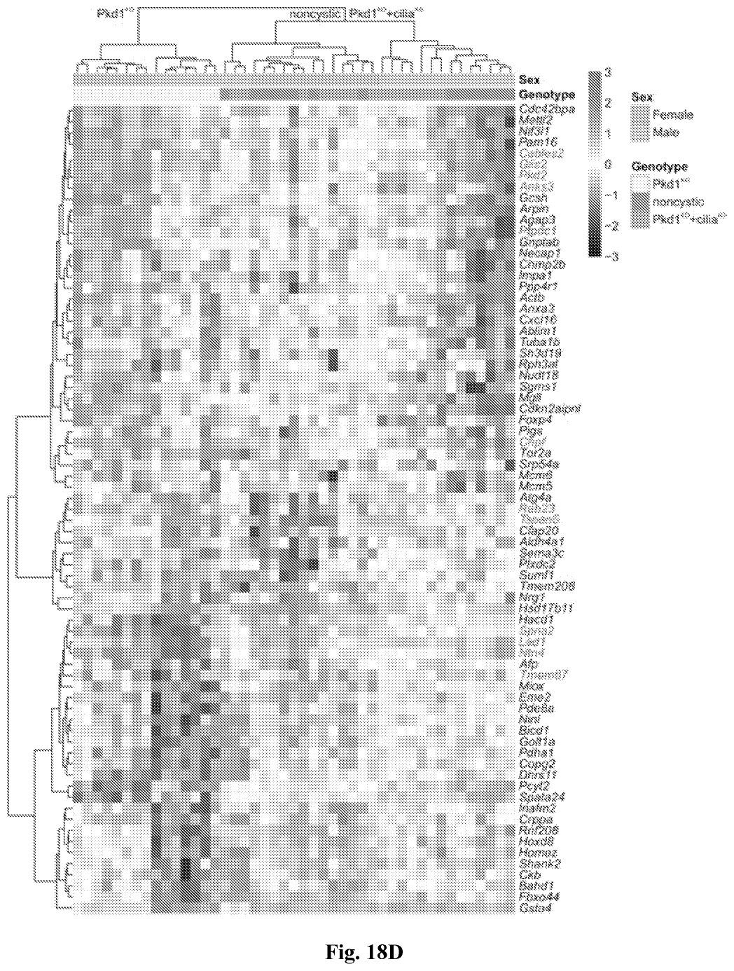

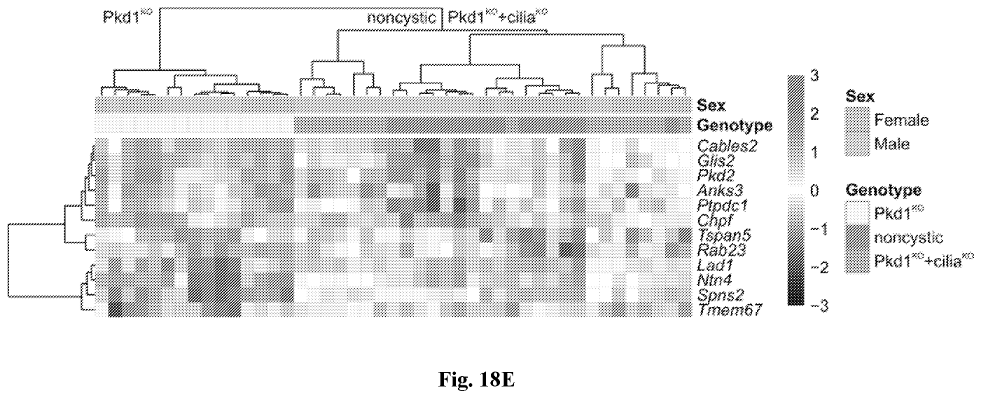

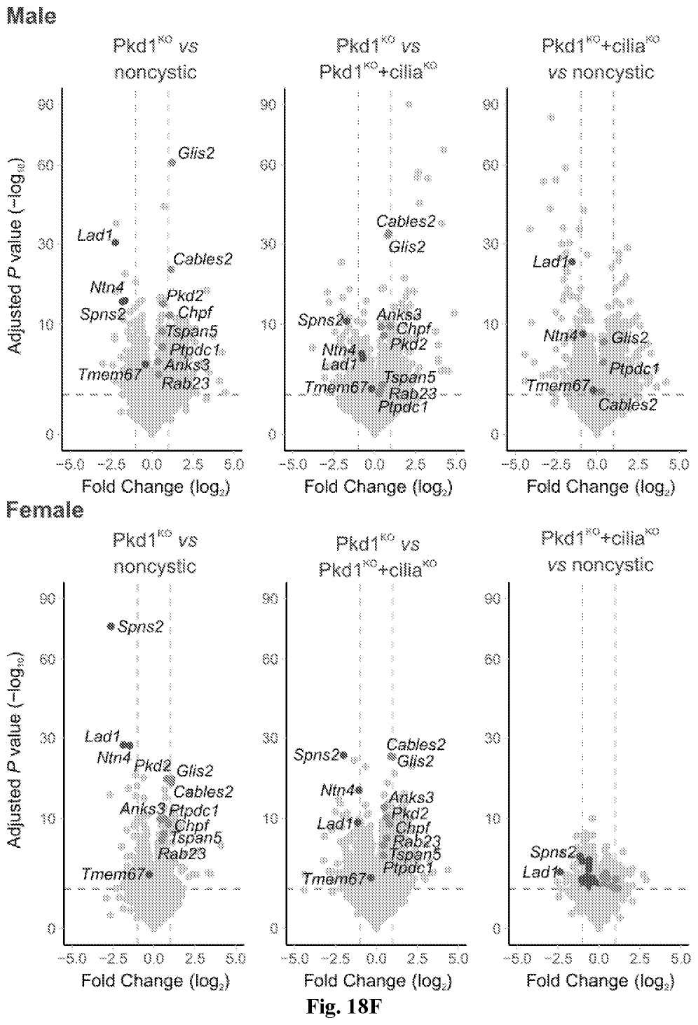

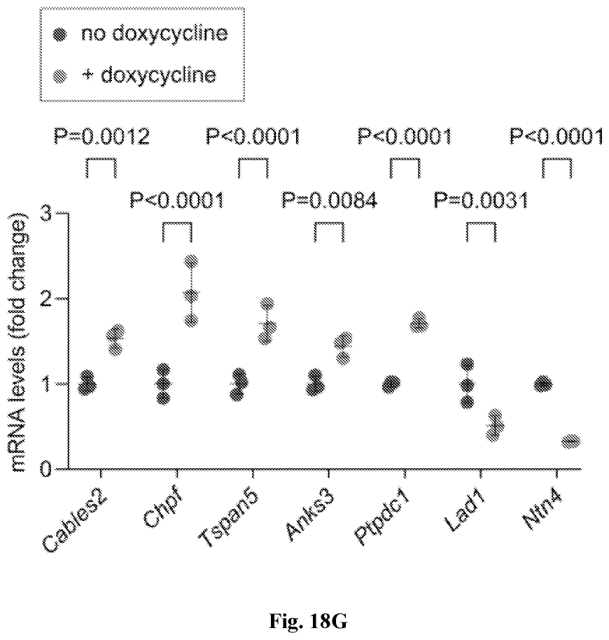

To discover early changes in gene expression signatures that are uniquely associated with loss of polycystin-1 and cyst formation in vivo, the present study used Translating Ribosome Affinity Purification (TRAP) to interrogate actively translating mRNA expression (the “translatome”) in a cell specific manner in mouse kidney tubules that were genetically induced to form cysts, but at a time point before cysts formed. To achieve cell specificity for kidney tubules, the present study used the Pax8^{rtTA}; TetO^{Cre} digenic doxycycline inducible kidney selective system to inactivate target genes and contemporaneously turn on expression of the L10a-EGFP ribosomal fusion protein used to isolate ribosomal complexes only from cells in which Cre recombinase had been active. To identify differentially expressed transcripts that are most specifically correlated to cyst formation, the present study combined analysis from mice with three genotypes: Pkd1 single mutants (Pkd1^{KO}) that are genetically destined to develop polycystic kidney disease, Pkd1 and Kif3a (Pkd1^{KO}+ cilia^{KO}) double knockouts that are protected from cyst growth despite inactivation of Pkd1, and Pkd1 heterozygous mice that behave like wild type (‘noncystic’) (FIG. 18A). To limit discovery to cell autonomous transcriptional changes following loss of polycystin-1 and avoid confounding by “outside-in” signaling from inflammatory and other responses that occur as cyst formation progresses, the present study performed TRAP RNASeq at a time point at which polycystin-1 and cilia had disappeared but cysts had not yet begun to form. Using comparisons amongst these three genotypes, the present study identified differentially expressed genes that showed significant ‘same relative direction change’ in Pkd1^{KO} when compared to both Pkd1^{KO}+ cilia^{KO} and noncystic controls—i.e., significantly upregulated in Pkd1^{KO} compared to both other genotypes and downregulated in Pkd1^{KO} compared to both (FIG. 18A). Finally, since there is significant sex dimorphism in cyst growth in Pkd1 adult mouse models, the present study prespecified independent evaluation of differentially expressed transcriptional profiles in male and female mice.

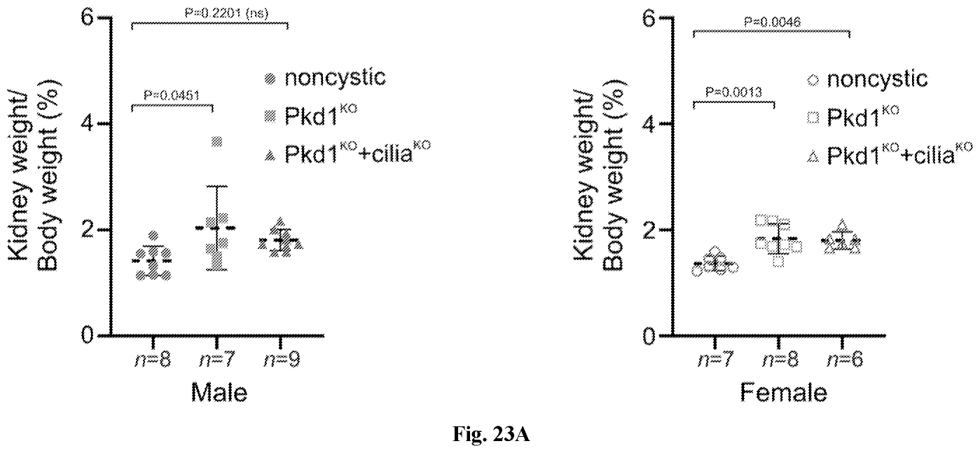

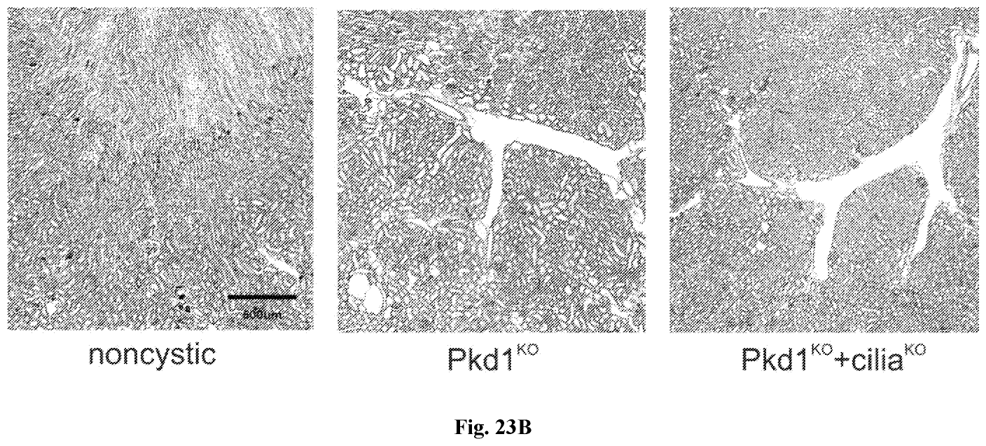

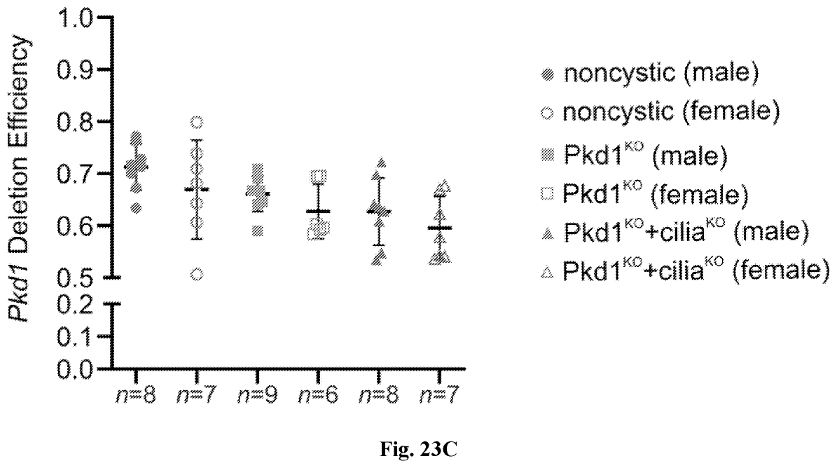

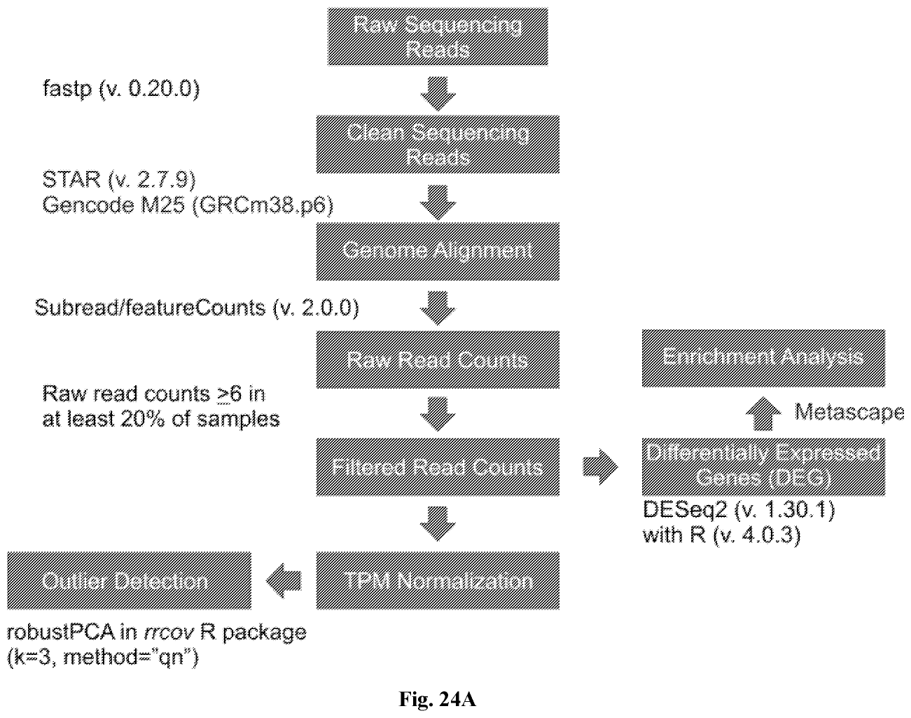

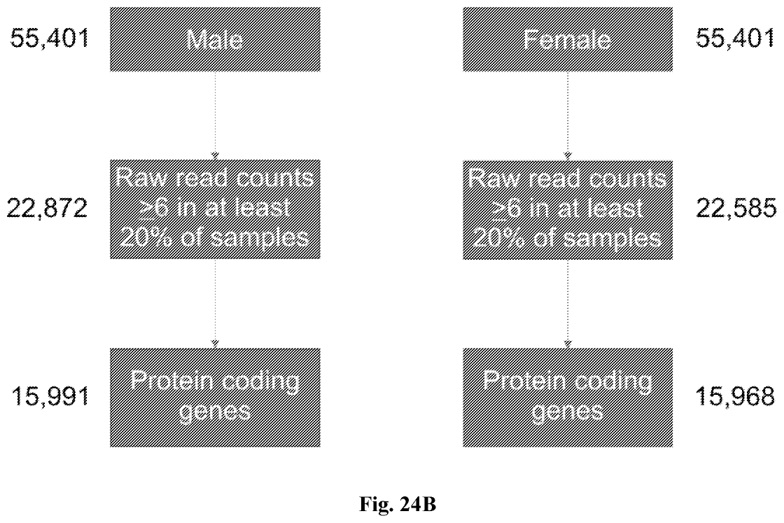

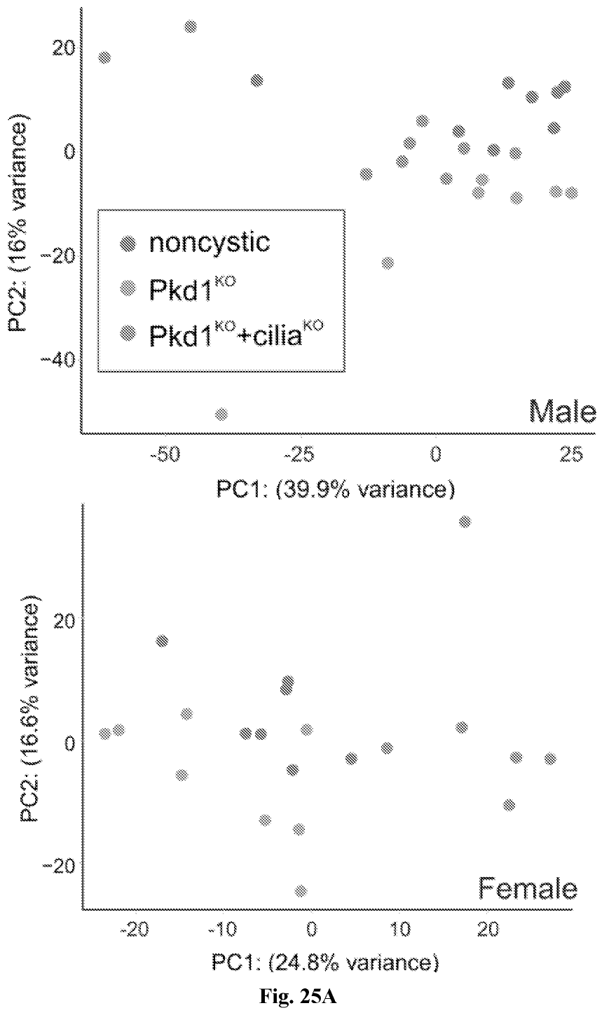

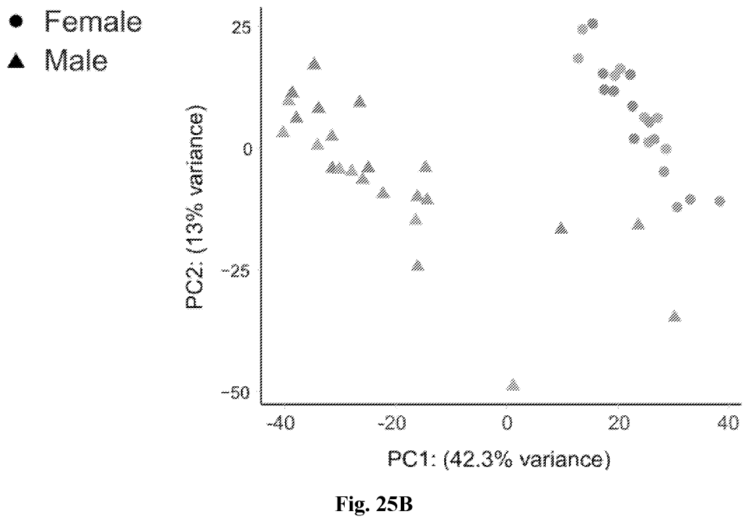

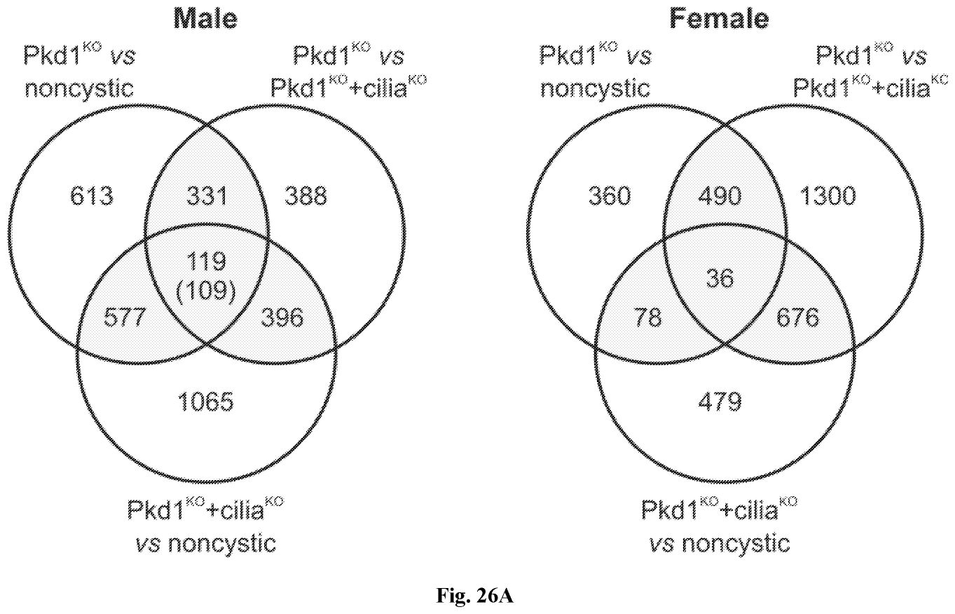

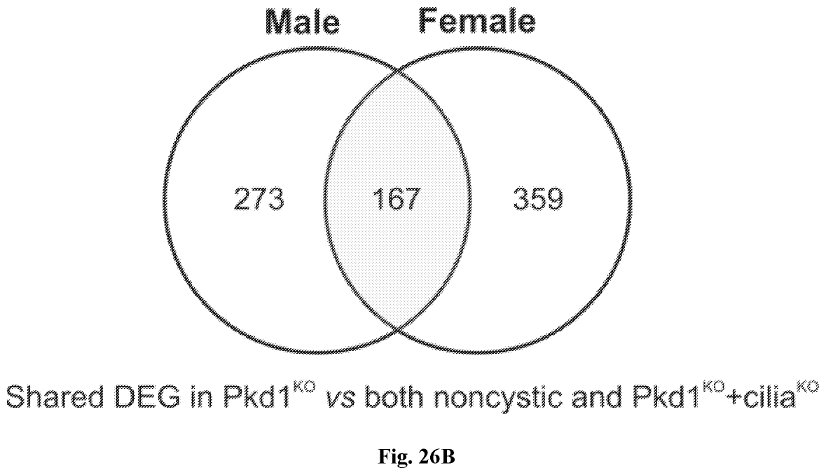

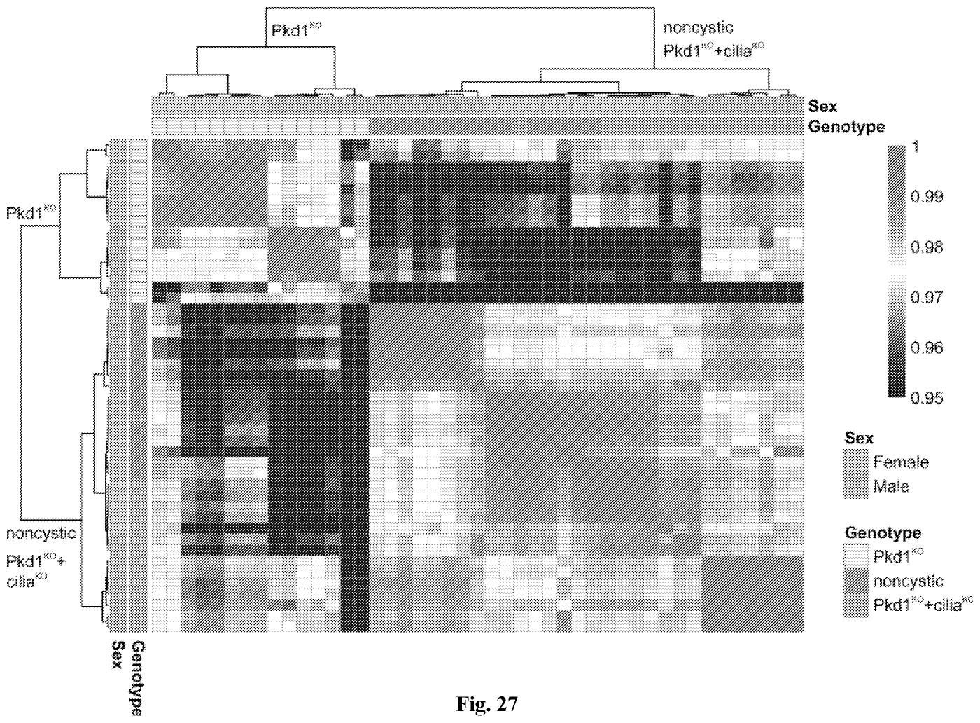

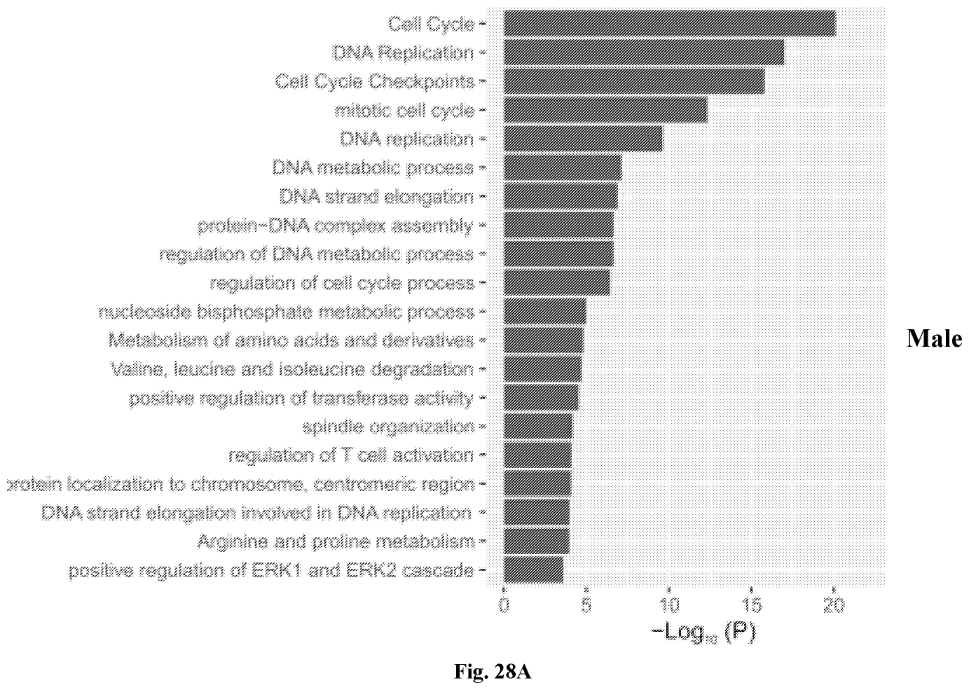

To achieve these objectives, the present study used mice with the following genotypes: Pkd1^{fl/fl}; R26^{Rpi10a}; Pax8^{rtTA}; TetO^{Cre} (Pkd1^{KO}), Pkd1^{fl/fl}; Kif3a^{fl/fl}; R26^{Rpi10a}; Pax8^{rtTA}; TtO^{Cre} (Pkd1^{KO}+ cilia^{KO}), Pkd1^{fl/fl}; R26^{RpI10a}; Pax8^{rtTA}; TetO^{Cre} (“noncystic”). All experimental mice were hemizygous for the R26^{Rpi10a}, Pax8^{rtTA} and TetO^{Cre} alleles to control for dosage effects. All mice received oral doxycycline from postnatal days 28 to 42 (P28-P42) to induce TetO^{Cre}. Fresh kidney tissue was harvested for ribosomal pulldown at P49 (7 weeks) when mice had minimal histologic changes associated with cyst formation (FIGS. 23A-23B). The present study used 6-8 mice for each genotype and sex, for a total of 45 TRAP RNASeq samples in the 6 groups with at least 100 million reads in each sample. The efficiency of Pkd1^{fl} allele deletion in kidneys across all genotypes and in both sexes was 60-70% (FIG. 23C). TRAP RNASeq raw sequences were processed and aligned to the mouse reference genome (FIG. 24A). Since the TRAP is directed at actively translating mRNA, the present study constrained the analysis to protein coding gene transcripts. TRAP identified 15,991 and 15,968 protein coding genes in male and female mice, respectively (FIG. 24B). The TRAP RNASeq data is available in a browsable format at https://pkdgenesandmetabolism.org/(reviewer username: pkduser; password: rc2pkd2022). Analysis by standard PCA and robustPCA did not suggest significant outliers (FIG. 25A and data not shown); however, sex differences were apparent (FIG. 25B). Differentially expressed protein coding genes (DEG) were identified using the DESeq2 package. The Benjamini-Hochberg procedure was used for multiple test adjustment with FDR<0.05 selected as the threshold for statistical significance. Pairwise comparison between Pkd1^{KO} single mutant and noncystic kidneys identified 1640 and 964 DEGs in male and female mice, respectively, whereas Pkd1^{KO} compared with Pkd1^{KO}+ cilia^{KO} double mutant kidneys identified 1234 and 2502 DEGs for male and female mice, respectively (FIG. 18B). Male and female mice respectively had 440 and 526 DEG that were differentially expressed with the ‘same relative direction of change’ in Pkd1^{KO} when compared to both Pkd1^{KO}+ cilia^{KO} double knockouts and noncystic controls (FIGS. 18B-18C and 26A). These two DEG sets are comprised of 799 unique genes since 167 DEG are shared between male and female mice (FIGS. 18B and 26B). Pearson correlation analysis of the 799 unique DEGs showed high correlation coefficients across all samples with particularly close grouping for all Pkd1^{KO} samples which further clustered based on sex (FIG. 27). The 167 shared DEGs follow a sex-independent ‘CDCA-pattern’ defined as being selectively dysregulated in cyst-prone Pkd1^{KO} tubule cells at the precystic stage when compared to both Pkd1^{KO}+ cilia^{KO} double mutants that lack propensity for cyst formation and noncystic controls. It was hypothesized that this gene set has a high likelihood of containing subsets of genes that define a transcriptional ‘signature’ for polycystin loss in vivo. The gene set likely also contains genes whose dysregulation is functionally related to in vivo cyst progression in ADPKD.

### Example 2-3: ‘CDCA-Pattern’ Transcriptional Candidates

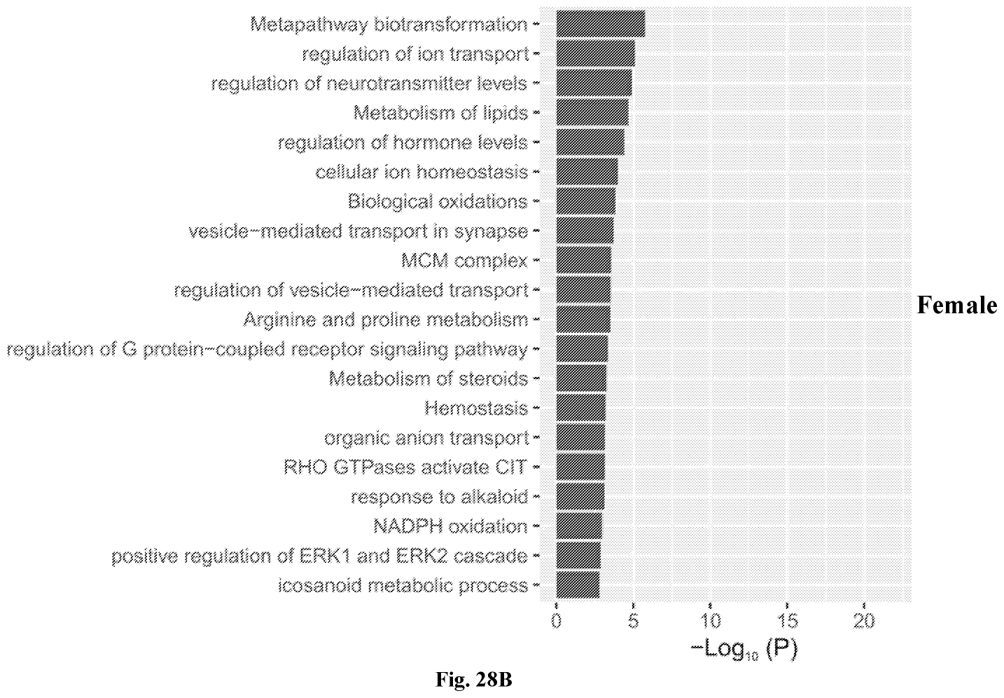

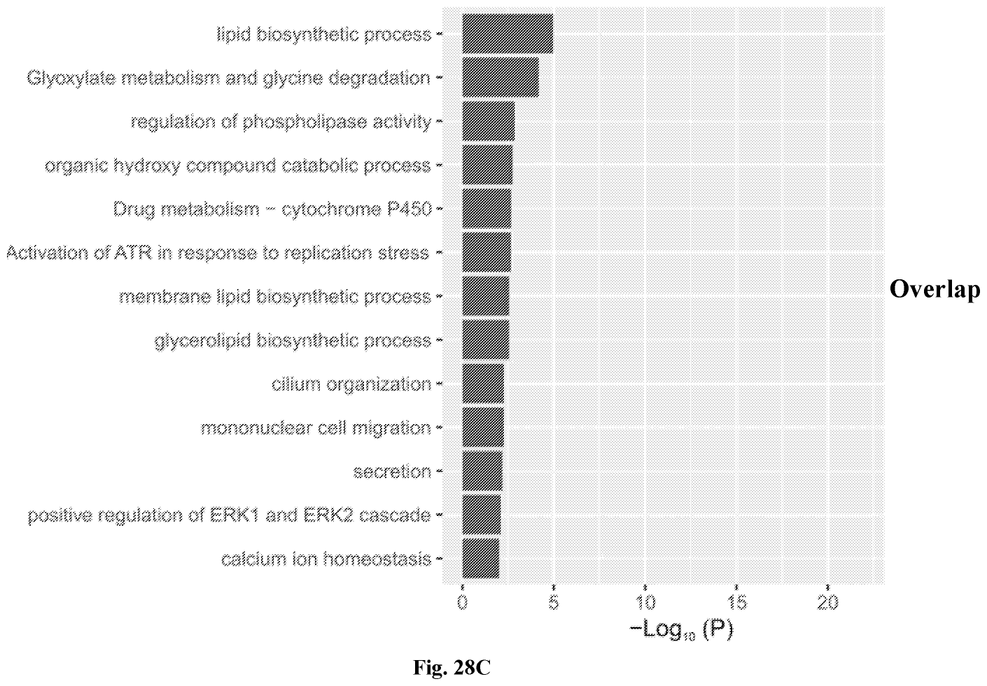

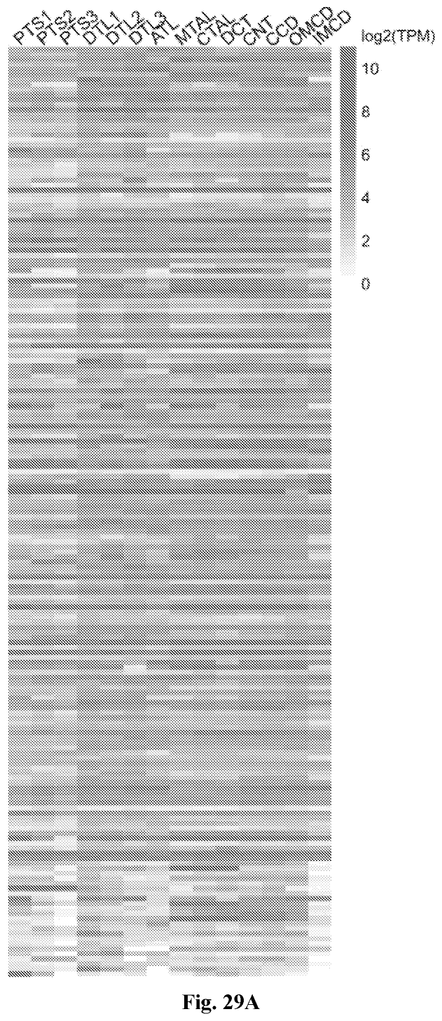

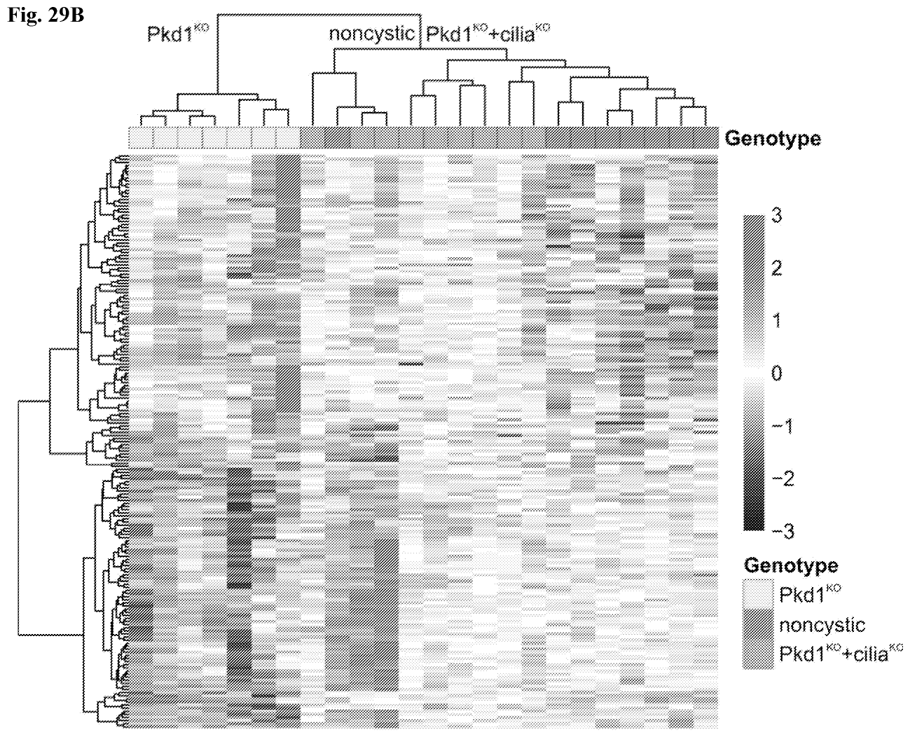

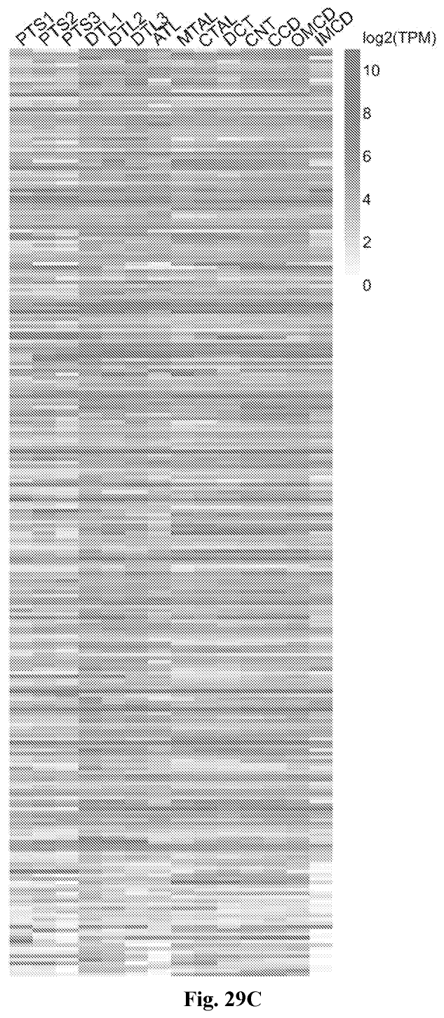

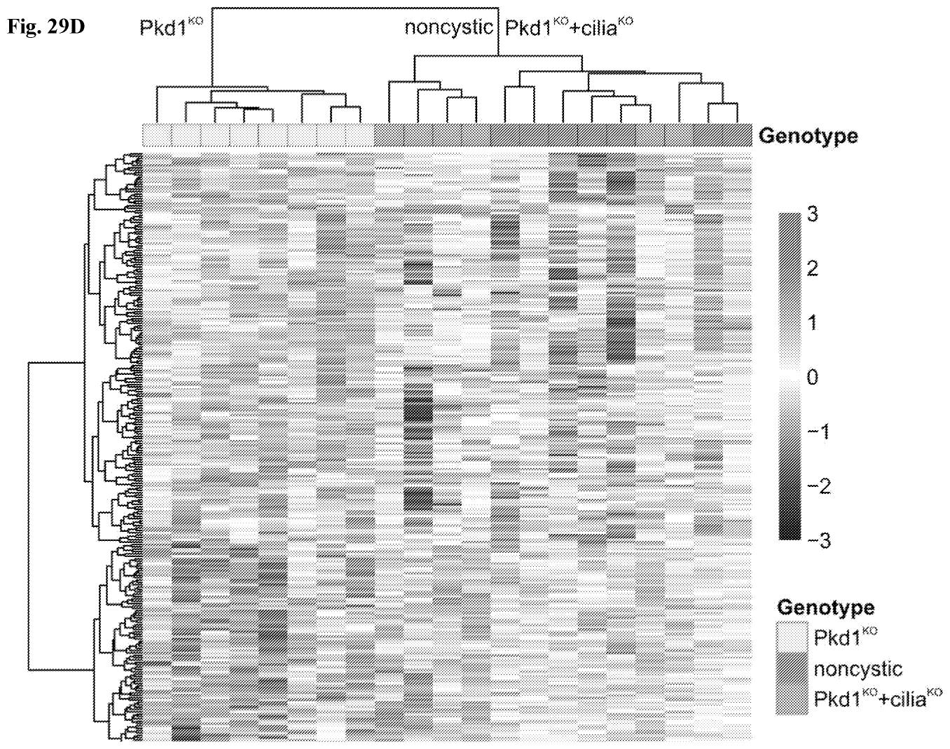

Biologic pathway enrichment analysis using Metascape applied to the male, female, and combined overlap ‘CDCA pattern’ gene sets with 440, 526 and 167 genes, respectively, showed a strong enrichment of cell cycle related processes in male mice only (FIGS. 28A-28C). The 167 gene overlap group showed enrichment primarily of metabolic pathways although “cilium organization” also appeared (FIGS. 28A-28C). Notably, these analyses did not identify pathways previously implicated in ADPKD pathogenesis, suggesting an opportunity for discovery of novel transcriptional responses to polycystin-1 function. The present study further prioritized the CDCA-pattern DEG based on biological knowledge. All kidney tubule segments and tubule epithelial cell types, with the likely exception of intercalated cells which lack cilia, have the capacity to form cysts that can be suppressed by inactivation of cilia. Therefore, the TRAP RNASeq DEG data was cross-referenced with microdissected nephron segment-specific bulk RNASeq expression data to identify transcripts expressed along the entire tubule as would be expected of candidate transcriptional changes related to CDCA. Based on these considerations the present study prioritized genes with expression in at least seven nephron segments where Pax8^{rtTA} is active (proximal tubule S1, S2, medullary thick ascending limb, cortical thick ascending limb, distal convoluted tubule, connecting tubule, cortical collecting duct). In addition, the role of genes with statistically significant differential expression but extremely low absolute expression in the kidney is uncertain. The present study therefore further limited the gene set to those with TPM>1.0 in all seven microdissected segments in normal kidney bulk RNASeq. These criteria yielded 185 out of 440 genes in male and 252 out of 526 genes in the female CDCA-pattern DEGs (FIGS. 29A-29D). The overlap between male and female for this subset of CDCA-pattern DEG contained 73 genes (FIG. 18D). This group of 73 CDCA pattern genes show robust expression in all nephron segments of the normal kidney, with 61 of 73 genes having TPM>1.0 in all 14 microdissected segments analyzed.

The biological relevance of elements of this gene set, which was selected without bias regarding functional roles, is supported by the inclusion of Pkd2 which is significantly upregulated in response to inactivation of Pkd1 in vivo (FIGS. 18D-18F). Several genes in this group have potential roles in cilia related functions or phenotypes including Glis2 (NPHP7), Ptpdc1, Anks3, and Rab23 which are significantly upregulated in Pkd1^{KO} and Tmem67 which is downregulated (FIGS. 18D-18F). Other genes on this list show strong statistical association with the Pkd1^{KO} genotype, albeit in the absence of clear functional hypotheses. These include Cables2, Chpf and Tspan5 which are upregulated with Pkd1^{KO} and Lad1, Ntn4, and Spns2 which are downregulated in Pkd1^{KO} relative to both other genotypes (FIGS. 18D-18F). As a preliminary assessment of the robustness of the in vivo transcriptional changes, the present study used primary cell cultures from kidneys of Pkd1^{fl/fl}; Pax8^{rtTA}; TetO^{Cre} (Pkd1^{KO}) mice. Cells were either treated with doxycycline to inactivate Pkd1 during cell culture or left untreated; all cells were grown under conditions to form cilia prior to analysis. Cells were examined by quantitative reverse transcriptase PCR (RT-PCR) to assess expression of Cables2, Chpf, Tspan5, Anks3, Ptpdc1 Lad1, and Ntn4. Expression of all 7 genes was significantly changed in the Pkd1 knockout cells relative to the non-knockout cells in the same direction as in the TRAP RNASeq, thereby recapitulating the polycystin-1 genotype dependent transcriptional changes found in vivo (FIG. 18G). The robust expression of these genes along the nephron, along with the strength of the association with the Pkd1^{KO} genotype in the CDCA pattern coupled with the reproducibility of expression changes in primary cell cultures suggest amongst these genes, a subset may comprise a transcriptional signature for polycystin function; beyond that, they present opportunities for discovery of novel functional relationships with cyst formation.

### Example 2-4: Glis2 is a Target of Polycystin Signaling In Vivo and In Vitro

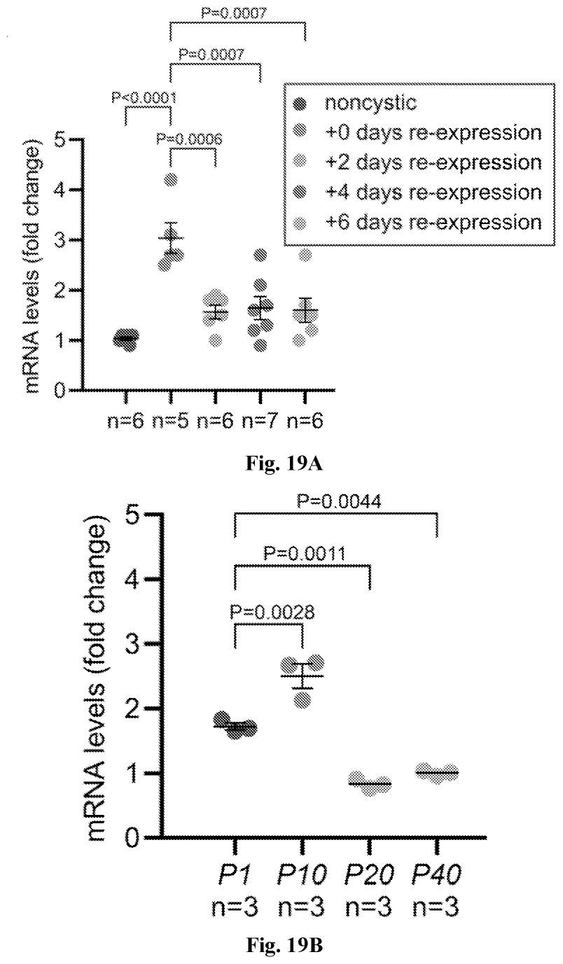

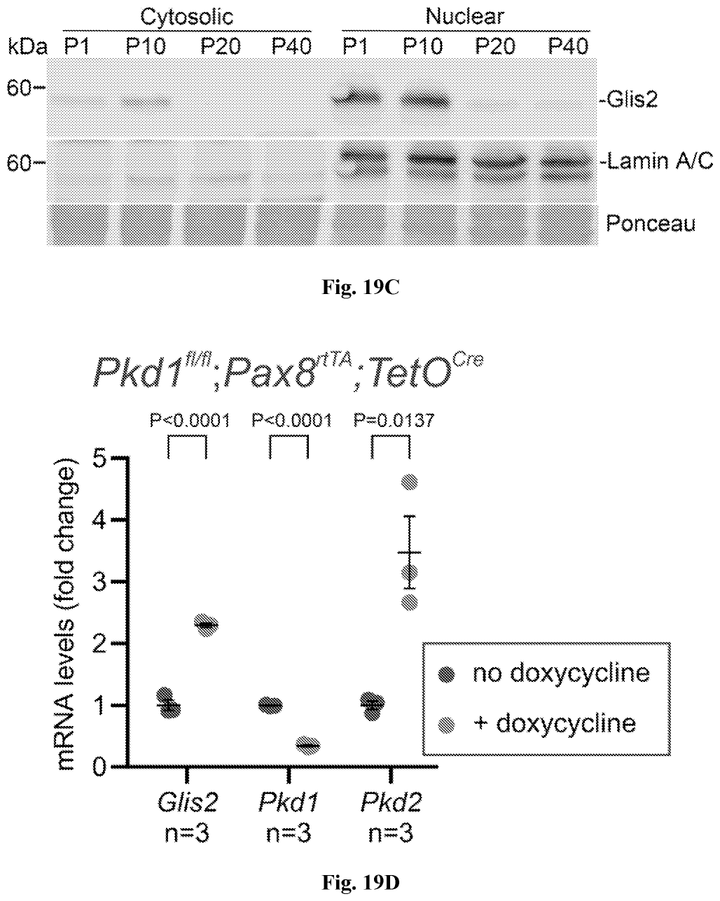

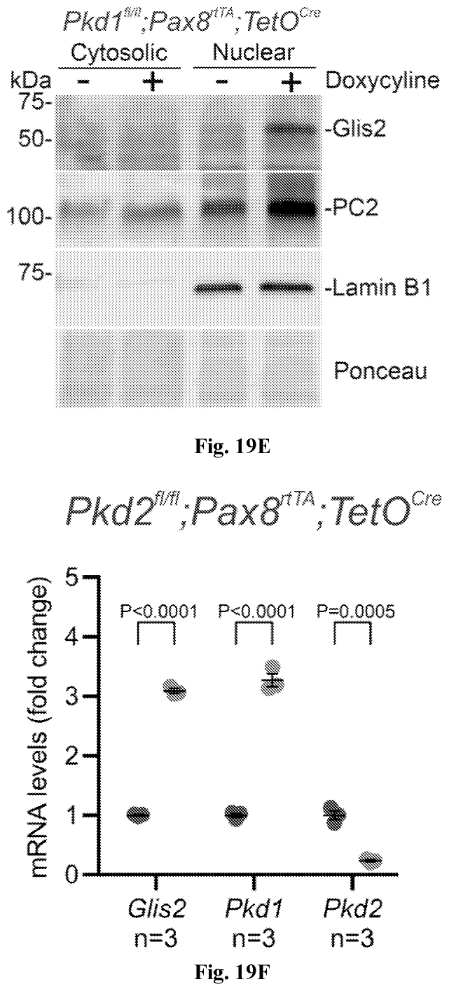

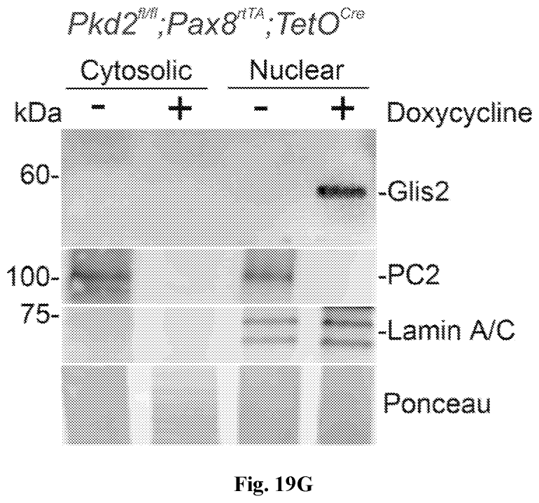

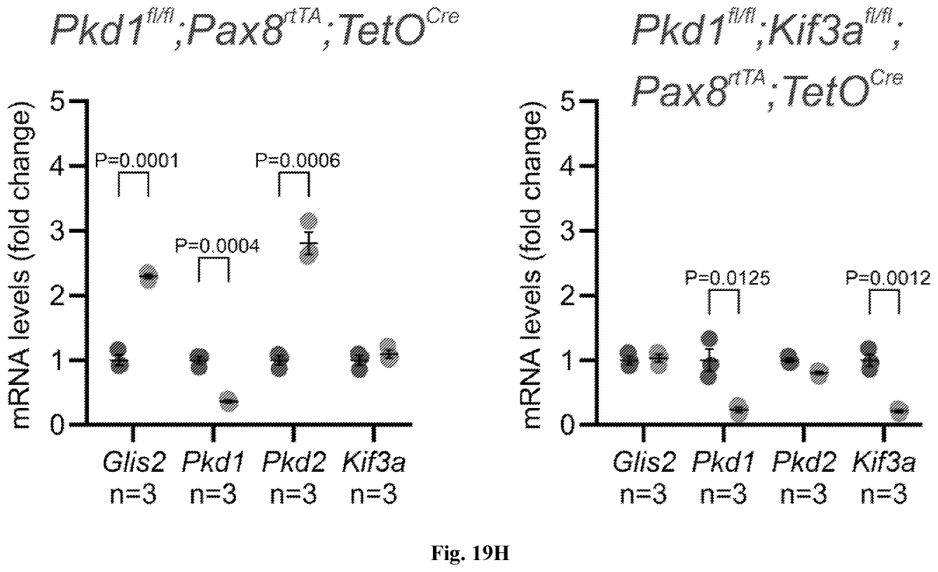

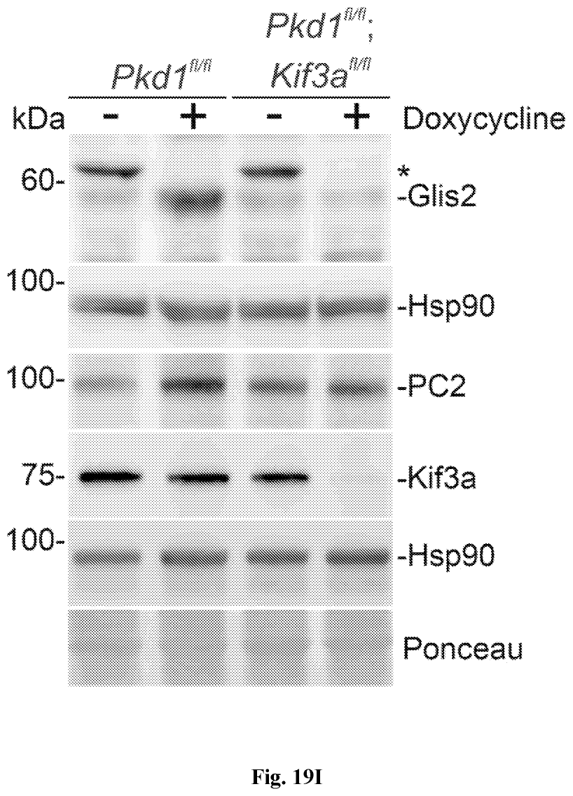

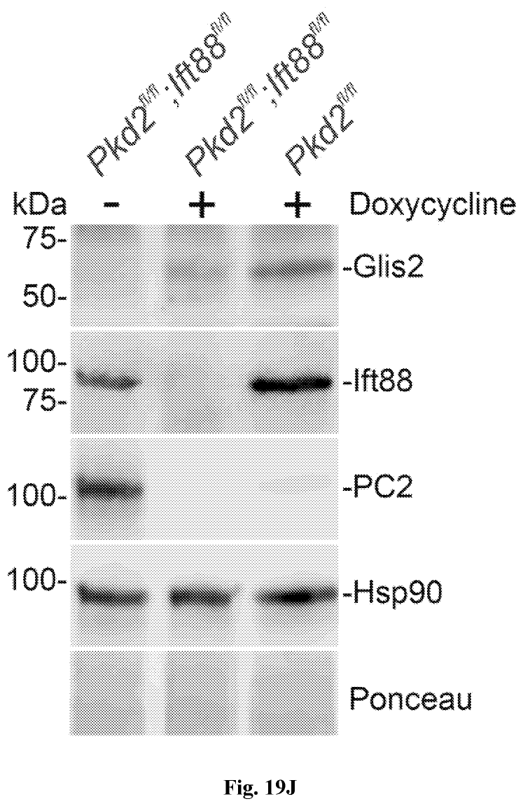

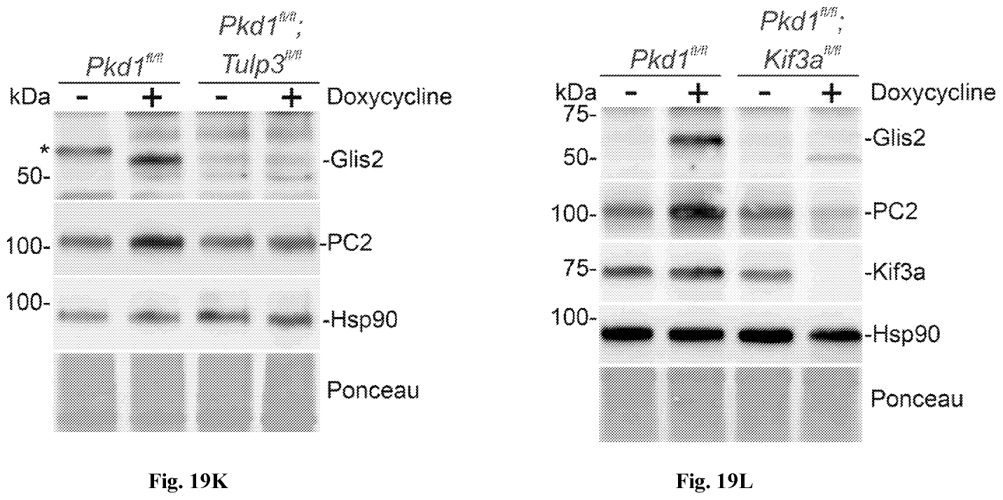

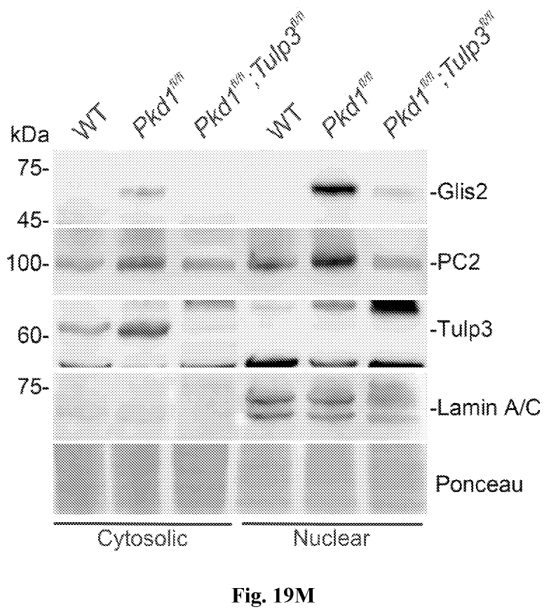

To explore potential functional relationships in this gene set, the present study selected Glis2 for further validation. Glis2 is among the most statistically significantly dysregulated genes in Pkd1^{KO} in both male and female mice compared to the other two genotype groups (FIGS. 19D-18F). It has broad expression across all segments of the nephron. Glis2 is most highly expressed in the kidney relative to other tissues and inactivation of Glis2 results in primarily a kidney phenotype. Finally, Glis2 is a causative gene for nephronophthisis, a ciliopathy related kidney disease. To determine whether the changes in Glis2 expression found in Pkd1 models are extensible to other preclinical models of ADPKD, the present study used a mouse model in which polycystic kidney disease resulting from inactivation of Pkd2 is rapidly reversed by re-expression of Pkd2. Glis2 expression was elevated in 13-week-old Pkd2 mutant kidneys showing that the transcriptional upregulation observed in Pkd1 knockouts extends to Pkd2 (FIG. 19A). Beginning two days after starting Pkd2 re-expression, Glis2 transcripts returned to levels that did not differ significantly from noncystic kidneys (FIG. 19A). This rapid normalization of Glis2 expression after PC2 re-expression parallels the rapid resolution of the polycystic kidney phenotype in vivo. Glis2 is an attractive candidate for CDCA-related functions.

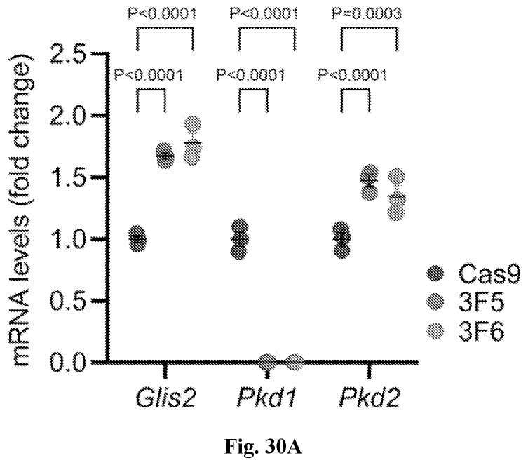

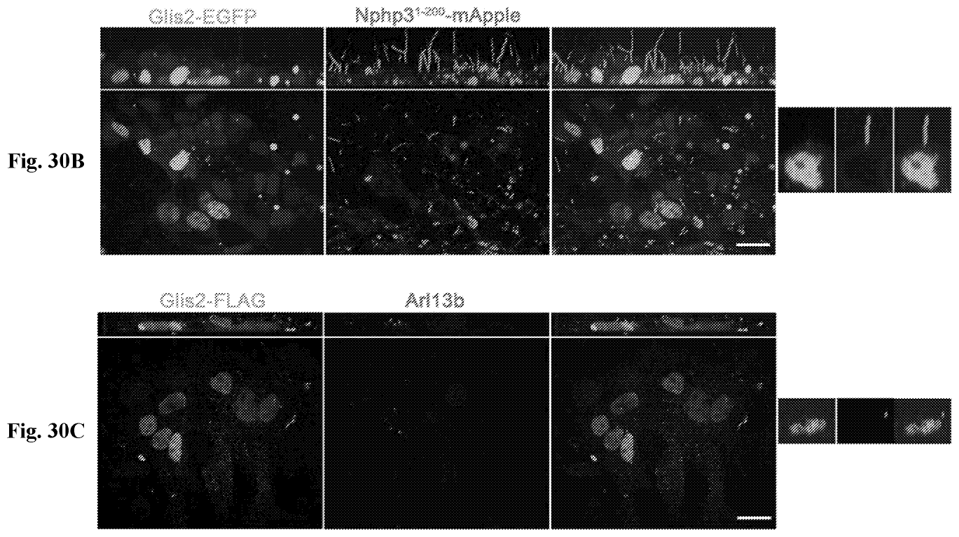

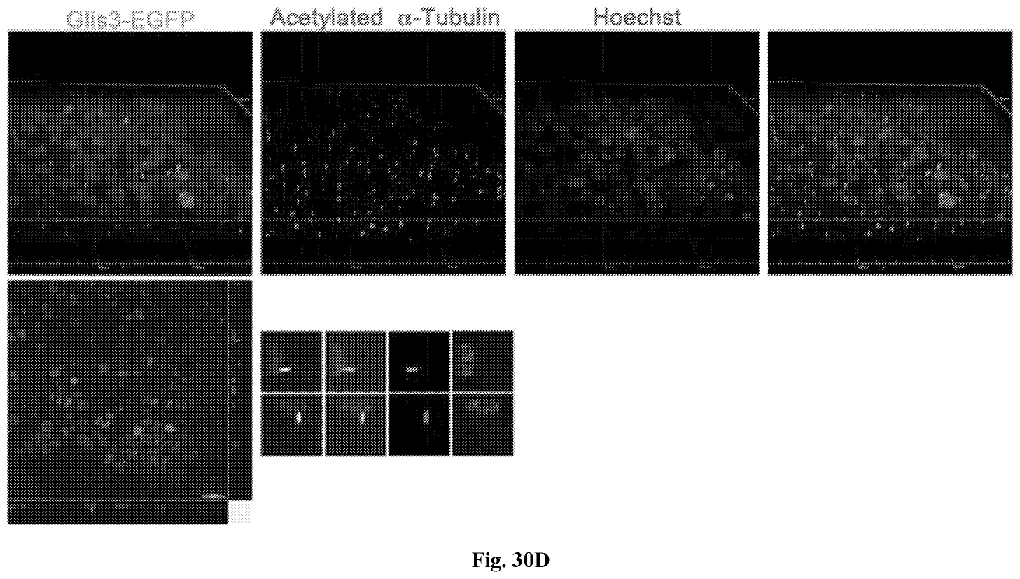

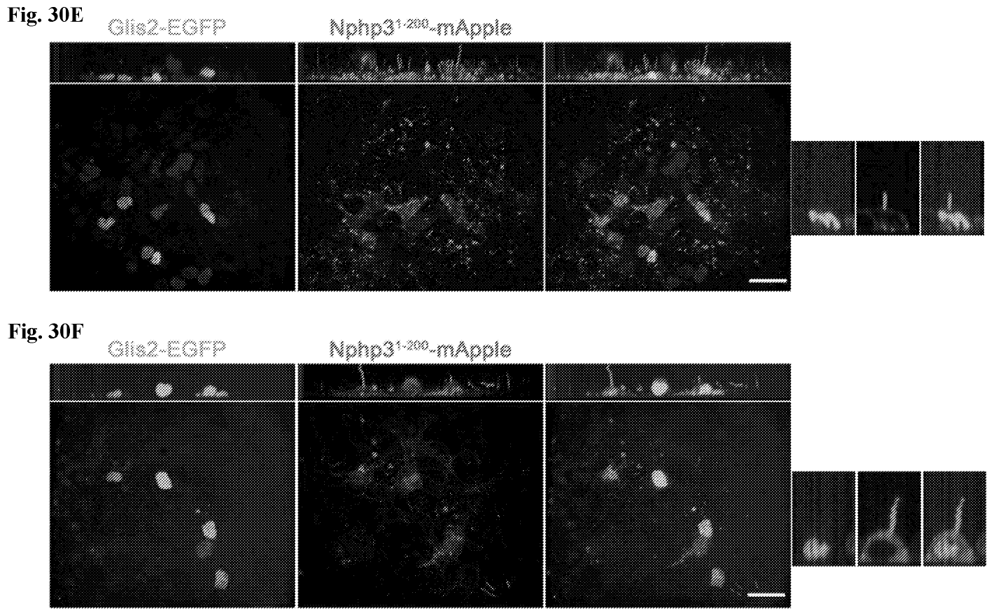

Glis2 protein had previously been reported to be localized in cilia so the present study sought to determine whether the loss of PC1 affected the presence of Glis2 in cilia. IMCD3 cells with knockout of Pkd1 grown under conditions to form cilia show upregulation of Glis2 and Pkd2 mRNA compared to wild type cells (FIG. 30A), mimicking the in vivo transcriptional changes in response to Pkd1 knockout observed in the TRAP RNASeq. This suggests that these cells should be adequate models for assessing PC1-dependent Glis2 subcellular expression. Unfixed ciliated IMCD3 cells expressing Glis2-EGFP and the fluorescent fusion protein cilia marker Nphp3⁽¹⁻²⁰⁰⁾-mApple under live cell imaging conditions showed nuclear localization of Glis2-EGFP but undetectable Glis2-EGFP expression in the Nphp3⁽¹⁻²⁰⁰⁾-mApple labeled cilia (FIG. 30B). Glis2-FLAG was similarly absent from cilia in fixed HEK293A cells, suggesting that neither cell type nor epitope label accounted for the absence from cilia (FIG. 30C). As an independent technical and positive control, Glis3-EGFP fusion protein was readily detectable in cilia in similar heterologous expression studies (FIG. 30D). Finally, the present study tested whether inactivation of PC1 resulted in Glis2 localization in cilia. IMCD3 cells with inactivation of PC1 also failed to show Glis2-EGFP expression in cilia (FIGS. 30E-30F). These data show that Glis2 is localized to the nucleus but is not detectable in cilia and the latter is unaffected by Pkd1 mutation status.

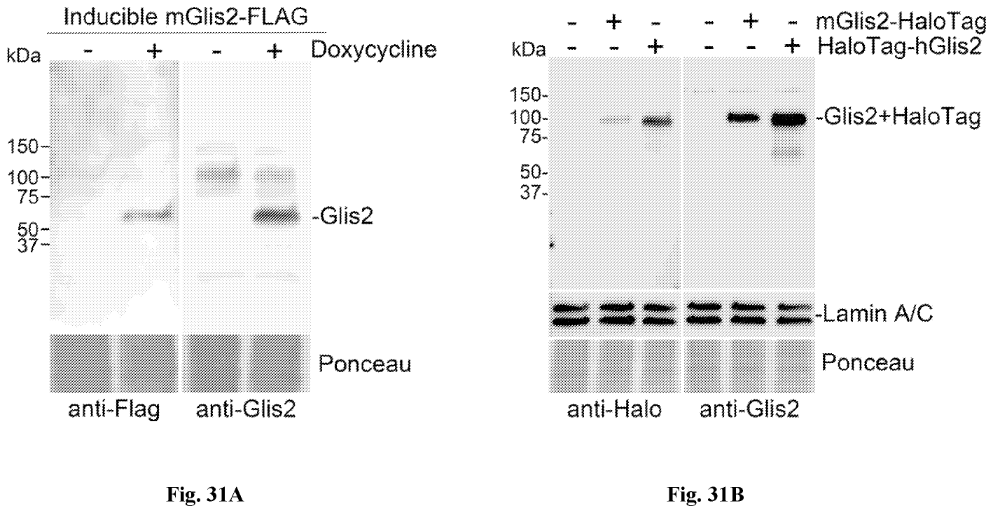

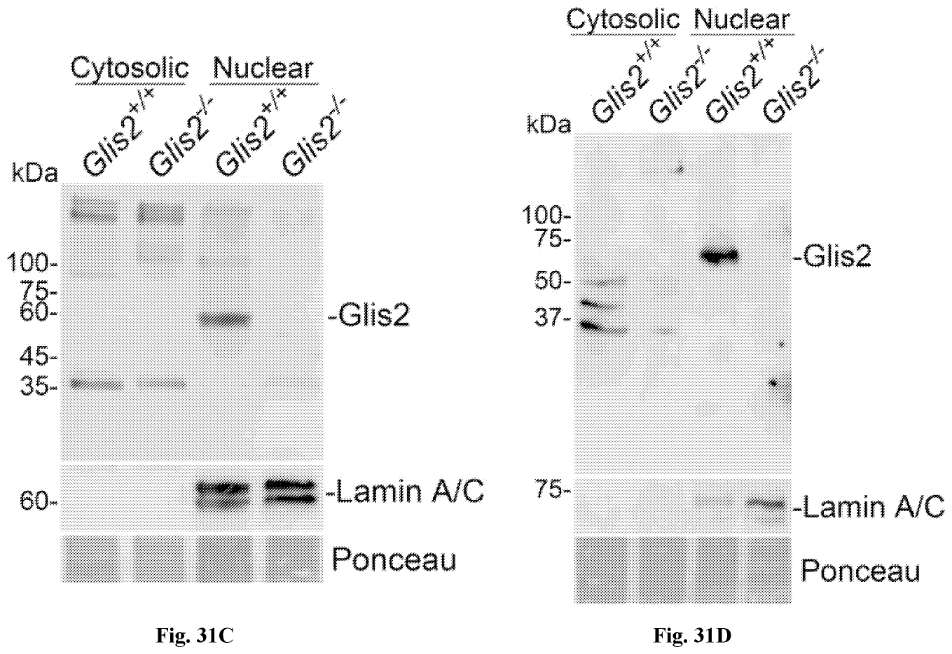

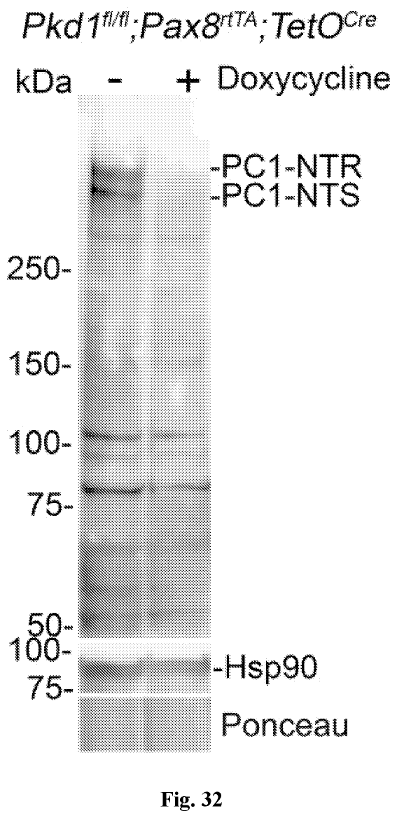

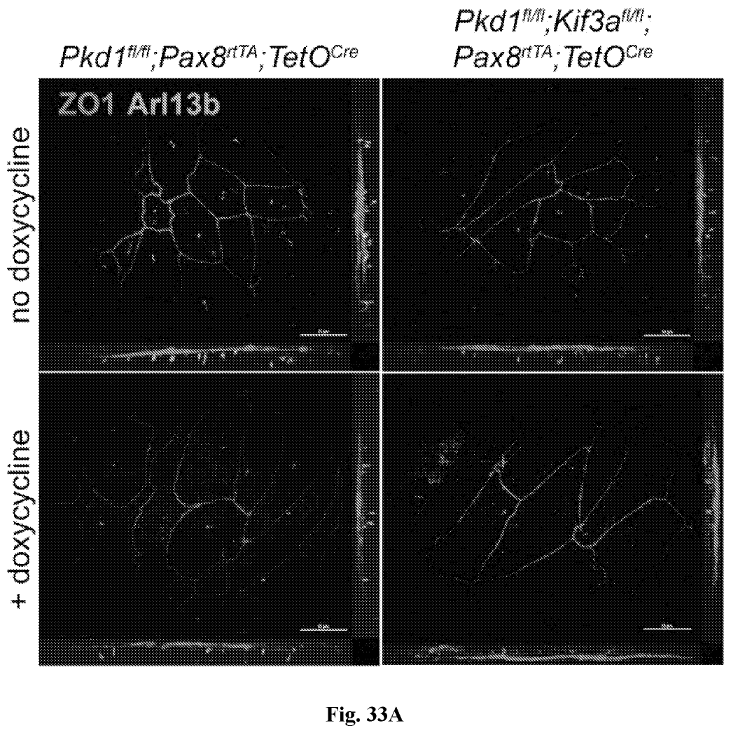

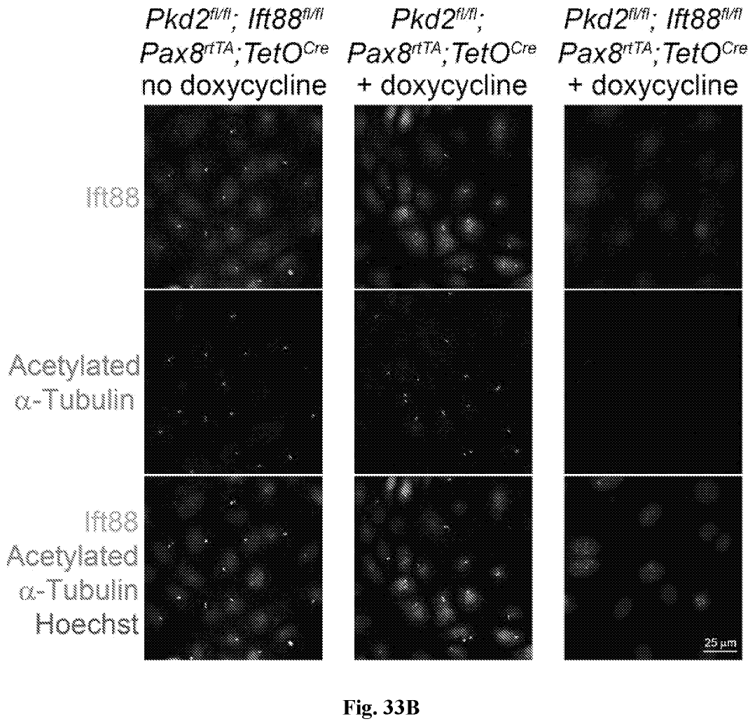

To allow comprehensive evaluation of changes in Glis2 protein expression, the present study developed and validated a polyclonal antibody to Glis2 (FIGS. 31A-31D). The anti-Glis2 antibody (YNG2) recognizes epitope tagged over-expressed mouse and human Glis2 by immunoblotting (FIGS. 31A-31B). The specificity of YNG2 is further indicated by detection of native Glis2 in nuclear fractions of cell lines and tissue lysates from kidneys of wild type mice, but not in kidney cell lines and lysates from Glis2 null mice (FIGS. 31C-31D). The bulk of Glis2 protein in both cultured cells and kidney tissue lysates is expressed in the nucleus, with little detected in the cytosolic fraction (FIGS. 31C-31D). The present study next used the developing kidney to evaluate correlation of native tissue mRNA and protein expression for Glis2. Glis2 showed elevated expression of both transcript and protein in postnatal kidneys at P1 and P10 followed by downregulation of both transcript and protein at P20 and P40 (FIGS. 19B-19C). Next, the present study examined Pkd genotype dependence of Glis2 expression in primary cells cultured from kidneys of mice with allele combinations that allowed for conditional inactivation of target genes following in vitro treatment with doxycycline. All cells were studied under conditions that promoted cilia formation prior to mRNA and protein extraction. Primary cell cultures from kidneys of Pkd1^{fl/fl}; Pax8^{rtTA}; TetO^{Cre} (FIGS. 19D-19E) and Pkd2^{fl/fl}; Pax8^{rtTA}; TetO^{Cre} (FIGS. 19F-19G) mice were treated with doxycycline in cell culture to induce inactivation of the respective Pkd gene (FIGS. 19G and 32). Glis2 transcript expression increased in both Pkd1 and Pkd2 knockout cells compared to cells not treated with doxycycline (FIGS. 19D and 19F). Doxycycline treatment alone, without Pkd gene knockout had no effect on Glis2 expression (data not shown). Pkd1 mutant primary cells also showed upregulation of Pkd2 transcripts, reproducing another feature of the in vivo TRAP RNASeq (FIG. 19D). Pkd1 transcript expression was upregulated following Pkd2 inactivation in the cell system suggesting possible coordinate transcriptional regulation of the polycystin genes (FIG. 19F). At the protein level, Glis2 showed increased nuclear expression following doxycycline induced Pkd1 or Pkd2 inactivation (FIGS. 19E and 19G). Primary cell cultures were also produced from kidneys of littermate mice with genotypes supporting inducible in vitro inactivation of Pkd1 alone or Pkd1 and Kif3a (Pkd1+ cilia) together. These cells required longer time in culture after doxycycline treatment to allow for disappearance of cilia (FIG. 33A). Cells with Pkd1 inactivation alone again showed upregulation of both transcript and protein expression for Glis2 and Pkd2 (FIGS. 19H and 19I). Cells with dual inactivation of Pkd1+ cilia showed no difference in expression of either Glis2 or Pkd2 compared to control cells without Pkd1 knockout (FIGS. 19H and 19I). Suppression of Glis2 protein expression was also observed with Pkd2 and Ift88 double knockout cells compared to Pkd2-only knockout primary cultures (FIGS. 19J and 33B). Adult inducible Pkd1 and Tulp3 double knockout mice have a similar suppressive effect on kidney cyst formation as Pkd1+ cilia knockouts, but without loss of cilia. Glis2 and PC2 protein expression in Pkd1; Tulp3 double knockout cells remained unchanged from non-knockout controls further supporting the strict correlation of Glis2 levels in vitro with cyst forming potential in vivo (FIG. 19K). Glis2 transcript and Glis2 protein expression are the first molecular markers to recapitulate the CDCA profile in vitro.

Finally, the present study performed in vivo validation experiments using mouse kidney tissues in two different models to determine whether changes in protein expression correlated with changes in transcript levels. In the first model, kidney tissue lysates from Pkd1^{fl/fl}; Pax8^{rtTA}; TetO^{Cre} mice showed increased Glis2 as well as PC2 expression following doxycycline induced Pkd1 knockout compared to mice with the same genotype that did not have doxycycline induced knockout (FIG. 19L). This knockout-dependent change in expression was abrogated in doxycycline treated Pkd1^{fl/fl}; Kif3a^{fl/fl}; Pax8^{rtTA}; TetO^{Cre} double knockout mouse kidney lysates (FIG. 19L). The increase in Glis2 expression following Pkd1 inactivation in Pkd1^{fl/fl}; Pax8^{rtTA}; TetO^{Cre} mice was primarily in the nuclear fraction of the tissue (FIG. 19M). In the second model, Glis2 protein upregulation in response to Pkd1^{KO} was markedly suppressed in Pkd1^{fl/fl}; Tulp3^{fl/fl}; Pax8^{rtTA}; TetO^{Cre} mice following dual gene inactivation that is known to prevent cyst progression (FIG. 19M). In aggregate, these data confirm the strong CDCA pattern correlation between Glis2 nuclear expression and propensity for cyst formation in vivo. It was concluded that polycystin dependent transcriptional changes in Glis2 manifest as correlated changes in protein expression with most of the protein showing nuclear localization in vivo and in vitro. Glis2 shows genotype and phenotype dependent changes in expression in both Pkd1 and Pkd2 primary kidney cells that recapitulate the expression changes discovered by the TRAP RNASeq studies in vivo. Ghs2 transcript and protein expression changes in primary cells are a surrogate indicator in vitro of polycystin function dependent cyst forming potential in vivo.

### Example 2-5: Inactivation of Glis2 Slows Cyst Progression in Mouse Models of ADPKD

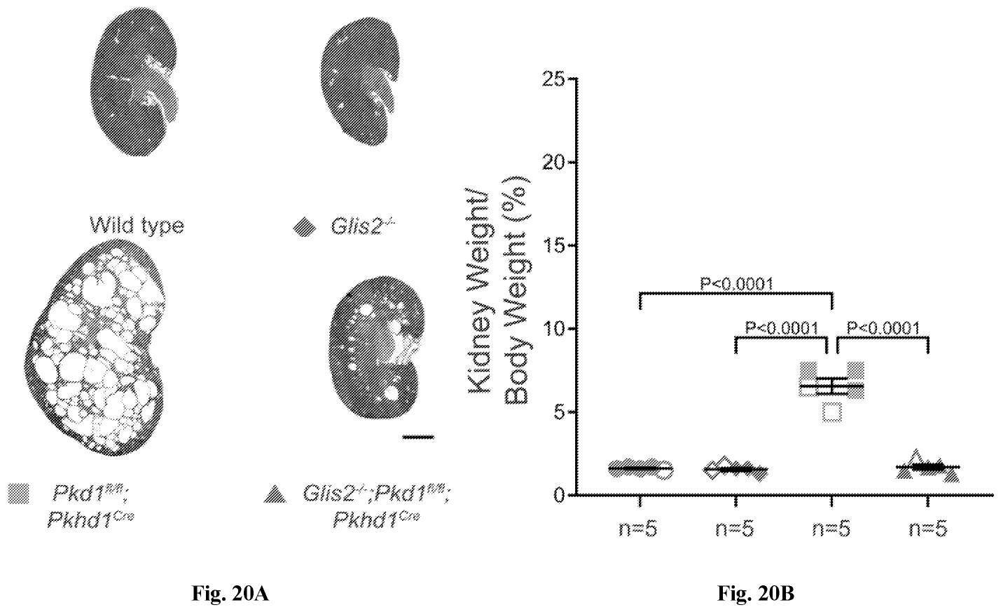

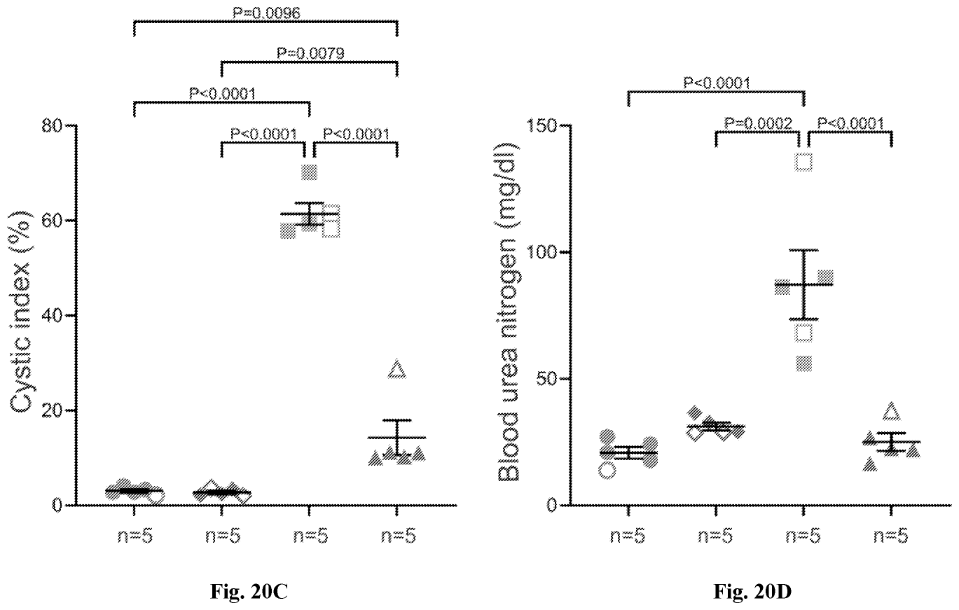

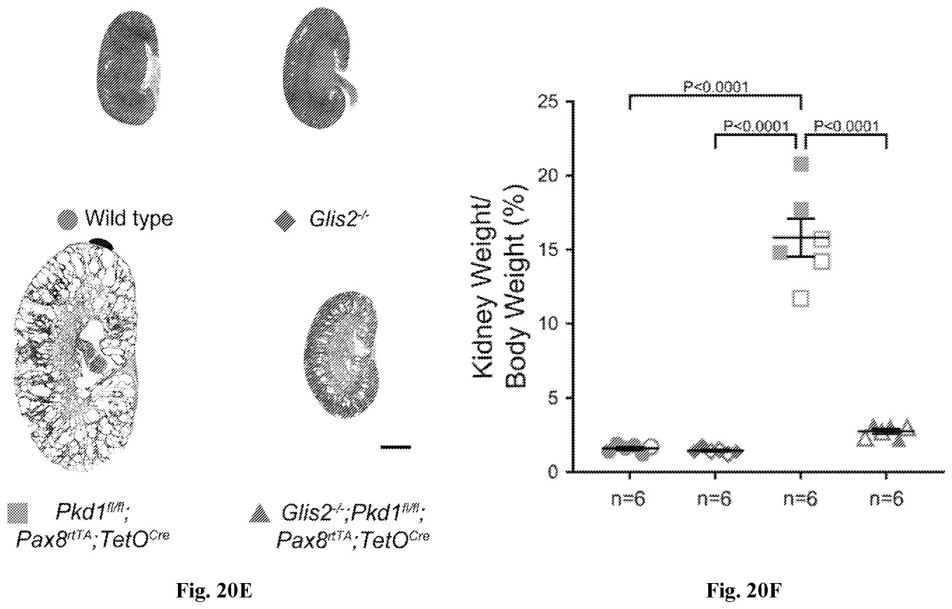

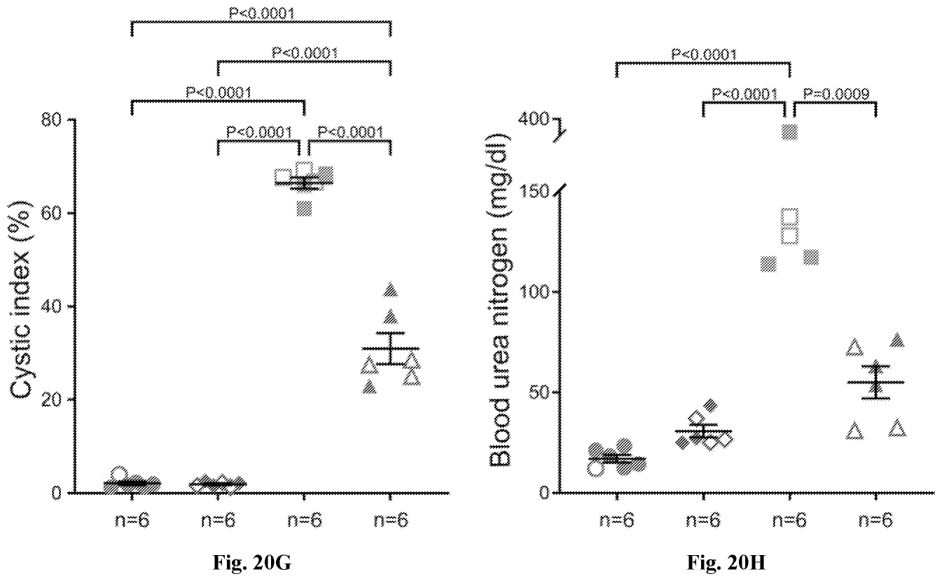

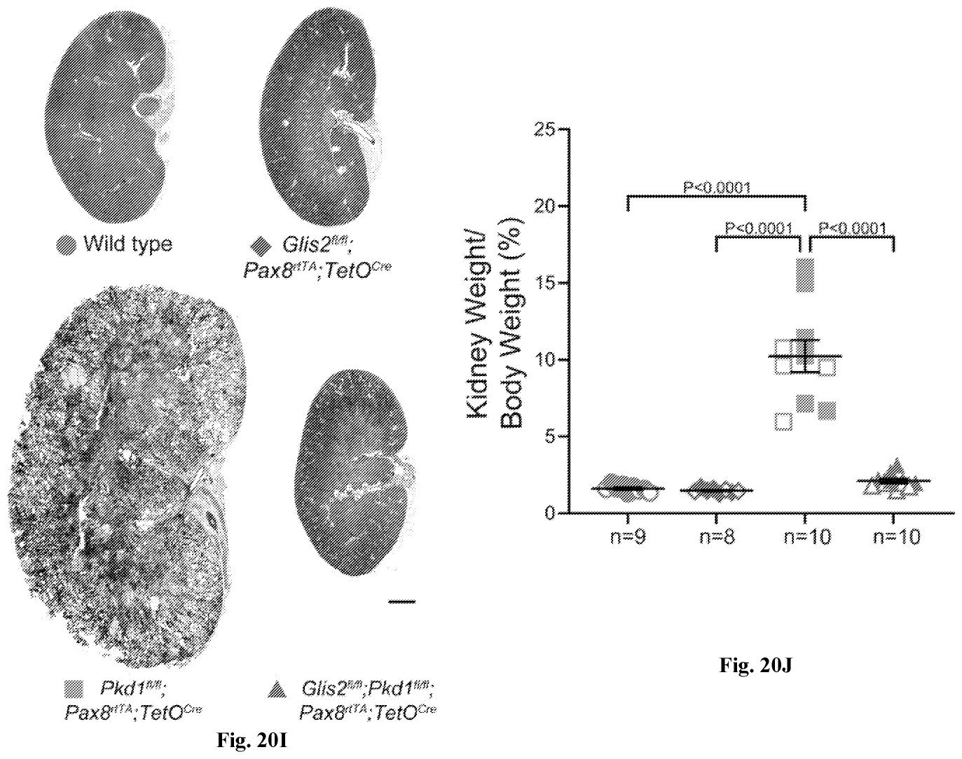

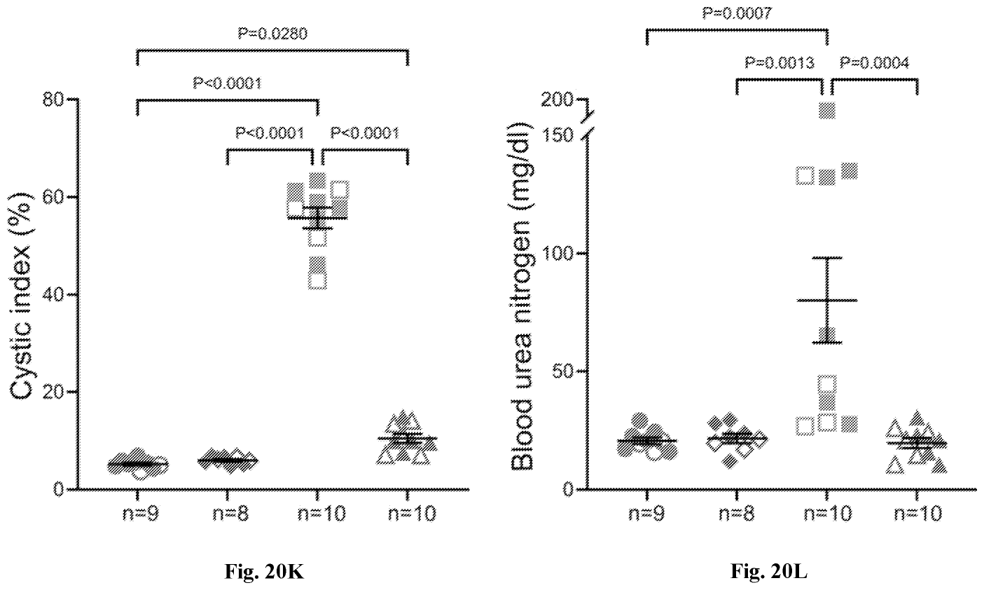

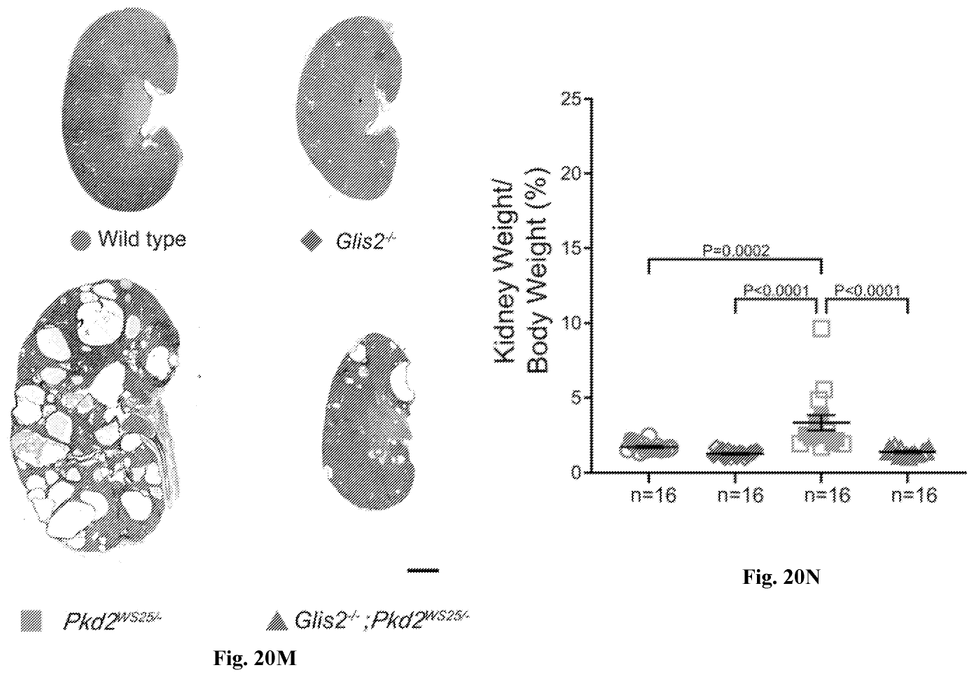

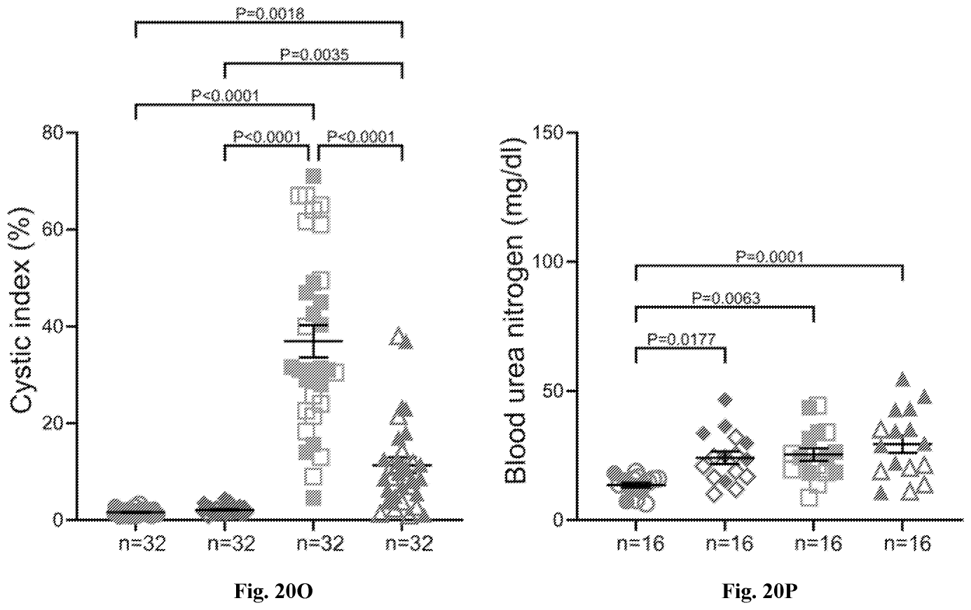

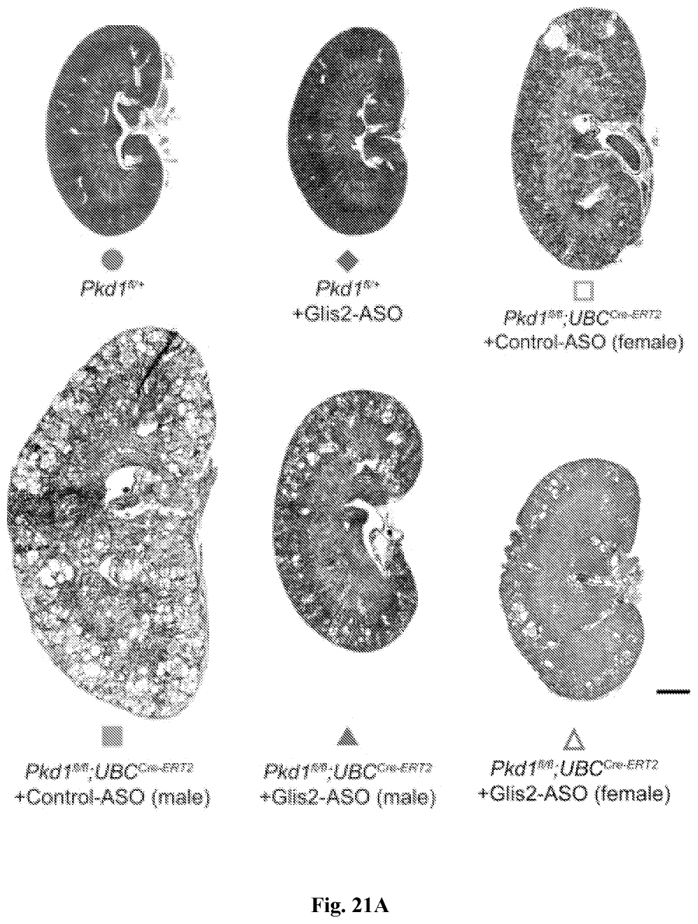

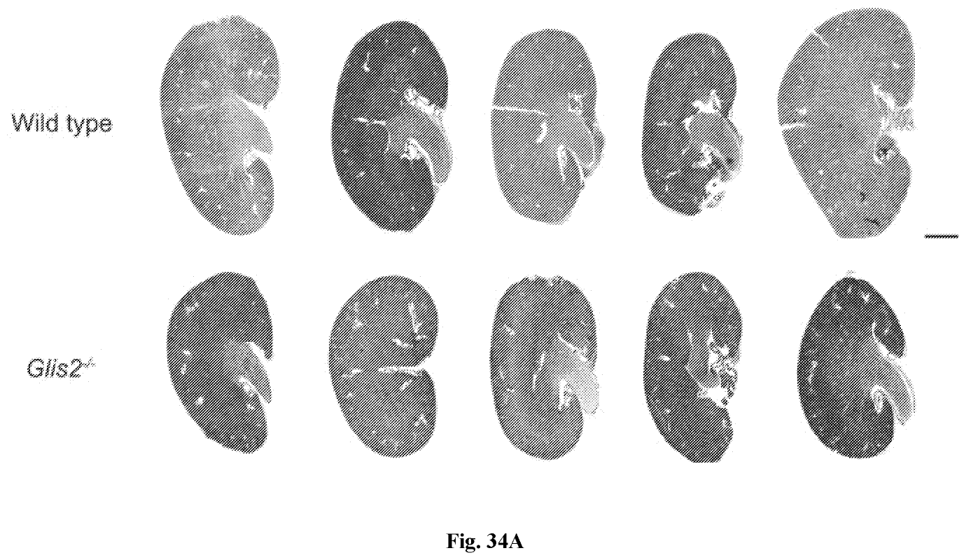

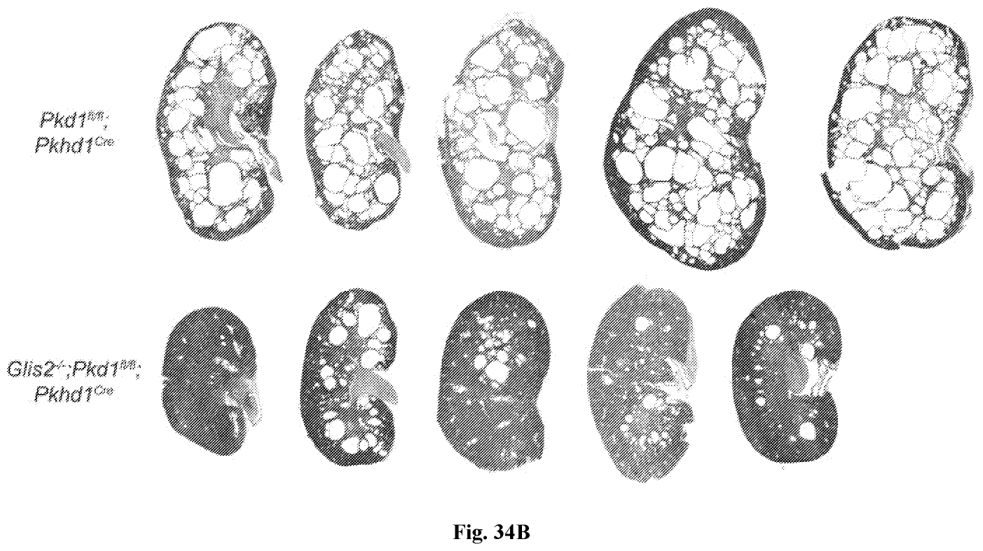

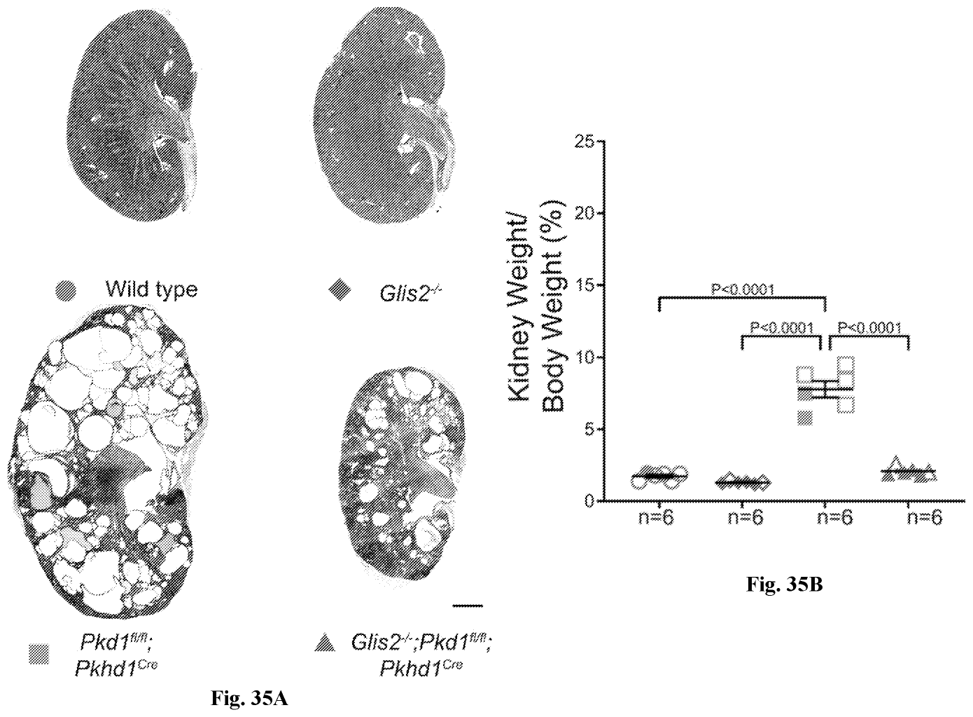

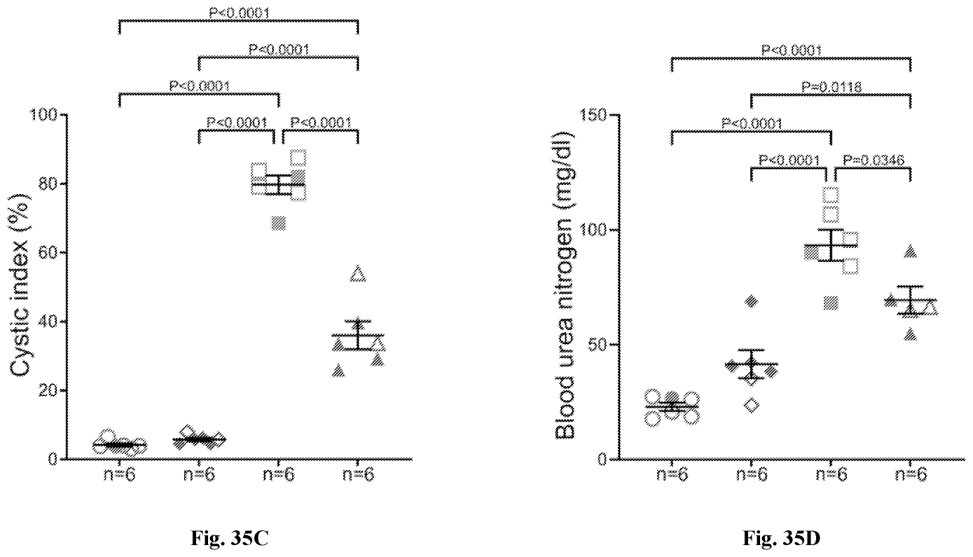

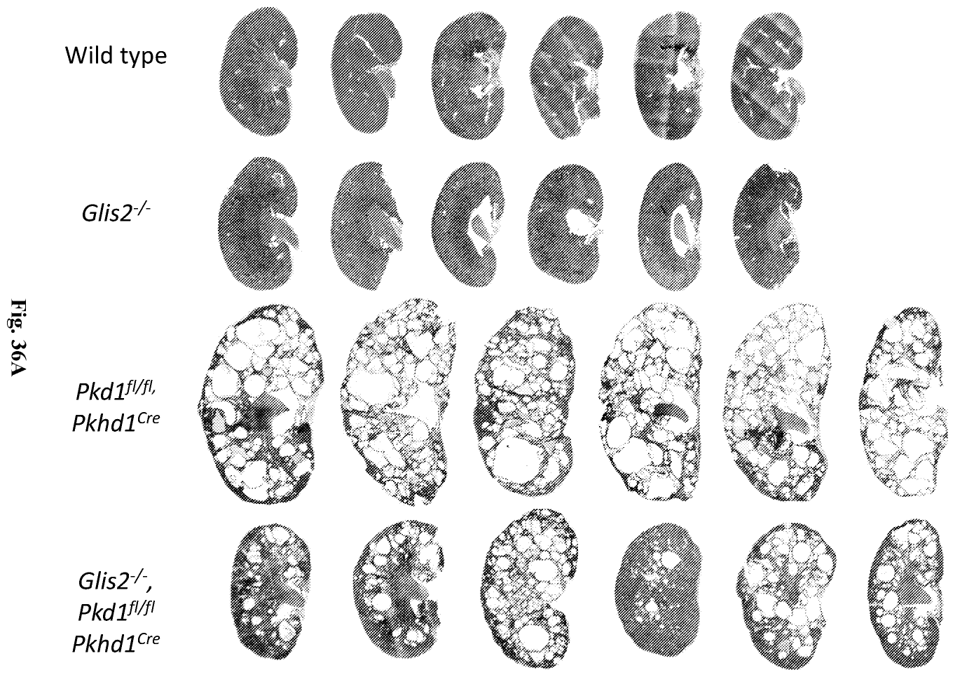

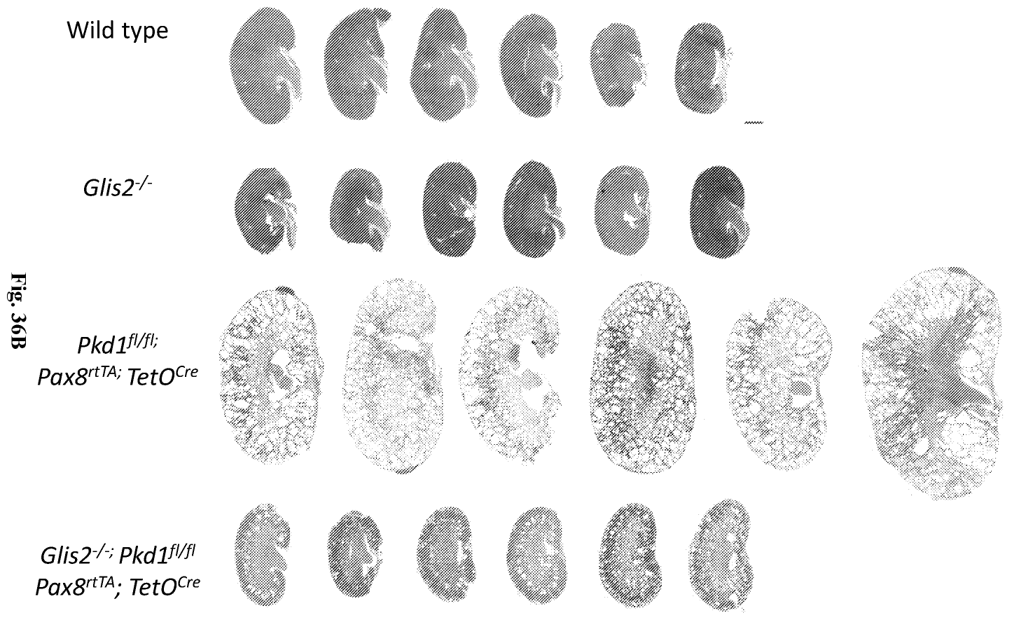

To investigate whether Glis2 had an epistatic role in ADPKD, the present study used a series of mouse allele combinations to determine whether inactivation of GIis2 affected polycystin-dependent cyst progression. The present study first introduced a knock-out allele of Gis2 into the early-onset Pkd1^{fl/fl}; Pkhd1^{Cre} mouse model (FIGS. 20A-20D and 34A-34B). Pkhd1^{Cre} results in complete inactivation of Pkd1 in principal cells of the collecting duct by P7. Mice have significant polycystic kidney disease at P24 manifest by the structural parameters of increased kidney-to-body weight ratio and percent cystic area (cystic index) and the kidney functional measure of increased blood urea nitrogen (BUN). Glis2 null mice at that age show subtle microscopic changes of mild cortical tubule atrophy and thickening of the glomerular basement membrane with normal BUN. Glis2^{-/-}; Pkd1^{fl/fl}; Pkhd1^{Cre} were significantly protected from polycystic kidney disease compared to Pkd1^{fl/fl}; Pkhd1^{Cre} mice at P24 (FIGS. 20A-20D and 34A-34B). The present study further evaluated the durability of this rescue by examining mice with these same allele combinations at P49 (FIGS. 35A-35D). Germline null Glis2 supported persistent relative improvement of polycystic kidney disease with normal kidney-to-body weight ratio and significantly milder cystic index and BUN elevation when compared to Pkd1^{fl/fl}; Pkhd1^{Cre} mice at P49. To examine whether the protective effect of Glis2 loss on cyst growth is also applicable to other nephron segments as would be expected of factors related to CDCA, Glis2^{-/-} knockout was combined with Pkd1^{fl/fl}; Pax8^{rtTA}; TetO^{Cre} in another early onset mouse model. Oral doxycycline was administered to nursing female mice from P0-P14 and kidneys of the progeny litter were analyzed at P14. Absence of Glis2 resulted in significant protection from cyst growth following Pkd1 inactivation as indicated by significant improvements in kidney-to-body weight ratio, cystic index and BUN levels (FIGS. 20E-20H and 36A-36B). These findings suggest that elevated expression of Glis2 is a permissive factor for cyst progression in vivo and absence of Glis2 improves renal prognosis in early onset ADPKD models based on Pkd1.

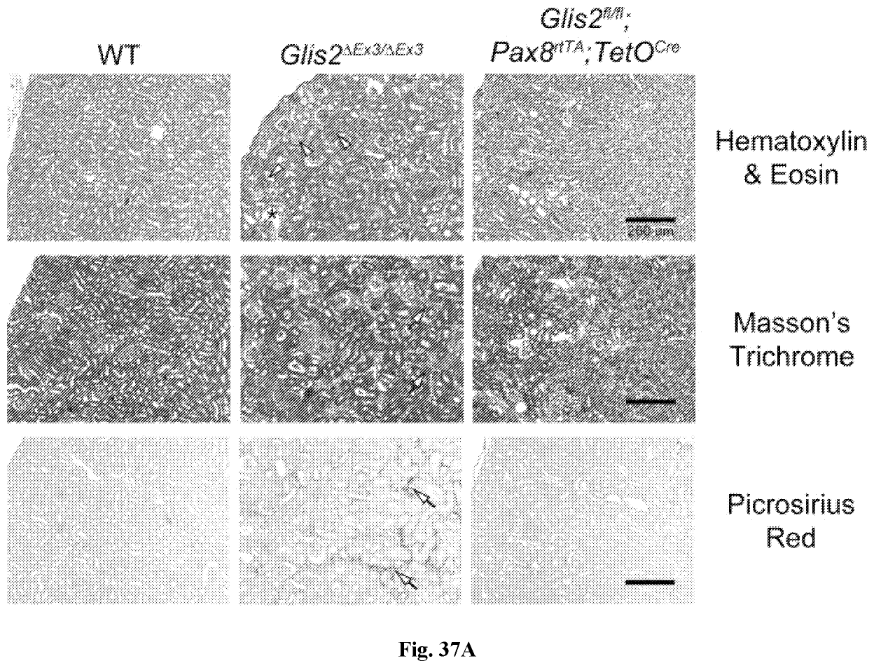

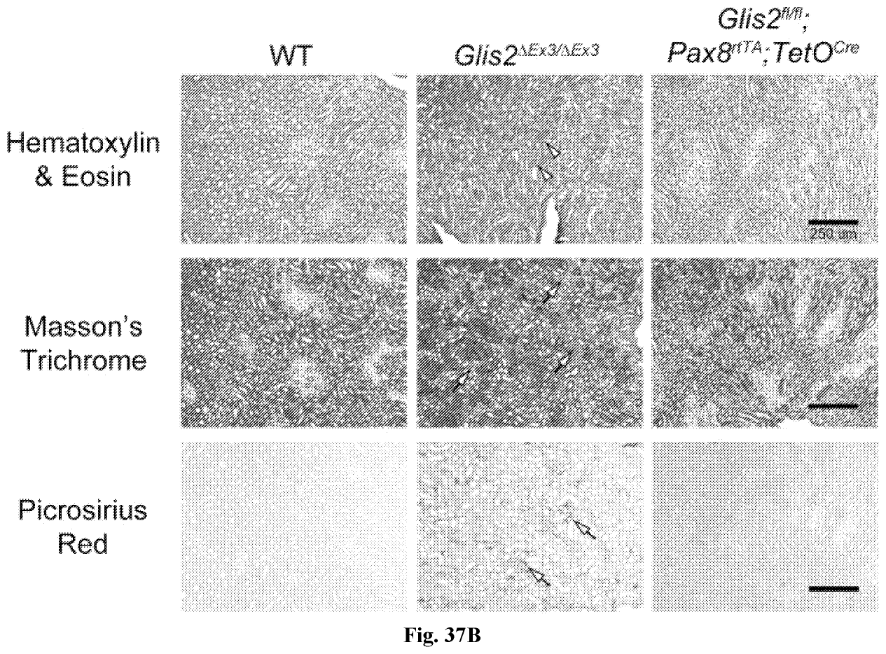

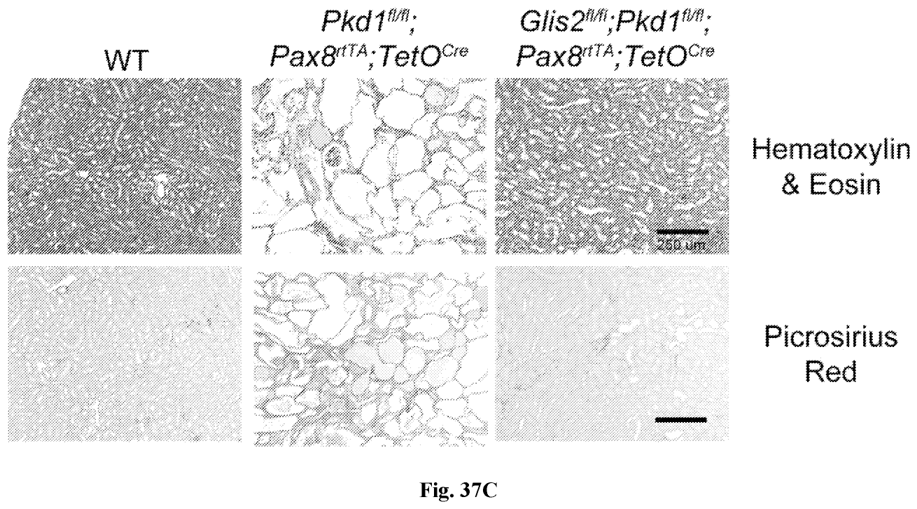

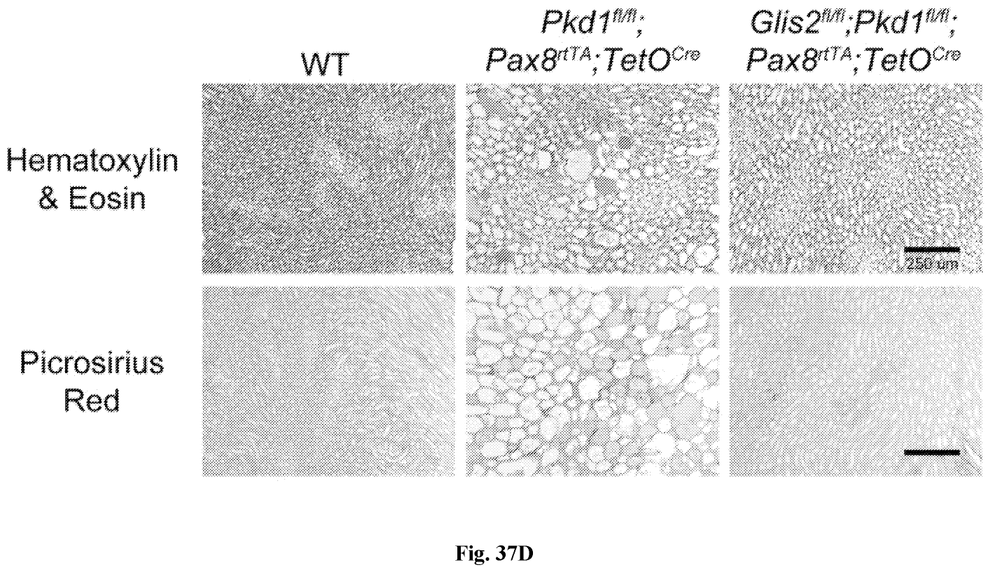

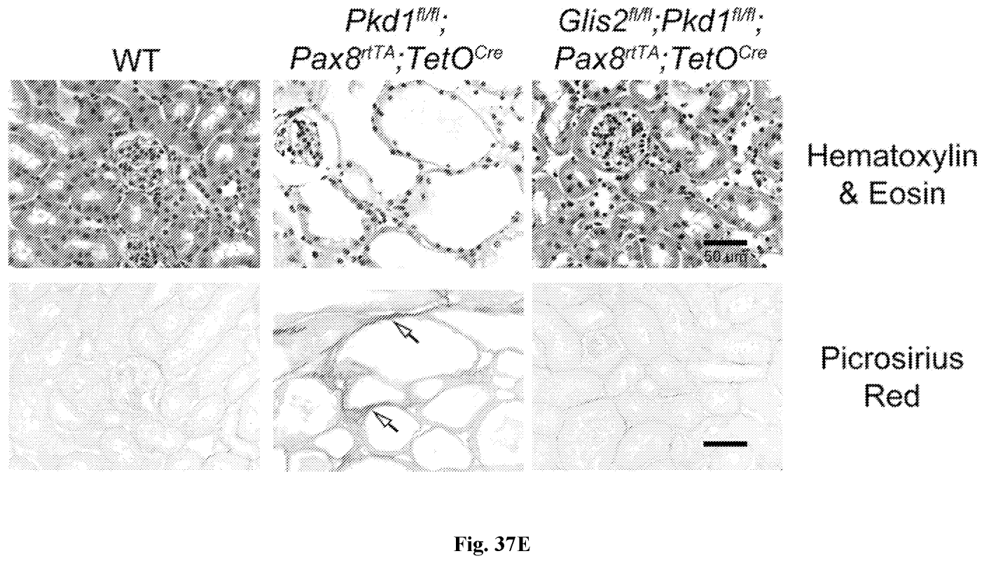

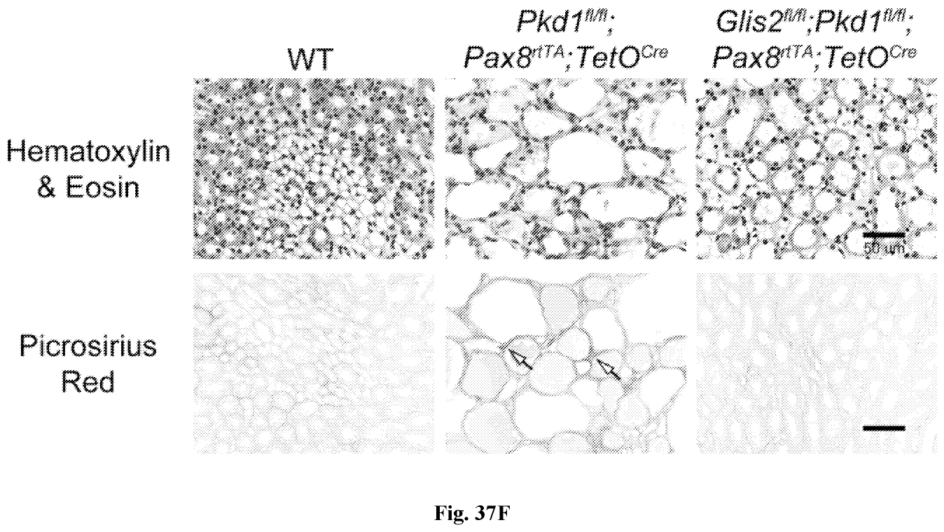

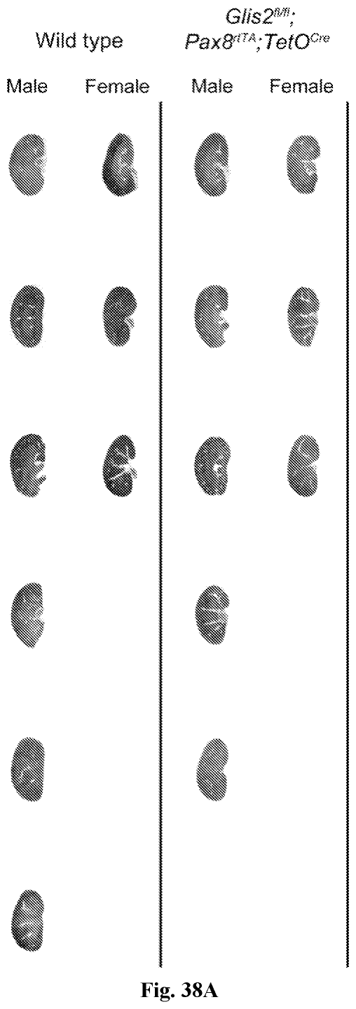

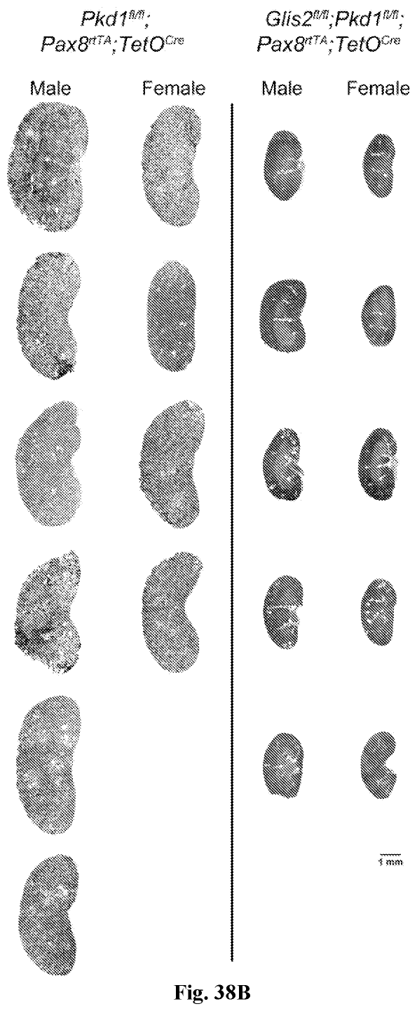

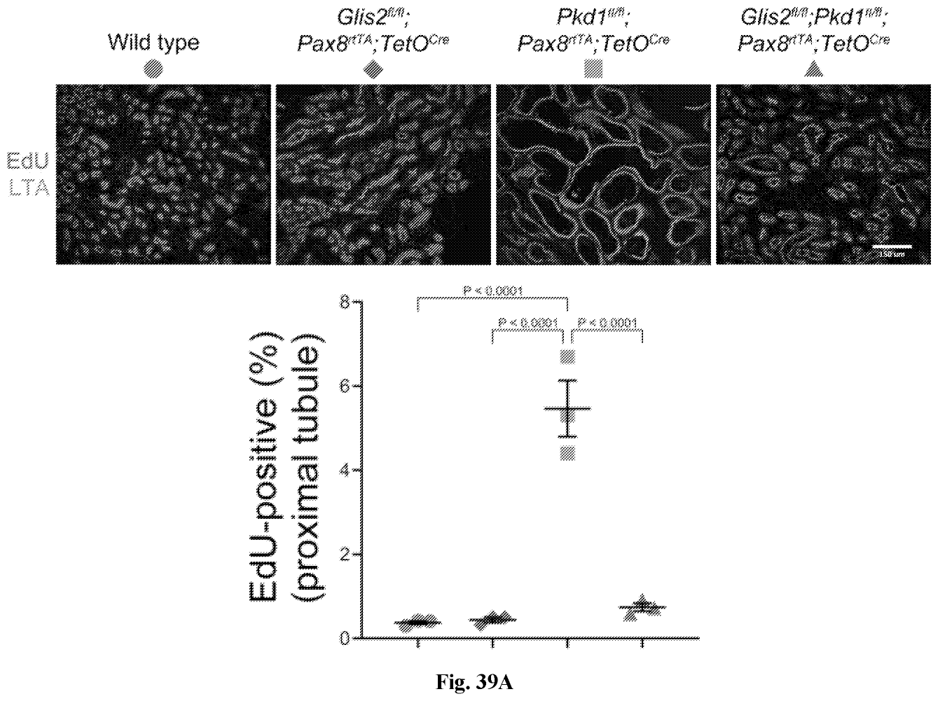

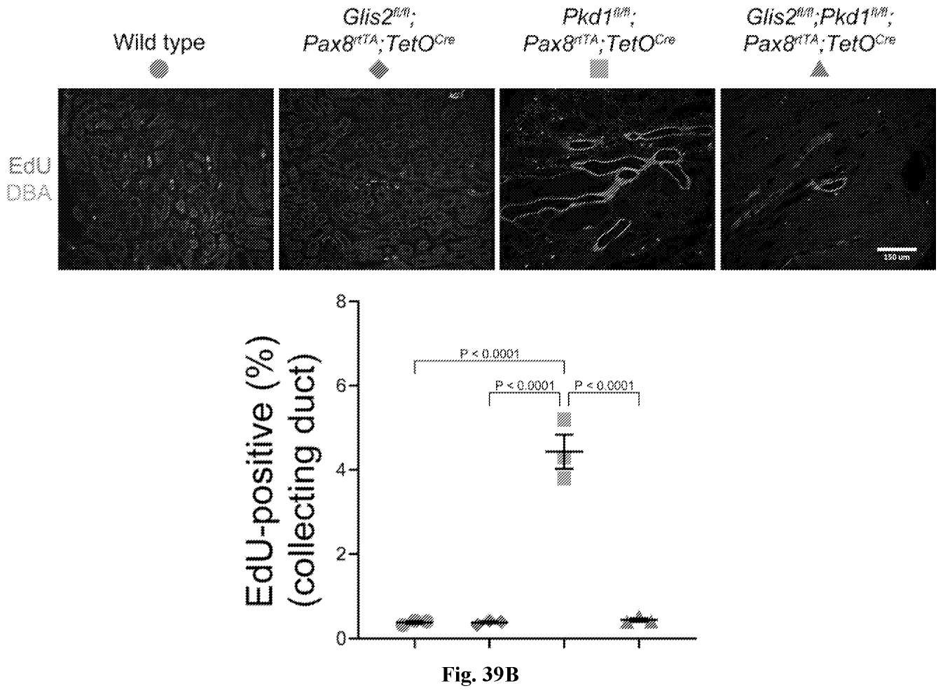

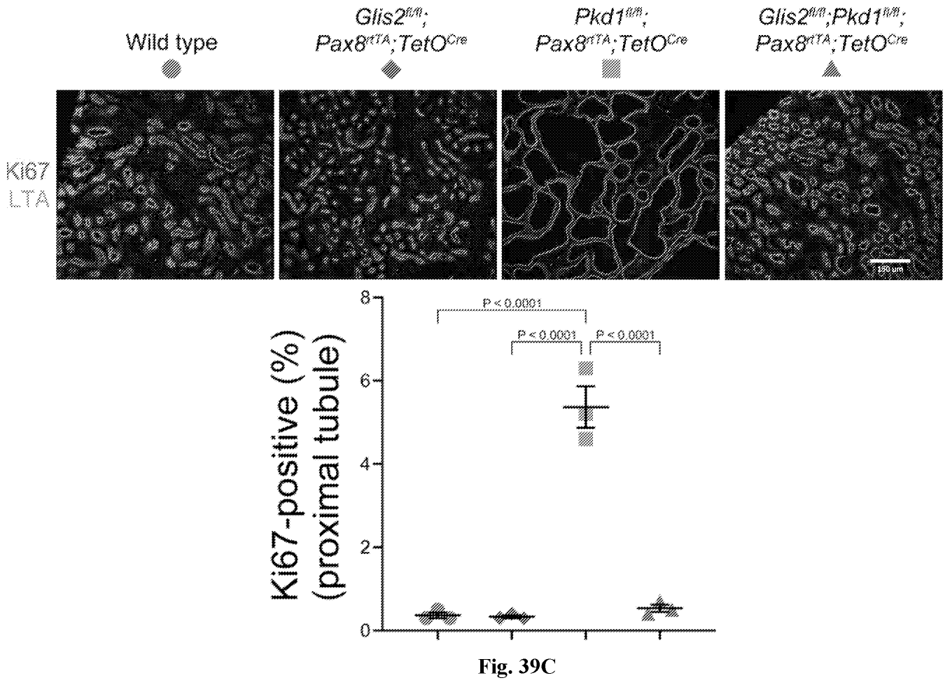

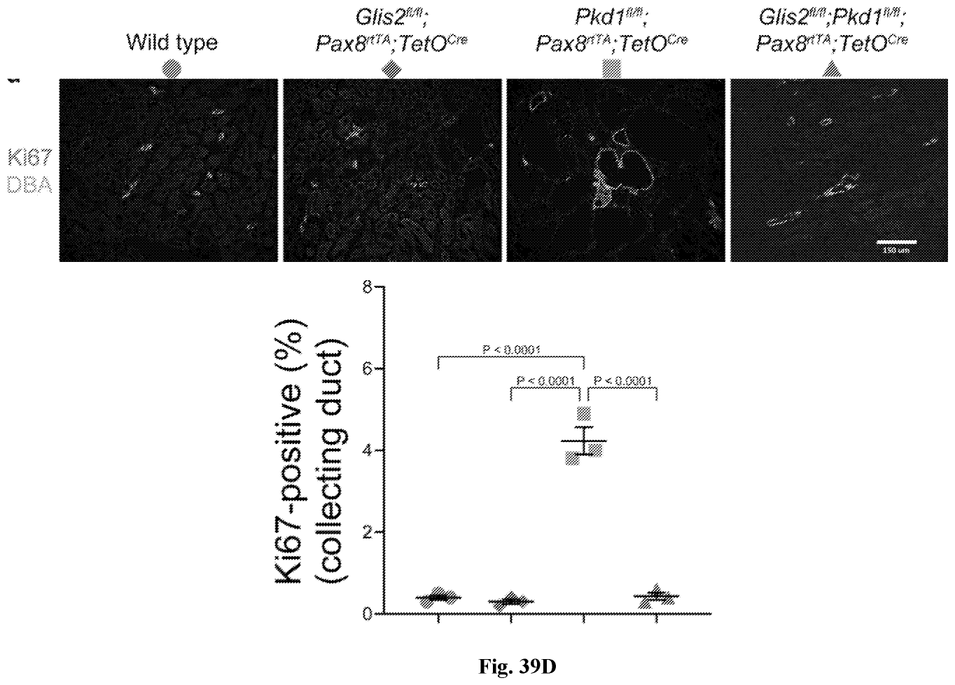

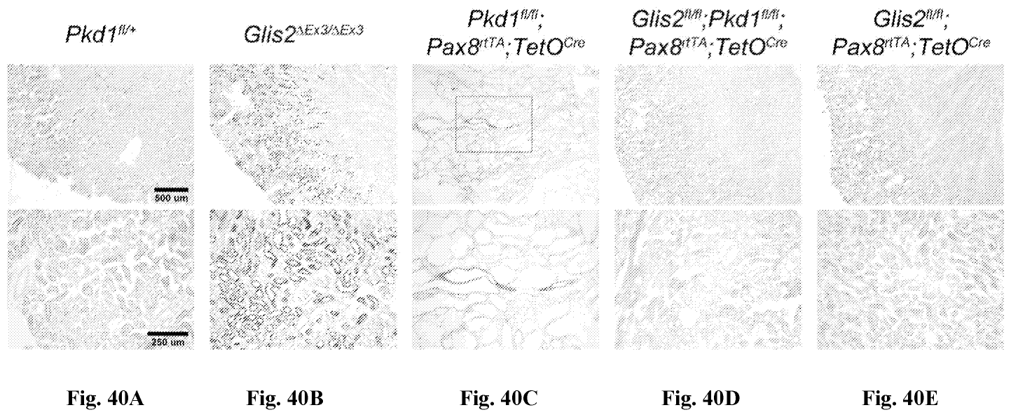

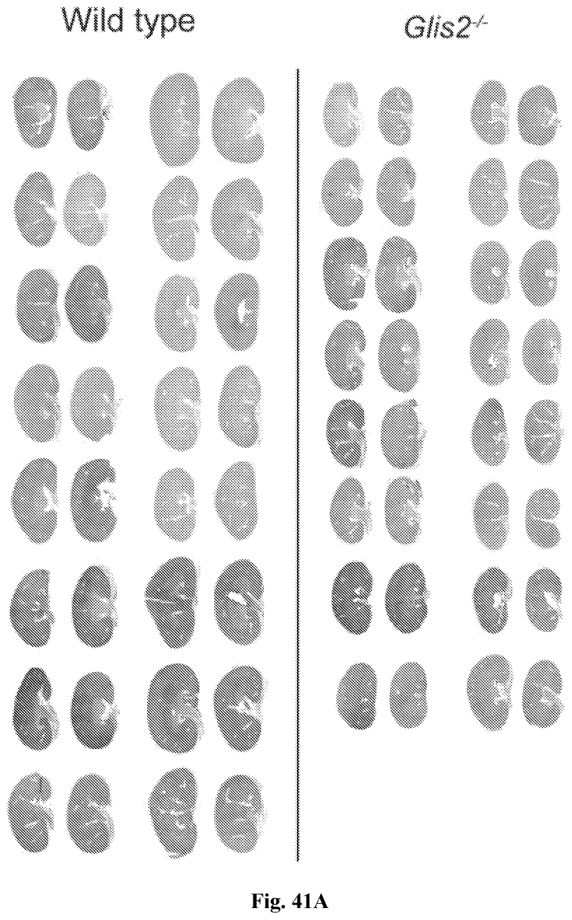

Cyst growth in adult onset ADPKD models is slower than in early onset models and may more closely reflect the course of ADPKD in most patients. Since germline null Glis2^{-/-} mice develop progressive nephronophthisis-related chronic kidney disease that is evident at 4 months age, the present study developed an inducible conditional Glis2^{fl/fl}; Pkd1^{fl/fl}; Pax8^{rtTA}; TetO^{Cre} model to evaluate the effects of contemporaneous Glis2 inactivation on cyst growth in later onset mouse models. All mice were administered doxycycline from P28-P42, and severity of polycystic kidney disease was examined at 18 weeks age. Unlike germline null Glis2 inactivation, inducible inactivation of Glis2 alone beginning at P28 did not result in any discernible kidney phenotype when assessed for structural changes and evidence of collagen deposition at 18 weeks (FIGS. 37A-37B). Pkd1^{fl/fl}; Pax8^{rtTA}; TetO^{Cre} mice at 18 weeks showed the expected structural and fibrotic changes of polycystic kidney disease along with elevated BUN indicative of kidney function impairment (FIGS. 20I-20L, 37C-37F and 38A-38B). Glis2 and Pkd1 double mutant mice showed a much milder phenotype than Pkd1 single mutants at 18 weeks age when evaluated by kidney-to-body weight ratio, cystic index, BUN and histologic criteria (FIGS. 20I-20L 37C-37F and 38A-38B). Glis2 and Pkd1 dual inactivation was accompanied normalization of kidney epithelial cell proliferation measured by EdU incorporation and Ki67 in both proximal and distal nephron segments compared to cystic Pkd1 single mutants (FIGS. 39A-39D). Germline inactivation of Gis2 also results in senescence in kidney epithelial cells. The present study used senescence-associated β-galactosidase (SA-β-gal) to detect senescent cells in kidneys of 18-week-old mice with either germline null or adult inducible inactivation of Glis2 (FIGS. 40A-40E). Glis2^{ΔEx3/ΔEx3} null mice showed extensive cortical SA-β-gal staining indicative of cellular senescence (FIGS. 40A-40B). Kidneys from Pkd1^{fl/fl}; Pax8^{rtTA}; TetO^{Cre} mice showed only occasional cysts with senescent cells (FIG. 40C). Adult Glis2^{fl/fl}; Pkd1^{fl/fl}; Pax8^{rtTA}; TetO^{Cre} double knockout or Glis2^{fl/fl}; Pax8^{rtTA}; TetO^{Cre} single knockout kidneys did not show any evidence of senescence (FIGS. 40D-40E). These findings indicate that the association of Glis2 inactivation with epithelial cell senescence in the kidney is a feature of germline null nephronophthisis phenotypes and not a feature of postnatal kidney tubule selective inactivation of Glis2. Overall, adult inducible in activation of Glis2 in kidney epithelial cells suppresses kidney cyst growth following Pkd1 inactivation without evidence of untoward effects on kidney structure or function.

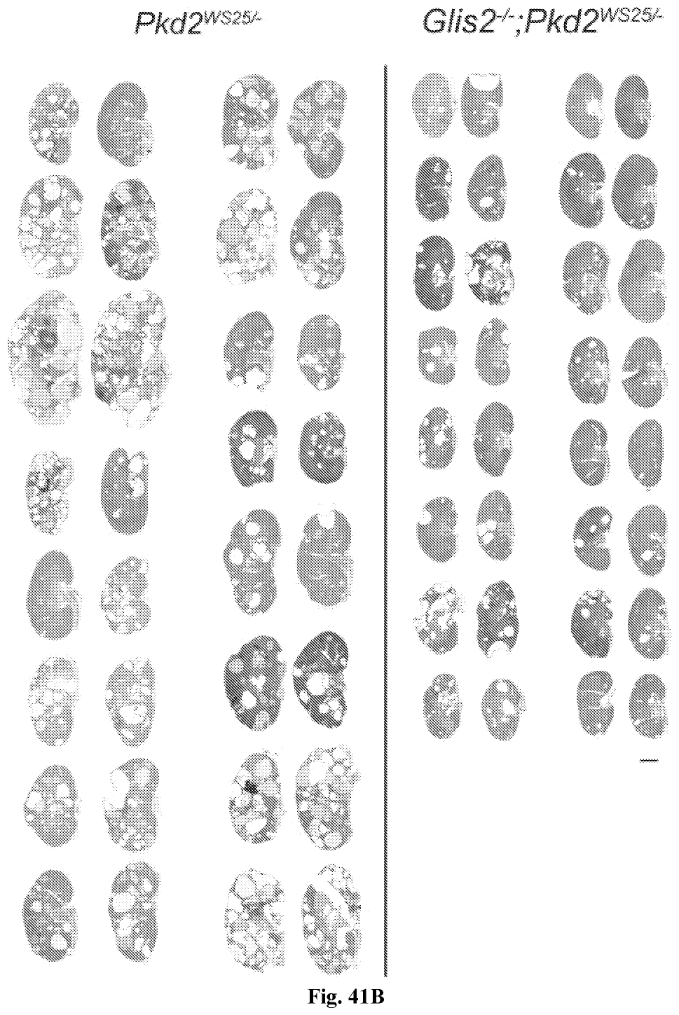

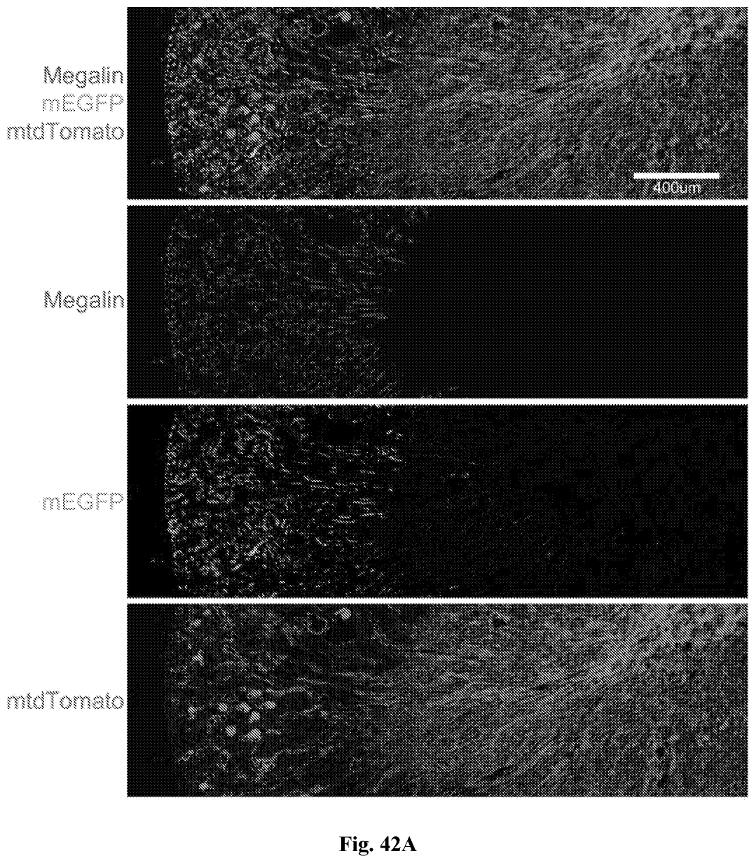

Finally, CDCA related factors are expected to affect ADPKD due to Pkd2 as well as Pkd1. The present study examined the effects of Gis2^{-/-} null alleles on polycystic kidney disease progression in the Pkd2^{WS25/-} model⁵³. The spontaneous stochastic second hit mutations in Pkd2^{WS25/-} most closely recapitulate the mechanism of human ADPKD. Since there is no Cre recombinase in this model, the present study used the Glis2^{-/-} null mice. The present study selected 14 weeks as the endpoint for the analysis to allow enough time for the highly variable Pkd2^{WS25/-} model to develop polycystic kidney disease while limiting the progressive chronic kidney disease that occurs in Glis2^{-/-} mice. Glis2^{-/-}; Pkd2^{WS25/-} mice showed reduced cyst growth in kidneys as reflected by significant reduction in kidney-to-body weight ratio and cystic index (FIGS. 20M-30P and FIGS. 41A-41B). The same degree of mild impairment of kidney function was observed in Glis2^{-/-}, Pkd2^{WS25/-}, and Glis2^{-/-}; Pkd2^{WS25/-} mice. The underlying impairment of kidney function caused by the Glis2^{-/-} null mutation precludes clear interpretation of this result, but the absence of an additive worsening of BUN in Glis2^{-/-}; Pkd2^{WS25/-} mice is permissive for the interpretation that renal impairment due to polycystic kidney disease was prevented with the reduction in cyst growth. Glis2 inactivation is effective in reducing polycystic kidney disease progression in adult models based on both Pkd1 and Pkd2. The Pkd genotype-dependent changes in Glis2 transcription and protein expression have a causative role in progression of polycystic kidney disease that can be ameliorated by inactivation of the Glis2 gene.

### Example 2-6: Glis2 is a Target for Therapy in ADPKD

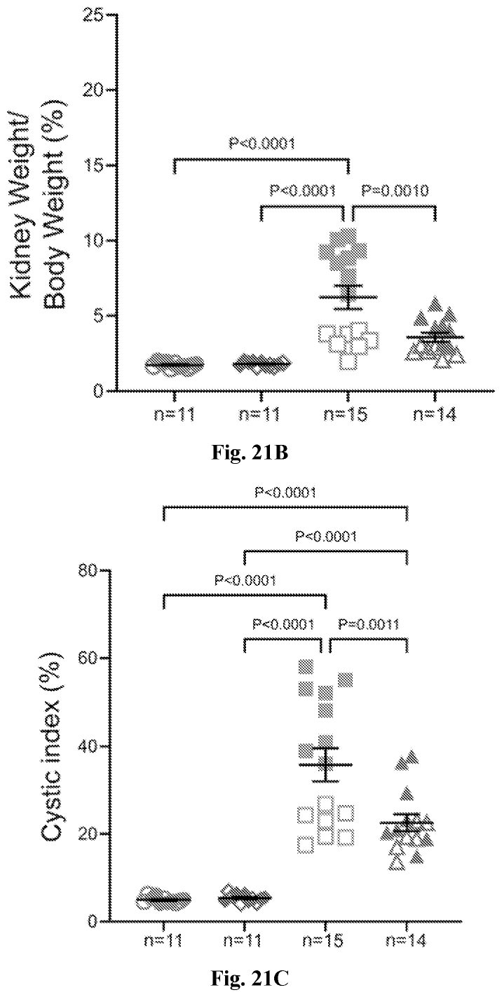

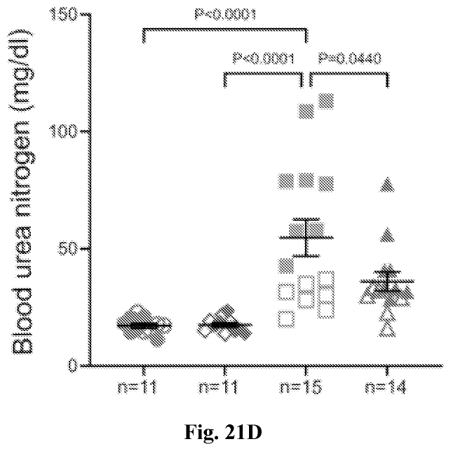

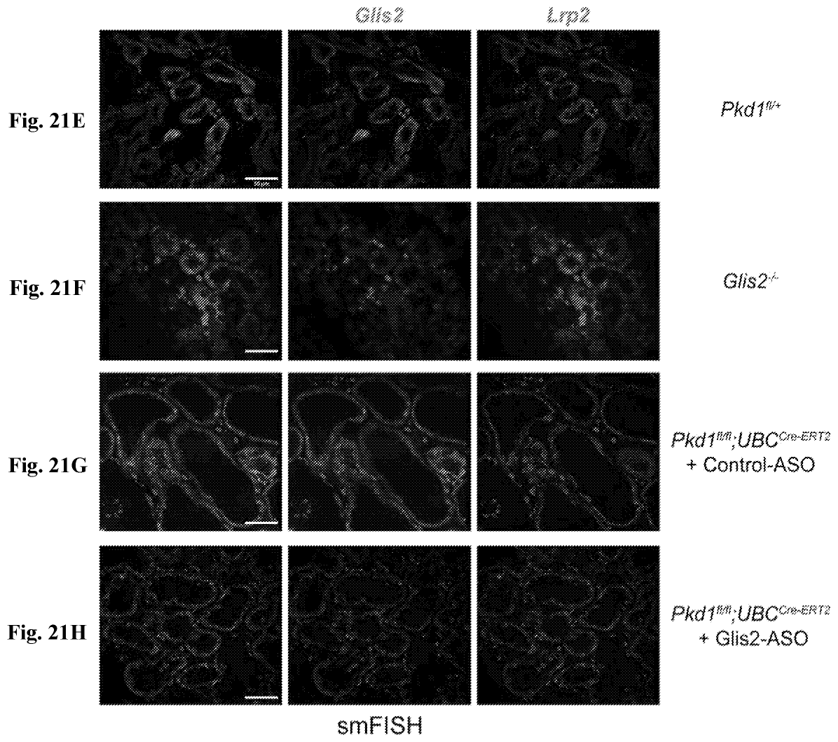

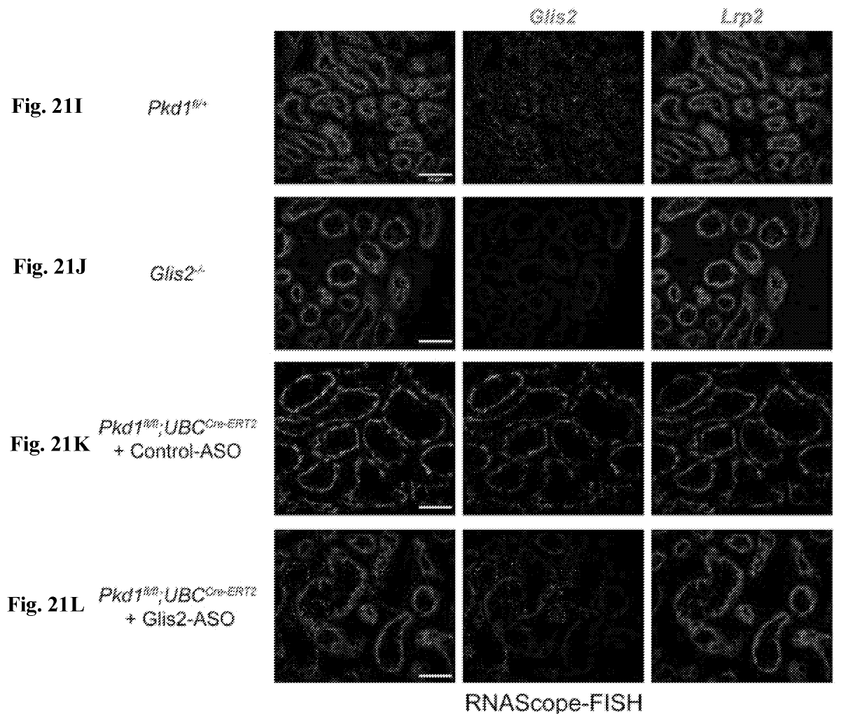

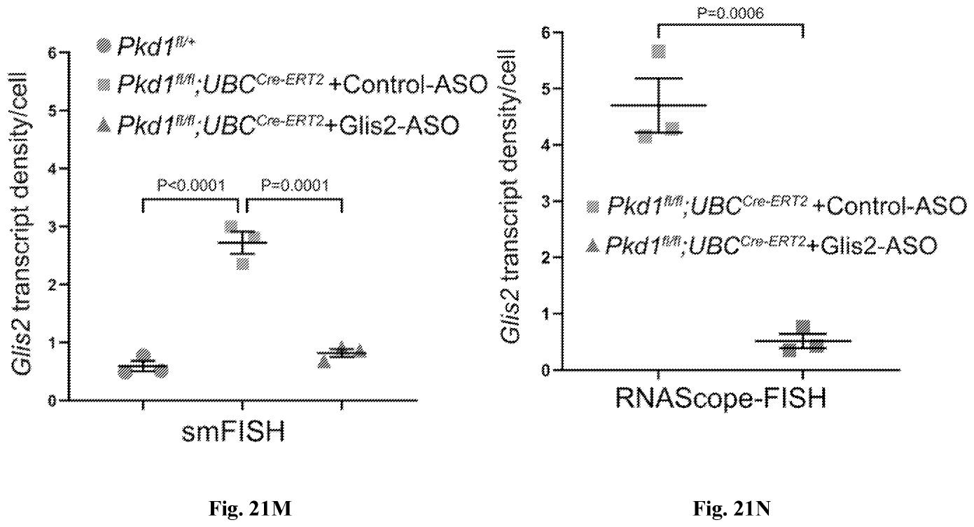

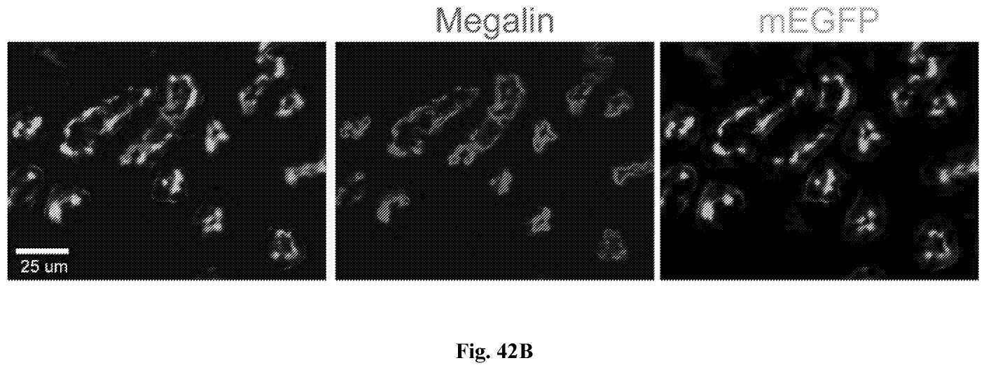

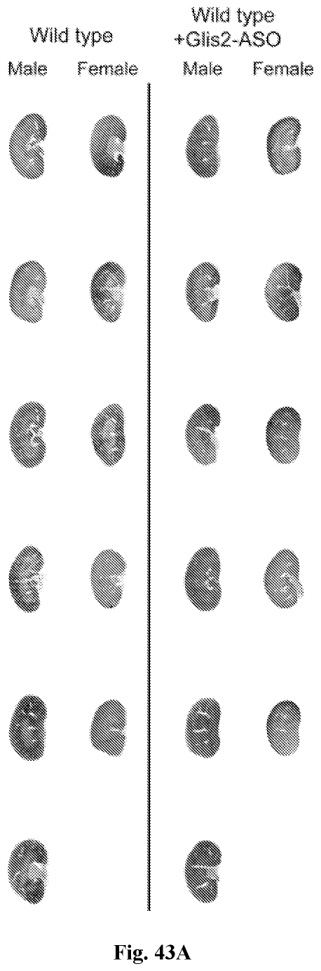

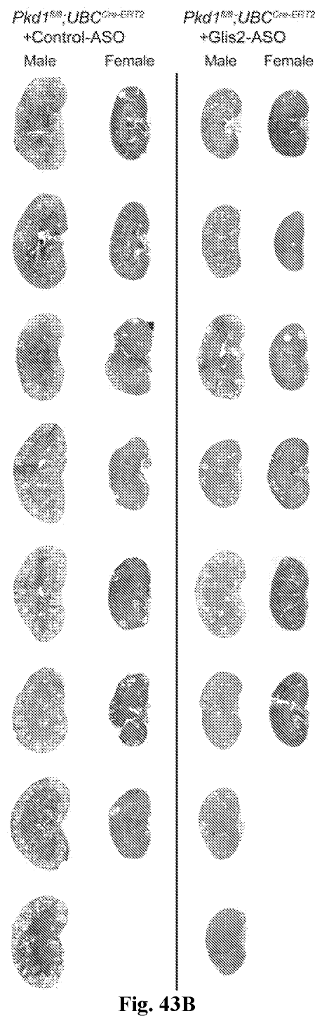

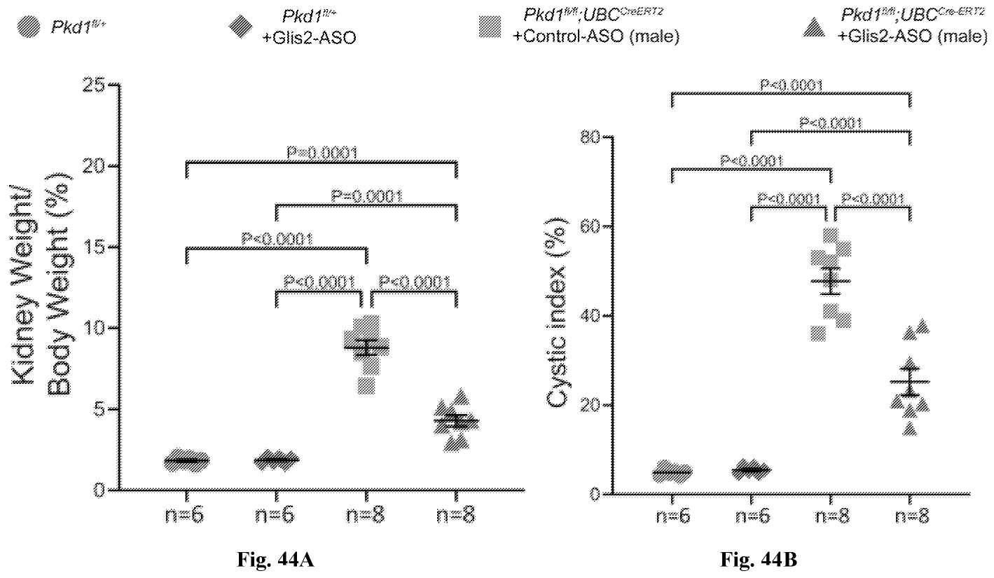

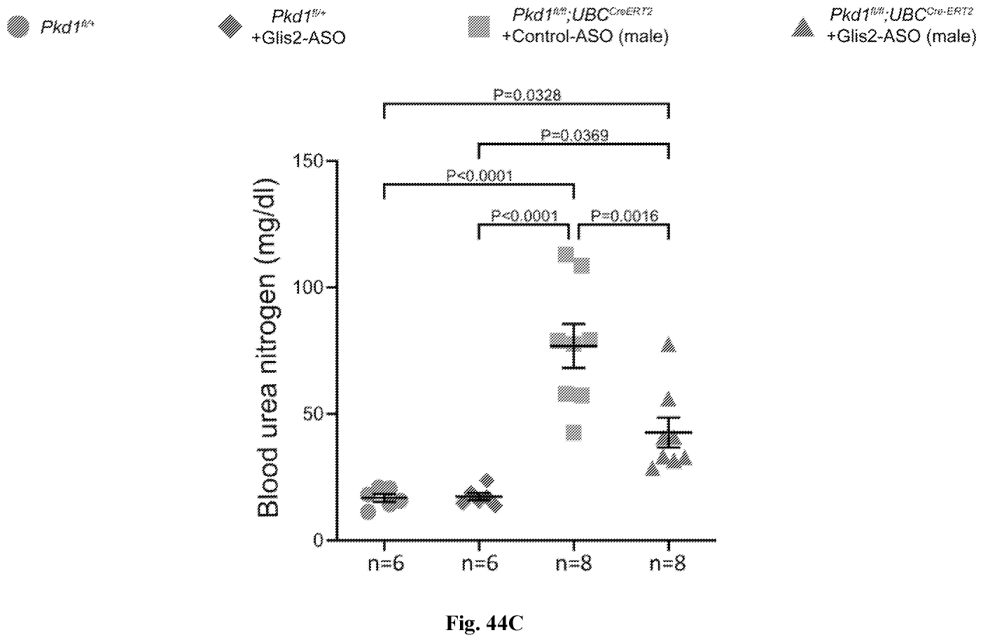

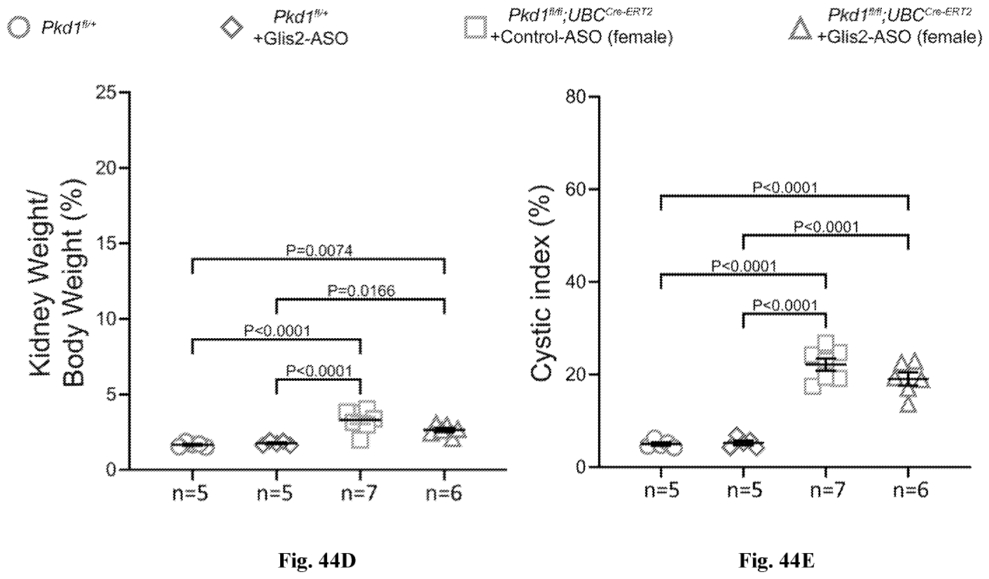

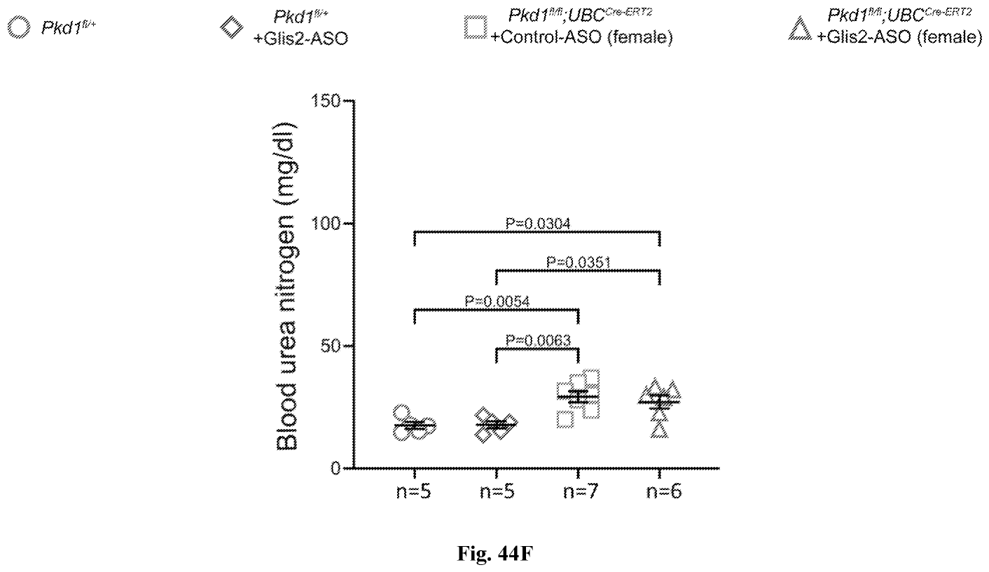

The present study next sought to evaluate whether Glis2 is a suitable target in preclinical pharmacological intervention to reduce polycystic kidney disease progression. The present study designed an optimized antisense oligonucleotide (ASO) targeting mouse Glis2 (Glis2-ASO) and a scrambled sequence control ASO (control-ASO). Since ASO are most effectively taken up in the proximal tubule in the kidney, the present study tested the Glis2-ASO effect in the tamoxifen inducible Pkd1^{fl/fl}; UBC^{Cre-ERT2} model which only has Cre activity in the proximal tubule in the kidney when evaluated with the ROSA^{mT/mG} reporter (FIGS. 42A-42B). Mice received daily intraperitoneal (IP) tamoxifen from P28-P35 to inactivate Pkd1 in proximal tubule cells. Mice were treated with control- or Glis2-ASO at a dose of 50 mg/kg by IP injection from 5 weeks of age to 18 weeks age. Mice were injected twice during week 5 and then once per week thereafter for a total of 15 doses; polycystic kidney disease progression was examined at 18 weeks of age. Kidneys of Glis2-ASO treated mice had significantly reduced kidney-to-body weight ratio, cystic index, and BUN (FIGS. 21A-21D and 43A-43B). The data showed statistically significant improvement with Glis2-ASO compared to control-ASO treatment with both sexes combined. However, the previously reported sex dimorphism in Pkd1 disease severity in adult mouse models notably affected the distribution of the results. Male mice developed severe polycystic kidney disease with mean kidney-to-body weight ratio ˜8.8% that was associated with mean cystic index ˜48% and BUN 77 mg/dL in control-ASO treated mice. These parameters were significantly improved by Glis2-ASO treatment to respective means of ˜4.3%, ˜25% and 43 mg/dL in the male mice (FIGS. 44A-44C). Female mice developed minimal polycystic kidney disease with mean kidney-to-body weight ratio of only ˜3.3%, cystic index ˜22% and BUN 29 mg/dL. These parameters all decreased slightly to ˜2.6%, ˜19% and 27 mg/dL, respectively, with treatment but the findings were not powered to achieve statistical significance in female mice given the very mild control-ASO treated disease state (FIGS. 44D-44F).

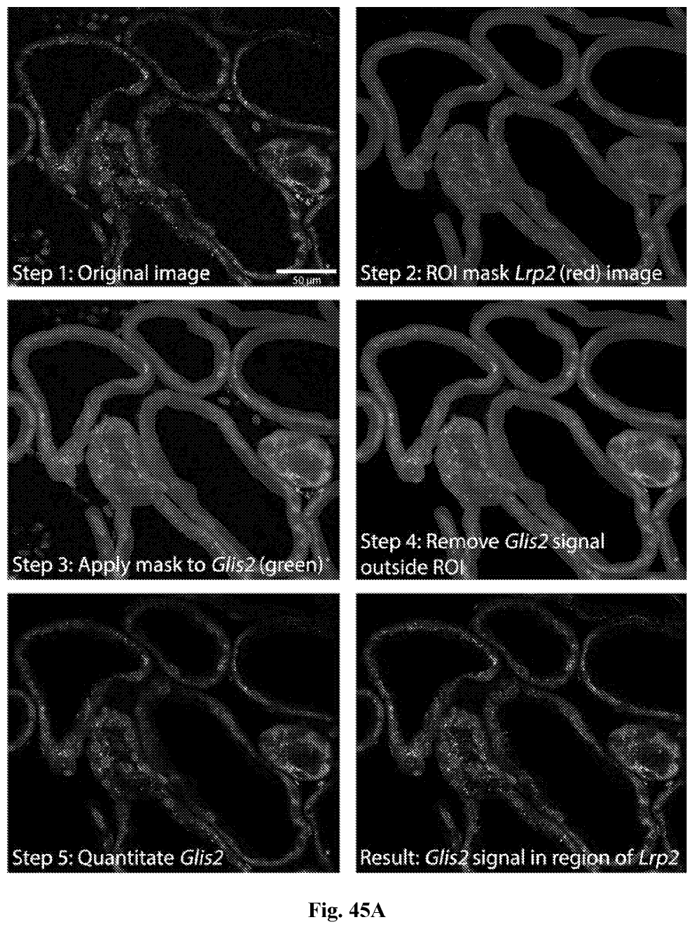

The present study assessed the “target engagement” efficiency of proximal tubule Glis2 mRNA depletion by the Glis2-ASO compared to control-ASO in 18-week-old kidneys of male mice using two independent semiquantitative fluorescent in situ hybridization (FISH) approaches—single molecule FISH (smFISH) and RNAScope-FISH (FIGS. 21E-21N and 45A-45B). Two color FISH with probes directed against the proximal tubule specific endocytic receptor megalin (Lrp2) allowed assessment of Glis2 mRNA expression in the region of proximal tubule cells (FIGS. 21E and 21I). The specificity of both FISH methods was confirmed by absence of hybridization of the Glis2 probes on Glis2^{-/-} null kidney tissue sections (FIGS. 21F-21J). Glis2-ASO treatment resulted in reduced Glis2 FISH signal compared to control-ASO treatment by both detection methods (FIGS. 21G, 21H, 21K and 21L). To quantitate these differences, regions of interest (ROI) near the proximal tubules in multiple images from kidney sections from control- and Glis2-ASO treated mice were manually selected based solely on the megalin FISH channel (FIGS. 45A-45B). The numbers of Glis2 FISH signal points and the number of DAPI stained nuclei as a surrogate for cell numbers in these ROI were quantified using CellProfiler and the Glis2 transcript density per cell was used for relative quantitation (FIGS. 21M and 21N). Treatment with the Glis2-ASO significantly decreased Glis2 mRNA in regions of the cortex expressing Lrp2 (i.e., proximal tubules) when measured by both smFISH and RNAScope-FISH (FIGS. 21M and 21N).

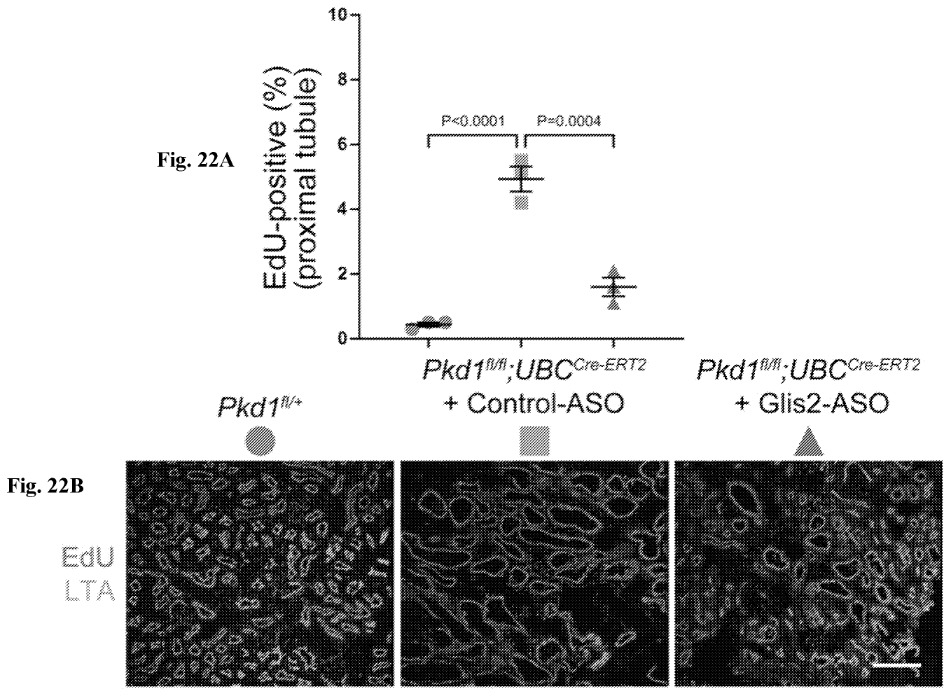

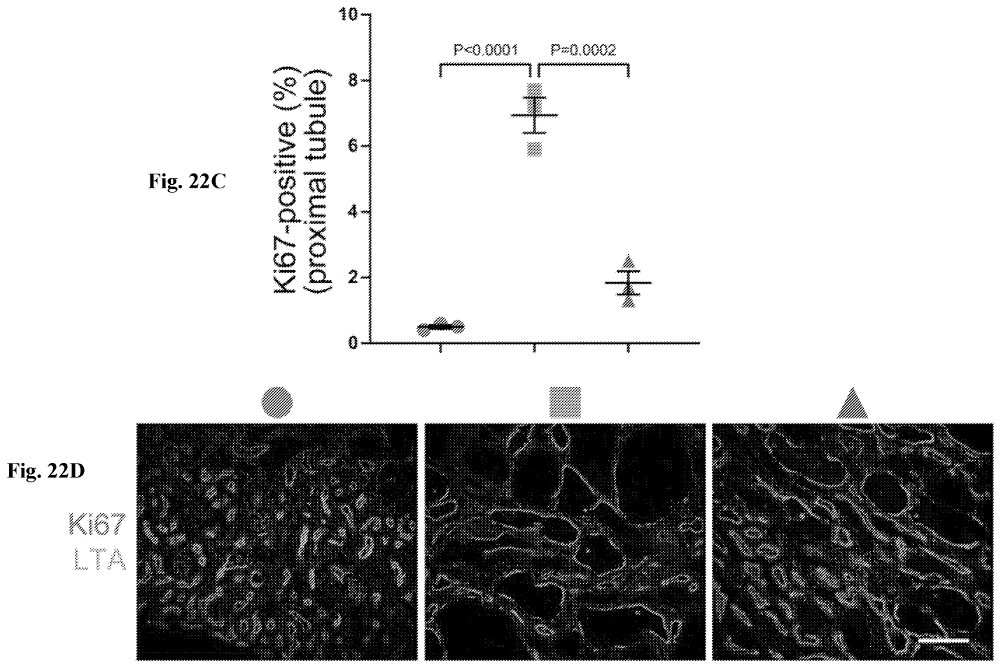

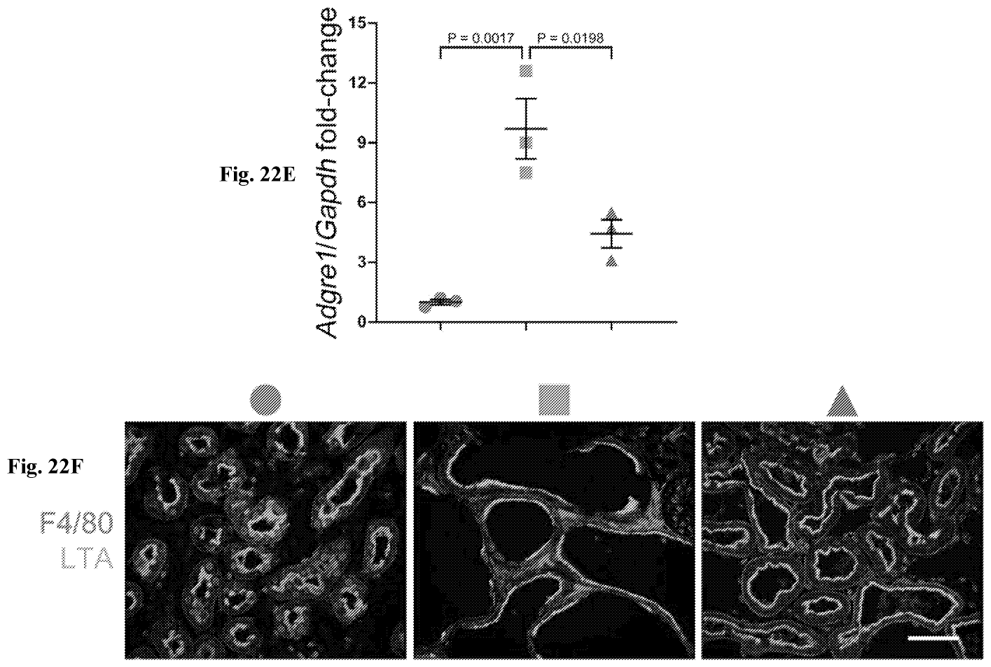

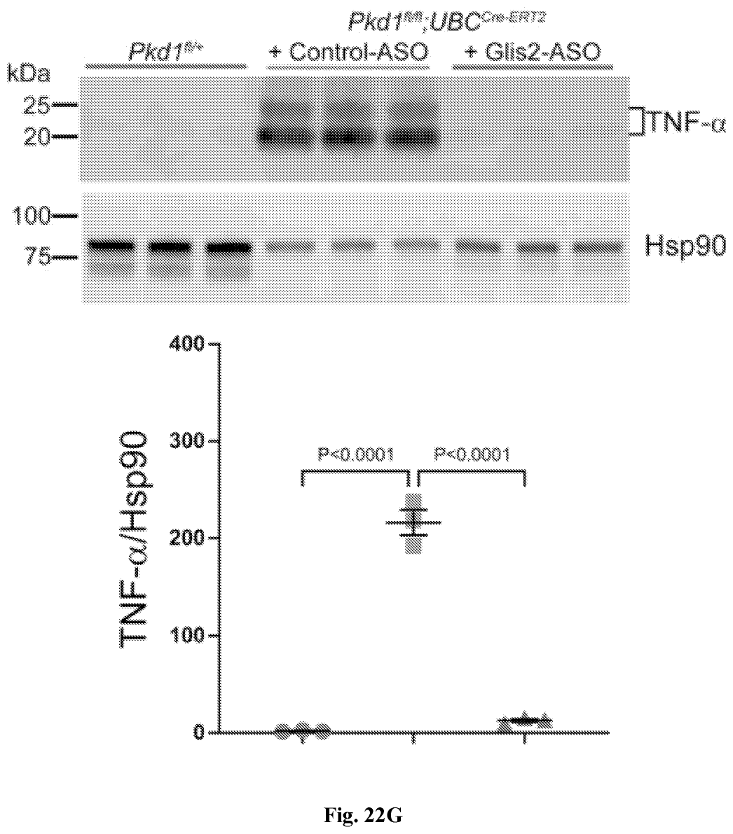

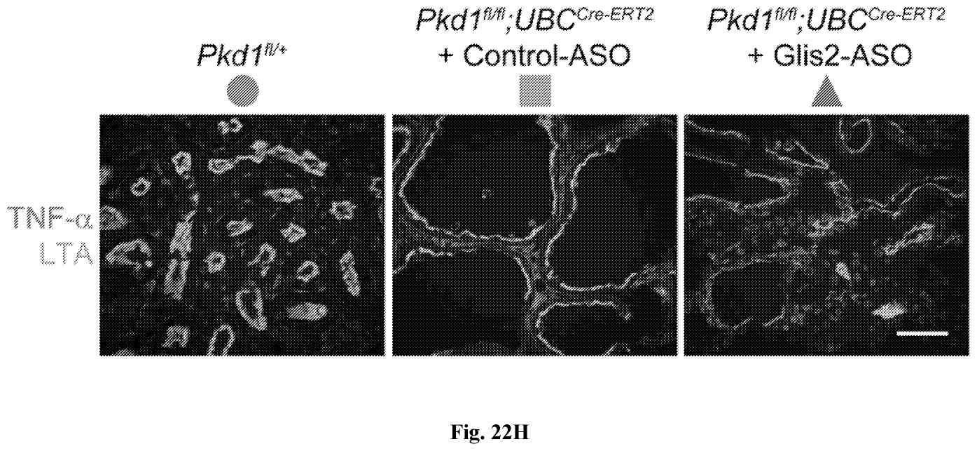

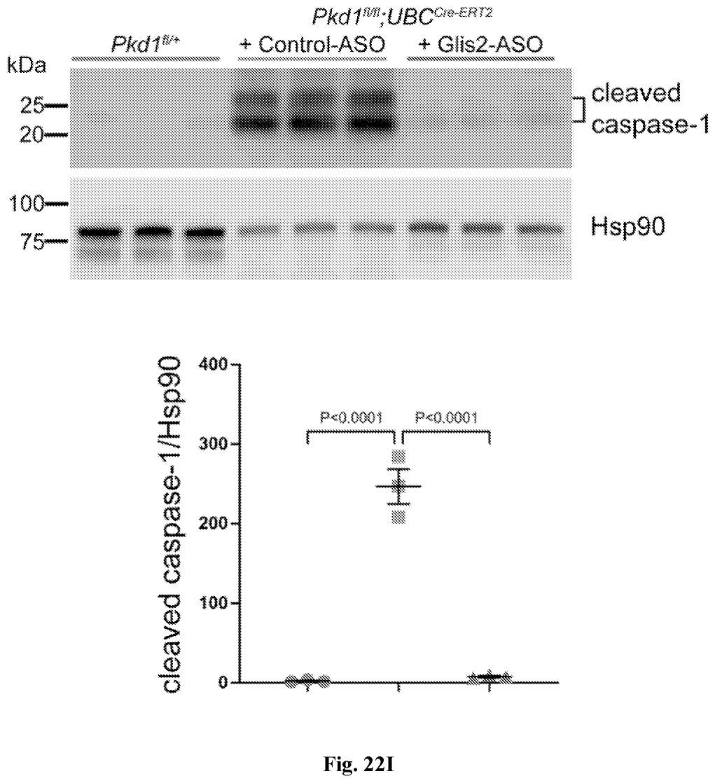

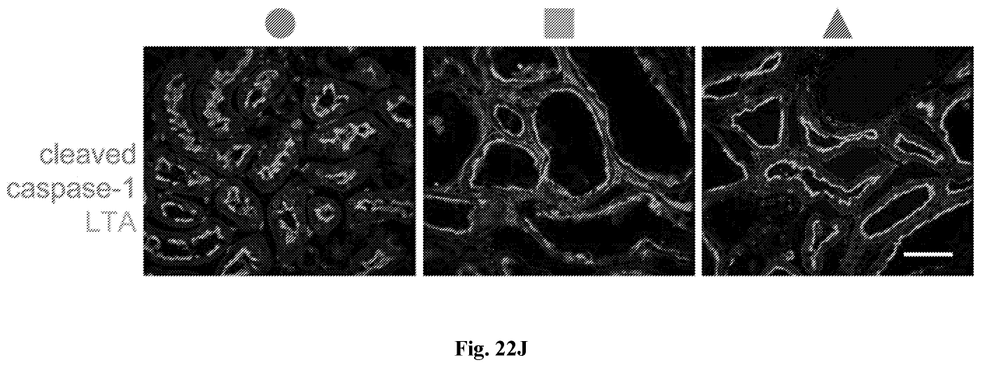

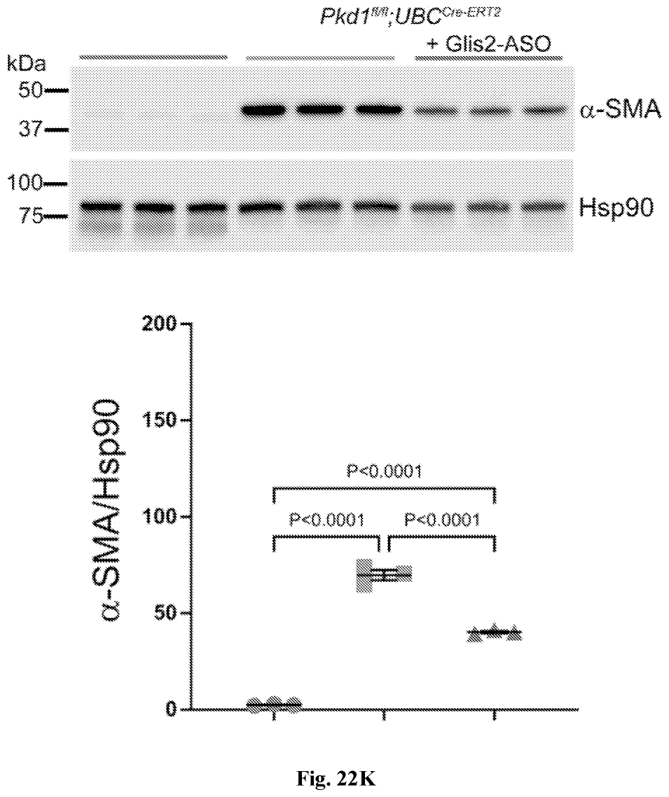

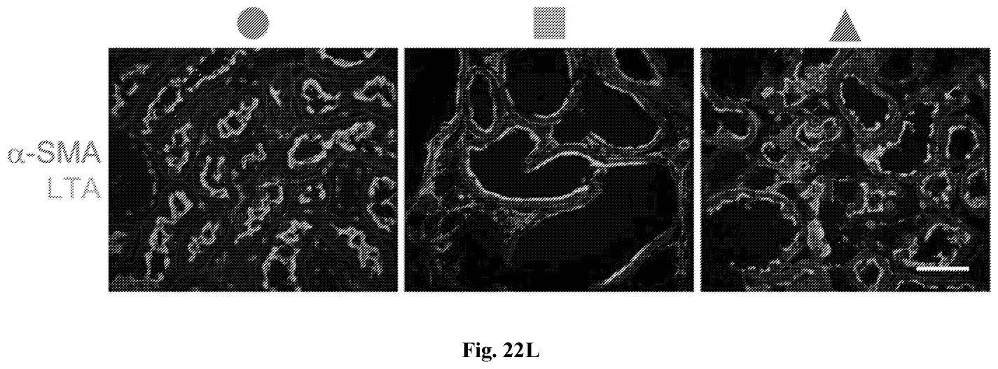

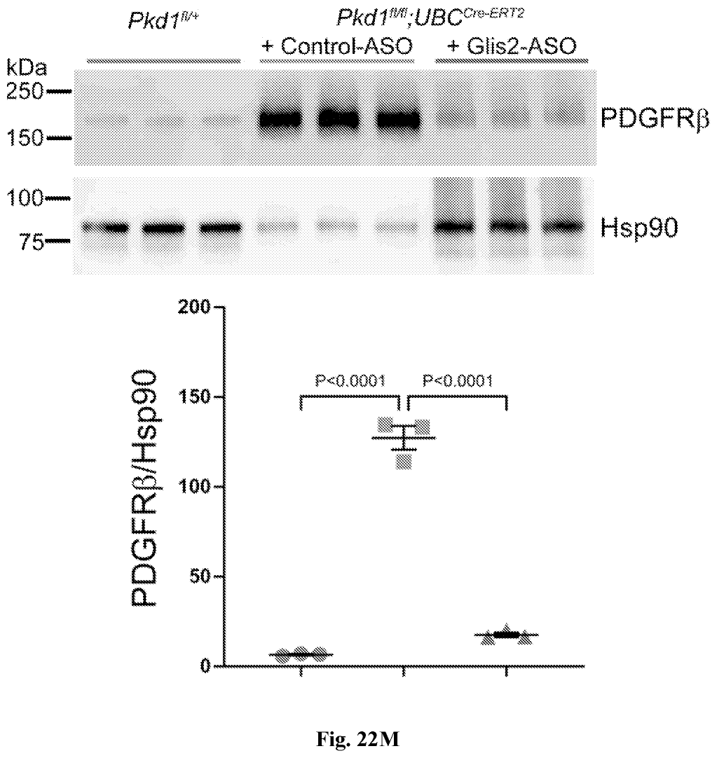

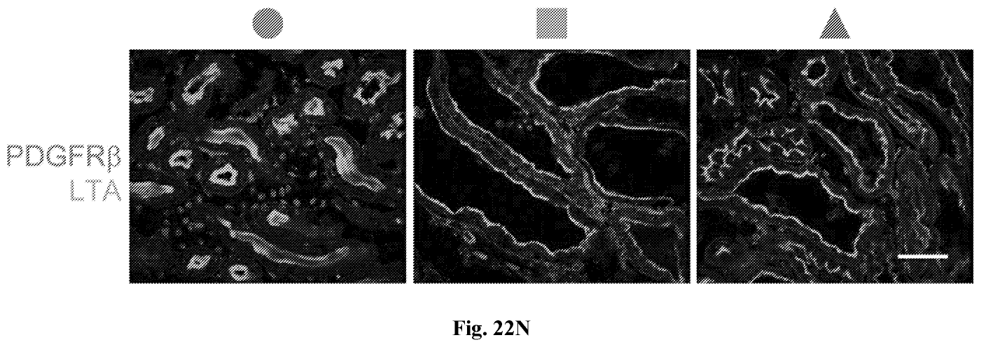

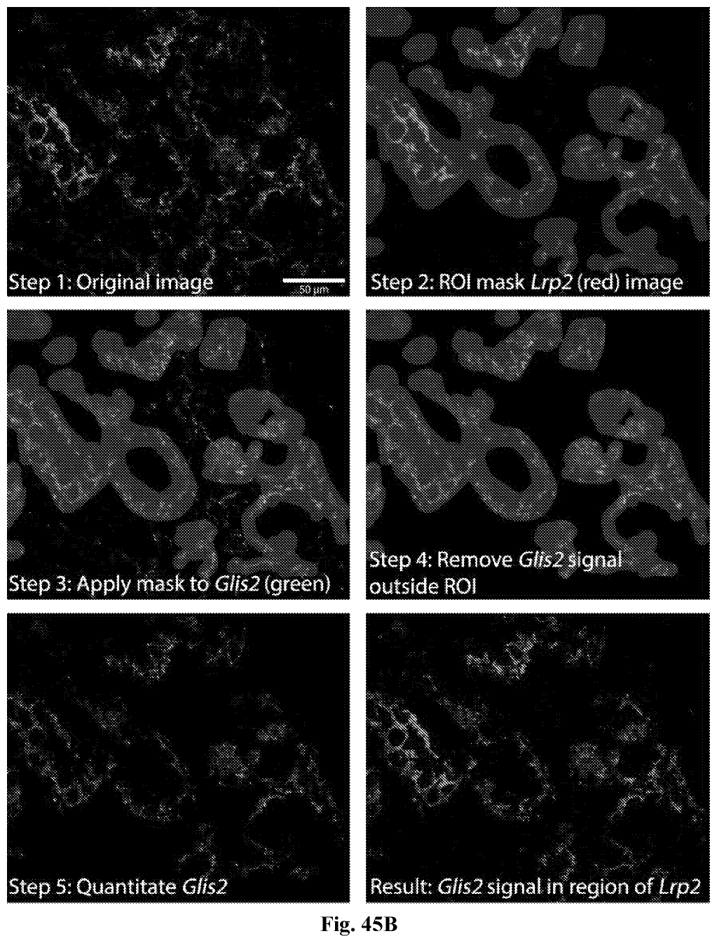

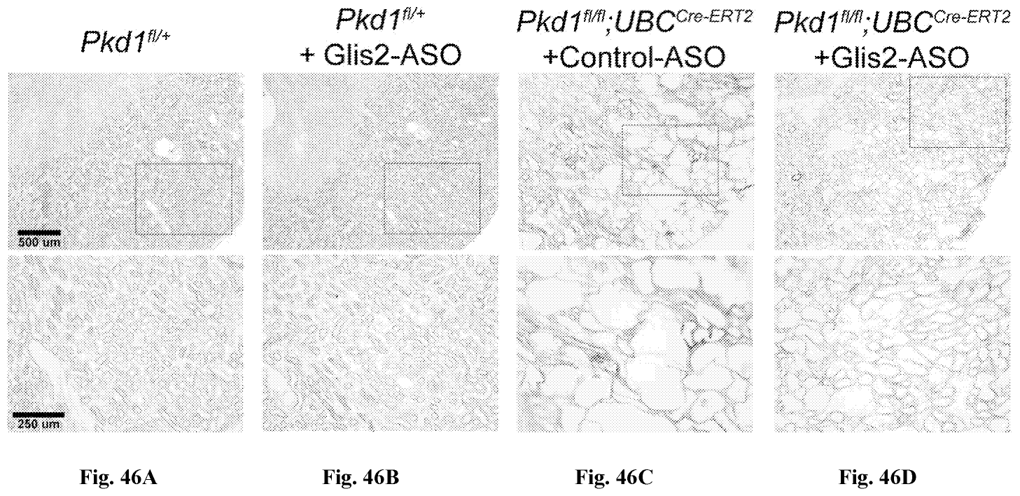

The present study assessed whether treatment with Glis2-ASO resulted in increased cell senescence. Glis2-ASO treatment in noncystic kidneys did not result in discernible senescence when evaluated by SA-β-gal staining (FIGS. 46A-46B). Kidneys from Pkd1^{fl/fl}; UBC^{Cre-ET2} mice treated with control-ASO showed again occasional cysts with senescent cells as previously noted in untreated mice with polycystic kidney disease (FIG. 46C). Glis2-ASO treated Pkd1^{fl/fl}; UBC^{Cre-ERT2} mice showed the presence of sporadic SA-β-gal senescent cell staining that was qualitatively reduced compared to untreated cystic kidneys indicating that Glis2-ASO did not result in increased cell senescence (FIG. 46D). Finally, the present study assessed several additional markers of polycystic kidney disease severity in response to Glis2-ASO treatment in male mice. Cyst cell proliferation is a common feature of ADPKD. Male Pkd1^{fl/fl}; UBC^{Cre-ERT2} polycystic mice treated with control-ASO showed increased proximal tubule cell proliferation measured by EdU incorporation and Ki67 staining, and this proliferation was significantly reduced in mice treated with Glis2-ASO (FIGS. 22A-22D). Polycystic kidneys are also characterized by inflammatory changes. The presence of increased interstitial macrophages in control-ASO treated cystic kidneys was reduced following Glis2-ASO treatment (FIGS. 22E and 22F). It was shown that the cytokine TNF-α and the NLRP3 inflammasome effector cleaved-caspase-1 are elevated in the interstitial regions of polycystic kidneys. In keeping with these observations, control-ASO treated cystic kidneys showed elevated levels of the TNF-α and cleaved caspase-1; both were reduced in mice treated with Glis2-ASO (FIGS. 22G-22J). Another hallmark of progressive polycystic kidney disease is tubulointerstitial fibrosis. The fibrotic response can be reduced or prevented in ADPKD but once it is established, it can result in irreversible kidney damage. Control-ASO treated kidneys showed increased myofibroblast activation by immunoblotting and immunohistochemistry for α-smooth muscle actin (α-SMA) and platelet-derived growth factor receptor p (PDGFRO) (FIGS. 22K-22N). Treatment with Glis2-ASO significantly reduced the levels of both markers of fibrosis compared to the control-ASO treatment (FIGS. 22K-22N). Overall, the studies presented validate a paradigm for in vivo transcriptome discovery in ADPKD preclinical models and show that upregulation of Glis2 discovered by this approach is a surrogate marker for polycystin function both in vivo and in vitro. Beyond being a marker for polycystin activity, Glis2 is shown in preclinical studies to be therapeutic target for treatment of ADPKD.

### Example 2-7

The polycystins were initially discovered as complex, novel proteins with uncertain function. They were subsequently localized to the primary cilium, a solitary nonmotile microtubule-based sensory organelle that projects into the lumen from the apical surface of most kidney tubule cells. The precise molecular composition and function of primary cilia and, by extension, of the polycystin complex in the metanephric kidney remains incompletely understood. While many known cellular pathways have been shown to be dysregulated in ADPKD, molecular mechanistic connections between those dysregulated pathways and polycystin function in cilia have been lacking. The novel nature of the polycystins and of their organellar compartment, coupled with the absence of biological mechanistic precedents for connecting these features to in vivo phenotypes, suggest that critical functional relationships in the polycystin effector pathways are yet to be discovered. The Hedgehog pathway provides a window into the potential complexity of the unknown elements in polycystin signaling. The polytopic transmembrane protein Patched serves as an inhibitory regulatory element on the ciliary membrane in Hedgehog signaling, much as polycystins on the cilia membrane curtail large scale remodeling of the adult kidney. Many of the molecular components downstream of Patched (e.g., Smoothened, Gli1-3, Sufu, etc.) were known from previous discoveries in Drosophila, a resource missing in polycystin signaling thereby necessitating discovery approaches in metanephric kidney models. In the current study, strategies to produce an unbiased discovery platform based on early tubule cell-specific transcriptional changes directly related to the in vivo propensity for Pkd1 dependent cyst formation were adopted. First, the present study used TRAP RNASeq to selectively capture the kidney tubule cell type-specific in vivo translatome confined to cells with Pkd1 inactivation. Second, the present study used a tripartite comparison of genotypes that included Pkd1^{KO}+ cilia^{KO} double knockouts to enhance specificity for transcriptional changes associated with cyst formation which does not occur in Pkd1^{KO}+ cilia^{KO} kidneys despite inactivation of Pkd1. Third, the present study selected a time point before discernible cystic tubule dilation following Pkd1 inactivation to reduce the impact of non-cell autonomous secondary “outside-in” signals. To increase the utility of these TRAP RNASeq and related data to the broader research community, the present study have set up the “Metabolism and Genomics in Cystic Kidney (MAGICK)” (https://pkdgenesandmetabolism.org/) web site making all of the data readily browsable [reviewer username: pkduser; password: rc2pkd2022]. The in vivo TRAP translatome derived in these studies should offer broad opportunities for mechanistic discovery in polycystin- and cilia-related kidney phenotypes.

The interpretation of the TRAP translatome was further refined based on biological context. Data is presented with sex as a variable to account for the sex dimorphism in disease severity that is a unique feature of adult Pkd1 mouse models. Sex dimorphism is not observed in humans or adult Pkd2 mouse models. Male mice showed a strong cell cycle related transcriptional signature at 7 weeks, similar to what the present study observed at 10 weeks in bulk RNASeq studies using Pkd2 models. The earlier appearance of the enhanced cyst cell proliferation phenotype in males is correlated with the more rapid polycystic kidney disease progression in male mice. Overall, female mice appear to be protected from ADPKD progression in adult Pkd1 models, so the male TRAP data at the 7-week timepoint is likely more broadly applicable to other models of ADPKD. Nonetheless, since female mice also form cysts, the earliest transcriptional changes downstream of Pkd1 inactivation are expected to be present in both male and female mice. The set of 167 DEG common to both sexes that follow the CDCA transcriptional pattern represent a high confidence set of early ADPKD-related in vivo alterations in the translatome. The CDCA pattern DEG were further prioritized based on expression along the entire tubule with TPM>1. The latter set of 71 DEG contained several genes with cilia related functions as well as undefined functions but high degrees of association with the CDCA pattern.

The finding that among nearly 17,000 transcripts sequenced, the 71 identified by the above unbiased paradigm included Pkd2 provides a degree of assurance that this set of DEG contain transcripts with significant functional relevance to ADPKD pathobiology. The present study found that a subset of these retained Pkd1 genotype-dependent differential expression in primary ciliated cell cultures. The concordance of these finding with the in vivo cell specific transcriptional changes suggests that a set of these DEG can be used as a “transcriptional signature” for cyst related polycystin function in vitro. This transcriptional signature can also be applied to validation of other polycystic kidney disease model systems such as organoids.

Glis2 was selected for in depth validation based on its robust expression along the entire nephron, its association with a ciliopathic nephronophthisis phenotype and its strong statistical association with the CDCA pattern TRAP gene set. Glis2 had not previously been considered a target of CDCA activity or an effector of polycystin-related functional pathways in ADPKD. The finding and validation of Glis2 in these roles highlights the value of the unbiased in vivo TRAP RNASeq approach to fill gaps in understanding of ADPKD mechanisms. Although Glis2 has been implicated in an array of biological functions, it is best characterized in its role in the mammalian kidney. The present study found that Glis2 protein expression is highest in the first two postnatal weeks in the mouse kidney. This pattern parallels the developmental regulation of polycystin-1 expression and the developmental switch in the pace of cyst growth following Pkd1 inactivation. Initial reports suggested that Glis2 may be localized in cilia in addition to the nucleus, offering an attractive parallel with Gli transcription factors in the Hedgehog pathway; however, the cilia localization of Glis2 was not corroborated in later studies, in the cilia proteome and by the data. Glis2^{mice develop progressive nephronophthisis-like atrophic tubulointerstitial fibrotic kidney disease associated with tubule cell senescence, but with no reported extra-renal defects. Similarly, patients with NPHP}7 develop early onset end stage kidney disease, but extra-renal manifestations have not been reported. In the studies, kidney selective conditional inactivation of Glis2 or systemic treatment with Glis2-ASO did not result in any discernible kidney phenotype. The absence of tubulointerstitial disease and cellular senescence in Glis2 adult inactivation and inhibition models suggests that these are features may be associated with development manifestation of germline null mutations in Glis2. Glis2 has been reported to be upregulated in mouse models of nonalcoholic steatohepatitis (NASH), and knockdown has reversed NASH associated transcriptional programs. Overall, these data leave open the possibility that pharmacological inhibition of Glis2 in adults may be well tolerated both systemically and in the kidney.

One of the factors limiting progress in ADPKD research and drug discovery is the lack of well validated in vitro model systems that reliably recapitulate in vivo polycystic kidney disease-related molecular phenotypes. Cultured cells are an adequate vehicle for assessing trafficking and biophysical properties of polycystin proteins. Interrogation of channel activities of the polycystin complexes in cilia are better suited to understanding processes “upstream” of CDCA. More complex cellular structural phenotypes, such as cyst growth in three-dimensional cell culture systems or organoids are pleiotropic processes that result from a spectrum of biological events that are not readily reduced to unique molecular events directly related to ADPKD. Optimal in vitro systems would read out “downstream” molecular elements of polycystin function in a manner that can be recapitulates the same molecular responses in vivo and can be readily quantified in vitro. For example, since inactivation of cilia suppresses cyst formation in vivo, a validated downstream in vitro phenotype should show expected changes upon loss of polycystins in ciliated cells and those changes should be suppressed when cilia are also inactivated in vitro. The present study found that Glis2 transcript and nuclear protein expression fulfils these criteria and therefore is a validated functional readout for ADPKD-relevant polycystin function in vitro. As noted above, the TRAP RNASeq dataset contains other potential transcriptional changes that can be tested in vitro under similar conditions to define a “multigenic transcriptional signature” for polycystin function as it relates specifically to polycystic kidney disease, agnostic to knowledge of specific mechanisms. Beyond the transcriptional changes, the present study was also able to show that the primary cell culture systems developed herein, grown under conditions that allow cilia formation, recapitulate elements of the in vivo cilia dependent transcriptional phenotype associated with cyst formation. Mutations to either Pkd1 or Pkd2 resulted in increased Glis2 expression in vitro and dual inactivation of polycystins and cilia (Kif3a, lft88) or cilia function (Tulp3) abrogated increased expression of Glis2 transcript and nuclear protein. Glis2 and Pkd2 transcripts also increased in IMCD3 cells with Pkd1 knockout grown to ciliated conditions indicating that at least Glis2 and Pkd2, and perhaps other genes in a transcriptional signature, may function as an in vitro readout not just in primary cells, but in cell lines as well. It is proposed that appropriately constructed kidney epithelial cell cultures, validated using the TRAP RNASeq-based transcriptional signatures or protein expression changes (e.g., Glis2), will be a useful in vitro system for suppressor screens of CDCA activity downstream of polycystin inactivation. The discovery of the value of both the primary cells as an assay system and at least Glis2 as a dependable, biologically relevant readout, should facilitate in vitro discovery screens for modulators of polycystin function.

A transcriptional change related directly to polycystin function as provided by Glis2 does not necessarily indicate functional importance. The present study established the functional importance of Glis2 in ADPKD pathogenesis through a series of genetic epistasis experiments in mouse models of ADPKD. Inactivation of Glis2 suppressed cyst formation in two early onset models of ADPKD based on Pkd1 and two adult models based on either Pkd1 or Pkd2. The protection in the adult inducible model of Pkd1 was particularly complete and could be interpreted in the absence of confounding by the nephronophthisis phenotype that occurs in germline Glis2^{-/-} mice. The present study also addressed an inherent limitation of models that use the Cre/loxP system which result in simultaneous inactivation of large swaths of contiguous cells along the kidney tubule. This mechanism differs from the more spatially discrete sporadic somatic inactivation events thought to underlie cyst initiation in human ADPKD. The present study used the Pkd2^{WS25/-} mice which develop cysts following sporadic random second hit recombination events akin to the human disease to extend validation that Glis2 inactivation suppresses cyst formation in non-Cre dependent models. These genetic interaction studies establish Glis2 not only as a marker for CDCA, but also as an effector of CDCA. Knowledge of a second critical molecular component of CDCA downstream of polycystins, i.e., Glis2, offers a significant step forward in devising strategies to fill in the remaining gaps.

The present study assessed whether Glis2 is a suitable therapeutic target in preclinical model systems. The present study found that an ASO directed against Gis2 was effective in reducing polycystic kidney disease severity and attendant tissue level changes including cyst cell proliferation and inflammatory and pro-fibrotic changes in the kidney. These findings suggest that inhibition of Gis2 in a range amenable to pharmacologic rather than genome level genetic inactivation can significantly impact the course of ADPKD in an orthologous gene model system. Furthermore, a drug with specificity for Glis2 may be tolerated given the lack of known extra-renal effects in both knockout mice and patients with NPHP7. There are a few notable elements to the preclinical study design. Since it is anticipated that the ASO could be most effective in reducing Glis2 transcripts in the proximal tubule, the present study selected a model in which Pkd1 was only inactivated in the proximal tubule in the kidney. Given that the Glis2 dual genetic inactivation data was effective in reducing cysts in the proximal and distal nephron, it was expected that compounds able to achieve Glis2 inhibition along the entire nephron would likewise be effective along the entire nephron. Another consequence of the choice of a proximal tubule inactivation model was that it accentuated the sex differences in disease progression. As a result, the effects of ASO therapy were most pronounced in male mice. However, the efficacy of Glis2 genetic inactivation in both sexes in models where sex dimorphism was less apparent supports the expectation that the ASO will also be effective in female mice with more advanced disease. Finally, the present study began treatment with ASO soon after inactivation of Pkd1 before cyst had already formed. This choice eliminated potential variation in drug availability in more advanced cysts that may not receive glomerular filtrate. This was beneficial in a proof-of-concept studies, but future studies should determine if inhibition of Glis2 at later stages of ADPKD can nonetheless slow disease progression.

Overall, this study fills several gaps in the ADPKD field. First, it provides a novel translatome dataset that most closely defines the early, cell autonomous in vivo changes associated with polycystic kidney disease. Second, it identifies Glis2 transcript and protein as an in vivo and in vitro biomarker of polycystin function specifically related to polycystic kidney disease progression. Third, it defines primary cells cultured from mouse kidneys as a well validated in vitro system in which to assay Glis2 expression as an indicator of polycystin function. Fourth, it establishes Glis2 as a functional target of CDCA cyst promoting activity following polycystin inactivation. Finally, it offers a proof of concept that pharmacological targeting of Glis2 or its protein product can suppress polycystic kidney disease based on studies in preclinical models.

### Example 2-8: Materials and Methods

### Mouse Strains and Procedures

Following strains of mice were used in this study: Pkd1^{fl} (Shibazaki et al. Hum. Mol. Genet. 17, 1505-1516 (2008)), Pkd2^{fl} (Nishio et al. J. Am. Soc. Nephrol. 21, 295-302 (2010)), Kif3a^{fl} (Marszalek et al., Proc Natl Acad Sci USA 96, 5043-5048 (1999)), Pax8^{rtTA} (JAX Strain #007176), TetO^{Cre} (JAX Strain #006234), Tulp3^{fl} (Legue et al., Curr Biol 29, 803-812 e805 (2019)) (kindly provided by Karle Liem, Yale University), Pkd2^{+/-} (Wu et al. Nat. Genet. 24, 75-78 (2000)), Pkd2^{WS25} (Wu et al. Cell 93, 177-188 (1998)), Ift88_{fl} (JAX Strain #022409) (Haycraft et al. Development 134, 307-316 (2007)), Glis2^{-/-} (Kim et al., Molecular and cellular biology 28, 2358-2367 (2008)), UBC^{Cre-ERT2} (JAX Strain #008085), Pkhd1^{Cre} (Patel et al. Hum Mol Genet 17, 1578-1590 (2008)), ACTB^{Cre} (JAX Strain #003376), ACTB^{Flp} (JAX Strain #005703), ROSA^{mT/mG} (JAX Strain #007676), R26^{Rpl10a} (JAX Strain #024750). Glis2^{fl} mice were produced in this study. Three Glis2-targeted Glis2^{tm1a(EUCOMM)Hmgu} embryonic stem (ES) cell lines (HEPD0539_2_B10; HEPD0539_2_D09; HEPD0539_2_G11) were obtained from the European Mouse Mutant Cell Repository (EuMMCR, Munich, Germany). All three ES cell lines have identically targeted Glis2, which in its final form has exon 3 is flanked by loxP sites. Two ES cell clones (HEPD0539_2_B10, HEPD0539_2_D09) were expanded and injected into blastocysts to obtain founders. F1 progeny with the Glis2 targeted allele were crossed with ACTB^{FLP} mice to remove the LacZ-neomycin cassette through the germline. An exon 3 deleted Glis2^{Ex3-} null allele was obtained from Glis2^{fl} by mating with ACTB^{Cre}. Mating strategies for allele combination and doxycycline and tamoxifen Cre induction protocols have been previously described (Ma et al., Nat Genet 45, 1004-1012 (2013) and Dong et al. Nat Genet 53, 1649-1663 (2021)). All strains were backcrossed at least four generations with C57BL/6J and are expected to be at least 90% congenic C57BL/6J. Mice of both sexes were used. All animals were maintained in secure, intact, clean, fully assembled, barcoded, static micro-isolator cages. Temperature was maintained between 20° C. and 26.1° C. and humidity between 30% and 70%, with 12 hour:12 hour light:dark cycles. All animals were used in accordance with scientific, humane, and ethical principles and in compliance with regulations approved by the Yale University Institutional Animal Care and Use Committee (IACUC). Genotyping was done on DNA isolated from toe clips. Mice were euthanized according to standard protocols approved by Yale IACUC. Blood was collected using ventricular puncture. Sera were separated using Plasma Separator Tubes with lithium heparin (BD Biosciences, BD Vacutainer, Cat. No. 364606). Serum urea nitrogen was analyzed by the George M. O'Brien Kidney Center at Yale. One kidney was snap-frozen for protein and mRNA extraction, and the other kidney was fixed in situ by perfusion through the heart with 4% paraformaldehyde (PFA, MP Biomedicals, Cat. no. 0215014601) in 1×PBS.

The primers for genotyping the mouse alleles are presented in the table below. It was noted that the PCR genotyping primers for the Pkd2^{WS25} allele which was previously only genotyped by Southern blotting (Wu et al. Cell 93, 177-188 (1998)). The same forward primer is combined with the Pkd2^{WS25} reverse primer to detect the Pkd2^{WS25} allele with the Pkd2⁻ reverse primer which detects the Pkd2 null allele in a manner that distinguishes it from Pkd2^{WS25}.

### Genotyping Primers

| Gene | Forward Primer | Reverse Primer |
|---|---|---|
| Pkhd1^{Cre} | 5′-CTGGTTGTCATTGGCCAGG | 5′-GCATCGACCGGTAATGCAGGC |
| | (SEQ ID NO 4) | (SEQ ID NO 5) |
| Pax8^{rtTA} | 5′-CCATGTCTAGACTGGACAAGA | 5′-CTCCAGGCCACATATGATTAG |
| | (SEQ ID NO 6) | (SEQ ID NO 7) |
| TetO^{Cre} | 5′-GCAGAGCTCGTTTAGTGAAC | 5′-TCGACCAGTTTAGTTACCC |
| | (SEQ ID NO 8) | (SEQ ID NO 9) |
| Pkd1^{fl} | 5′-CACAACCACTTCCTGCTTGGTG | 5′-CCAGCATTCTCGACCCACAAG |
| | (SEQ ID NO 10) | (SEQ ID NO 11) |
| Pkd2^{fl} | 5′-GGGTGCTGAAGAGATGGTTC | 5′-TCCACAAAAGCTGCAATGAA |
| | (SEQ ID NO 12) | (SEQ ID NO 13) |
| Tulp3^{fl} | 5′-CCATTTGTGAGGGTTGCTTT | 5′-GTTTTGTGCTGGGGATGGTA |
| | (SEQ ID NO 14) | (SEQ ID NO 15) |
| Ift88^{fl} | 5′-GACCACCTTTTTAGCCTCCTG | 5′-AGGGAAGGGACTTAGGAATGA |
| | (SEQ ID NO 16) | (SEQ ID NO 17) |
| Kif3a^{fl} | 5′-TCTGTGAGTTTGTGACCAGCC | 5′-GGTGGGAGCTGCAAGAGGG |
| | (SEQ ID NO 18) | (SEQ ID NO 19) |
| Glis2^{fl} | 5′-ACTGGGGGTAGGGTCCTAGA | 5′-CCCTGTCACCATCTCCAACT |
| | (SEQ ID NO 20) | (SEQ ID NO 21) |
| UBC^{Cre-ERT2} | 5′-GACGTCACCCGTTCTGTTG | 5′-AGGCAAATTTTGGTGTACGG |
| | (SEQ ID NO 22) | (SEQ ID NO 23) |
| ACTB^{Cre} | 5′-AGGTTCGTTCACTCATGGA | 5′-TCGACCAGTTTAGTTACCC |
| | (SEQ ID NO 24) | (SEQ ID NO 25) |
| ACTB^{Flp} | 5′-CACTGATATTGTAAGTAGTTTGC | 5′-CTAGTGCGAAGTAGTGATCAGG |
| | (SEQ ID NO 26) | (SEQ ID NO 27) |
| R26^{Rpl10a} | 5′-CGTGTTCGTGCAAGTTGAGT | 5′-ATTGCATCGCATTGTCTGAG |
| (knockin) | (SEQ ID NO 28) | (SEQ ID NO 29) |
| R26^{Rpl10a} | 5′-CGTGTTCGTGCAAGTTGAGT | 5′-CCGAAAATCTGTGGGAAGTC |
| (WT) | (SEQ ID NO 30) | (SEQ ID NO 31) |
| Pkd2^{WS25} | 5′- | 5′-CTGATTCACATGCCCCAGGT |
| | GTGCTACTTCCATTTGTCACGTCCTGC | (SEQ ID NO 33) |
| | (SEQ ID NO 32) | |
| Pkd2^{▬} | 5′- | 5′-GGGAAAACCATGGAAAATCG |
| with | GTGCTACTTCCATTTGTCACGTCCTGC | (SEQ ID NO 35) |
| Pkd2^{WS25} | (SEQ ID NO 34) | |
| Glis2⁺ (WT) | 5′-CAGTCTGCCTGGCCTGCCATTAC | 5′-CAGACCCTGGTGGAATGACCAC |
| | (SEQ ID NO 36) | (SEQ ID NO 37) |
| Glis2^{▬} | 5′-TTCTATCGCCTTCTTGACGAG | 5′-GCGCCTTTCCAATGAGTCTTC |
| | (SEQ ID NO 38) | (SEQ ID NO 39) |
| Glis2^{Ex3+} | 5′-ACTGGGGGTAGGGTCCTAGA | 5′-CCCTGTCACCATCTCCAACT |
| (WT) | (SEQ ID NO 40) | (SEQ ID NO 41) |
| Glis2^{Ex3▬} | 5′-TCGTATAGCATACATTATACGAA | 5′-AATAGCGAAGTGGCTGGAAA |
| | (SEQ ID NO 42) | (SEQ ID NO 43) |
| ROSA^{mT/mG} | 5′-TAGAGCTTGCGGAACCCTTC | 5′-CTTTAAGCCTGCCCAGAAGA |
| (knockin) | (SEQ ID NO 44) | (SEQ ID NO 45) |
| ROSA^{mT/mG} | 5′-AGGGAGCTGCAGTGGAGTAG | 5′-CTTTAAGCCTGCCCAGAAGA |
| (WT) | (SEQ ID NO 46) | (SEQ ID NO 47) |

Copy Number Determination of Pax8^{rtTA} and TetO^{Cre}

Mouse genomic DNA was diluted to 50 ng/μl and mixed with iTaq Universal SYBR Green Supermix (BioRad, Cat. no. 1725121) and qPCR primers specific to Pax8^{rtTA} or TetO^{Cre} and 18S rRNA. The primer sequences used were:

Pax8E1QF1
(SEQ ID NO 48)
5′-GGGAAGAGAAGGGTTGAAGG-3′
Pax8E1QR1
(SEQ ID NO 49)
5′-ACTCAGCAGGCCAGGAAGTA-3′
TetCreQF2
(SEQ ID NO 50)
5′-CACGCTGTTTTGACCTCCAT-3′
TetCreQR2
(SEQ ID NO 51)
5′-CTCTGCCCCTCGACTCTAGA-3′

qPCR was performed with a CFX96 Touch Real-Time PCR Detection System (BioRad). The expression of Pax8^{rtTA} and TetO^{Cre} was normalized to 18S by the 2^{-ΔΔCT} method.

### Pkd1 Deletion Efficiency in Kidney Tissues

Mice were euthanized and one kidney was snap-frozen in liquid nitrogen. DNA was extracted with DNeasy Blood & Tissue kit (Qiagen, Cat. no. 69504). DNA concentration was measured by Nanodrop 2000 (Thermo Fisher Scientific, Cat. no. ND2000). An equal starting amount of DNA (50 ng/μl) was used for all samples. A common forward primer (Pkd1X1F1) was designed to the region upstream of the first loxP site within intron 1. A reverse primer (PkdlX1R1) was designed to the region immediately downstream of the first loxP site also within intron 1. A second reverse primer (Pkd1X2R4) was designed to the region downstream of the second loxP site within intron 4. The following primer were used in Pkd1 deletion efficiency assay:

Pkd1X1F1:
(SEQ ID NO 52)
5′-TCACGGAAGAGCAGCCTGCCTT-3′
Pkd1X1R1:
(SEQ ID NO 53)
5′-TCTGTGTACTGGGGCACAGCCT-3′
Pkd1X2R4:
(SEQ ID NO 54)
5′-AGCACCTGAGCTGTTGTCAGGG-3′

Cre activation results in deletion of exons 2 to 4 of Pkd1. The product from primer set Pkd1X1F1/Pkd1X1R1 yields a 119 bp PCR in wild type (no loxP) DNA and a 319 bp in the undeleted loxP allele. Pkd1X1F1/PkdlX2R4 will result in a PCR product of 425 bp only obtained after Cre mediated recombination. The PCR products were resolved on a 1.5% (w/v) agarose gel (Sigma-Aldrich, Cat. no. A9539), imaged with ChemiDoc XRS+Imaging System (BioRad, Cat. No. 1708265) and the intensity of the PCR bands in captured TIFF images were quantitated using ImageJ (NIH). A ratio of the densitometric intensity of the loxP-deleted 425 bp band to the sum of the densitometric intensities of the loxP-undeleted 319 bp band plus the deleted 425 bp band was taken as indicating the deletion efficiency for each sample.

### Translating Ribosome Affinity Purification (TRAP)

TRAP was performed largely by the method described by Heiman et al. (Nature protocols 9, 1282-1291 (2014)) with a few modifications. Briefly, fresh kidney in lysis buffer [20 mM HEPES-KOH (Fisher Scientific, Cat. no. AAJ16924AE), 5 mM MgCl₂ (Thermo Fisher Scientific, Cat. no. AM9530G), 150 mM KCl (Thermo Fisher Scientific, Cat. no. AM9640G), 0.5 mM DTT (AmericanBio, Cat. no. AB00490-00005), Mini Complete EDTA-Free Protease inhibitor (Roche, Cat. no. 11873580001), 100 μg/ml Cycloheximide (Sigma-Aldrich, Cat. no. C7698), 40 U/ml RNasin Plus Ribonuclease Inhibitor (Promega, Cat. no. N2615), and 20 U/ml SUPERase·In RNase Inhibitor (Thermo Fisher Scientific, Cat. no. AM2696)] was homogenized using a bead mill homogenizer (Precellys® evolution, Bertin Instruments) at 5500 rpm for 20 seconds twice with 1 minute ice-bath in between. The homogenate was then centrifuged at 2,000 xg at 4° C. for 10 minutes and the soluble fraction was mixed with 10% NP-40 (Thermo Fisher Scientific, Cat. no. 28324) prior centrifugation at 20,000×g at 4° C. for 10 minutes. The supernatants of the homogenates were mixed with anti-GFP monoclonal antibodies (Monoclonal Antibody Core Facility, Memorial Sloan-Kettering Cancer Center, New York, Cat. no. Clone: 19C8 and 19F7) pre-conjugated to magnetic beads (Dynabeads MyOne Streptavidin T1, Thermo Fisher Scientific, Cat. no. 65601) for overnight immunoprecipitation at 4° C. with gentle rotation. The magnet bound fraction was washed three times with 1 M KCl buffer containing 20 mM HEPES-KOH (pH 7.4), 5 mM MgCl₂, 1 M KCl, 1% NP-40, 0.5 mM DTT and 100 μg/ml Cycloheximide. Any residual DNA was removed with DNase I digestion (Qiagen, Cat. no. 79254) and RNA was isolated with RNeasy MinElute columns (Qiagen, Cat. no. 74204). The RNA eluted in RNase-free water were stored at −80° C. until sequencing. mRNA library preparation and sequencing were performed by the Yale Center for Genome Analysis. The sequencing library was prepared using the ribosomal depletion method (KAPA RNA HyperPrep Kit with RiboErase, Roche) and sequencing was run on Illumina's NovaSeq 6000 platform using 150 bp paired end reads with read depth 100 million reads per sample.

### TRAPseq Analysis

Raw TRAPSeq fastq files were processed using fastq tool (version 0.20.0) (Chen et al., Bioinformatics 34, i884-i890 (2018)). Sequencing reads with low-quality bases were trimmed or filtered with the default settings. Cleaned reads were aligned using STAR (version 2.7.9) (Dobin et al., Bioinformatics 29, 15-21 (2013)) with the mouse reference genome gencode version GRCm38.p6 with vM25 gene annotation. Expression quantification for aligned reads was performed using featureCounts (version 2.0.0) (Liao et al., Bioinformatics 30, 923-930 (2014)). The present study used an expression threshold of ≥6 read counts in at least 20% of samples for each sex; genes not meeting this expression threshold were eliminated from downstream analyses. The filtered read counts matrix was normalized by the transcripts per million (TPM) method. Detection of differentially expressed genes was performed using R package DESeq2 (version 1.30.1) (Love et al., Genome Biol 15, 550 (2014)) and the Benjamini-Hochberg procedure was used for multiple test correction with FDR≤0.05 used as the significance threshold for detection of differentially expressed genes (DEG). The DEGs in the Pkd1KO mutant that when compared to the noncystic and Pkd1KO+ ciliaKO have the same change direction were defined as ‘CDCA pattern’ DEGs. Heatmaps were generated using the ‘pheatmap’ package in R, with genes and samples hierarchically clustered using the Pearson correlation method. For the heatmaps generated from microdissected kidney tubule bulk RNASeq data (Chen et al., J Am Soc Nephrol (2021)), CDCA pattern genes were first ranked based on how many of the 14 microdissected tubule segments showed expression TPM>1.0, and then further sorted by their significance level (adjusted p-value) in the comparison between Pkd1^{KO} and noncystic male samples. The analysis pipeline scripts are available at: https://github.com/StefanSomloLab/TRAPseq.

### Primary Cell Culture

Primary cells from kidney were isolated using enzymatic digestion procedures. Briefly, adult mouse kidney tissue was collected and minced into small pieces maintaining sterile conditions. Minced tissue pieces were transferred to GentleMACS C-tube (Miltenyi Biotech, Cat. no. 130093237) containing 5 ml of freshly prepared dissociation buffer (DMEM containing D, P, A, Y enzymes). The C-tube was inserted into GentleMACS Octo-Dissociator (Miltenyi Biotech, Cat. no. 130096427) and run with pre-set program ‘37 multi E 01’ (run time of 30 mins). After cell dissociation, 5 ml of DMEM with 10% FBS was added to stop digestion, and the mixture was passed through 70 μm strainer followed by centrifugation at 400×g for 10 minutes. The cell pellet was resuspended in REGM medium (Lonza, REGM Renal Epithelial Cell Growth Medium BulletKit, Cat. no. cc-3190) and cells were directly seeded for experiments. Cells were let attach for 48 hours and then treated with doxycycline (Sigma, Cat. no. D9891-100G) 1 μg/ml for 72 hrs, followed by serum starvation in 0.10% FBS containing media for 24 hours prior to preparation of protein or RNA. For quality control purposes, cells were seeded in parallel, treated similarly to experimental conditions and tested to assess Cre dependent deletion by genotyping after doxycycline treatment. All experiments were done with freshly isolated primary cells without passaging. This reduced the chance of increasing fibroblast overgrowth in the culture. Fibroblast overgrowth was observed in passaged primary cells. Epithelial composition of isolated primary cells was tested by immunofluorescence for epithelial, fibroblasts markers and lineage markers, e.g. ZO1, COL1A1, PAX2 and PAX8 (data not shown).

### Cell Lines

Cell line generation from primary cells was performed using lentivirus of SV40 large T antigen (Gentarget, Cat. no. LVP016-Hygro). Hygromycin antibiotic was used for selection of the transduced and transformed cells. Cell lines were validated by genotyping and by protein analysis for target alleles. IMCD3 cells were cultured in DMEM/F12 (Thermo Fisher Scientific, Cat. no. 10565042) with 5% FBS. IMCD3-3F5, 3F6 Pkd1 KO clones and the Cas9 parental line have been described previously (Decuypere et al., Int J Mol Sci 22(2021)). IMCD3 cells were transduced with lentiviruses containing Glis2-EGFP or Glis3-EGFP to generate stable cells. HEK293T were cultured in DMEM high glucose (Thermo Fisher Scientific, Cat. no. 11965092) with 10% FBS. Cells were either transiently transfected with indicated plasmids or stable expression was performed using lentiviruses of the indicated constructs and appropriate selection antibiotic. All cell lines were serum starved 24 hours in 0.1% FBS media prior experimentation.

### Generation of Anti-Glis2 Antibodies

Anti-Glis2 antibodies (named YNG2) were custom synthesized by Covance (now Labcorp Drug Development, NC, USA). Two rabbits were injected with 500 μg of each of two peptides corresponding to amino acids 31-46 and 462-476 of the mouse Glis2 sequence (NCBI Accession NP_112461) followed by three rounds of boosting. Serum was collected after final boost and antibodies were obtained from the pooled serum by peptide antigen affinity purification.

### Plasmids, Transient Transfection, and Lentiviral Infection

Mouse Glis2 (NCBI Accession NM_031184) was cloned into several vectors with different epitope tag combinations: pLenti CMV GFP Blast (Addgene, Cat. no. #17445) with N-terminal V5 tag and C-terminal EGFP tag; pLVX-TetOne (Takara, Cat. no. 631846) with C-term FLAG tag; pHTC HaloTag® CMV-neo was used to clone Glis2 with C-term Halo tag. Human GLIS2 (NCBI Accession NM_032575) was purchased as GLIS2-HaloTag® human ORF in pFN21A (Promega, Cat. no. FHC03277). Mouse Glis3 (NCBI Accession NM_175459) was cloned into pLenti CMV GFP Blast with C-term EGFP tag. The cilia marker NPHP3⁽¹⁻²⁰⁰⁾-mApple was made by amplifying a fragment corresponding to amino acid 1-200 of mouse Nphp3 (NCBI Accession NM_028721.3) from mouse kidney cDNA and combining it in-frame with mApple using PCR, followed by subcloning into a pCDH-Hygro vector which was modified from pCDH-EF1-MCS-IRES-Puro (System Biosciences). All constructs were validated by sequencing and immunoblot expression analysis. Transient transfection of HEK293T cells was done with Lipofectamine 2000 (Thermo Fisher Scientific, Cat. no. 11668030). Stable gene expression was achieved using lentivirus transduction and selection with appropriate antibiotic.

### Protein Preparation, Electrophoresis and Immunoblotting

Total lysates from cell pellets or tissues were prepared for immunoblotting using 1× Red Loading Buffer Pack (Cell Signal Technology, Cat. no. 7723S) according to manufacturer's instructions. Briefly, cell pellets or tissue were suspended in the buffer without DTT and sonicated three times, each in 10 pulses of 1-2 seconds, with a probe sonicator (Misonix, XL-2000; power setting 6). Protein amounts were quantified using a bicinchoninic acid assay (BCA) kit (Thermo Fisher Scientific, Cat. no. 23225). DTT was added to normalized concentrations of protein and samples were boiled for 5 minutes prior to SDS-PAGE. Fractionation of cells or tissue into cytosolic and nuclear parts was done using either NE-PER™ Nuclear and Cytoplasmic Extraction Reagents (Thermo, Cat. no. 78833) with manufacturer's instructions or by a non-kit method. For the latter, cells pellets were resuspended in ice cold lysis buffer containing 100 mM Tris pH 7.4, 20 mM NaCl, 10 mM EDTA, 0.1% Triton X100 and incubated on ice for 5 minutes. For fractionation of tissues, tissue in the same lysis buffer was homogenized in a bead mill homogenizer (Precellys® evolution, Bertin Instruments) at 6800 rpm for 30 seconds twice with 1 minute ice-bath in between. Cell or tissue lysates were resuspended and centrifuged at 21,000×g for 5 minutes. The supernatant was transferred to a new tube and comprised the cytosolic fraction. The pellet was resuspended in 1× Red Loading buffer and followed the protein extraction steps described above.

Lysates were run on 4-20% Mini-PROTEAN TGS Precast Protein gels (Biorad, Cat. no. 4561094) for all except for PC1 western blots. PC1 western blots were run on NuPAGE 3-8% Tris-Acetate 1.5 mm Mini Protein gels (Thermo Fisher Scientific, Cat. no. EA0378BOX). Proteins were transferred either on Nitrocellulose membrane (Biorad, Cat. no. 1620115) or PVDF membrane (Biorad, Cat. no. 1620177). Precision Plus Protein™ Dual Color Standards (Biorad, Cat. no. 1610374) or PureView Prestained Protein Ladder (Azura Genomics, Cat. no. AZ-1142-2) were used as size markers. Membrane stripping for re-probing was done using Restore PLUS Western Blot Stripping Buffer (Thermo Fisher Scientific, Cat. no. 46430). Briefly, membranes were washed with running water multiple times. Then, membranes were incubated with buffer enough to cover the membrane at 55° C. for 10 mins followed by washing with running water multiple times. Membranes were then blocked with 5% non-fat dry milk for 10 minutes, followed by incubation with primary and secondary antibodies. Chemiluminescent signals from membranes were determined using either Clarity Western ECL Substrate (Biorad, Cat. no. 1705060) or SuperSignal Femto Maximum Sensitivity Substrate (Thermo Scientific, Cat. no. 34095) on Licor Odyssey Fc.

### Antibodies Used

| | Antibody | | | Dilution | Dilution |
|---|---|---|---|---|---|
| Target | name | Company | Cat. no. | (IB) | (IF) |
| PC1 | 7e12 | Santa Cruz | sc-130554 | 1:1000 | NA |
| | | Biotechnology | | | |
| PC2 | YCC2 | Cai et al. | NA | 1:2000 | NA |
| | | (J. Biol. Chem. | | | |
| | | 274, 28557-28565 | | | |
| | | (1999)) | | | |
| GLIS2 | YNG2 | This study | NA | 1:40,000 | 1:1000 |
| KIF3a | D7G3 | Cell Signaling | 8507S | 1:1000 | NA |
| | | Technology | | | |
| IFT88 | IFT88 | Proteintech | 13967-1- | 1:1000 | 1:500 |
| | Polyclonal | | AP | | |
| | antibody | | | | |
| TULP3 | Tulp3 | Proteintech | 13637-1- | 1:1000 | 1:500 |
| | Polyclonal | | AP | | |
| | antibody | | | | |
| ZO1 | ZO1 | Proteintech | 21773-1- | NA | 1:500 |
| | Polyclonal | | AP | | |
| | antibody | | | | |
| ZO1 | ZO-1 | Proteintech | 66452-1-Ig | NA | 1:500 |
| | Monoclonal | | | | |
| | antibody | | | | |
| Acetylated a- | AF647 | Santa Cruz | sc-23950 | NA | 1:1000 |
| Tubulin | | Biotechnology | | | |
| ARL13b | ARL13b | Proteintech | 66739-1-Ig | NA | 1:1000 |
| | Mouse | | | | |
| | Monoclonal | | | | |
| | Antibody | | | | |
| HSP90 | HSP90 | Cell Signaling | 4877S | 1:1000 | NA |
| | Antibody | Technology | | | |
| | #4874 | | | | |
| LAMIN A/C | Lamin A/C | Cell Signaling | 2032S | 1:1000 | NA |
| | Antibody | Technology | | | |
| | #2032 | | | | |
| LAMIN B1 | Lamin B1 | Proteintech | 12987-1- | 1:1000 | NA |
| | Polyclonal | | AP | | |
| | antibody | | | | |
| Anti-FLAG | Mouse | Sigma-Aldrich | F1804 | 1:500 | NA |
| | monoclonal | | | | |
| | ANTI- | | | | |
| | FLAG ® M2 | | | | |
| | antibody | | | | |
| Caspase-1 | Mouse anti- | Adipogen | AG-20B- | 1:1000 | 1:200 |
| | cleaved | International | 0042-C100 | | |
| | caspase-1 | | | | |
| TNFα | Mouse anti- | Santa Cruz | SC-52746 | 1:1000 | 1:200 |
| | TNFα | Biotechnology | | | |
| α-SMA | Rabbit anti-α- | Abcam | ab5694 | 1:1000 | 1:200 |
| | SMA | | | | |
| PDGFR-β | Rabbit anti- | Abcam | ab32570 | 1:1000 | 1:200 |
| | PDGFR-β | | | | |
| F4/80 | Rat anti-F4/80 | AbD Serotec | MCA497R | NA | 1:200 |
| Ki-67 | Rabbit | Thermo Fisher | RM 9106- | NA | 1:200 |
| | monoclonal | Scientific | S1 | | |
| | anti-Ki-67 | | | | |
| Megalin | Rabbit anti- | 21 | NA | NA | 1:500 |
| | megalin | | | | |
| Dolichos | FITC DBA | Vector | FITC (FL- | NA | 1:50 |
| biflorus | or | Laboratories | 1031) | | |
| agglutinin | Rhodamine | | Rhodamine | | |
| (DBA) | DBA | | (RL-1032) | | |
| Lotus | FITC LTA | Vector | FL-1321 | NA | 1:200 |
| tetragonolobus | | Laboratories | | | |
| agglutinin | | | | | |
| (LTA) | | | | | |
| Secondary | Alexa-488, | Molecular | | NA | 1:500 |
| antibodies | Alexa-594 | Probes Thermo | | | |
| | and Alexa- | FIsher | | | |
| | 647 | | | | |
| | conjugated | | | | |
| | secondary | | | | |
| | antibodies | | | | |
| Hoechst 33342 | Nuclear stain | Molecular | H-3570 | NA | 1:5000 |
| | | Probes Thermo | | | |
| | | Fisher | | | |

RNa Isolation and qRT-PCR

Total RNA from kidney and cells was isolated using either RNeasy Mini Kit (Qiagen, Cat. no. 74104) or Trizol (Themo Fisher Scientific, Cat. no. 15596026) based method followed by column purification. Cells were serum starved overnight before the day of RNA preparation. For tissue homogenization, kidneys were homogenized in Trizol by bead mill homogenizer (Precellys® evolution, Bertin Instruments) at 6800 rpm for 30 seconds twice with 30 seconds in ice-bath in between. Two g of total RNA was used for cDNA synthesis using iScript cDNA synthesis kit (Biorad, Cat. no. 1708890). Quantitative RT-PCR was performed using iTaq Universal SYBR green Supermix (Biorad, Cat. no. 18064022) or AzuraView Green Fast qPCR Blue Mix, LoRox (Azura Genomics, Cat. no. AZ2320) in CFX96 Touch Real-Time PCR detection system (Biorad, USA). (Gapdh or 18s was used for normalization.

qRT-PCR Primers

| Gene | Forward Primer | Reverse Primer |
|---|---|---|
| Pkd1 | 5′- | 5′- |
| | CTAGACCTGTCCCACAACCTA | GCAAACACGCCTTCTTCTAATGT |
| | (SEQ ID NO 55) | (SEQ ID NO 56) |
| Pkd2 | 5′- | 5′- |
| | GGGGAACAAGACTCATGGAAG | GCCGTAGGTCAAGATGCACAA |
| | (SEQ ID NO 57) | (SEQ ID NO 58) |
| Glis2 | 5′- | 5′-TGACAGCAGTATCGAGCATCC |
| | GTGTCGATGGGCCAAGTGTAA | (SEQ ID NO 60) |
| | (SEQ ID NO 59) | |
| Kif3a | 5′- | 5′-GTTCCCCTCATTTCATCCACG |
| | ATGCCGATCAATAAGTCGGAG | (SEQ ID NO 62) |
| | A | |
| | (SEQ ID NO 61) | |
| Cables2 | 5′- | 5′- |
| | AGGAGACGAGTCACATCTCAG | TGTGTCATACTGCCTCATGTTC |
| | (SEQ ID NO 63) | (SEQ ID NO 64) |
| Chpf | 5′- | 5′- |
| | AGTGCCTGATGCCACCTATAC | CGGCCAAGATAGAGATGGGTTG |
| | (SEQ ID NO 65) | (SEQ ID NO 66) |
| Tspan5 | 5′-GGGAAGCACTACAAGGGTCC | 5′- |
| | (SEQ ID NO 67) | CCACAGTCCGATTCCAAGAAA |
| | | (SEQ ID NO 68) |
| Anks3 | 5′-AGCTCAGCGATGAAGCCAG | 5′- |
| | (SEQ ID NO 69) | CTGTGTGAAGATCCAAGGGGA |
| | | (SEQ ID NO 70) |
| Ptpdc1 | 5′- | 5′- |
| | GGTCACTGACAATATCCTGGC | AGGTAGGTGAAACCACTTTCTTG |
| | (SEQ ID NO 71) | (SEQ ID NO 72) |
| Lad1 | 5′- | 5′-CTGTGGTTGAACTCAGGTTGC |
| | ATGTCGGTCAGCAGAAAGGAC | (SEQ ID NO 74) |
| | (SEQ ID NO 73) | |
| Ntn4 | 5′-GCAGGCTTGAATGGAGTAGC | 5′-GCAGCGTTGCATTTATCACAC |
| | (SEQ ID NO 75) | (SEQ ID NO 76) |
| Gapdh | 5′- | 5′- |
| | AGGTCGGTGTGAACGGATTTG | TGTAGACCATGTAGTTGAGGTC |
| | (SEQ ID NO 77) | A |
| | | (SEQ ID NO 78) |
| 18s | 5′-GTAACCCGTTGAACCCCATT | 5′-CCATCCAATCGGTAGTAGCG |
| | (SEQ ID NO 79) | (SEQ ID NO 80) |
| Adgre1 | 5′-AGTACGATGTGGGGCTTTTG | 5′-ACTCCTGGGCCTTGAAAGTT |
| (F4/80) | (SEQ ID NO 81) | (SEQ ID NO 82) |

### Immunocytochemistry and Cystic Index Measurement

Immunocytochemistry was performed on primary cells, TMCD3 and BIEK293T as per standard protocols. Cells were plated on 4-well chambered dishes (Cellvis, Cat. no. C4-1.5P). All adhered cells were washed gently two times with 1×PBS followed by fixation with 4% PFA in PBS for 20 minutes. Cells were washed 3 times with 1×PBS and permeabilized with 0.25% Triton X100 for 5 minutes followed by blocking with 5% BSA in PBS. Primary antibody was diluted in 5% BSA and incubated at room temperature for 1 hour. Cells were washed 3 times with PBS followed by secondary antibody in 5% BSA for 1 hour at room temperature. Cells were washed another 3 times with PBS and Hoechst was added to stain nuclei. Microscopy of non-fixed live cells was done on adhered cells. Cells were washed in sterile conditions in 1×PBS and imaging was done on cells with 1×PBS to reduce autofluorescence from media. All images were acquired on Nikon Eclipse Ti (Nikon Instruments Inc, Japan) equipped with Yokogawa CSU-W1 spinning disc and Andor lasers (Andor Technology, UK). For movies, gamma correction was done to reduce background noise. For tissue immunohistochemistry, cryosections (5-7 μm) were used according to standard procedures. Cystic index was calculated as previously described (Ma et al., Nat Genet 45, 1004-1012 (2013))¹⁰ using frontal plane sections of kidneys processed for hematoxylin and eosin and scanned by light microscopy under the control of MetaMorph software (Universal Imaging, version 7.7).

### Blood Urea Nitrogen (BUN) Measurements

BUN was measured at the George M. O'Brien Kidney Center at Yale Core using Stanbio Urea Nitrogen (BUN) Procedure 0580 quantitative colorimetric determination of urea nitrogen in serum and plasma. The method is based on the diacetylmonoxime (DAM) methodology. Samples were read on the Excel Chemistry Analyzer (Stanbio Laboratory, Boerne Texas).

### Proliferation Assays

Proliferation was measured by EdU incorporation and Ki67 staining. For EdU incorporation, mice received 50 mg/kg of 5-ethynyl-2′-deoxyuridine (Invitrogen, Cat. no. A10044) by intraperitoneal injection 4 hours before euthanasia. Kidneys were fixed with 4% PFA overnight and embedded in OCT after 30% sucrose infiltration and processed for immunofluorescence. EdU staining was performed with Click-iT™ EdU Imaging Kit with Alexa Fluor™ 647 (Invitrogen, Cat. no. C10086). KI-67 expression in kidney was detected with rabbit anti-Ki67 antibody (Thermo Fisher Scientific, RM-9106-S1, dilution 1:200). Numbers of EdU and Ki67 positive nuclei were counted amongst at least 1,000 DBA or LTA positive cells per animal.

### Senescence Associated-β-Galactosidase (SA-β-Gal) Staining in Tissue

SA-β-Gal staining was done using the Senescence β-Galactosidase Staining Kit (Cell Signaling, Cat. no. 9860), adapted for tissue, according to manufacturer's instructions. Briefly, kidneys were collected after perfusing mice with 4% PFA in 1×PBS followed by fixation in 4% PFA at 4° C. overnight. Following fixation, kidneys were treated with 30% sucrose solution in 1×PBS at 4° C. overnight and embedded in OCT compound. Frozen sections (5-7 μm) were air dried for 15 minutes and washed with 1×PBS three times, then incubated with fresh SA-β-Gal staining solution at 37° C. for 24 hours. Sections were counterstained with Nuclear Fast Red solution (Statlab Medical Products, STNFR100).

### RNAScope Multiplex Fluorescent In Situ Hybridization (RNAScope-FISH)

RNAScope® Multiplex Fluorescent Kit v2 (Advanced Cell Diagnostics) was used to perform RNA-probe based fluorescent in situ hybridization (FISH) according to the manufacturer's instructions. Briefly, 5-7 μm thick Tissue-Tek OCT Compound (Sakura, Cat. no. 4583) embedded cryosections of mouse kidneys were fixed in 4% PFA for 15 mins at 4° C. prior to serial dehydration for 5 mins each with 50%, 70% and 100% ethanol. Sections were then treated with hydrogen peroxide for 10 mins at room temperature, followed by incubation in RNAscope target retrieval solution for 15 mins at 98-102° C. and then treated with RNAscope Protease III for 30 mins at 40° C. RNA-specific probes targeting mouse Glis2 (Mm-Glis2, Cat. no. 405621) and mouse megalin (Mm-Lrp2, Cat. no. 425881; Advance Cell Diagnostics Inc., USA) were incubated for 2 hours at 40° C. Sections were counterstained with DAPI, and mounted with fluorescent mounting media with anti-fading agent DABCO (Fluoro Gel with DABCO, EMS, USA). All the slides were kept at 4° C. prior to image acquisition. Images were obtained using a confocal microscope (Nikon Eclipse Ti, Japan). under the control of NIS-Elements AR 4.30.02 software (Nikon).

Single Molecule Fluorescence In Situ Hybridization (smFISH)

Kidneys were removed and fixed in 4% PFA in 1×PBS for 4 hours and subsequently incubated in a 3000 sucrose, 400 PFA in 1×PBS solution at 4° C. overnight with gentle agitation. Fixed kidneys were embedded in Tissue-Tek OCT Compound (Sakura, Cat. no. 4583) and 5 μm thick sections placed onto poly L-lysine coated coverslips. Probe libraries for Glis2 and Lrp2 were designed using the Stellaris FISH Probe Design Software (Biosearch Technologies) with the following sequences:

| GLIS2 | | LRP2 | |
|---|---|---|---|
| PROBE | GLIS2 PROBE SEQUENCE | PROBE | LRP2 PROBE SEQUENCE |
| PROBE 1 | 5′- | PROBE 1 | 5′- |
| | TTGGTGATGCTCAGCTTTAG | | CACAGCGAAAATTCCCACTG |
| | (SEQ ID NO 83) | | (SEQ ID NO 116) |
| PROBE 2 | 5′- | PROBE 2 | 5′- |
| | TCGATGCAAAGCATGATGC | | AGTGTCATCCAAACAGTCTC |
| | C (SEQ ID NO 84) | | (SEQ ID NO 117) |
| PROBE 3 | 5′- | PROBE 3 | 5′- |
| | GGAAACCTGGTGGTGGAGA | | TCGGAACAATCCTTATCCTG |
| | G (SEQ ID NO 85) | | (SEQ ID NO 118) |
| PROBE 4 | 5′- | PROBE 4 | 5′- |
| | TCAGGGAATTTGGGGTTCA | | GTACTCGATGGGAACACACT |
| | G (SEQ ID NO 86) | | (SEQ ID NO 119) |
| PROBE 5 | 5′- | PROBE 5 | 5′- |
| | CAAGGGGGCTGCAGAAAAG | | AATAGCAGTTTCTCTCGTCA |
| | C (SEQ ID NO 87) | | (SEQ ID NO 120) |
| PROBE 6 | 5′- | PROBE 6 | 5′- |
| | GAGGGTGGTGACAAGCTGA | | GTCATGGTCACAGACATAGG |
| | G (SEQ ID NO 88) | | (SEQ ID NO 121) |
| PROBE 7 | 5′- | PROBE 7 | 5′- |
| | CAGTAGGCAGTGGAGGCAA | | CATCGCTGTTATCTTCACAG |
| | G (SEQ ID NO 89) | | (SEQ ID NO 122) |
| PROBE 8 | 5′- | PROBE 8 | 5′- |
| | AACCCAGGGGCAAGAAGAA | | CCATTCTCTTGGGTAACATG |
| | C (SEQ ID NO 90) | | (SEQ ID NO 123) |
| PROBE 9 | 5′- | PROBE 9 | 5′- |
| | ATCGACACACCAGCTGCTT | | CGGGAACTCCATCACAAACT |
| | G (SEQ ID NO 91) | | (SEQ ID NO 124) |
| PROBE 10 | 5′- | PROBE 10 | 5′- |
| | GCTCAAAGAGCTGGTTACA | | CCAGGATATAACCTTCTTCA |
| | C (SEQ ID NO 92) | | (SEQ ID NO 125) |
| PROBE 11 | 5′- | PROBE 11 | 5′- |
| | TCGTTGACATGGTCAACCA | | TCGTTGGATTTGCAATGCTG |
| | G (SEQ ID NO 93) | | (SEQ ID NO 126) |
| PROBE 12 | 5′- | PROBE 12 | 5′- |
| | CTGTTCAGGCTTGACATGAT | | CAAATCCCGACCATTAGAGA |
| | (SEQ ID NO 94) | | (SEQ ID NO 127) |
| PROBE 13 | 5′- | PROBE 13 | 5′- |
| | CCAATGACAGCAGTATCGA | | TTTCTTCCATGAAGATCTCC |
| | G (SEQ ID NO 95) | | (SEQ ID NO 128) |
| PROBE 14 | 5′- | PROBE 14 | 5′- |
| | TCTTGTACCTGGCATTGAAG | | ATCAAGGGCAATTCCTCTAG |
| | (SEQ ID NO 96) | | (SEQ ID NO 129) |
| PROBE 15 | 5′- | PROBE 15 | 5′- |
| | TGTGTCCGGATGTGAATGA | | GATTGCTACCATCCATGAAG |
| | G (SEQ ID NO 97) | | (SEQ ID NO 130) |
| PROBE 16 | 5′- | PROBE 16 | 5′- |
| | TTGTGGATCTTCAGGTTCTC | | CGCTTGGATACAAGATCCAG |
| | (SEQ ID NO 98) | | (SEQ ID NO 131) |
| PROBE 17 | 5′- | PROBE 17 | 5′- |
| | TGGAGTAACGCTTGTTGCA | | AAACGGATGAGGGACGAGGG |
| | G (SEQ ID NO 99) | | (SEQ ID NO 132) |
| PROBE 18 | 5′- | PROBE 18 | 5′- |
| | TGTGCTTGAAGCGGTCACT | | ATGGTAAACTGTCACTCCGA |
| | G (SEQ ID NO 100) | | (SEQ ID NO 133) |
| PROBE 19 | 5′- | PROBE 19 | 5′- |
| | TTGTCTACGTAGTGGGTACG | | CAGCCGTTTGTGATGAGAAG |
| | (SEQ ID NO 101) | | (SEQ ID NO 134) |
| PROBE 20 | 5′- | PROBE 20 | 5′- |
| | CGGGCATCTTGCAGTAGTA | | AGAGATTCCTTGAGATCCAG |
| | G (SEQ ID NO 102) | | (SEQ ID NO 135) |
| PROBE 21 | 5′- | PROBE 21 | 5′- |
| | TCCGTGTAACGCTTGTGACA | | CAGCTTCATGACAGTGACAC |
| | (SEQ ID NO 103) | | (SEQ ID NO 136) |
| PROBE 22 | 5′- | PROBE 22 | 5′- |
| | TGATGTGTTTGCGCAGTGA | | TCGAGGGTTGTTTAAGTTGC |
| | A (SEQ ID NO 104) | | (SEQ ID NO 137) |
| PROBE 23 | 5′- | PROBE 23 | 5′- |
| | ATGTGACACAAAGTGGCCA | | GACTATGGGCATTAGGTGAG |
| | T (SEQ ID NO 105) | | (SEQ ID NO 138) |
| PROBE 24 | 5′- | PROBE 24 | 5′- |
| | GACATAGGAGCCACTGTCA | | AACGGTGAGTCCAAACGGAT |
| | G (SEQ ID NO 106) | | (SEQ ID NO 139) |
| PROBE 25 | 5′- | PROBE 25 | 5′- |
| | TTCGGGATGATGATCTGAG | | GTTTGGAACTGGGAAGCAGA |
| | C (SEQ ID NO 107) | | (SEQ ID NO 140) |
| PROBE 26 | 5′- | PROBE 26 | 5′- |
| | GAGGTAGAGGTAATGGCAG | | CTCTGAAGCTTCATTCCATA |
| | G (SEQ ID NO 108) | | (SEQ ID NO 141) |
| PROBE 27 | 5′- | PROBE 27 | 5′- |
| | CAGAGCACTGAGGTCAAGG | | CTGTTGTCATGGCAATCATC |
| | G (SEQ ID NO 109) | | (SEQ ID NO 142) |
| PROBE 28 | 5′- | PROBE 28 | 5′- |
| | CAAAGGGTGAGGGCAACAG | | CAGTCATTCTGTTTGTCACA |
| | C (SEQ ID NO 110) | | (SEQ ID NO 143) |
| PROBE 29 | 5′- | PROBE 29 | 5′- |
| | AGGAGAGAAACCACAGGCA | | CACTCTTTGGGGATACACAT |
| | G (SEQ ID NO 111) | | (SEQ ID NO 144) |
| PROBE 30 | 5′- | PROBE 30 | 5′- |
| | TGTGGACAGGTCCAGTACA | | CTGATCCATCCGAACAATCA |
| | G (SEQ ID NO 112) | | (SEQ ID NO 145) |
| PROBE 31 | 5′- | PROBE 31 | 5′- |
| | GAGTTAACACCTCTGGACT | | CTTGAACTGAGAGCTTGTGC |
| | G (SEQ ID NO 113) | | (SEQ ID NO 146) |
| PROBE 32 | 5′- | PROBE 32 | 5′- |
| | ACAGACCCTGGTGGAATGA | | TAGATGCAGTTTCCATTGTC |
| | C (SEQ ID NO 114) | | (SEQ ID NO 147) |
| PROBE 33 | 5′- | PROBE 33 | 5′- |
| | TTTACCACAGCTGGTTTGAG | | AAAAGGCTGGGTAGGACAGT |
| | (SEQ ID NO 115) | | (SEQ ID NO 148) |
| | | PROBE 34 | 5′- |
| | | | TTTGTCACGACTGGCTACAA |
| | | | (SEQ ID NO 149) |
| | | PROBE 35 | 5′- |
| | | | TTTACCCTCCAATAAGTCAC |
| | | | (SEQ ID NO 150) |
| | | PROBE 36 | 5′- |
| | | | TTGTCGGTTCCGTTTTGAAA |
| | | | (SEQ ID NO 151) |
| | | PROBE 37 | 5′- |
| | | | TATCCAGTCTACTGCAATCA |
| | | | (SEQ ID NO 152) |
| | | PROBE 38 | 5′- |
| | | | TCTGTCCAGTAAATGTTGCG |
| | | | (SEQ ID NO 153) |
| | | PROBE 39 | 5′- |
| | | | TACATTGTCACCCATTCTAG |
| | | | (SEQ ID NO 154) |
| | | PROBE 40 | 5′- |
| | | | CCATGAAGTAGATGAGGCGA |
| | | | (SEQ ID NO 155) |
| | | PROBE 41 | 5′- |
| | | | TGGGGAACGCTGTACATTAC |
| | | | (SEQ ID NO 156) |
| | | PROBE 42 | 5′- |
| | | | TCACTCTGTGAATTTCACCT |
| | | | (SEQ ID NO 157) |
| | | PROBE 43 | 5′- |
| | | | GGAGTGTCAACACCTCAATG |
| | | | (SEQ ID NO 158) |
| | | PROBE 44 | 5′- |
| | | | TAATCAGGGTTTTCCCATAC |
| | | | (SEQ ID NO 159) |
| | | PROBE 45 | 5′- |
| | | | TAAAGGAAGGAGCCGTGGAC |
| | | | (SEQ ID NO 160) |
| | | PROBE 46 | 5′- |
| | | | ATTGGAAGAGTCAGCAGCAT |
| | | | (SEQ ID NO 161) |
| | | PROBE 47 | 5′- |
| | | | AGGCAGCATGGAAACGACTA |
| | | | (SEQ ID NO 162) |
| | | PROBE 48 | 5′- |
| | | | GTGATCAGACAACTCCAAGC |
| | | | (SEQ ID NO 163) |

Probes for Glis2 were coupled to Quasar® 670 Dye and probes for Lrp2 (megalin) were coupled to CAL Fluor® Red 590 Dye. Kidney sections were hybridized with smFISH probe sets based on the Stellaris RNA FISH protocol (Biosearch Technologies). Images were obtained using a confocal microscope (Nikon ECLIPSE Ti, Japan). under the control of NIS-Elements AR 4.30.02 software (Nikon). A pipeline in CellProfiler²² was written that segmented and identified Megalin puncta, Glis2 puncta and nuclear signal. The intensity of the Glis2 puncta normalized to the number of nuclei were used to calculate signal intensity.

### Mouse Glis2 Antisense Oligonucleotide (Glis2 ASO) Development, Characterization, and Administration

The Glis2 ASOs were targeting murine Glis2 were designed, synthesized, and tested in a series of in vitro and in vivo screens and evaluated for efficacy and tolerability in 8-10-week-old male C57BL/6J mice at a dose of 50 mg/kg per week for 3 weeks. ASOs that displayed robust reduction of kidney Glis2 mRNA without inducing elevations in plasma transaminases and mRNA biomarkers of renal tubular injury (KIM-1 and NGAL) were selected for additional in vivo studies in models of ADPKD. The Glis2 ASO identified from the screening exercise evaluated in the disease models had the following sequence: 5′-CCTTATAAGCTTCTGC-3′ (SEQ ID NO 164). Additionally, a control ASO was used: 5′-ACGATAACGGTCAGTA (SEQ ID NO 165), with the underlined sequences indicated the 2′,4′-constrained ethyl-D-ribose (cEt) modified bases. ASO was dissolved and diluted in sterile D-PBS and sterilized by filtration through a 0.22 μm filter. ASO was administered by intraperitoneal injection (IP) at a dose of 50 mg/kg beginning at 5 weeks of age following the end of tamoxifen induction (P28-P35). ASO was administered twice during week 5 (initial week), and once a week from 6 weeks of age through 18 weeks age. Mice were euthanized 1 day after the final dose.

### Sample Size and Statistics

Sample size and power calculations were performed prospectively using STPLAN (v.4.5; University of Texas MD Anderson Cancer Center). Calculations were based on the following inputs: a significance level (α) as 0.05 (one sided) and 0.80 power (1−β) were selected as the threshold. For KW:BW ratio in Pkd1^{fl/fl}; Pax8^{rtTA}, TetO^{Cre} mice at age 18 weeks after receiving oral doxycycline from P28-42, a mean of 10.2% with s.d.±3.3% was empirically derived from the earlier data. The alternative hypothesis was that

Glis2^{fl/fl}; Pkd1^{fl/fl}; Pax8^{rtTA}; TetO^{Cre} mice would result in at least a 40% decrease in the KW:BW ratio. Under these expectations, 6 mice could achieve 80% power to detect the difference between the null hypothesis and the alternative hypothesis. For KW:BW ratio in Pkd1^{fl/fl}; UBC^{CreERT2} mice at age 18 weeks after receiving tamoxifen from P28-35, a mean of 6.2% with s.d.±3.0% was empirically derived from the earlier data. The alternative hypothesis was that Pkd1^{fl/fl}; UBC^{CreERT2}+Glis2-ASO mice would result in at least a 40% decrease in the KW:BW ratio. Under these expectations, 11 mice could achieve 80% power to detect the difference between the null hypothesis and the alternative hypothesis.

Most of the quantitative data were analyzed using one-way analysis of variance (ANOVA) followed by Tukey's multiple-comparison test using GraphPad Prism 9.5.1 software. The data of FIGS. 19D, 19F and 19H were analyzed using two-tailed, unpaired Student's t-test. The data in FIG. 27A were analyzed using two-way ANOVA followed by Dunnett's multiple-comparison test. All data are presented as mean±s.e.m. P<0.05 was considered the threshold for significance throughout. Exact P values are provided for all P>0.0001.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

### ENUMERATED EMBODIMENTS

The following enumerated embodiments are provided, the numbering of which is not to be construed as designating levels of importance:

Embodiment 1: A method of treating, ameliorating and/or preventing a polycystic kidney disease caused by or involving a mutant PKD1 or PKD2 in a subject in need thereof, the method comprising: administering to the subject an effective amount of a compound that downregulates GLIS2.

Embodiment 2: The method of Embodiment 1, wherein the polycystic kidney disease is an autosomal dominant polycystic kidney disease (ADPKD).

Embodiment 3: The method of any one of Embodiments 1-2, wherein the compound that downregulates GLIS2 comprises at least one selected from the group consisting of: a protein inhibitor of GLIS2; a nucleic acid that down regulates GLIS2 by RNA interference, and/or an expression vector expressing the nucleic acid that down regulates GLIS2 by RNA interference; a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; and a trans-dominant negative mutant protein of GLIS2, and/or an expression vector that expresses the trans-dominant negative mutant protein of GLIS2.

Embodiment 4: The method of any one of Embodiments 1-3, wherein the compound that downregulates GLIS2 comprises an antisense oligonucleotide (ASO), an siRNA, or an shRNA downregulating GLIS2 expression, and/or an expression vector expressing the ASO, siRNA or shRNA.

Embodiment 5: The method of any one of Embodiments 1-4, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

Embodiment 6: A composition for treating, ameliorating and/or preventing a polycystic kidney disease caused by or involving a mutant PKD1 or PKD2 in a subject in need thereof, comprising: a compound that downregulates GLIS2; and a pharmaceutically acceptable carrier.

Embodiment 7: The composition of Embodiment 6, wherein the polycystic kidney disease is an autosomal dominant polycystic kidney disease (ADPKD).

Embodiment 8: The composition of any one of Embodiments 6-7, wherein the compound that downregulates GLIS2 comprises at least one selected from the group consisting of: a protein inhibitor of GLIS2; a nucleic acid that down regulates GLIS2 by RNA interference, and/or an expression vector expressing the nucleic acid that down regulates GLIS2 by RNA interference; a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; and a trans-dominant negative mutant protein of GLIS2, and/or an expression vector that expresses the trans-dominant negative mutant protein of GLIS2.

Embodiment 9: The composition of any one of Embodiments 6-8, wherein the compound that downregulates GLIS2 comprises an antisense oligonucleotide (ASO), an siRNA, or an shRNA downregulating GLIS2 expression, and/or an expression vector expressing the ASO, siRNA or shRNA.

Embodiment 10: The composition of any one of Embodiments 6-9, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

Embodiment 11: A kit for treating, ameliorating and/or preventing a polycystic kidney disease caused by or involves a mutant PKD1 or PKD2 in a subject in need thereof, comprising: a compound that downregulates GLIS2; and a manual instructing that the compound that downregulates GLIS2 is to be administer to the subject in an effective amount.

Embodiment 12: The kit of Embodiment 11, wherein the polycystic kidney disease is an autosomal dominant polycystic kidney disease (ADPKD).

Embodiment 13: The kit of any one of Embodiments 11-12, wherein the compound that downregulates GLIS2 comprises at least one selected from the group consisting of: a protein inhibitor of GLIS2; a nucleic acid that down regulates GLIS2 by RNA interference, and/or an expression vector expressing the nucleic acid that down regulates GLIS2 by RNA interference; a ribozyme that down regulates GLIS2, and/or an expression vector expressing the ribozyme; an expression vector comprising an expression cassette, wherein the expression cassette expresses CRISPR components that down regulates GLIS2 by CRISPR knockout or CRISPR knock down; and a trans-dominant negative mutant protein of GLIS2, and/or an expression vector that expresses the trans-dominant negative mutant protein of GLIS2.

Embodiment 14: The kit of any one of Embodiments 11-13, wherein the compound that downregulates GLIS2 comprises an antisense oligonucleotide (ASO), an siRNA, or an shRNA downregulating GLIS2 expression, and/or an expression vector expressing the ASO, siRNA or shRNA.

Embodiment 15: The kit of any one of Embodiments 11-14, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

Embodiment 16: A method of screening suppressors of downstream effects of loss of PKD1 or PKD2, comprising: detecting a first GLIS2 expression level in the nucleus of a cell expressing PKD1 and PKD2; contacting the cell with a compound; and detecting a second GLIS2 expression level in the nucleus of the cell, wherein a difference between the first GLIS2 expression level and the second GLIS2 expression level indicates that the compound is a suppressors of downstream effects of loss of PKD1 or PKD2.

Embodiment 17: The method of Embodiment 16, wherein the cell is a kidney cell or a kidney-derived cell.

Embodiment 18: The method of any one of Embodiments 16-17, wherein the cell is a primary cell or a cell line.

Embodiment 19: The method of any one of Embodiments 16-18, the cell is a mammalian cell or a mammalian cell-derived cell, optionally a human cell or a human cell-derived cell.

Embodiment 20: The method of any one of Embodiment 16-20, wherein that the second GLIS2 expression level is lower or higher than the first GLIS2 expression level indicates that compound is a suppressors of downstream effects of loss of PKD1 or PKD2.

1. A method of treating, ameliorating and/or preventing a polycystic kidney disease caused by or involving a mutant PKD1 or PKD2 in a subject in need thereof, the method comprising:
administering to the subject an effective amount of a compound that downregulates GLIS2.

2. The method of claim 1, wherein the polycystic kidney disease is an autosomal dominant polycystic kidney disease (ADPKD).

3. The method of claim 1, wherein the compound that downregulates GLIS2 comprises at least one selected from the group consisting of:
a protein inhibitor of GLIS2;
a nucleic acid that down regulates GLIS2 by RNA interference, and/or an expression vector expressing the nucleic acid that down regulates GLIS2 by RNA interference;
a ribozyme that down regulates GLIS2, and/or an expression vector expressing the ribozyme;
an expression vector comprising an expression cassette, wherein the expression cassette expresses CRISPR components that down regulates GLIS2 by CRISPR knockout or CRISPR knock down; and
a trans-dominant negative mutant protein of GLIS2, and/or an expression vector that expresses the trans-dominant negative mutant protein of GLIS2.

4. The method of claim 1, wherein the compound that downregulates GLIS2 comprises an antisense oligonucleotide (ASO), an siRNA, or an shRNA downregulating GLIS2 expression, and/or an expression vector expressing the ASO, siRNA or shRNA.

5. The method of claim 1, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

6. A composition for treating, ameliorating and/or preventing a polycystic kidney disease caused by or involving a mutant PKD1 or PKD2 in a subject in need thereof, comprising:
a compound that downregulates GLIS2; and
a pharmaceutically acceptable carrier.

7. The composition of claim 6, wherein the polycystic kidney disease is an autosomal dominant polycystic kidney disease (ADPKD).

8. The composition of claim 6, wherein the compound that downregulates GLIS2 comprises at least one selected from the group consisting of:
a protein inhibitor of GLIS2;
a nucleic acid that down regulates GLIS2 by RNA interference, and/or an expression vector expressing the nucleic acid that down regulates GLIS2 by RNA interference;
a ribozyme that down regulates GLIS2, and/or an expression vector expressing the ribozyme;
an expression vector comprising an expression cassette, wherein the expression cassette expresses CRISPR components that down regulates GLIS2 by CRISPR knockout or CRISPR knock down; and
a trans-dominant negative mutant protein of GLIS2, and/or an expression vector that expresses the trans-dominant negative mutant protein of GLIS2.

9. The composition of claim 6, wherein the compound that downregulates GLIS2 comprises an antisense oligonucleotide (ASO), an siRNA, or an shRNA downregulating GLIS2 expression, and/or an expression vector expressing the ASO, siRNA or shRNA.

10. The composition of claim 6, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

11. A kit for treating, ameliorating and/or preventing a polycystic kidney disease caused by or involving a mutant PKD1 or PKD2 in a subject in need thereof, comprising:
a compound that downregulates GLIS2; and
a manual instructing that the compound that downregulates GLIS2 is to be administer to the subject in an effective amount.

12. The kit of claim 11, wherein the polycystic kidney disease is an autosomal dominant polycystic kidney disease (ADPKD).

13. The kit of claim 11, wherein the compound that downregulates GLIS2 comprises at least one selected from the group consisting of:
a protein inhibitor of GLIS2;
a nucleic acid that down regulates GLIS2 by RNA interference, and/or an expression vector expressing the nucleic acid that down regulates GLIS2 by RNA interference;
a ribozyme that down regulates GLIS2, and/or an expression vector expressing the ribozyme;
an expression vector comprising an expression cassette, wherein the expression cassette expresses CRISPR components that down regulates GLIS2 by CRISPR knockout or CRISPR knock down; and
a trans-dominant negative mutant protein of GLIS2, and/or an expression vector that expresses the trans-dominant negative mutant protein of GLIS2.

14. The kit of claim 11, wherein the compound that downregulates GLIS2 comprises an antisense oligonucleotide (ASO), an siRNA, or an shRNA downregulating GLIS2 expression, and/or an expression vector expressing the ASO, siRNA or shRNA.

15. The kit of claim 11, wherein the compound that downregulates GLIS2 comprises a monoclonal antibody against GLIS2, a polyclonal antibody against GLIS2, antigen binding fragments thereof, an expression vector expressing the antibody or fragments thereof, or combinations thereof.

16. A method of screening suppressors of downstream effects of loss of PKD1 or PKD2, comprising:
detecting a first GLIS2 expression level in the nucleus of a cell expressing PKD1 and PKD2;
contacting the cell with a compound; and
detecting a second GLIS2 expression level in the nucleus of the cell,
wherein a difference between the first GLIS2 expression level and the second GLIS2 expression level indicates that the compound is a suppressors of downstream effects of loss of PKD1 or PKD2.

17. The method of claim 16, wherein the cell is a kidney cell or a kidney-derived cell.

18. The method of claim 16, wherein the cell is a primary cell or a cell line.

19. The method of claim 16, the cell is a mammalian cell or a mammalian cell-derived cell, optionally a human cell or a human cell-derived cell.

20. The method of claim 16, wherein that the second GLIS2 expression level is lower or higher than the first GLIS2 expression level indicates that compound is a suppressors of downstream effects of loss of PKD1 or PKD2.
